(19) United States
(12) Patent Application Publication
(10) Pub. No.: US 2025/0011303 A1
(43) Pub. Date: Jan. 9, 2025

(54) SMALL MOLECULE MODULATORS OF IL-17A, METHODS OF MAKING AND METHODS OF USING THEREOF

(71) Applicant: ASCLETIS BIOSCIENCE CO., LTD, Hangzhou (CN)

(72) Inventors: Bin Liang, Hangzhou (CN); Bailing Yang, Hangzhou (CN); Donghuang Liu, Hangzhou (CN); Jinzi Jason Wu, Hangzhou (CN)

(21) Appl. No.: 18/667,567

(22) Filed: May 17, 2024

Related U.S. Application Data
(60) Provisional application No. 63/520,980, filed on Aug. 22, 2023.
(60) Provisional application No. 63/504,649, filed on May 26, 2023.

(30) Foreign Application Priority Data
May 18, 2023 (CN) 202310573188.8
Aug. 11, 2023 (CN) 202311013032.0

(51) Int. Cl. C07D 401/14; A61K 31/397; A61K 31/4155; A61K 31/4725; A61K 31/538; A61K 31/5386; C07D 405/06; C07D 405/12; C07D 405/14; C07D 413/14; C07D 493/10; C07D 498/10

(52) U.S. Cl. CPC C07D 401/14; A61K 31/397; A61K 31/4155; A61K 31/4725; A61K 31/538; A61K 31/5386; C07D 405/06; C07D 405/12; C07D 405/14; C07D 413/14; C07D 493/10; C07D 498/10

(57) ABSTRACT

The present application describes compounds of IL-17A modulator that are useful for treating an IL-17A mediated inflammatory syndrome, disorder, or disease.

### CROSS REFERENCE TO RELATED APPLICATIONS

This application claims the benefit of U.S. provisional application No. 63/504,649 entitled “Small molecule modulators of IL-17A, methods of making and methods of using thereof,” filed May 26, 2023 and U.S. provisional application No. 63/520,980 entitled “Small molecule modulators of IL-17A, methods of making and methods of using thereof,” filed Aug. 22, 2023, with the U.S. Patent and Trademark Office, which are incorporated by reference herein in their entireties.

### FIELD

The field relates to pharmaceuticals and medicine, in particular to compounds, pharmaceutical compositions thereof which modulate interleukin-17A, and therapeutic uses of such compounds, for example, in treating and/or ameliorating an IL-17A mediated inflammatory syndrome, disorder, or disease.

### BACKGROUND

The interleukin-17 (IL-17) cytokines family (comprising IL-17A through IL-17 F), promotes the maintenance of both adaptive and innate immunity. The released cytokines act through their membrane-bound IL-17 receptor (IL-17RA to IL-17RE), and activate the IL-17 signal pathway. Dysregulation expression of IL-17 may contribute to inflammatory and autoimmune diseases such as psoriasis, psoriatic arthritis, rheumatoid arthritis, and multiple sclerosis.

IL-17A is the best investigated IL 17 family member. It is well established as a pro-inflammatory cytokine, which plays a pivotal role in immune and autoimmune related diseases including psoriasis, asthma, psoriatic arthritis, and rheumatoid arthritis. IL-17A forms homodimers or heterodimers with IL-17A or IL-17F and is a major cytokine mainly secreted from Th17 cells. It signals through its membrane-bound receptors, IL-17RA and IL-17RC, and modulates IL-17A signaling pathway and triggers multiple inflammatory and immune responses. Thus, IL-17A has emerged as a major topic of interest for treating inflammatory-associated diseases.

Antagonizing IL-17A/IL-17RA protein-protein interaction (PPI) was hypothesized to reduce overexaggerated inflammation in autoimmune diseases. There are several ways to block IL-17A signaling by targeting IL-17A proteins or receptors. Clinically, several monoclonal antibodies (mAbs) are already approved for different immunological diseases. While no oral small molecule IL-17A inhibitors have progressed into late stage clinical trials yet, they are in an attractive area for discovery as their development may broaden treatment options for many patients without access to biologics. Accordingly, there is a need for new small molecule IL-17A modulators (e.g., inhibitors).

### SUMMARY

Disclosed herein are novel compounds, and pharmaceutical compositions thereof, which modulate interleukin-17A. Also disclosed herein is the therapeutic use of such compounds, for example, in treating and/or ameliorating an IL-17A mediated inflammatory syndrome, disorder, or disease. One aspect of the present application relates to a compound of Formula I:

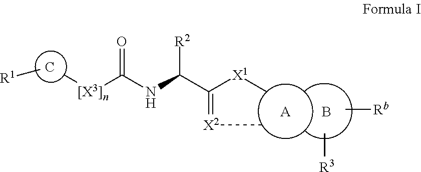

pharmaceutically acceptable salts thereof, deuterium substitutes thereof, and isomers thereof,
wherein Ring C is aryl, 5-6 membered heteroaryl, C₃₋₁₀ cycloalkyl, C₃₋₁₀ heterocyclyl, R^{x}—C₃₋₁₀ cycloalkyl, R^{x}—C₃₋₁₀ heterocyclyl, R^{x}-aryl and R^{x}-5-6 membered heteroaryl; wherein R^{x} is halo or —C₁₋₆ alkyl;
wherein Ring C is optionally substituted with one or more R¹;
wherein each R¹ is independently selected from the group consisting of OH, CN, C₁₋₆ alkyl, C₁₋₆ haloalkyl, C₂₋₆ alkenyl, C₂₋₆ alkynyl, —O—C₁₋₆ alkyl, —O—C₁₋₆ haloalkyl, —CO—C₁₋₆ alkyl, —CO—C₁₋₆ haloalkyl, —CO—NR^{3a}R^{3b}, C₃₋₁₀ cycloalkyl, C₃₋₁₀ heterocyclyl, C₆₋₁₀ aryl, and 5-10 membered heteroaryl;
wherein each R¹ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —S(O)—C₁₋₆ alkyl, —S(O)₂—C₁₋₆ alkyl and —S—C₁₋₆ alkyl;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X is NH, O, or S;
wherein X² is N, O, or S; and wherein X² is optionally attached to an atom in Ring A and forms another ring next to Ring A;
wherein X³ is O or —CR^{c}R^{d}; and wherein each of R^{c} and R^{d} is independently selected from the group consisting of H, halo and —C₁₋₆ alkyl;
wherein Ring A is selected from the group consisting of

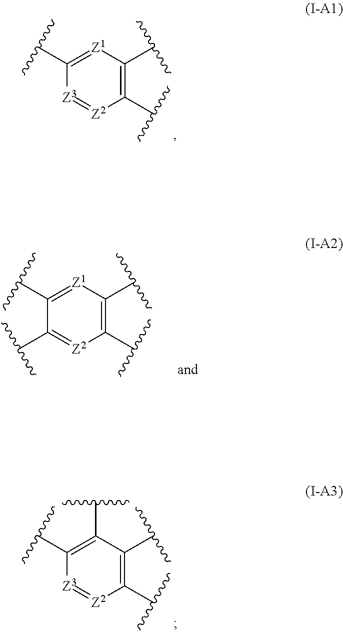

wherein each of Z¹, Z² and Z³ is independently selected from the group consisting of —N═, —N(═O)═ and —C(R^{a})═, wherein R^{a} is selected from the group consisting of H, F, Cl, Br, I, —C₁₋₆ alkyl, C₁₋₆ haloalkyl, C₂₋₆ alkenyl, C₂₋₆ alkynyl, OH, CN, —O—C₁₋₆ alkyl, —O—C₁₋₆ haloalkyl, C₃₋₁₀ cycloalkyl, C₃₋₁₀ heterocyclyl, aryl and 5-10 membered heteroaryl;
wherein Ring B, which is fused to Ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated or unsaturated bridged-ring;
wherein Ring B is optionally substituted with one or more R^{b}, each of R^{b} is independently selected from the group consisting of CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —NR^{3a}—CO—C₁₋₆ alkyl, —NR^{3a}—COOC₁₋₆ alkyl, —NR^{3a}—CO—C₁₋₆ haloalkyl, —NR^{3a}—CO—NR^{3a}R^{3b}, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more substituents each independently selected from the group consisting of halo, CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, C₂₋₆ alkenyl, C₂₋₆ alkynyl, C₃₋₁₀ cycloalkyl, heterocyclyl, C₆₋₁₀ aryl, and 5-10 membered heteroaryl; wherein Ring B is substituted with R³, wherein R³ is selected from the group consisting of —CONR^{3a}R^{3b}, —S(O)—NR^{3a}R^{3b}, —S(O)₂—NR^{3a}R^{3b}, —C₀₋₆ alkyl-CONR^{3a}R^{3b}, —C₀₋₆ alkyl-NR^{3a}R^{3b}, —N(R^{3a})—COR^{3a}, —N(R^{3a})—CONR^{3a}R^{3b}, —C₀₋₆ alkyl-SONR^{3a}R^{3b}, —C₀₋₆ alkyl-SO₂NR^{3a}R^{3b}, —C(S)—NR^{3a}R^{3b}, CN, 5-10 membered heteroaryl, —COR^{3a}, —P(O)OR^{3a}(OR^{3b}), —S(O)—C₁₋₆ alkyl, —S(O)₂—C₁₋₆ alkyl, C₁₋₆ alkyl, —C₁₋₆ alkyl-C₃₋₁₀ cycloalkyl, —O—C₁₋₆ alkyl, aryl, heterocyclyl, alkylheterocyclyl, alkylaryl and alkylcycloalkyl; wherein the 5-10 membered heteroaryl, the —S(O)—C₁₋₆ alkyl, the —S(O)₂—C₁₋₆ alkyl, the C₁₋₆ alkyl, the —C₁₋₆ alkyl-C₃₋₁₀ cycloalkyl, the —O—C₁₋₆ alkyl, the aryl, the heterocyclyl, the alkylheterocyclyl, the alkylaryl or the alkylcycloalkyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl; wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; or R^{3a} and R^{3b} are optionally combined to form a C₃₋₁₀ cycloalkyl, a heterocyclyl, a C₆₋₁₀ aryl, or a 5-10 membered heteroaryl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, and C₁₋₆ haloalkyl.

In another aspect, the present invention relates to the use of a compound of Formula (T) a pharmaceutically acceptable salt thereof, a deuterium substitute thereof, or an isomer thereof in the manufacture of a medicament for treating and/or ameliorating an IL-17A mediated inflammatory syndrome, disorder, or disease.

### DETAILED DESCRIPTION

### I. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cu-v” or “Cu-Cv” indicates that the following group has from u to v carbon atoms, where u and v are integers. For example, “C₁₋₆ alkyl” or “C₁-C₆ alkyl” indicates that the alkyl group has from 1 to 6 carbon atoms.

“Alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₆ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-Propyl, —CH(CH)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃). Alkyl groups can be unsubstituted or substituted.

“Alkoxy” refers to the group —O-alkyl, where alkyl is as defined above. For example, C₁₋₄ alkoxy refers to an —O-alkyl group having 1 to 4 carbons. Alkoxy groups can be unsubstituted or substituted.

“Alkoxyalkyl” is an alkoxy group attached to an alkyl as defined above. For example, C₂₋₆ alkoxyalkyl includes —CH₂—OMe, —CH₂—O-iPr, —CH₂—CH₂—OMe, —CH₂—CH₂—O—CH₂—CH₃, and —CH₂—CH₂—O-tBu. Alkoxyalkyl groups can be unsubstituted or substituted.

“Alkenyl” is a monovalent or divalent linear or branched hydrocarbon radical with at least one carbon-carbon double bond. For example, an alkenyl group can have 2 to 8 carbon atoms (i.e., C₂₋₈ alkenyl) or 2 to 6 carbon atoms (i.e., C₂₋₆ alkenyl) or 2 to 4 carbon atoms (i.e., C₂₋₄ alkenyl). Examples of alkenyl groups include, but are not limited to, ethenyl (—CH═CH₂), allyl (—CH₂CH═CH₂), and —CH₂—CH═CH—CH₃. Alkenyl groups can be unsubstituted or substituted.

“Alkynyl” is a monovalent or divalent linear or branched hydrocarbon radical with at least one carbon-carbon triple bond. For example, an alkynyl group can have 2 to 8 carbon atoms (i.e., C₂₋₈ alkynyl) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH₂C≡CH), and —CH₂—C≡C—CH₃. Alkynyl groups can be unsubstituted or substituted.

“Halogen” refers to fluoro (—F), chloro (—Cl), bromo (—Br) and iodo (—I).

“Haloalkyl” is an alkyl as defined herein, wherein one or more hydrogen atoms of the alkyl are independently replaced by a halogen, which may be the same or different, such that the alkyl is divalent. The alkyl group and the halogen can be any of those described above. In some embodiments, the haloalkyl defines the number of carbon atoms in the alkyl portion, e.g., C₁₋₄ haloalkyl includes CF₃, CH₂F, CHF₂, CH₂CF₃, CH₂CH₂CF₃, CCl₂CH₂CH₂CH₃, and C(CH₃)₂(CF₂H). Haloalkyl groups can be unsubstituted or substituted.

“Haloalkoxy” is an alkoxy as defined herein, wherein one or more hydrogen atoms of the alkyl in the alkyloxy are independently replaced by a halogen, which may be the same or different, such that the alkyl is divalent. The alkoxy group and the halogen can be any of those described above. In some embodiments, the haloalkoxy defines the number of carbon atoms in the alkyl portion, e.g., C₁₋₄ haloalkoxy includes OCF₃, OCH₂F, OCH₂CF₃, OCH₂CH₂CF₃, OCCl₂CH₂CH₂CH₃, and OC(CH₃)₂(CF₂H). Haloalkoxy groups can be unsubstituted or substituted.

“Cycloalkyl” is a monovalent or divalent single all carbon ring or a multiple condensed all carbon ring system wherein the ring in each instance is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary single ring cycloalkyl groups include cyclopropyl, cyclobutyl, cyclopentyl, cyclohexyl, cyclohexenyl, cycloheptyl, cycloheptenyl, and cyclooctyl. Cycloalkyl also includes multiple condensed ring systems (e.g., ring systems comprising 2 rings) having about 7 to 12 carbon atoms. The rings of the multiple condensed ring system can be connected to each other via fused, spiro, or bridged bonds when allowed by valency requirements. Exemplary multiple ring cycloalkyl groups include octahydropentalene, bicyclo[2.2.1]heptane, bicyclo[2.2.2]octane, bicyclo[2.2.2]oct-2-ene, and spiro[2.5]octane. Cycloalkyl groups can be unsubstituted or substituted.

Alkylcycloalkyl” refers to an alkyl as defined herein, wherein one or more hydrogen atoms of the alkyl are independently replaced by a cycloalkyl group, which may be the same or different. The alkyl group and the cycloalkyl group can be any of those described above. In some embodiments, the number of carbon atoms in the alkyl and cycloalkyl portion can be designated separately, e.g., C₁₋₆ alkyl-C₃₋₁₂ cycloalkyl. Alkylcycloalkyl groups can be unsubstituted or substituted.

“Aryl” as used herein refers to a monovalent or divalent single all carbon aromatic ring or a multiple condensed all carbon ring system wherein the ring is aromatic. For example, in some embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which multiple rings are aromatic. The rings of the multiple condensed ring system can be connected to each other via fused bonds when allowed by valency requirements. It is also understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl. Non-limiting examples of aryl groups include, but are not limited to, phenyl, naphthyl, anthracenyl, and the like. Aryl groups can be unsubstituted or substituted.

“Alkylaryl” refers to an alkyl as defined herein, wherein one or more hydrogen atoms of the alkyl are independently replaced by an aryl group, which may be the same or different. The alkyl group and the aryl group can be any of those described above, such that the alkyl is divalent. In some embodiments, an alkylaryl group has 7 to 24 carbon atoms, 7 to 16 carbon atoms, 7 to 13 carbon atoms, or 7 to 11 carbon atoms. An alkylaryl group defined by the number of carbon atoms refers to the total number of carbon atoms present in the constitutive alkyl and aryl groups combined. For example, C₇ alkylaryl refers to benzyl, while C₁₁ alkylaryl includes 1-methylnaphthyl and n-pentylphenyl. In some embodiments the number of carbon atoms in the alkyl and aryl portion can be designated separately, e.g., C₁₋₆ alkyl-C₆₋₁₀ aryl. Non-limiting examples of alkylaryl groups include, but are not limited to, benzyl, 2,2-dimethylphenyl, n-pentylphenyl, 1-methylnaphthyl, 2-ethylnaphthyl, and the like. Alkylaryl groups can be unsubstituted or substituted.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular (i.e., ring-shaped) heteroatom selected from oxygen, nitrogen, and sulfur). Unless otherwise specified, a heterocyclyl group has from 3 to about 20 annular atoms, for example from 3 to 12 annular atoms, for example from 4 to 12 annular atoms, 4 to 10 annular atoms, or 3 to 8 annular atoms, or 3 to 6 annular atoms, or 3 to 5 annular atoms, or 4 to 6 annular atoms, or 4 to 5 annular atoms, preferably, a heterocyclyl group has from 3 to about 20 annular atoms. Thus, the term includes single saturated or partially unsaturated rings (e.g., 3, 4, 5, 6 or 7-membered rings) having from about 1 to 6 annular carbon atoms and from about 1 to 4 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. Heterocyclyl groups can be unsubstituted or substituted.

“Alkylheterocyclyl” refers to an alkyl as defined herein, wherein one or more hydrogen atoms of the alkyl are independently replaced by a heterocyclyl group, which may be the same or different. The alkyl group and the heterocyclyl group can be any of those described above, such that the alkyl is divalent. In some embodiments, the number of atoms in the alkyl and heterocyclyl portion can be designated separately, e.g., C₁₋₆ alkyl-3-12 membered heterocyclyl having one to four heteroatoms each independently N, O, or S. Alkylheterocyclyl groups can be unsubstituted or substituted.

“5-10 membered heteroaryl” refers to a single aromatic ring that has at least one atom other than carbon in the ring, wherein the atom is selected from the group consisting of oxygen, nitrogen and sulfur; “5-10 membered heteroaryl” also includes multiple condensed ring systems that have at least one such aromatic ring, which multiple condensed ring systems are further described below. Thus, “5-10 membered heteroaryl” includes single aromatic rings of from about 1 to 9 carbon atoms and about 1-4 heteroatoms selected from the group consisting of oxygen, nitrogen and sulfur. The sulfur and nitrogen atoms may also be present in an oxidized form provided the ring is aromatic. Exemplary 5-10 membered heteroaryl ring systems include but are not limited to pyridyl, pyrimidinyl, oxazolyl or furyl. “5-10 membered heteroaryl” also includes multiple condensed ring systems (e.g., ring systems comprising 2, 3 or 4 rings) wherein a 5-10 membered heteroaryl group, as defined above, is condensed with one or more rings selected from 5-10 membered heteroaryls (to form for example 1,8-naphthyridinyl) and aryls (to form, for example, benzimidazolyl or indazolyl) to form the multiple condensed ring system. Thus, a 5-10 membered heteroaryl (a single aromatic ring or multiple condensed ring system) can have about 1-20 carbon atoms and about 1-6 heteroatoms within the 5-10 membered heteroaryl ring. For example, tetrazolyl has 1 carbon atom and 4 nitrogen heteroatoms within the ring. The rings of the multiple condensed ring system can be connected to each other via fused bonds when allowed by valency requirements. It is to be understood that the individual rings of the multiple condensed ring system may be connected in any order relative to one another. It is to be understood that the point of attachment for a 5-10 membered heteroaryl or 5-10 membered heteroaryl multiple condensed ring system can be at any suitable atom of the 5-10 membered heteroaryl or 5-10 membered heteroaryl multiple condensed ring system including a carbon atom and a heteroatom (e.g., a nitrogen). It also to be understood that when a reference is made to a certain atom-range membered (e.g., a 5-10 membered heteroaryl), the atom range is for the total ring atoms of the 5-10 membered heteroaryl and includes carbon atoms and heteroatoms. It is also to be understood that the rings of the multiple condensed ring system may include an aryl ring fused to a heterocyclic ring with saturated or partially unsaturated bonds (e.g., 3, 4, 5, 6 or 7-membered rings) having from about 1 to 9 annular carbon atoms and from about 1 to 4 annular heteroatoms selected from the group consisting of oxygen, nitrogen and sulfur in the ring. For example, a 5-10 membered heteroaryl includes thiazolyl and a 5-10 membered heteroaryl includes quinolinyl. Exemplary 5-10 membered heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, benzofuranyl, benzimidazolyl, thianaphthenyl, pyrrolo[2,3-b]pyridinyl, quinazolinyl-4(3H)-one, triazolyl, and tetrazolyl. 5-10 membered heteroaryl groups can be unsubstituted or substituted.

“Alkylheteroaryl” refers to an alkyl as defined herein, wherein one or more hydrogen atoms of the alkyl are independently replaced by a heteroaryl group, which may be the same or different, such that the alkyl is divalent. The alkyl group and the heteroaryl group can be any of those described above. In some embodiments, the number of atoms in the alkyl and heteroaryl portion are designated separately, e.g., C₁₋₆ alkyl-5-10 membered heteroaryl having one to four heteroatoms each independently N, O, or S. Alkylheteroaryl groups can be unsubstituted or substituted.

“Oxo” as used herein refers to ═O.

“Substituted” as used herein refers to wherein one or more hydrogen atoms of the group are independently replaced by one or more substituents (e.g., 1, 2, 3, or 4 or more) as indicated.

A “compound of the present application” includes compounds disclosed herein, for example a compound of the present application includes compounds of Formula I, including the compounds of the Examples. In some embodiments, a “compound of the present application” includes compounds of Formula I.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present application is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present application within seconds or minutes. In some embodiments, a unit dose of a compound of the present application is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present application. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the subject.

Provided are also pharmaceutically acceptable salts, hydrates, solvates, tautomeric forms, polymorphs, and prodrugs of the compounds described herein.

Pharmaceutically acceptable” or “physiologically acceptable” refers to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21st Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and N(C₁-C₄ alkyl)₄₊. Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom (also referred to as 2H or D), in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium. Provided are also compounds described herein or pharmaceutically acceptable salts, isomers, or a mixture thereof, in which from 1 to n hydrogen atoms attached to a carbon atom may be replaced independently by 1 to n corresponding isotopes. Examples of isotopes that can be incorporated into the disclosed compounds also include isotopes of hydrogen, carbon, nitrogen, oxygen, phosphorous, fluorine, chlorine, and iodine, such as ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, tautomer, diastereomers, and other stereoisomeric forms that may be defined, in terms of absolute stereochemistry, as (R)- or (S)- or, as (D)- or (L)- for amino acids, as well as deuterated analogs thereof. The chemical formula shown in the present application is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Stereoisomer” as used herein refers to a compound made up of the same atoms bonded by the same bonds but having different three-dimensional structures, which are not interchangeable. The present application contemplates various stereoisomers and mixtures thereof and includes “enantiomers”, which refers to two stereoisomers whose molecules are non-superimposable mirror images of one another.

“Tautomer” as used herein refers to a proton shift from one atom of a molecule to another atom of the same molecule. In some embodiments, the present application includes tautomers of said compounds.

“Solvate” as used herein refers to the result of the interaction of a solvent and a compound. Solvates of salts of the compounds described herein are also provided. Hydrates of the compounds described herein are also provided.

“Hydrate” as used herein refers to a compound of the disclosure that is chemically associated with one or more molecules of water.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Prodrug” as used herein refers to a derivative of a drug that upon administration to the human body is converted to the parent drug according to some chemical or enzymatic pathway. In some embodiments, a prodrug is a biologically inactive derivative of a drug that upon administration to the human body is converted to the biologically active parent drug according to some chemical or enzymatic pathway.

“Treatment” or “treat” or “treating” as used herein refers to an approach for obtaining beneficial or desired results. For purposes of the present application, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

### II. Compounds

One aspect of the present application relates to a compound of Formula I:

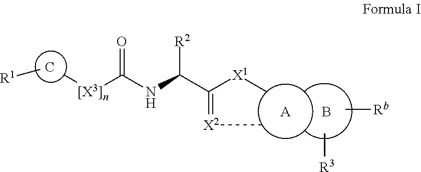

pharmaceutically acceptable salts thereof, deuterium substitutes thereof, and isomers thereof,
wherein Ring C is aryl, 5-6 membered heteroaryl, C₃₋₁₀ cycloalkyl, C₃₋₁₀ heterocyclyl, R^{x}—C₃₋₁₀ cycloalkyl, R^{x}—C₃₋₁₀ heterocyclyl, R^{x}-aryl and R^{x}-5-6 membered heteroaryl; wherein R is halo or —C₁₋₆ alkyl;
wherein Ring C is optionally substituted with one or more R¹;
wherein each R¹ is independently selected from the group consisting of OH, CN, C₁₋₆ alkyl, C₁₋₆ haloalkyl, C₂₋₆ alkenyl, C₂₋₆ alkynyl, —O—C₁₋₆ alkyl, —O—C₁₋₆ haloalkyl, —CO—C₁₋₆ alkyl, —CO—C₁₋₆ haloalkyl, —CO—NR^{3a}R^{3b}, C₃₋₁₀ cycloalkyl, C₃₋₁₀ heterocyclyl, C₆₋₁₀ aryl, and 5-10 membered heteroaryl;
wherein each R¹ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —S(O)—C₁₋₆ alkyl, —S(O)₂—C₁₋₆ alkyl and —S—C₁₋₆ alkyl;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X¹ is NH, O, or S;
wherein X² is N, O, or S; and wherein X² is optionally attached to an atom in Ring A and forms another ring next to Ring A;
wherein X³ is O or —CR^{c}R^{d}; and wherein each of R^{c} and R^{d} is independently selected from the group consisting of H, halo and —C₁₋₆ alkyl;
wherein Ring A is selected from the group consisting of

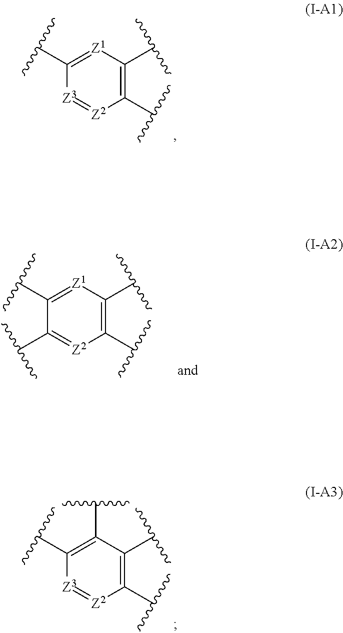

wherein each of Z¹, Z² and Z³ is independently selected from the group consisting of —N═, —N(═O)═ and —C(R^{a})═, wherein R^{a} is selected from the group consisting of H, F, Cl, Br, I, —C₁₋₆ alkyl. C₁₋₆ haloalkyl, C₂₋₆ alkenyl, C₂₋₆ alkynyl, OH, CN, —O—C₁₋₆ alkyl, —O—C₁₋₆ haloalkyl, C₃₋₁₀ cycloalkyl, C₃₋₁₀ heterocyclyl, aryl and 5-10 membered heteroaryl,
wherein Ring B, which is fused to Ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated or unsaturated bridged-ring;
wherein Ring B is optionally substituted with one or more R^{b}, each of R^{b} is independently selected from the group consisting of CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —NR^{3a}—CO—C₁₋₆ alkyl, —NR^{3a}, —COOC₁₋₆ alkyl, —NR^{3a}—CO—C₁₋₆ haloalkyl, —NR^{3a}—CO—NR^{3a}R^{3b}, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more substituents each independently selected from the group consisting of halo, CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, C₂₋₆ alkenyl, C₂₋₆ alkynyl, C₃₋₁₀ cycloalkyl, heterocyclyl, C₆₋₁₀ aryl, and 5-10 membered heteroaryl; wherein Ring B is substituted with R³, wherein R³ is selected from the group consisting of —CONR^{3a}R^{3b}, —S(O)—NR^{3a}R^{3b}, —S(O)₂—NR^{3a}R^{3b}, —C₀₋₆ alkyl-CONR^{3a}R^{3b}, —C₁₋₆ alkyl-NR^{3a}R^{3b}, —N(R^{3a})—COR^{3a}, —N(R^{3a})—CONR^{3a}R^{3b}, —C₀₋₆ alkyl-SONR^{3a}R^{3b}, —C₀₋₆ alkyl-SO₂NR^{3a}R^{3b}, —C(S)—NR^{3a}R^{3b}, CN, 5-10 membered heteroaryl, —COR^{3a}, —P(O(OR^{3a})OR^{3b}), —S(O)—C₁₋₆alkyl, —S(O)₂—C₁₋₆ alkyl, C₁₋₆ alkyl, —C₁₋₆ alkyl-C₃₋₁₀ cycloalkyl, —O—C₁₋₆ alkyl, aryl, heterocyclyl, alkylheterocyclyl, alkylaryl and alkylcycloalkyl; wherein the 5-10 membered heteroaryl, the —S(O)—C₁₋₆ alkyl, the —S(O)₂—C₁₋₆ alkyl, the C₁₋₆ alkyl, the —C₁₋₆ alkyl-C₃₋₁₀ cycloalkyl, the —O—C₁₋₆ alkyl, the aryl, the heterocyclyl, the alkylheterocyclyl, the alkylaryl or the alkylcycloalkyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl;
wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; or R^{3a} and R^{3b} are optionally combined to form a C₃₋₁₀ cycloalkyl, a heterocyclyl, a C₆₋₁₀ aryl, or a 5-10 membered heteroaryl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, and C₁₋₆ haloalkyl.

In some embodiments, Ring B is fused to Ring A and selected from the group consisting of:

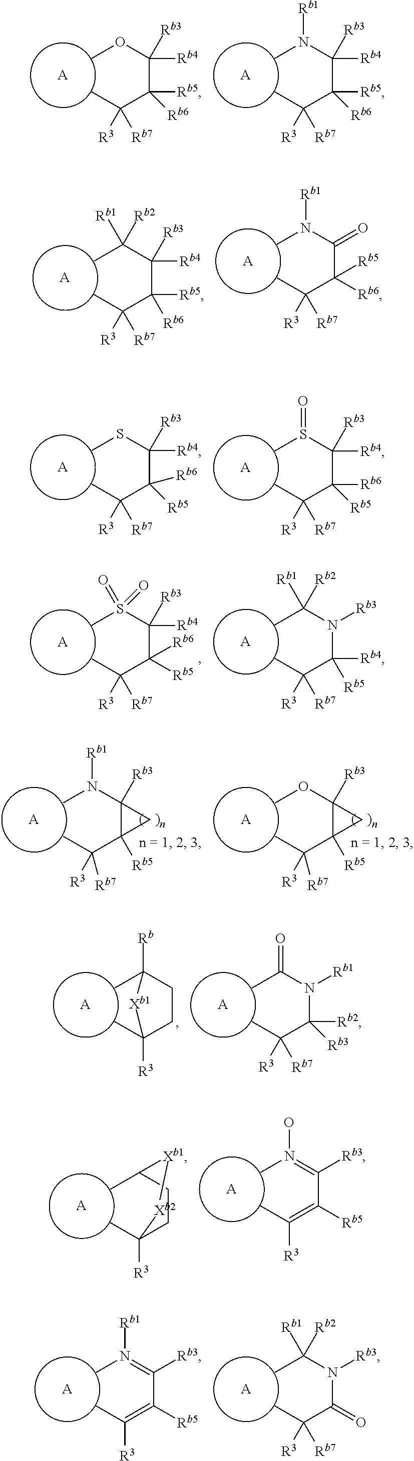

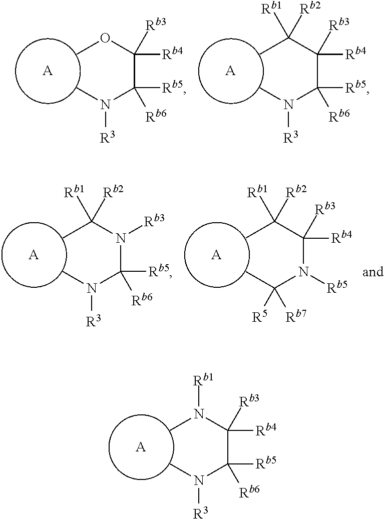

wherein, each of R^{b1}, R^{b2}, R^{b3}, R^{b4}, R^{b5}, R^{b6} and R^{b7} is independently selected from the group consisting of —C₁₋₆ alkyl, —C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}—C₂₋₆ alkenyl, —C₂₋₆ alkynyl, —C₃₋₁₀ cycloalkyl, heterocyclyl, C₆₋₁₀ aryl, 5-10 membered heteroaryl, —C₁₋₆ alkyl-C₃₋₁₀ cycloalkyl, —C₁₋₆ alkyl-heterocyclyl, —C₁₋₆ alkyl-C₆₋₁₀ aryl, —C₁₋₆ alkyl-5-10 membered heteroaryl, H, F, —CH₃, —CHF₂, —CF₃, and OH; wherein any two of R^{b1}, R^{b2}, R^{b3}, R^{b4}, R^{b5}, R^{b6}, R^{b7} and R³ are optionally combined to form a C₃₋₁₀ cycloalkyl or a heterocyclyl or a C₆₋₁₀ aryl or 5-10 membered heteroaryl, and wherein each of X^{b1} and X^{b2} is independently C, N, O or S.

In some embodiments, each of R^{b1} and R^{b2} is independently selected from the group consisting of:

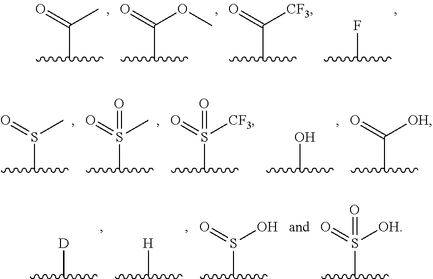

In some embodiments, the R³ group is selected from the group consisting of: —CONR^{3a}R^{3b}, —S(O)—NR^{3a}R^{3b}, —S(O)₂—NR^{3a}R^{3b}, —C₁₋₆ alkyl-CONR^{3a}R^{3b}, —C₁₋₆ alkyl-NR^{3a}R^{3b}, —N(R^{3a})—COR^{3a}, —N(R^{3a})—CONR^{3a}R^{3b}, —C₁₋₆ alkyl-SONR^{3a}R^{3b}, —C₁₋₆ alkyl-SO₂NR^{3a}R^{3b}, CN, —COR^{3a}, —O—C₁₋₆ alkyl; wherein the R³ is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

In some embodiments, the R³ group is selected from the group consisting of: —CONR^{3a}R^{3b}, —C₁₋₆ alkyl-CONR^{3a}R^{3b}, —C₁₋₆ alkyl-NR^{3a}R^{3b}, —N(R^{3a})—COR^{3a}, —N(R^{3a})—CONR^{3a}R^{3b}, —COR^{3a}.

In some embodiments, the R^{3a} and R^{3b} are independently selected from the group consisting of H, C₁₋₆ alkyl, C₁₋₆ haloalkyl, —C₂₋₆ alkenyl, —C₂₋₆ alkynyl, C₀₋₆ alkyl-C₃₋₆ cycloalkyl, and 5-6 membered heterocyclyl; or R^{3a} and R^{3b} are optionally combined to form a C₃₋₁₀ cycloalkyl, a heterocyclyl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl and the heterocyclyl is optionally substituted with one or more substituents each independently selected from the group consisting of halo, C₁₋₆ alkyl, and C₁₋₆ haloalkyl.

In some embodiments, the R^{3a} and R^{3b} are independently selected from the group consisting of H, C₁₋₆ alkyl, and C₁₋₆ haloalkyl.

In some embodiments, the R^{3a} and R^{3b} are optionally combined to form a C₃₋₁₀ cycloalkyl, a heterocyclyl, a C₆₋₁₀ aryl, or a 5-10 membered heteroaryl; the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, —O—C₁₋₆ alkyl, and C₁₋₆ haloalkyl.

In some embodiments, the R³ group is

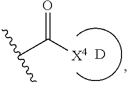

wherein the Ring D is C₃₋₁₀ cycloalkyl or 3-10 membered heterocyclyl; wherein X⁴ is N or CH; wherein the Ring D is optionally substituted with oxo, halo, C₁₋₆ alkyl, —O—C₁₋₆ alkyl, and C₁₋₆ haloalkyl; wherein heterocyclyl has one to four heteroatoms, each is independently N, O, or S.

In some embodiments, the R³ group is selected from the group consisting of

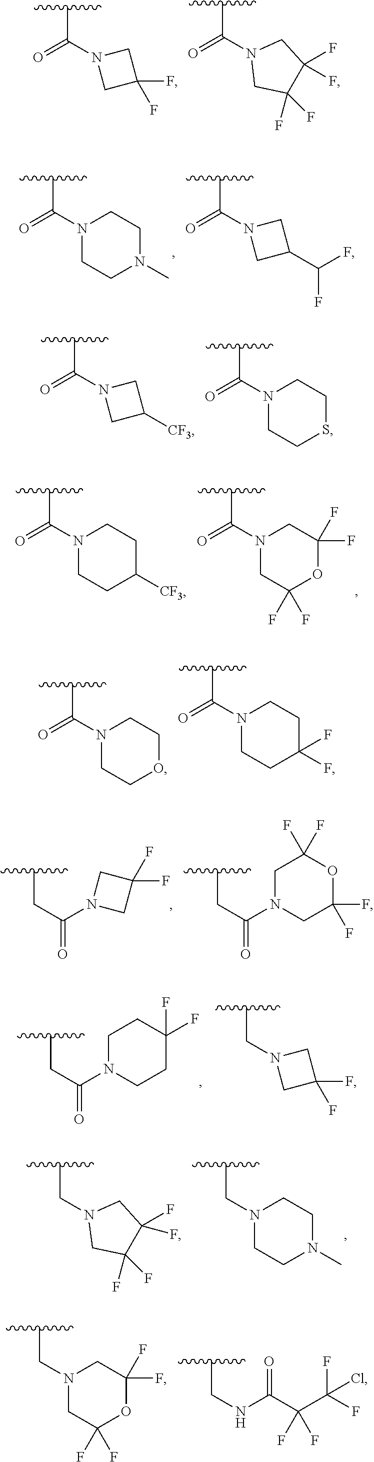

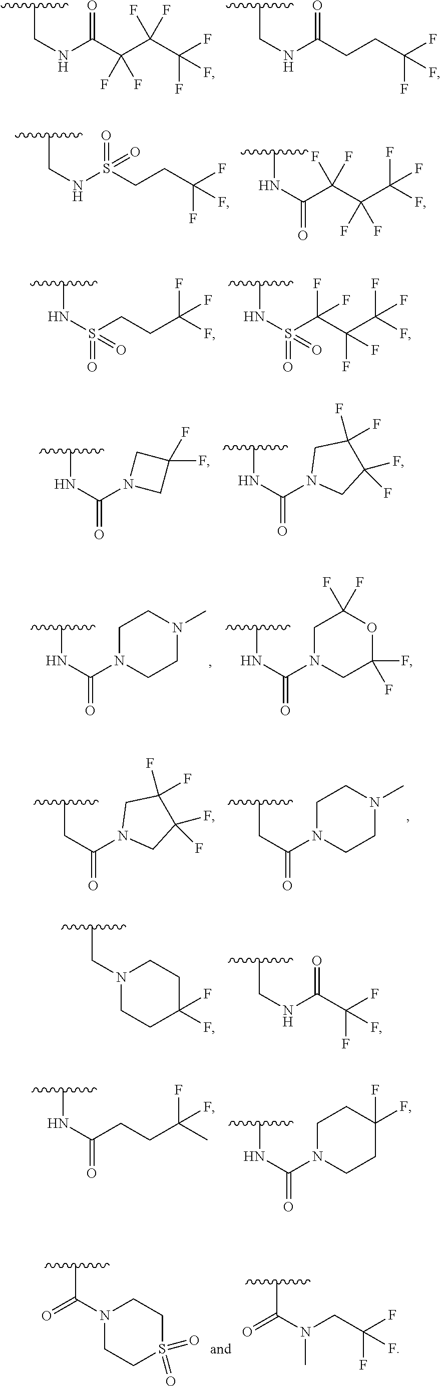

In some embodiments, Ring C is selected from the group consisting of aryl, 5-6 membered heteroaryl and C₃₋₁₀ cycloalkyl.

In some embodiments, Ring C is 5-6 membered heteroaryl.

In some embodiments, Ring C is selected from the group consisting of

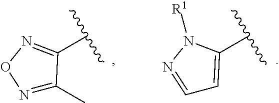

In some embodiments, Ring C is

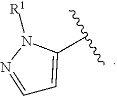

The compound of claim 1, pharmaceutically acceptable salts thereof, deuterium substitutes thereof, and isomers thereof, Ring C is C₃₋₁₀ cycloalkyl; wherein the cycloalkyl is optionally substituted with one or more substituents each independently selected from the group consisting of halo, C₁₋₆ alkyl and C₁₋₆ haloalkyl.

In some embodiments, X³ is O.

In some embodiments, X³ is —CR^{c}R^{d}.

In some embodiments, R^{b} is independently selected from the group consisting of H, C₁₋₆ alkyl, C₁₋₆ haloalkyl, —O—C₁₋₆ alkyl, —O—C₁₋₆ haloalkyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, F and OH; wherein two R^{b} groups are optionally combined to form a C₃₋₁₀ cycloalkyl or a heterocyclyl; wherein each R^{b} is optionally substituted with one or more substituents each independently selected from the group consisting of halo, C₁₋₆ alkyl, C₁₋₆ haloalkyl, —O—C₁₋₆ alkyl, —O—C₁₋₆ haloalkyl, C₃₋₁₀ cycloalkyl, heterocyclyl.

In some embodiments, R^{b} is independently selected from the group consisting of H, C₁₋₆ alkyl, C₁₋₆ haloalkyl, —O—C₁₋₆ alkyl, —O—C₁₋₆ haloalkyl, C₃₋₁₀ cycloalkyl, heterocyclyl, —C₁₋₆ alkyl —C₃₋₁₀ cycloalkyl, —C₁₋₆ alkyl-heterocyclyl; wherein two R^{b} groups are optionally combined to form a C₃₋₁₀ cycloalkyl or a heterocyclyl; wherein each R^{b} is optionally substituted with one or more substituents each independently selected from the group consisting of halo, C₁₋₆ alkyl, C₁₋₆ haloalkyl, —O—C₁₋₆ alkyl, —O—C₁₋₆ haloalkyl, C₃₋₁₀ cycloalkyl, heterocyclyl.

In some embodiments, R¹ is independently selected from the group consisting of halogen, oxo, —C₁₋₆ alkyl, —C₁₋₆ alkoxy, —C₁₋₆ haloalkoxy, —C₁₋₆ haloalkyl and —C₃₋₁₀ cycloalkyl.

In some embodiments, R¹ is independently selected from the group consisting of halogen, —C₁₋₆ alkyl, —C₁₋₆ haloalkyl and —C₃₋₁₀ cycloalkyl.

In some embodiments, R¹ is independently selected from the group consisting of —C₁₋₆ alkyl, —C₁₋₆ haloalkyl and —C₃₋₁₀ cycloalkyl.

In some embodiments, R² is —C₀₋₁ alkyl-C₃₋₉ cycloalkyl; wherein the —C₀₋₁ alkyl-C₃₋₉ cycloalkyl is optionally substituted with one or more substituents each independently selected from the group consisting of C₃₋₆ cycloalkyl, halo, CH₃, CHF₂, CF₃, OH and oxo.

In some embodiments, R² is selected from the group consisting of:

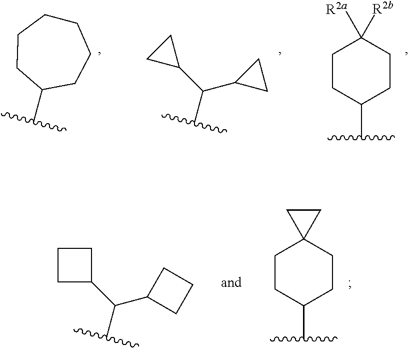

wherein R^{2a} and R^{2b} is selected from the group consisting of H, halo, C₁₋₃ alkyl;
   and the R² is optionally substituted with one or more halo and C₁₋₆ alkyl.

In some embodiments, R² is selected from the group consisting of

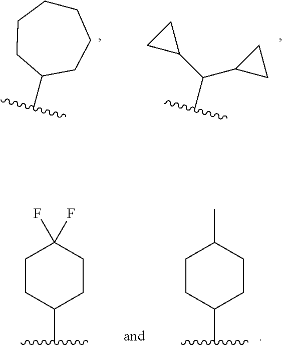

In some embodiments, R² is selected from the group consisting of

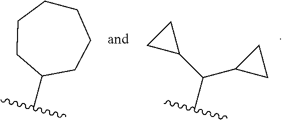 [SMILES: *C1CCCCCC1 ; *C(C1CC1)C1CC1]

In some embodiments, R³ is selected from the group consisting of the following structures:

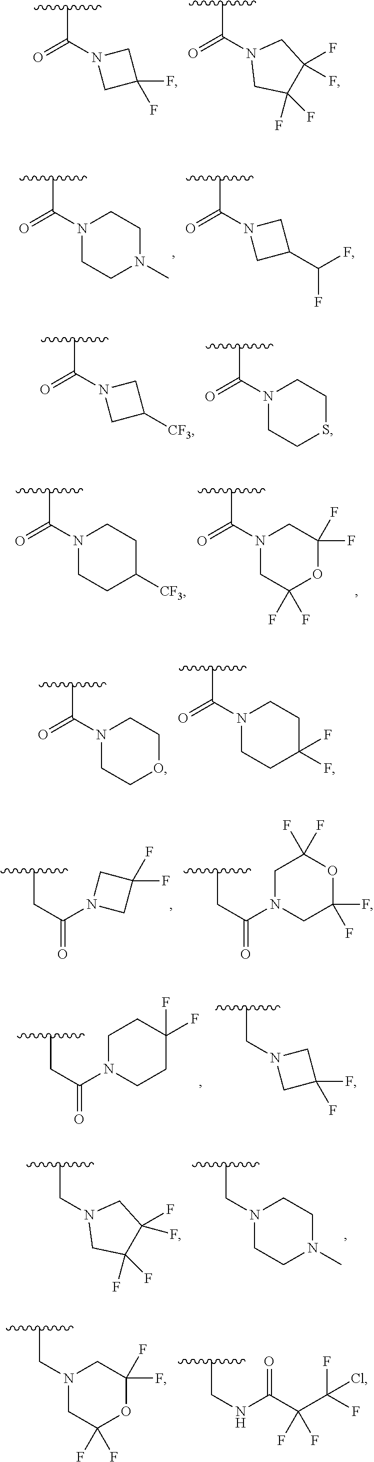

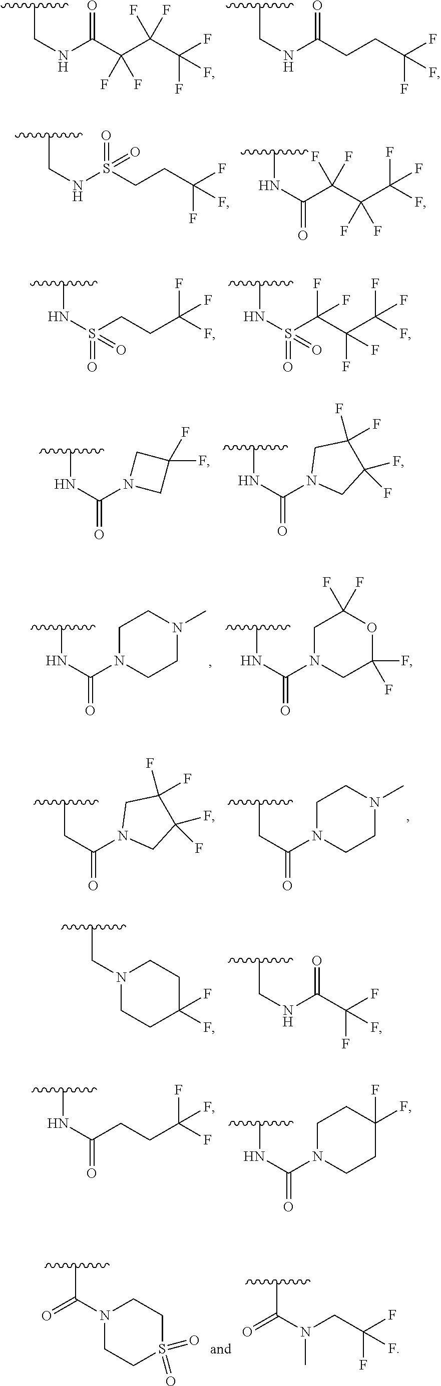

In some embodiments, the compound of the present application is selected from the group consisting of the following compounds of Formula I-A-1, Formula I-A-2, Formula I-A-3 and Formula I-A-4:

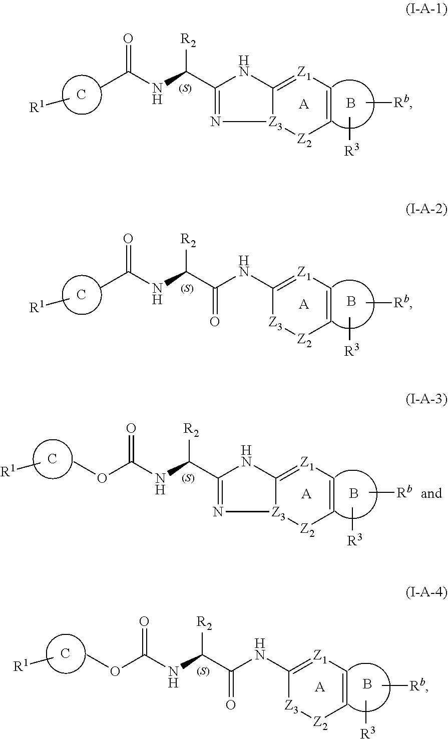

pharmaceutically acceptable salts thereof, deuterium substitutes thereof, and isomers thereof.

In some embodiments, the compound of the present application is selected from the group consisting of the following compounds of Formula I-A-5, Formula I-A-6, Formula I-A-7 and Formula I-A-8:

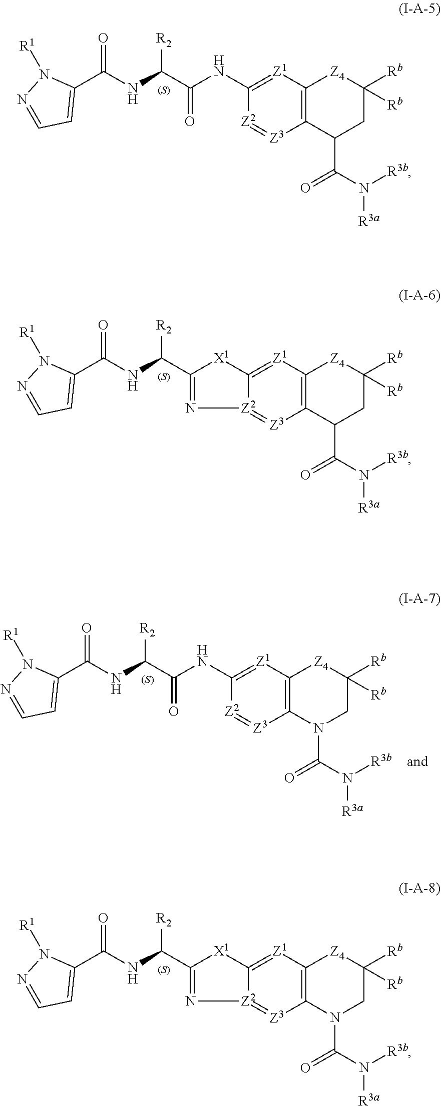

wherein Z⁴ is O, S, S═O, S(═O)₂, or NR^{b1};
wherein X¹ is O, S, or NH; and
wherein Z¹, Z² or Z³ is independently N or —C—Ra.

In some embodiments, the compound of the present application is selected from the group consisting of the following compounds of Formula I-A-9, Formula I-A-10, Formula I-A-11 and Formula I-A-12:

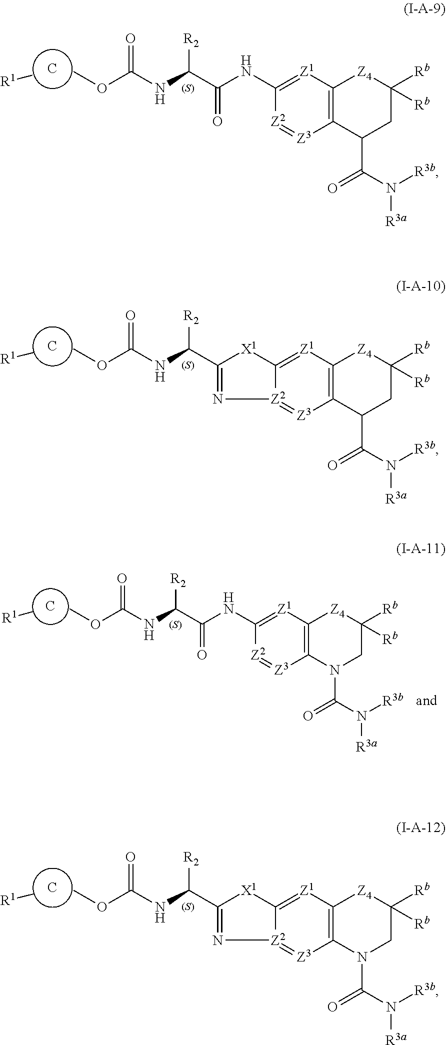

wherein Z⁴ is O, S, S═O, S(═O)₂, or NR^{b1};
wherein X¹ is O, S, or NH;
wherein Z¹, Z² or Z³ is independently N or —C—R^{a};
wherein Ring C is aryl, 5-6 membered heteroaryl, C₃₋₁₀ cycloalkyl, C₁₋₁₀ heterocyclyl, —C₁₋₆ alkyl-C₃₋₁₀ cycloalkyl, —C₁₋₆ alkyl-C₃₋₁₀ heterocyclyl, —C₁₋₆ alkyl-aryl, or —C₁₋₆ alkyl-5-6 membered heteroaryl; wherein the aryl, the 5-6 membered heteroaryl, the C₃₋₁₀ cycloalkyl, the C₃₋₁₀ heterocyclyl, the —C₁₋₆ alkyl-C₃₋₁₀ cycloalkyl, the —C₁₋₆ alkyl-C₃₋₁₀ heterocyclyl, the —C₁₋₆ alkyl-aryl, or the —C₁₋₆ alkyl-5-6 membered heteroaryl is optionally substituted with one or more substituents each independently selected from the group consisting of halo, C₁₋₆ alkyl, C₁₋₆ haloalkyl, C₁₋₆ haloalkoxy and C₁₋₆ alkoxy.

In some embodiments, the compound of the present application is selected from the group consisting of the following compounds Formula I-A-13:

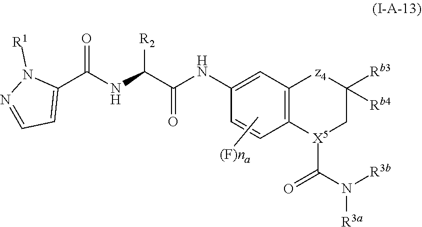

wherein Z⁴ is O, —C(R^{b1}R^{b2})—, —N(R^{b1})—;
X⁵ is N or CH;
wherein R^{b1}, R^{b2}, R^{b3} and R^{b4} is independently selected from the group consisting of —C₁₋₆ alkyl, —C₁₋₆ haloalkyl, O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —C₂₋₆ alkenyl, —C₂₋₆ alkynyl, —C₃₋₁₀ cycloalkyl, heterocyclyl, C₆₋₁₀ aryl, 5-10 membered heteroaryl, —C₁₋₆ alkyl-C₃₋₁₀ cycloalkyl, —C₁₋₆ alkyl-heterocyclyl, —C₁₋₆ alkyl-C₆₋₁₀ aryl, —C₁₋₆ alkyl-5-10 membered heteroaryl, H, F, —CH₃, —CHF₂, —CF₃, and OH; and R^{b3} and R^{b4} are optionally combined to form a C₃₋₁₀ cycloalkyl or a heterocyclyl; wherein the C₃₋₁₀ cycloalkyl or heterocyclyl is optionally substituted with one or more substituents each independently selected from the group consisting of oxo, halo, C₁₋₆ alkyl, C₁₋₆ haloalkyl, —O—C₁₋₆ alkyl and —O—C₁₋₆ haloalkyl;
wherein the R^{3a} and R^{3b} is independently selected from the group consisting of H, C₁₋₆ alkyl, C₁₋₆ haloalkyl, —C₂₋₆ alkenyl, —C₂₋₆ alkynyl, C₀₋₆ alkyl-C₃₋₆ cycloalkyl, and 5-6 membered heterocyclyl; or R^{3a} and R^{3b} are optionally combined to form a C₃₋₁₀ cycloalkyl, a heterocyclyl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl and the heterocyclyl is optionally substituted with one or more substituents each independently selected from the group consisting of halo, C₁₋₆ alkyl, and C₁₋₆ haloalkyl;
wherein nₐ is 0, 1 and 2.

In some embodiments, R^{b3} and R^{b4} are independently selected from the group consisting of H, —C₁₋₆ alkyl, —C₁₋₆ haloalkyl, O—C₁₋₆ alkyl, —O—C₁₋₆ haloalkyl, —C₃₋₁₀ cycloalkyl, heterocyclyl, —C₁₋₆ alkyl-C₃₋₁₀ cycloalkyl, and —C₁₋₆ alkyl-heterocyclyl; and R^{b3} and R^{b4} are optionally combined to form a C₃₋₁₀ cycloalkyl or a heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

In some embodiments, R^{b4} is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, and —C₁₋₆ alkyl-heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

In some embodiments, R^{b4} is selected from the group consisting of —C₁₋₆ alkyl, —C₃₋₁₀ cycloalkyl and heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

In some embodiments, X⁵ is N.

In some embodiments, X⁵ is CH.

In some embodiments, the compound of the present application is selected from the group consisting of the following compounds of Formula I-A-18:

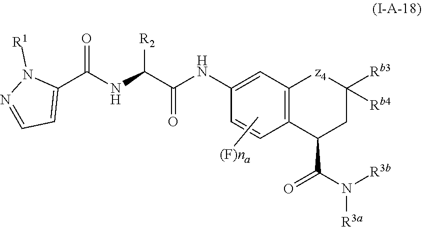

In some embodiments, the Z⁴ is selected from the group consisting of O and CH².

In some embodiments, the Z⁴ is O.

In some embodiments, the Z⁴ is CH₂.

In some embodiments, the compound of the present application is selected from the group consisting of the following compounds of Formula I-A-14:

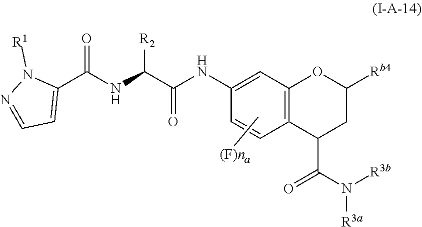

In some embodiments, nₐ is 0 and 1.

In some embodiments, R⁴ is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, —C₁₋₆ alkyl-heterocyclyl, H, and F; wherein the C₃₋₁₀ cycloalkyl or heterocyclyl is optionally substituted with one or more substituents each independently selected from the group consisting of halo, oxo, C₁₋₆ alkyl, C₁₋₆ haloalkyl, —O—C₁₋₆ alkyl and —O—C₁₋₆ haloalkyl.

In some embodiments, the compound of the present application is selected from the group consisting of the following compounds of Formula I-A-15:

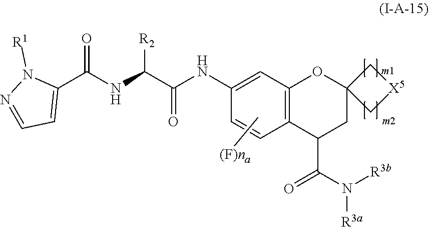

wherein X⁵ is selected from the group consisting of O, —N(R^{x5})—, —C(R^{X6}R^{X7})—, S, S(O) and S(O)₂;
m1 and m2 is 0, 1, 2 and 3;
nₐ is 0, 1 and 2;
wherein R^{x5} is selected from the group consisting of H, C₁₋₆ alkyl, C₁₋₆ haloalkyl, —C₃₋₁₀ cycloalkyl, —CO—C₁₋₆ alkyl and —CO—C₁₋₆ haloalkyl;
R^{x6} and R^{x7} is independently selected from the group consisting of H, halo, C₁₋₆ alkyl, C₁₋₆ haloalkyl.

In some embodiments, the compound of the present application is selected from the group consisting of the following compounds of Formula I-A-16:

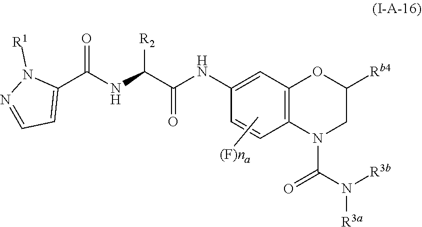

wherein nₐ is 0, 1 and 2.

In some embodiments, R^{b4} is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, —C₁₋₆ alkyl-heterocyclyl, H, and F; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

In some embodiments, the compound of the present application is selected from the group consisting of the following compounds of Formula I-A-17:

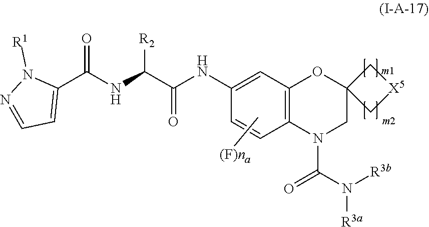

wherein X⁵ is O, —N(R^{x5})—, —C(R^{X6}R^{X7})—, S, S(O) and S(O)₂;
m1 and m2 is 0, 1, 2 and 3;
nₐ is 0, 1 and 2;
wherein R^{x5} is H, C₁₋₆ alkyl, C₁₋₆ haloalkyl, —CO—C₁₋₆ alkyl, —C₃₋₁₀ cycloalkyl and —CO—C₁₋₆ haloalkyl;
R^{x6} and R^{x7} is H, halo, C₁₋₆ alkyl, C₁₋₆ haloalkyl.

In some embodiments, the compound of the present application is selected from the group consisting of the following compounds of Formula I-A-19;

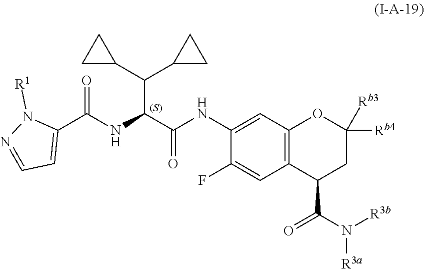

A compound of Formula II:

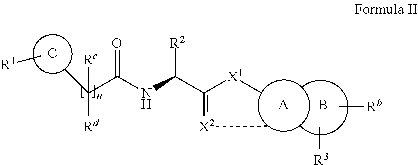

pharmaceutically acceptable salts thereof, deuterium substitutes thereof, and isomers thereof,
wherein Ring C is aryl or 5-6 membered heteroaryl;
wherein Ring C is optionally substituted with one or more R¹;
wherein R¹ is selected from the group consisting of C₁₋₆ alkyl, C₁₋₆ haloalkyl, C₂₋₆ alkenyl, C₂₋₆ alkynyl, C₃₋₁₀ cycloalkyl, C₃₋₁₀ heterocyclyl, C₆₋₁₀ aryl, and 5-10 membered heteroaryl;
wherein R¹ is optionally substituted with halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl;
wherein each of R^{c} and R^{d} is independently selected from the group consisting of H and halo;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X¹ is NH, O, or S;
wherein X² is N, O, or S; X² is optionally attached to an atom in ring A and forms another ring next to Ring A;
wherein Ring A is selected from the group consisting of

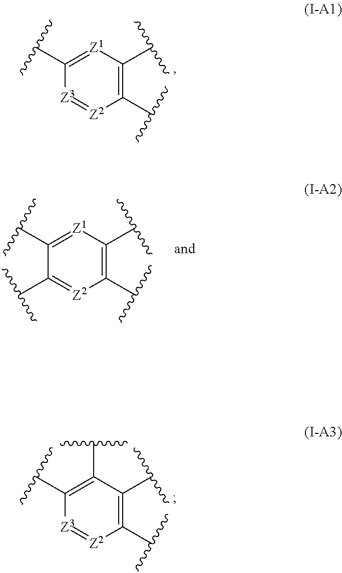

wherein each of Z¹, Z² and Z³ is independently selected from the group consisting of N, N═O, and —C—R^{a}, wherein R^{a} is selected from the group consisting of H, F, Cl, —CH₃, —CHF₂, —CF₃, OH, —OCH₃ and —OCF₃;
wherein Ring B, which is fused to ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated and unsaturated bridged-ring;
wherein Ring B is optionally substituted with one or more R^{b}, each of R^{b} is independently selected from the group consisting of C₁₋₆ alkyl, C₁₋₆ haloalkyl, —O—C₁₋₆ alkyl, —O—C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more halo;
wherein Ring B is substituted with R³, wherein R³ is selected from the group consisting of

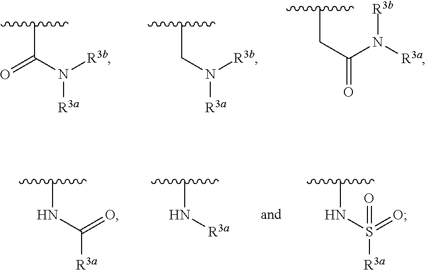

wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; wherein R^{3a} and R^{3b} are optionally combined to form a C₃₋₁₀ cycloalkyl, a heterocyclyl, a C₆₋₁₀ aryl, or a 5-10 membered heteroaryl.

A compound of Formula III:

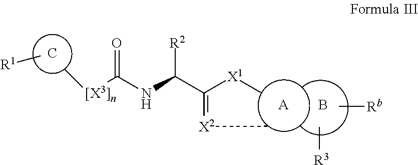

pharmaceutically acceptable salts thereof, deuterium substitutes thereof, and isomers thereof,
wherein Ring C is aryl, 5-6 membered heteroaryl, C₃₋₁₀ cycloalkyl, C₃₋₁₀ heterocyclyl, R^{x}—C₃₋₁₀ cycloalkyl, R^{x}—C₃₋₁₀ heterocyclyl, R^{x}-aryl and R^{x}-5-6 membered heteroaryl; wherein R^{x} is halo or —C₁₋₆ alkyl;
wherein Ring C is optionally substituted with one or more R¹;
wherein each R¹ is independently selected from the group consisting of OH, CN, C₁₋₆ alkyl, C₁₋₆ haloalkyl, C₂₋₆ alkenyl, C₂₋₆ alkynyl, —O—C₁₋₆ alkyl, —O—C₁₋₆ haloalkyl, —CO—C₁₋₆ alkyl, —CO—C₁₋₆ haloalkyl, —CO—NR^{3a}R^{3b}, C₃₋₁₀ cycloalkyl, C₃₋₁₀ heterocyclyl, C₆₋₁₀ aryl, and 5-10 membered heteroaryl;
wherein each R¹ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —S(O)—C₁₋₆ alkyl, —S(O)₂—C₁₋₆ alkyl and —S—C₁₋₆ alkyl;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X is NH, O, or S;
wherein X² is N, O, or S; and wherein X² is optionally attached to an atom in Ring A and forms another ring next to Ring A;
wherein X³ is O or —CR^{c}R^{d}; and wherein each of R^{c} and R^{d} is independently selected from the group consisting of H, halo and —C₁₋₆ alkyl;
wherein Ring A is selected from the group consisting of

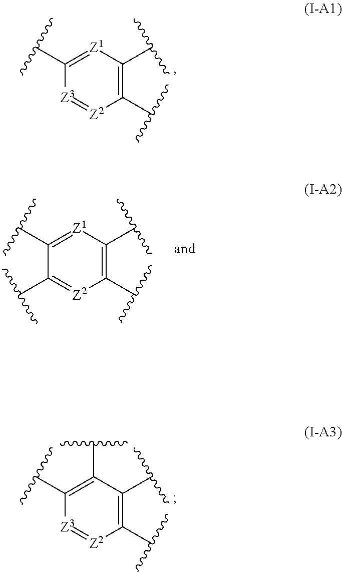

wherein each of Z¹, Z² and Z³ is independently selected from the group consisting of —N═, —N(═O)═ and —C(R^{a})═, wherein R^{a} is selected from the group consisting of H, F, Cl, Br, I, —C₁₋₆ alkyl, C₁₋₆ haloalkyl, C₂₋₆ alkenyl, C₂₋₆ alkynyl, OH, CN, —O—C₁₋₆ alkyl, —O—C₁₋₆ haloalkyl. C₃₋₁₀ cycloalkyl, C₃₋₁₀ heterocyclyl, aryl and 5-10 membered heteroaryl;
wherein Ring B, which is fused to Ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated or unsaturated bridged-ring;
wherein Ring B is optionally substituted with one or more R^{b}, each of R^{b} is independently selected from the group consisting of CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —NR^{3a}—CO—C₁₋₆ alkyl, —NR^{3a}—COOC₁₋₆ alkyl, —NR^{3a}—CO—C₁₋₆ haloalkyl, —NR^{3a}—CO—NR^{3a}R^{3b}, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more substituents each independently selected from the group consisting of halo, CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, C₂₋₆ alkenyl, C₂₋₆ alkynyl, C₃₋₁₀ cycloalkyl, heterocyclyl, C₆₋₁₀ aryl, and 5-10 membered heteroaryl;
wherein Ring B is substituted with R³, wherein R³ is selected from the group consisting of
—CONR^{3a}R^{3b}, —S(O)—NR^{3a}R^{3b}, —S(O)₂—NR^{3a}R^{3b}, —C₀₋₆ alkyl-CONR^{3a}R^{3b}, —C₀₋₆ alkyl-NR^{3a}R^{3b}, —N(R^{3a})—COR^{3a}, —N(R^{3a})—CONR^{3a}R^{3b}, —C₀₋₆ alkyl-SONR^{3a}R³, —C₀₋₆ alkyl-SO₂NR^{3a}R^{3b}, —C(S)—NR^{3a}R^{3b}, CN, 5-10 membered heteroaryl, —COR^{3a}, —P(O)(OR^{3a}(OR^{3b}), —S(O)—C₁₋₆ alkyl, —S(O)₂—C₁₋₆ alkyl, C₁₋₆ alkyl, —C₁₋₆ alkyl-C₃₋₁₀ cycloalkyl, —O—C₁₋₆ alkyl, aryl, heterocyclyl, alkylheterocyclyl, alkylaryl and alkylcycloalkyl; wherein the 5-10 membered heteroaryl, the —S(O)—C₁₋₆ alkyl, the —S(O)₂—C₁₋₆ alkyl, the C₁₋₆ alkyl, the —C₁₋₆ alkyl-C₃₋₁₀ cycloalkyl, the —O—C₁₋₆ alkyl, the aryl, the heterocyclyl, the alkylheterocyclyl, the alkylaryl or the alkylcycloalkyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl;
wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; or R^{3a} and R^{3b} are optionally combined to form a C₃₋₁₀ cycloalkyl, a heterocyclyl, a C₆₋₁₀ aryl, or a 5-10 membered heteroaryl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, and C₁₋₆ haloalkyl.

In some embodiments, the compound of the present application is selected from the group consisting of the following compounds:

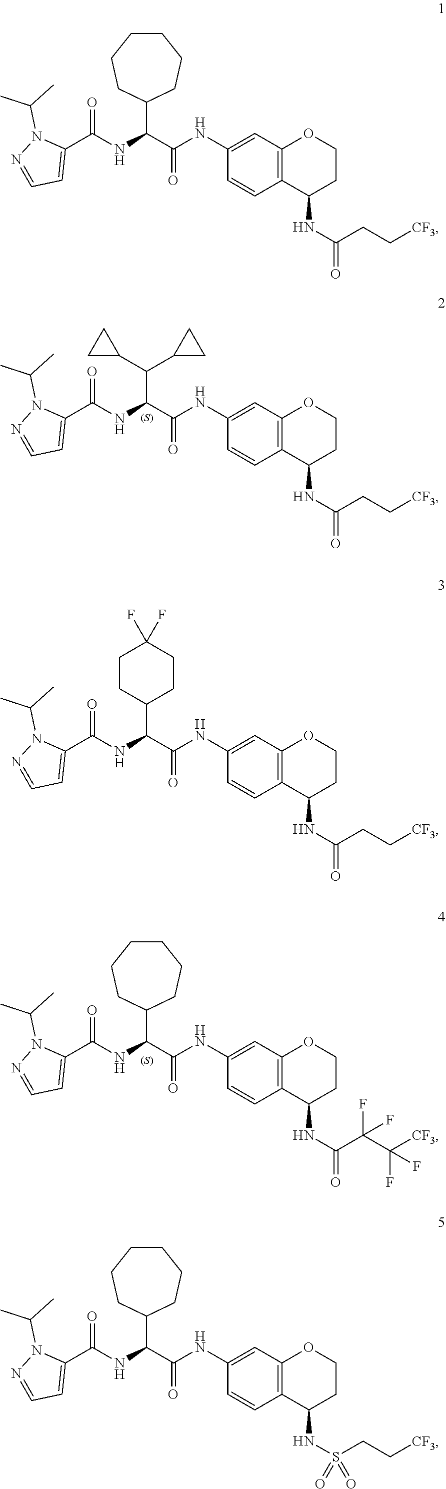

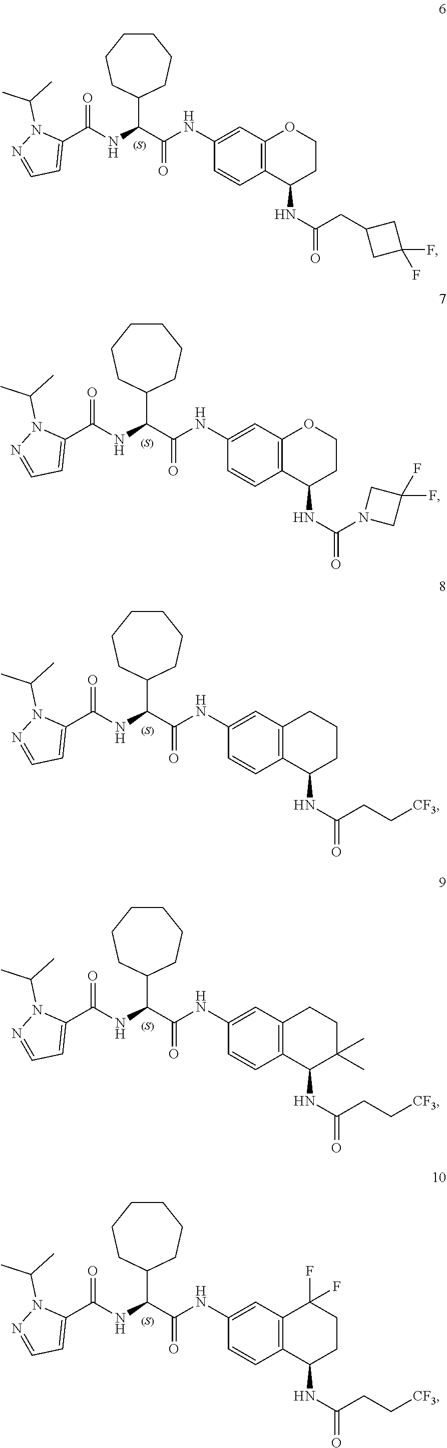

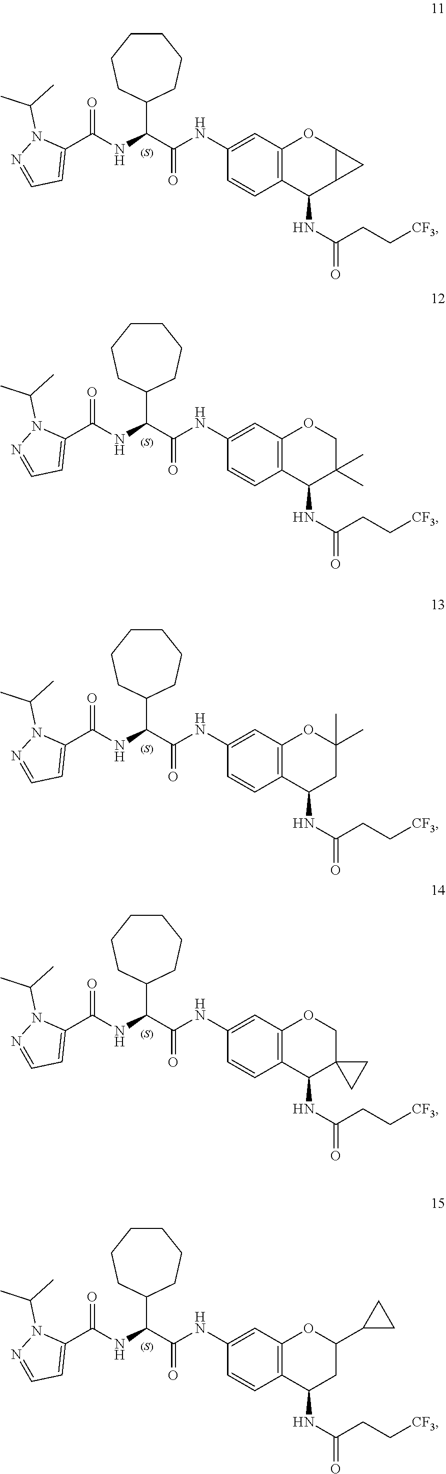

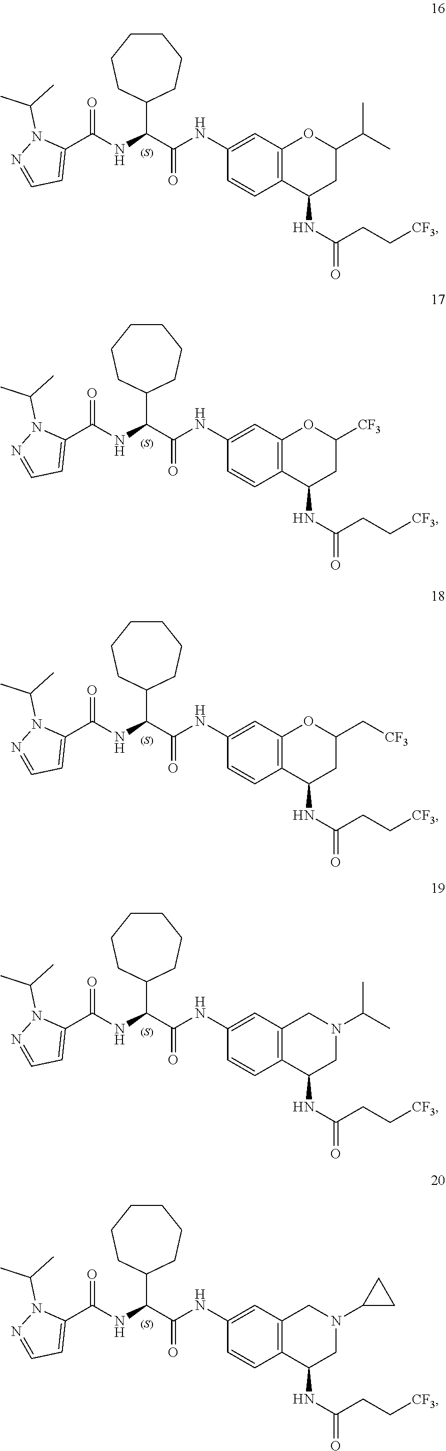

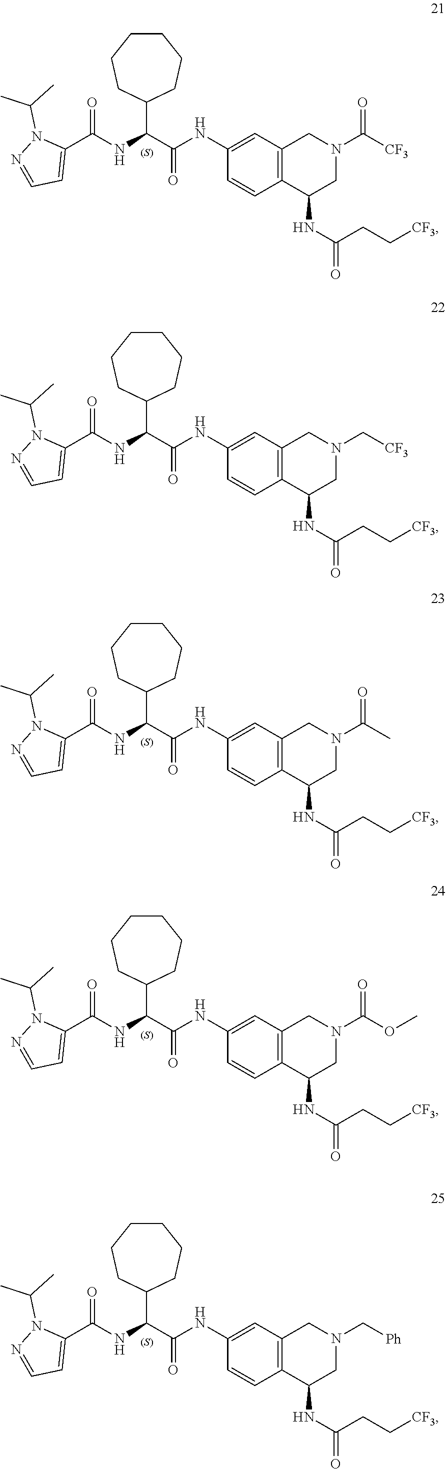

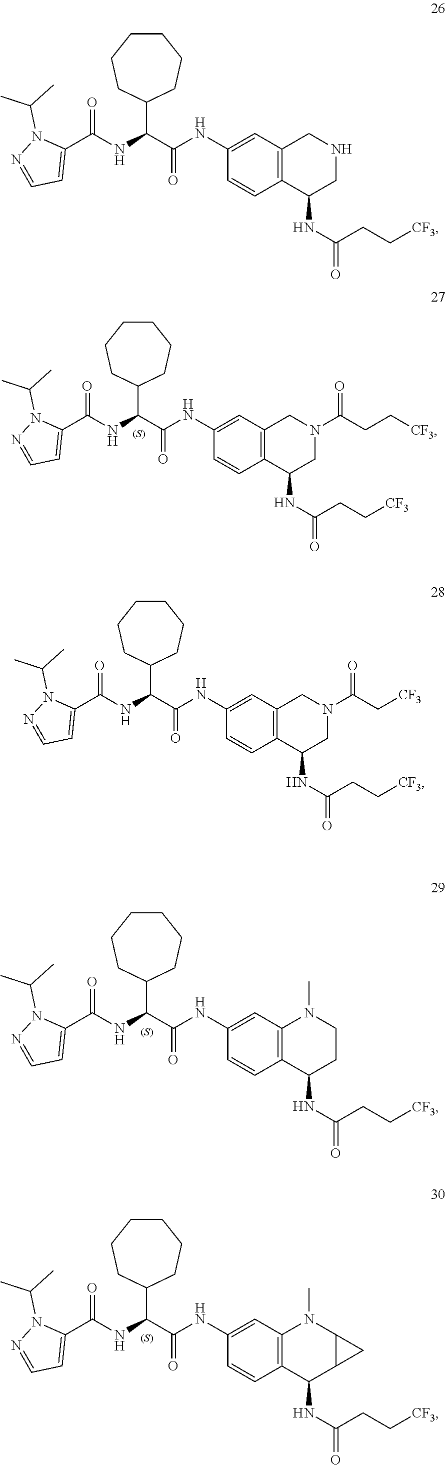

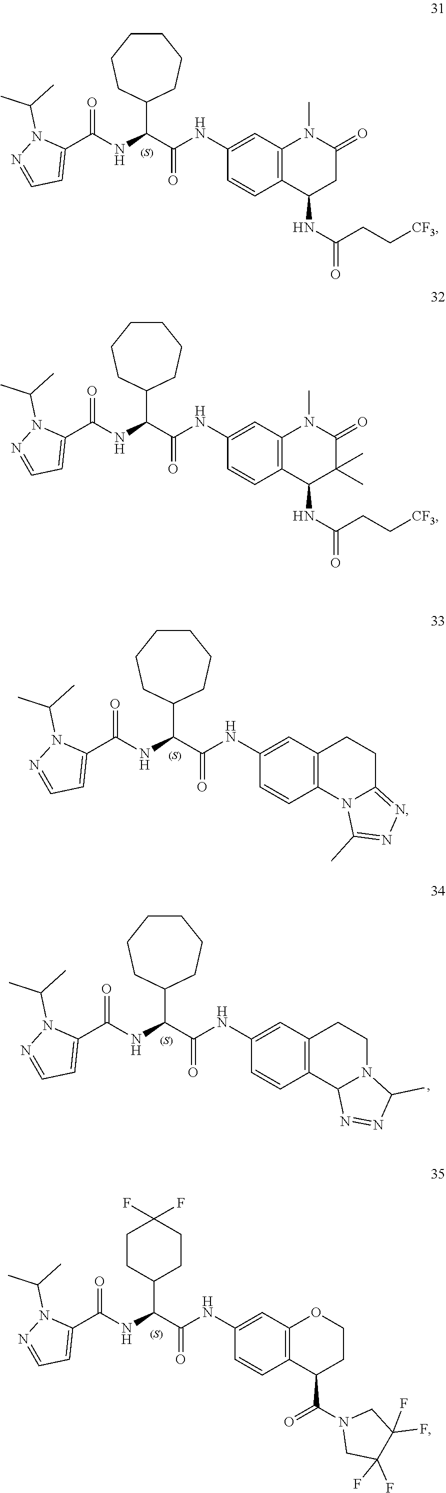

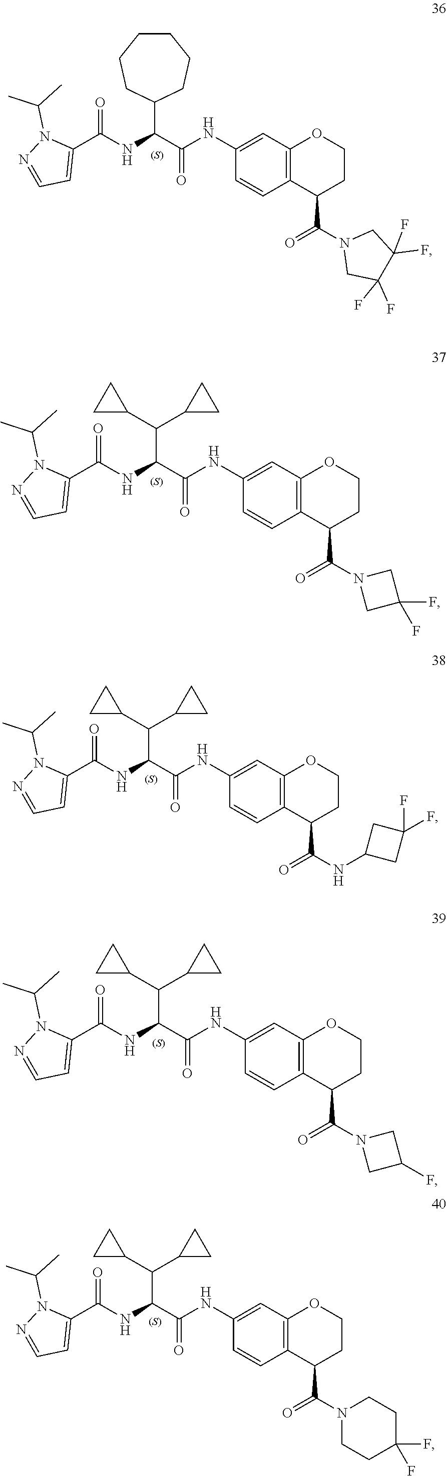

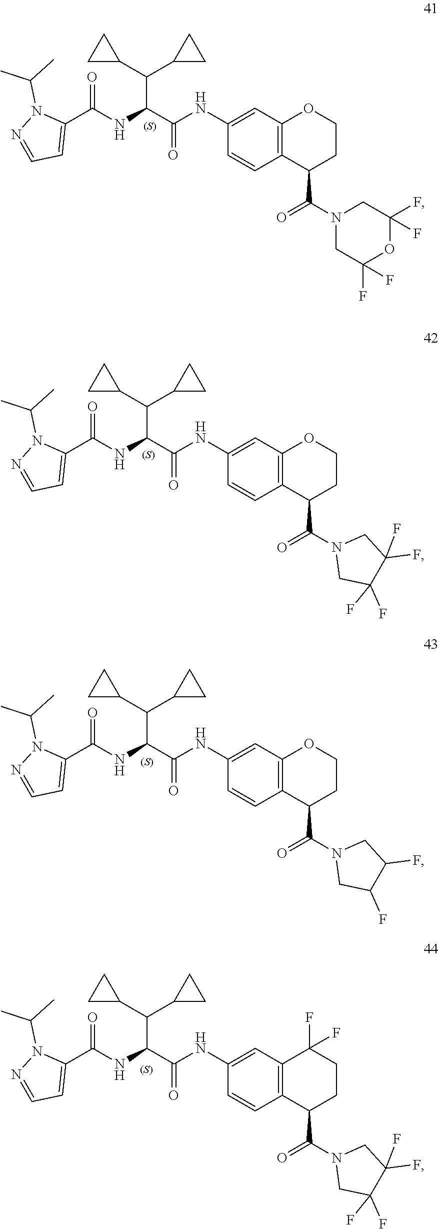

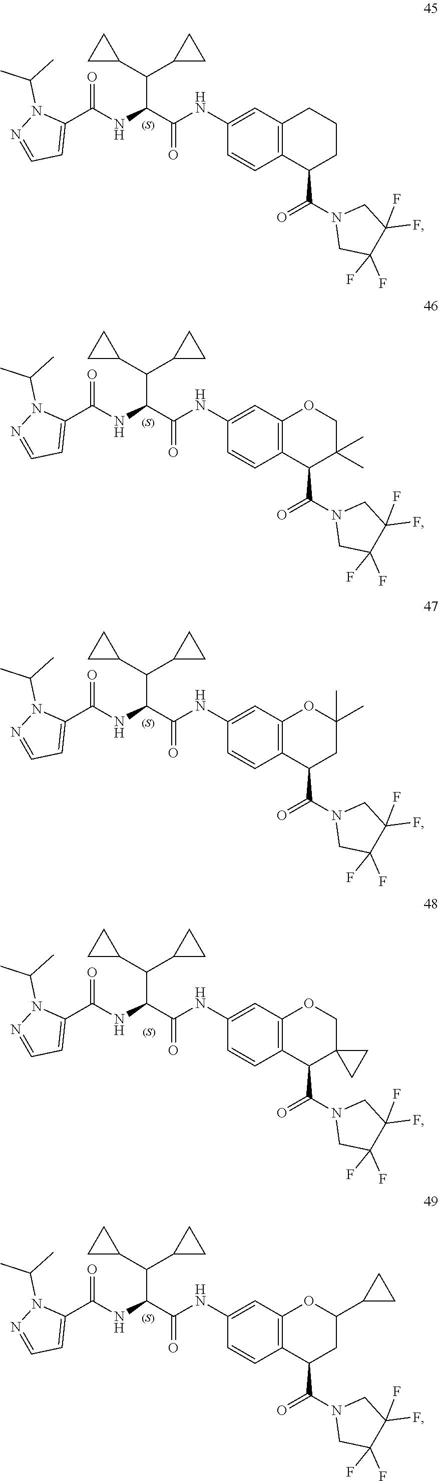

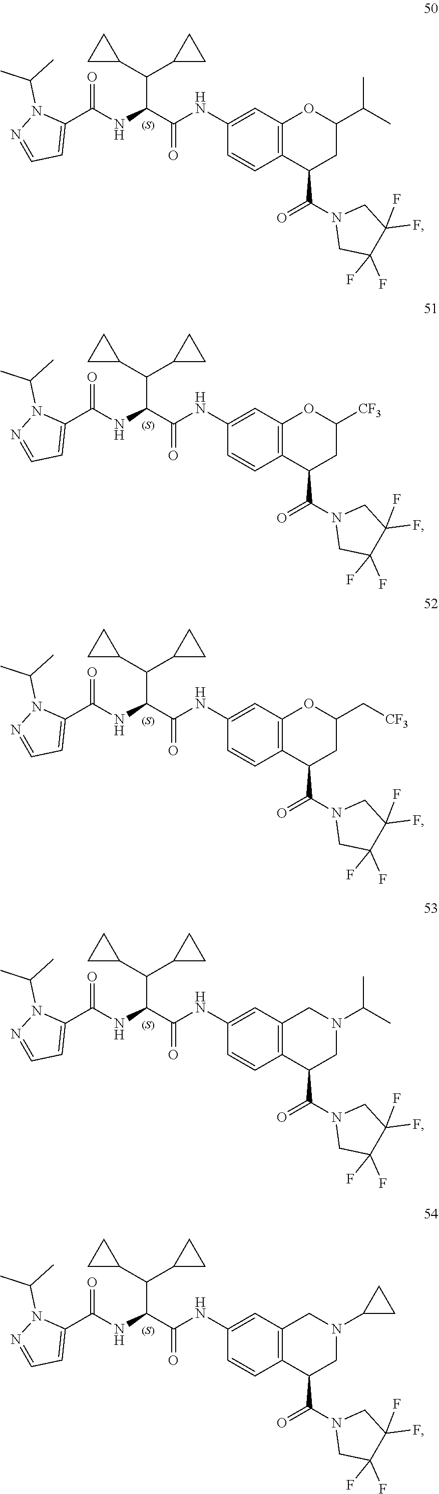

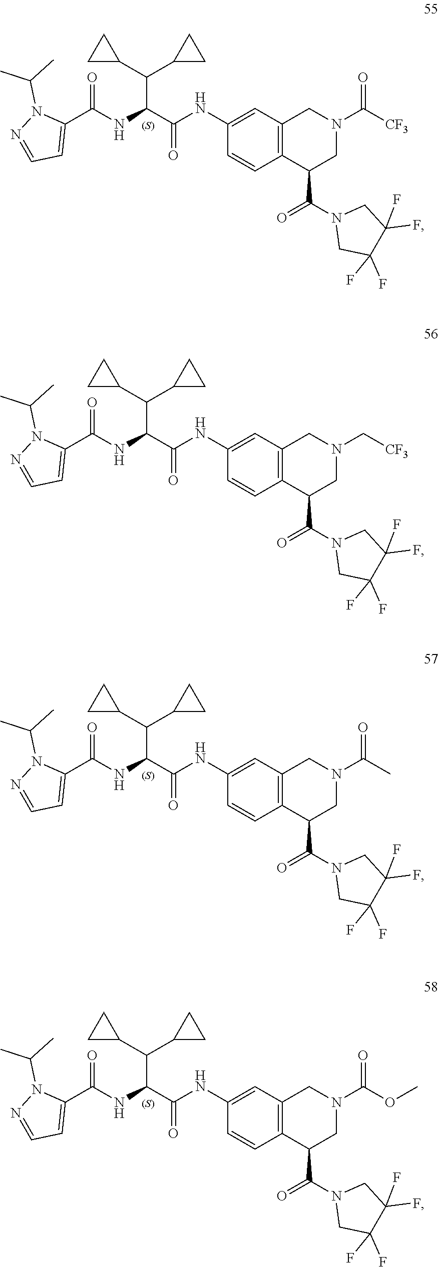

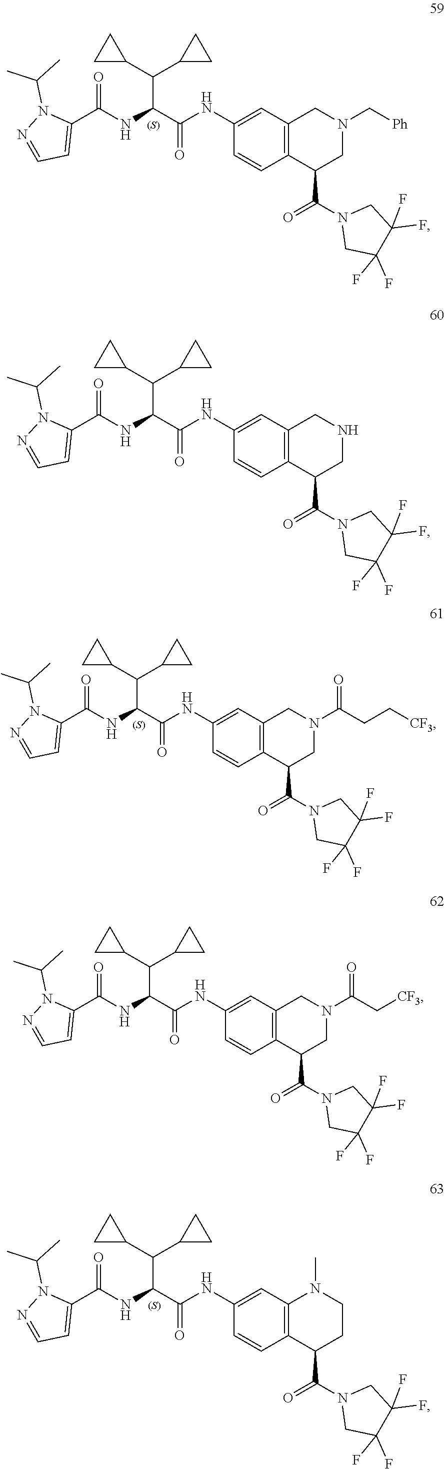

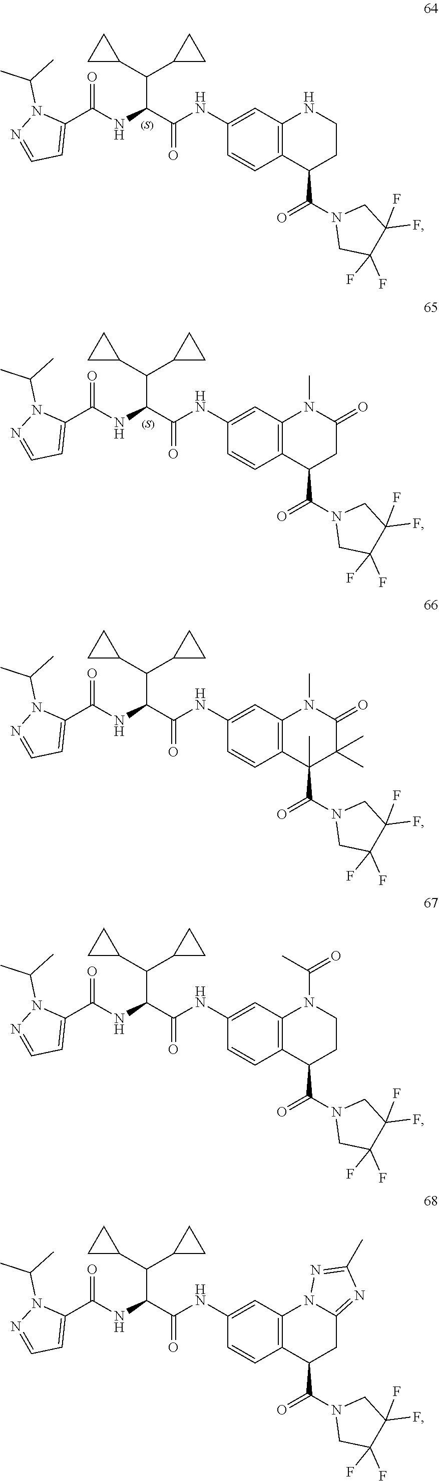

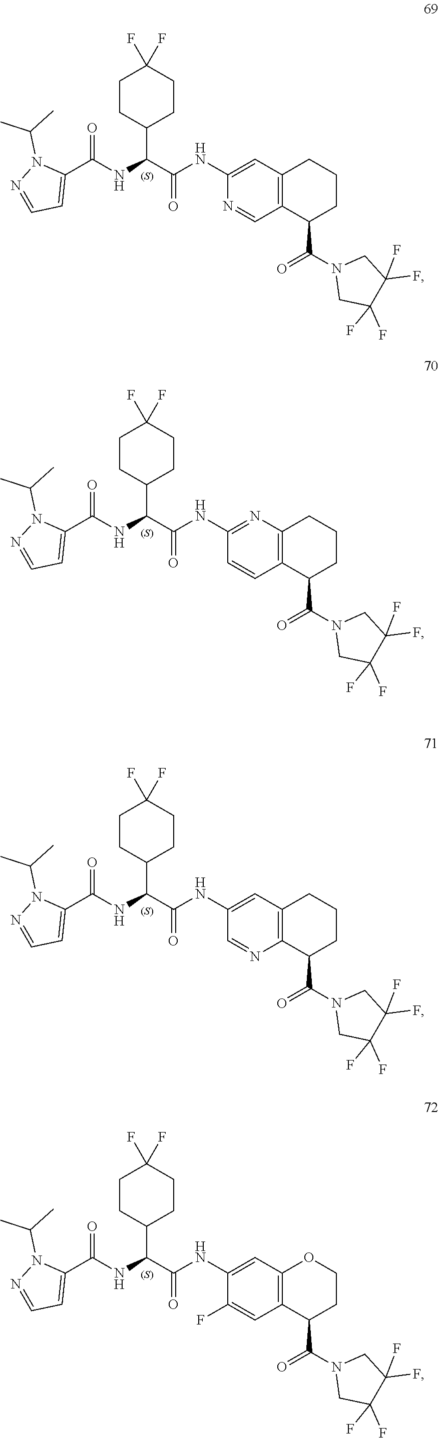

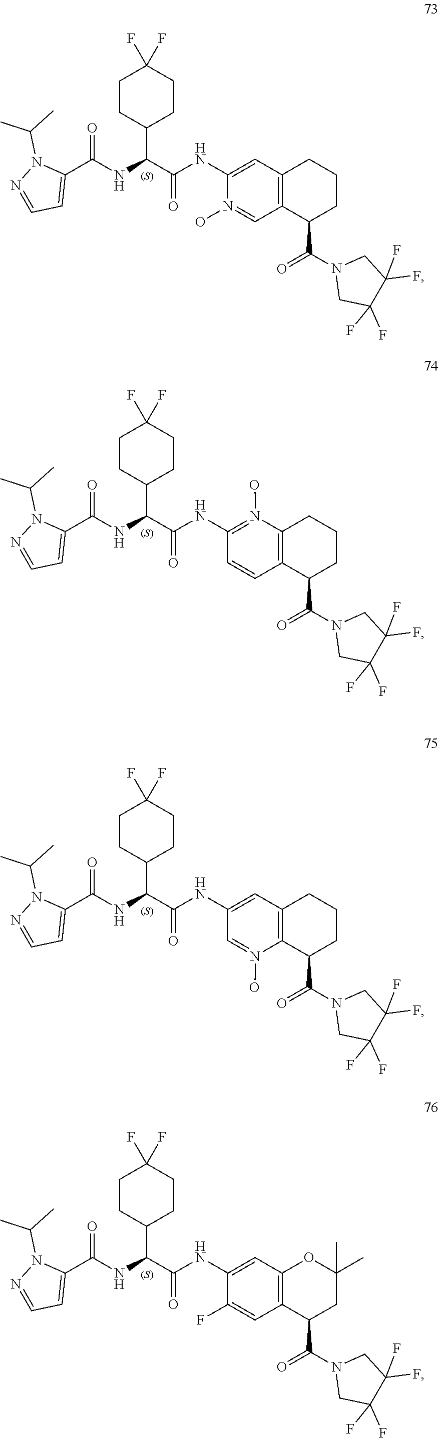

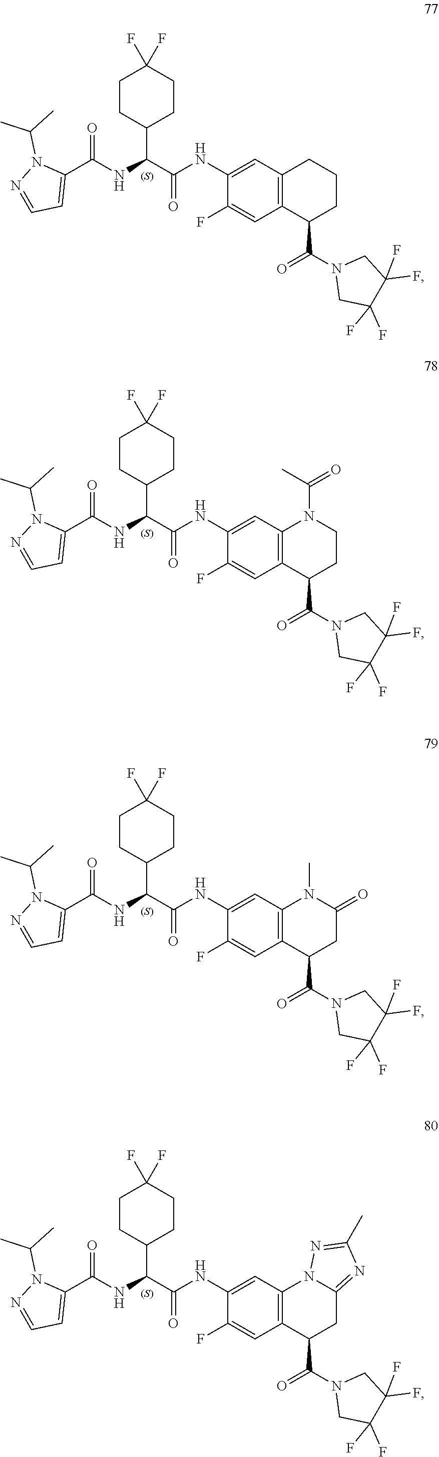

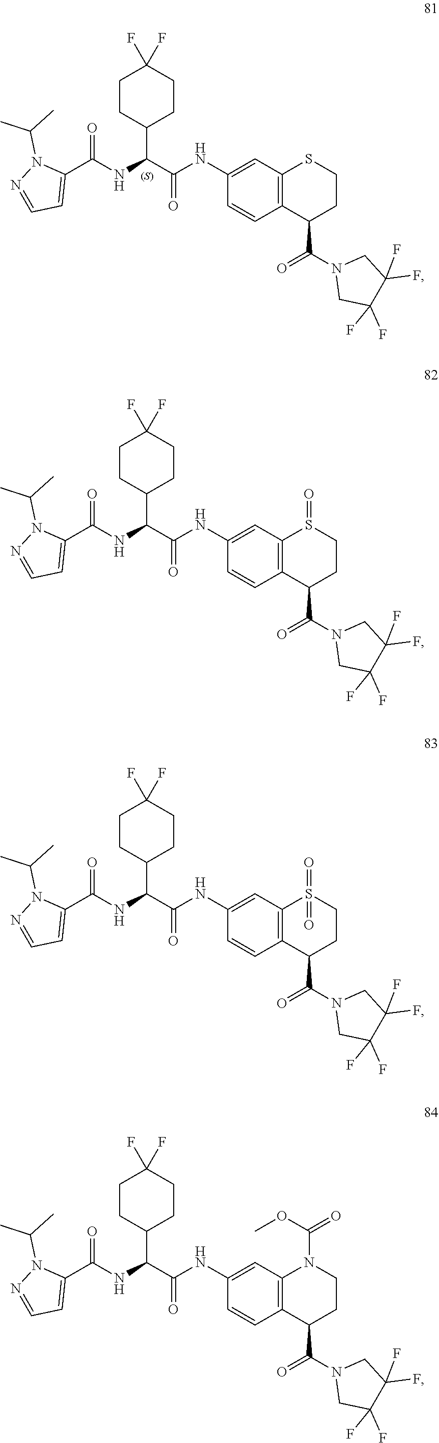

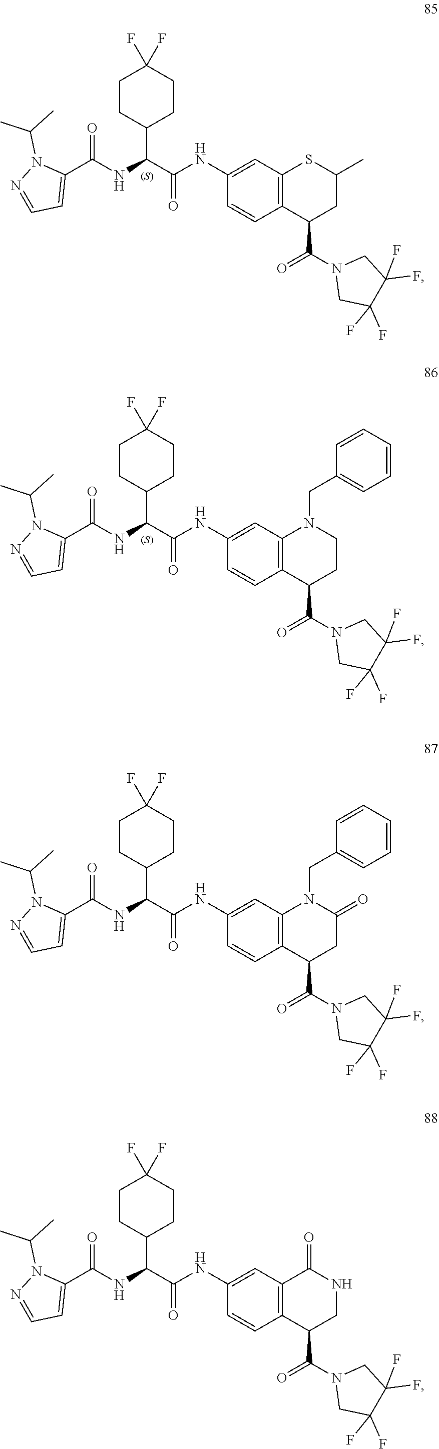

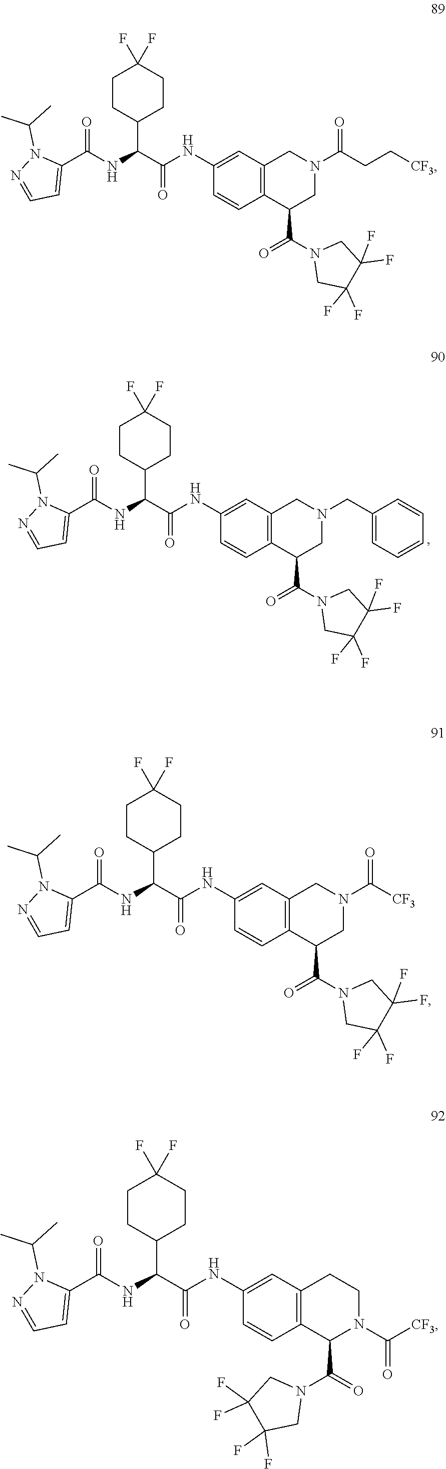 [SMILES: CC(C)n1nccc1C(=O)N[C@H](C(=O)Nc1ccc2c(c1)CN(C(=O)CCC(F)(F)F)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CN(Cc1ccccc1)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CN(C(=O)C(F)(F)F)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CCN(C(=O)C(F)(F)F)[C@H]2C(=O)N1CC(F)(F)C(F)(F)C1)C1CCC(F)(F)CC1]

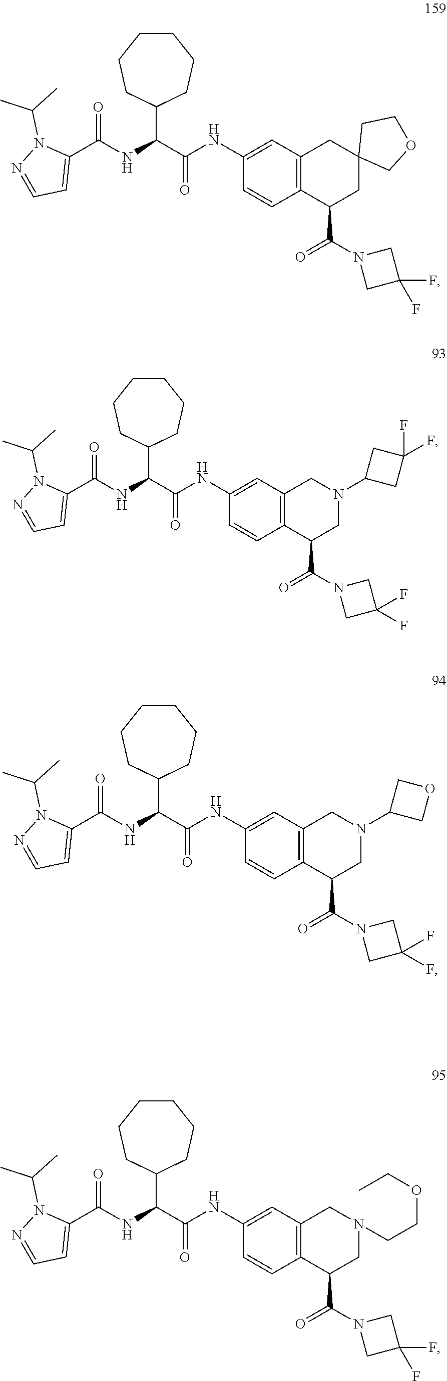 [SMILES: CC(C)n1nccc1C(=O)N[C@H](C(=O)Nc1ccc2c(c1)CC1(CCOC1)C[C@H]2C(=O)N1CC(F)(F)C1)C1CCCCCC1 ; CC(C)n1nccc1C(=O)N[C@H](C(=O)Nc1ccc2c(c1)CN(C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)CN(C1COC1)C[C@H]2C(=O)N1CC(F)(F)C1)C1CCCCCC1 ; CCOCCN1Cc2cc(NC(=O)[C@@H](NC(=O)c3ccnn3C(C)C)C3CCCCCC3)ccc2[C@H](C(=O)N2CC(F)(F)C2)C1]

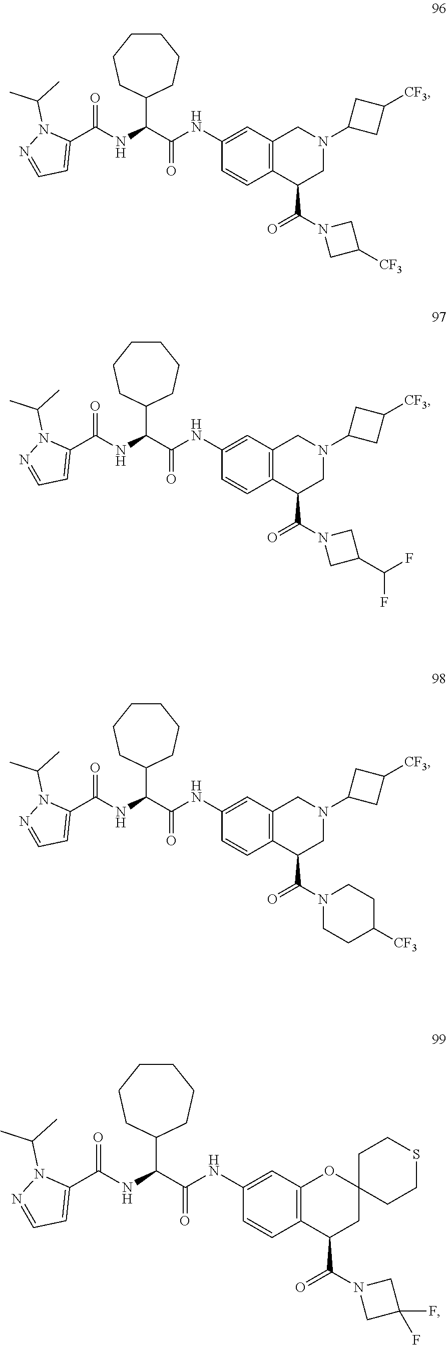 [SMILES: CC(C)n1nccc1C(=O)N[C@H](C(=O)Nc1ccc2c(c1)CN(C1CC(C(F)(F)F)C1)C[C@H]2C(=O)N1CC(C(F)(F)F)C1)C1CCCCCC1 ; CC(C)n1nccc1C(=O)N[C@H](C(=O)Nc1ccc2c(c1)CN(C1CC(C(F)(F)F)C1)C[C@H]2C(=O)N1CC(C(F)F)C1)C1CCCCCC1 ; CC(C)n1nccc1C(=O)N[C@H](C(=O)Nc1ccc2c(c1)CN(C1CC(C(F)(F)F)C1)C[C@H]2C(=O)N1CCC(C(F)(F)F)CC1)C1CCCCCC1 ; CC(C)n1nccc1C(=O)N[C@H](C(=O)Nc1ccc2c(c1)OC1(CCSCC1)C[C@H]2C(=O)N1CC(F)(F)C1)C1CCCCCC1]

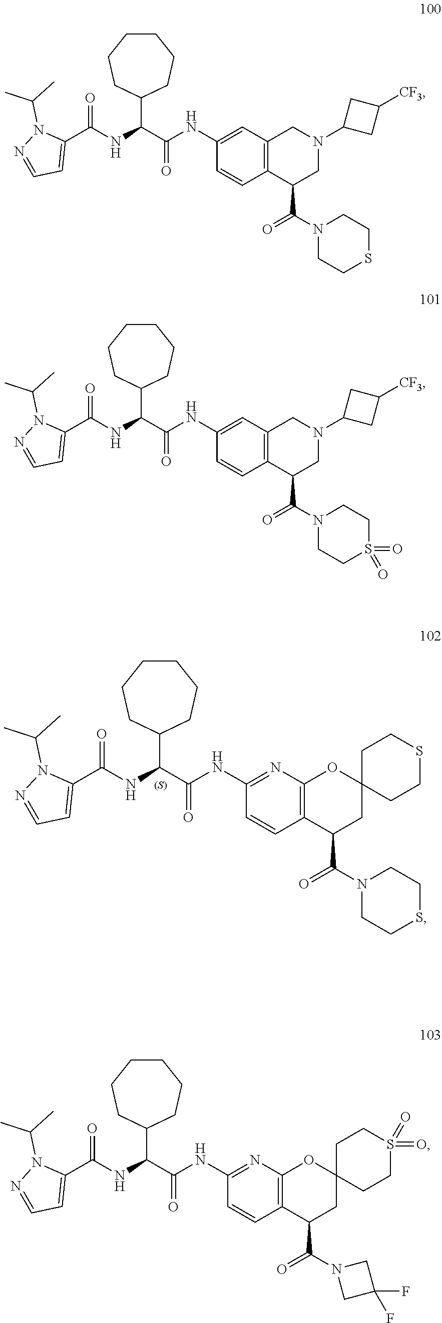

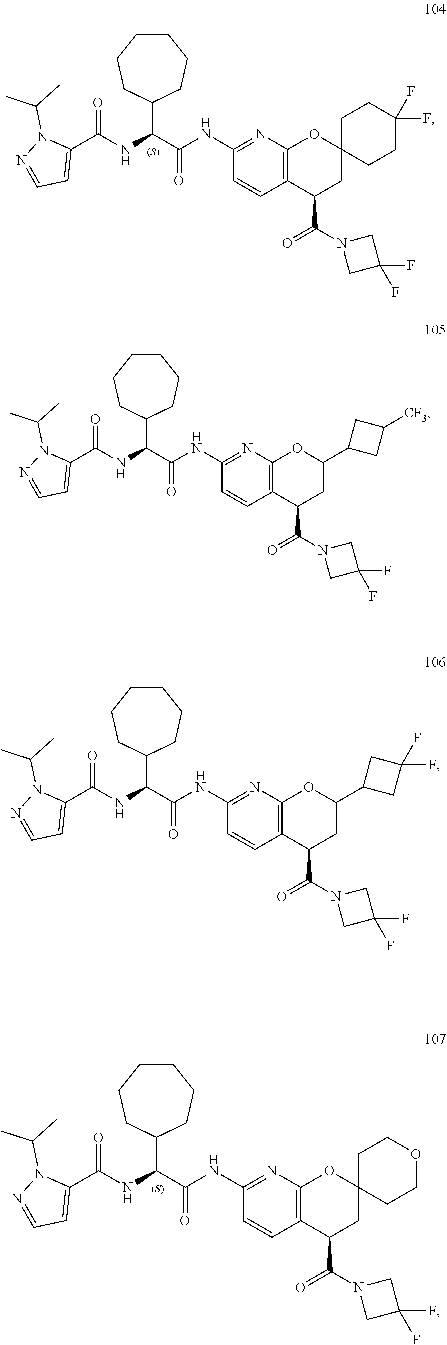

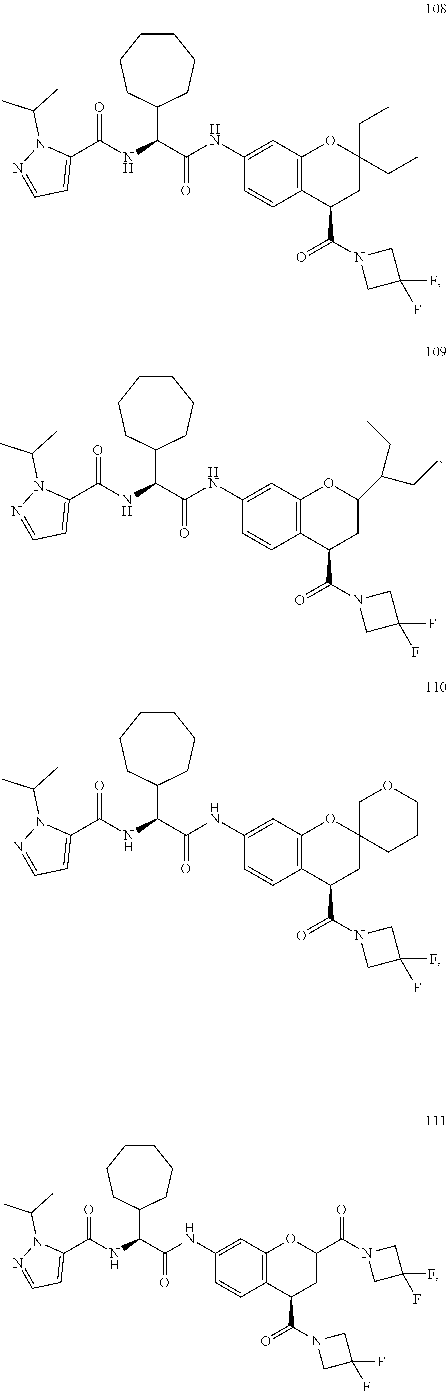

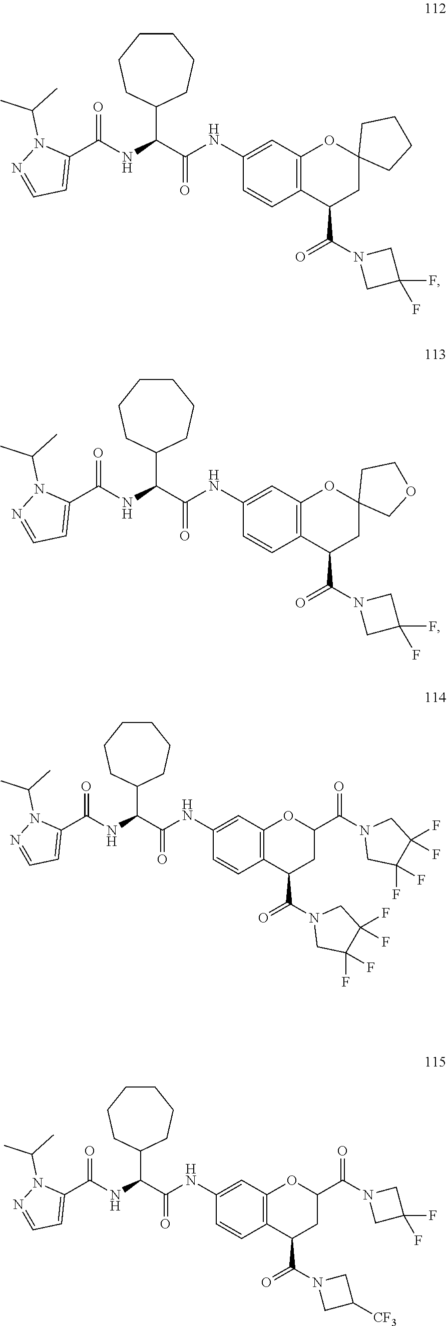 [SMILES: CC(C)n1nccc1C(=O)N[C@H](C(=O)Nc1ccc2c(c1)OC1(CCCC1)C[C@H]2C(=O)N1CC(F)(F)C1)C1CCCCCC1 ; CC(C)n1nccc1C(=O)N[C@H](C(=O)Nc1ccc2c(c1)OC1(CCOC1)C[C@H]2C(=O)N1CC(F)(F)C1)C1CCCCCC1 ; CC(C)n1nccc1C(=O)N[C@H](C(=O)Nc1ccc2c(c1)OC(C(=O)N1CC(F)(F)C(F)(F)C1)C[C@H]2C(=O)N1CC(F)(F)C(F)(F)C1)C1CCCCCC1 ; CC(C)n1nccc1C(=O)N[C@H](C(=O)Nc1ccc2c(c1)OC(C(=O)N1CC(F)(F)C1)C[C@H]2C(=O)N1CC(C(F)(F)F)C1)C1CCCCCC1]

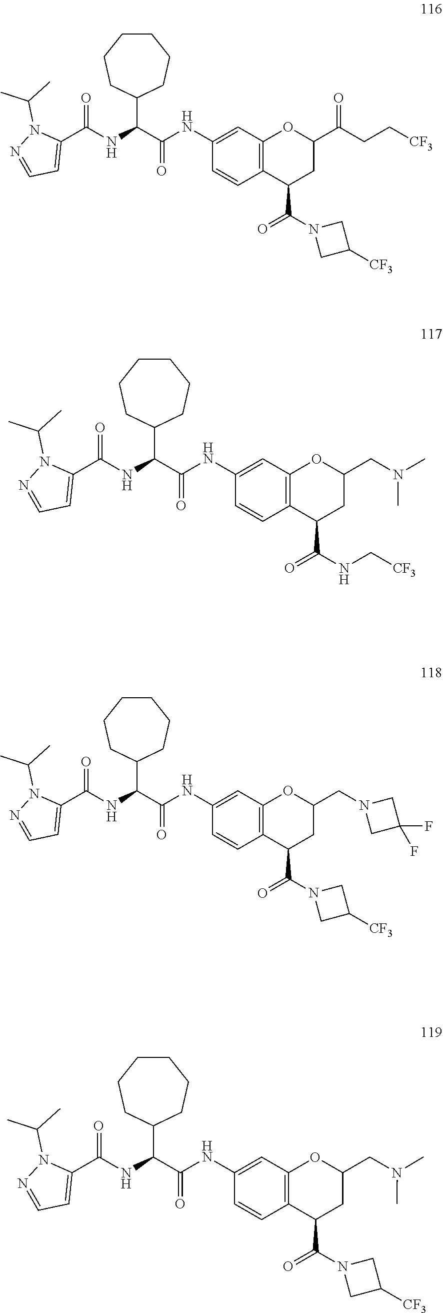

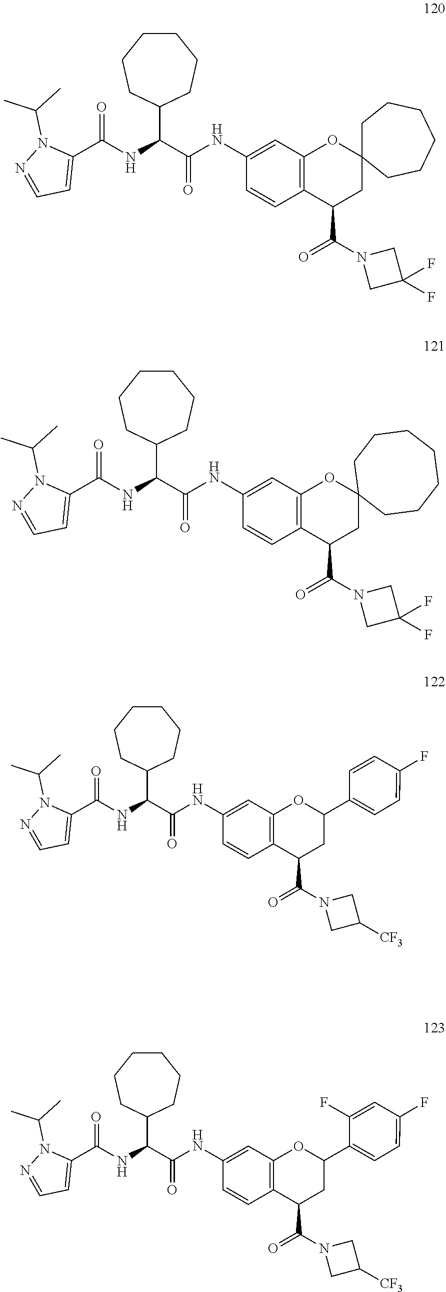 [SMILES: CC(C)n1nccc1C(=O)N[C@H](C(=O)Nc1ccc2c(c1)OC1(CCCCCC1)C[C@H]2C(=O)N1CC(F)(F)C1)C1CCCCCC1 ; CC(C)n1nccc1C(=O)N[C@H](C(=O)Nc1ccc2c(c1)OC1(CCCCCCC1)C[C@H]2C(=O)N1CC(F)(F)C1)C1CCCCCC1 ; CC(C)n1nccc1C(=O)N[C@H](C(=O)Nc1ccc2c(c1)OC(c1ccc(F)cc1)C[C@H]2C(=O)N1CC(C(F)(F)F)C1)C1CCCCCC1 ; CC(C)n1nccc1C(=O)N[C@H](C(=O)Nc1ccc2c(c1)OC(c1ccc(F)cc1F)C[C@H]2C(=O)N1CC(C(F)(F)F)C1)C1CCCCCC1]

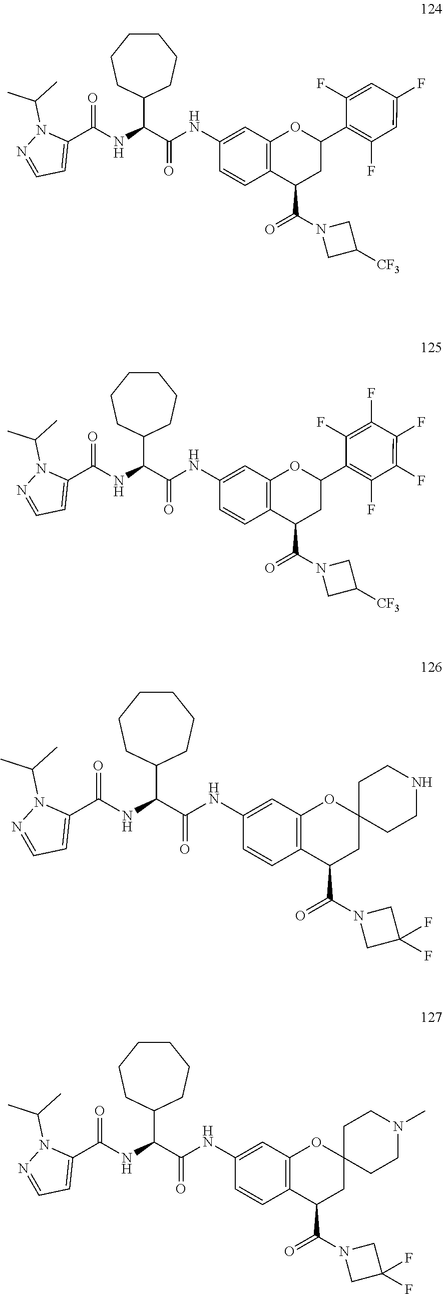 [SMILES: CC(C)n1nccc1C(=O)N[C@H](C(=O)Nc1ccc2c(c1)OC(c1c(F)cc(F)cc1F)C[C@H]2C(=O)N1CC(C(F)(F)F)C1)C1CCCCCC1 ; CC(C)n1nccc1C(=O)N[C@H](C(=O)Nc1ccc2c(c1)OC(c1c(F)c(F)c(F)c(F)c1F)C[C@H]2C(=O)N1CC(C(F)(F)F)C1)C1CCCCCC1 ; CC(C)n1nccc1C(=O)N[C@H](C(=O)Nc1ccc2c(c1)OC1(CCNCC1)C[C@H]2C(=O)N1CC(F)(F)C1)C1CCCCCC1 ; CC(C)n1nccc1C(=O)N[C@H](C(=O)Nc1ccc2c(c1)OC1(CCN(C)CC1)C[C@H]2C(=O)N1CC(F)(F)C1)C1CCCCCC1]

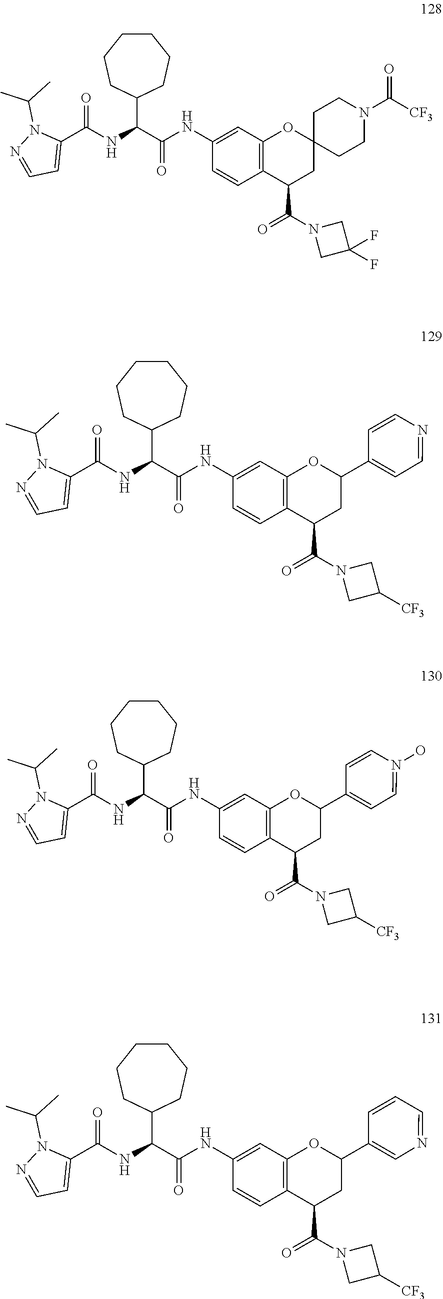

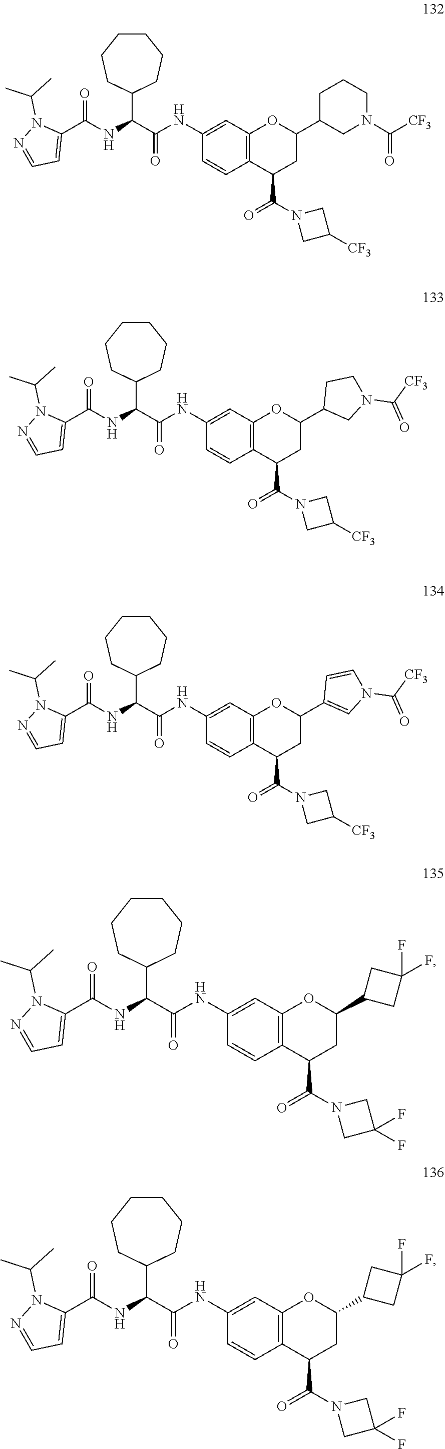

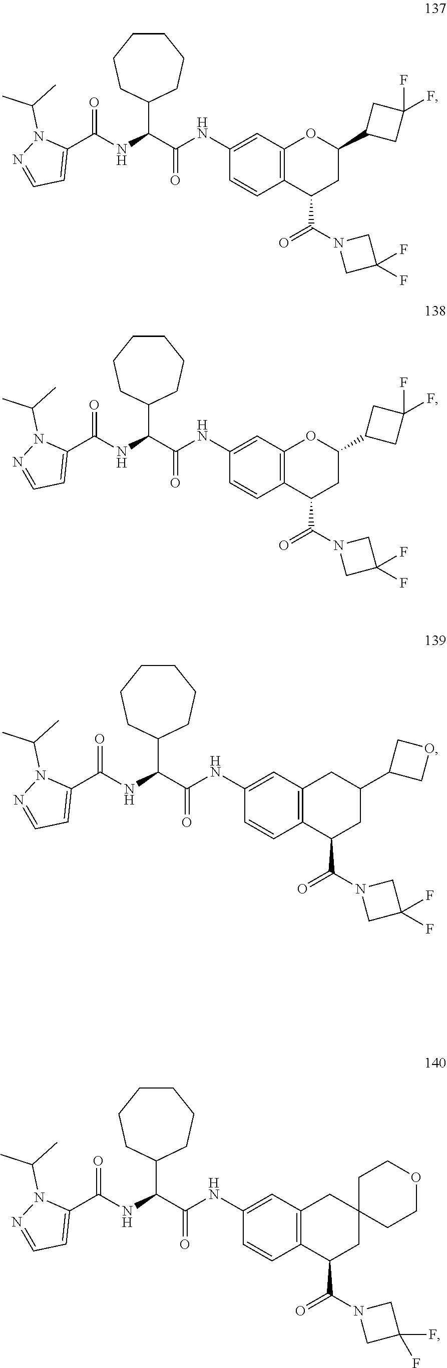 [SMILES: CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1 ; CC(C)n1nccc1C(=O)N[C@H](C(=O)Nc1ccc2c(c1)CC(C1COC1)C[C@H]2C(=O)N1CC(F)(F)C1)C1CCCCCC1 ; CC(C)n1nccc1C(=O)N[C@H](C(=O)Nc1ccc2c(c1)CC1(CCOCC1)C[C@H]2C(=O)N1CC(F)(F)C1)C1CCCCCC1]

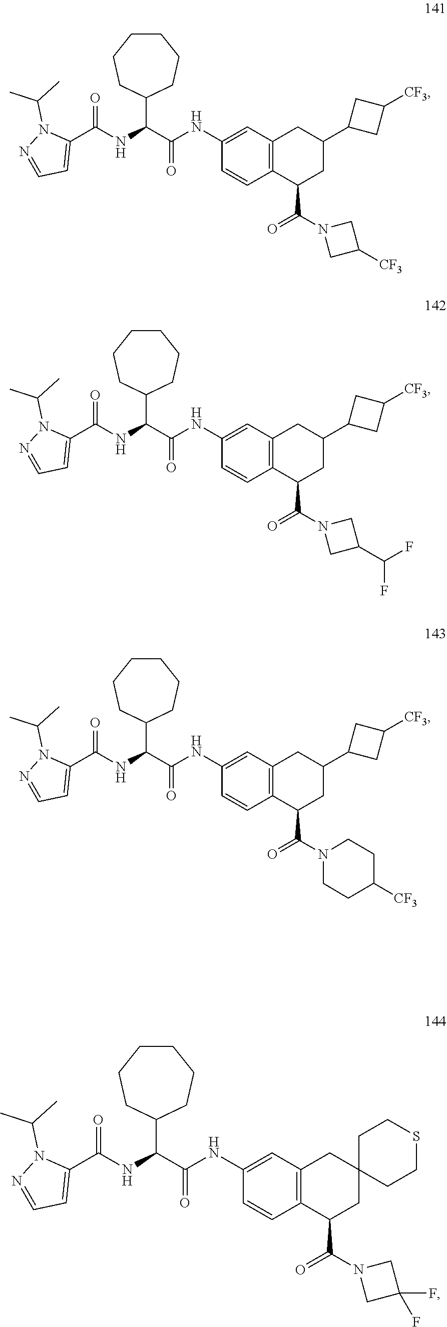 [SMILES: CC(C)n1nccc1C(=O)N[C@H](C(=O)Nc1ccc2c(c1)CC(C1CC(C(F)(F)F)C1)C[C@H]2C(=O)N1CC(C(F)(F)F)C1)C1CCCCCC1 ; CC(C)n1nccc1C(=O)N[C@H](C(=O)Nc1ccc2c(c1)CC(C1CC(C(F)(F)F)C1)C[C@H]2C(=O)N1CC(C(F)F)C1)C1CCCCCC1 ; CC(C)n1nccc1C(=O)N[C@H](C(=O)Nc1ccc2c(c1)CC(C1CC(C(F)(F)F)C1)C[C@H]2C(=O)N1CCC(C(F)(F)F)CC1)C1CCCCCC1 ; CC(C)n1nccc1C(=O)N[C@H](C(=O)Nc1ccc2c(c1)CC1(CCSCC1)C[C@H]2C(=O)N1CC(F)(F)C1)C1CCCCCC1]

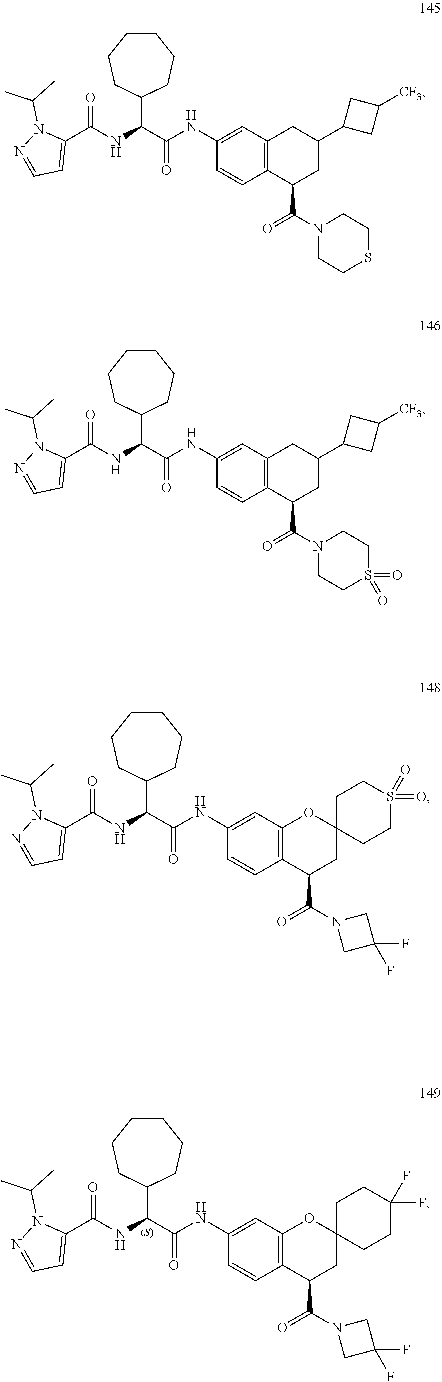

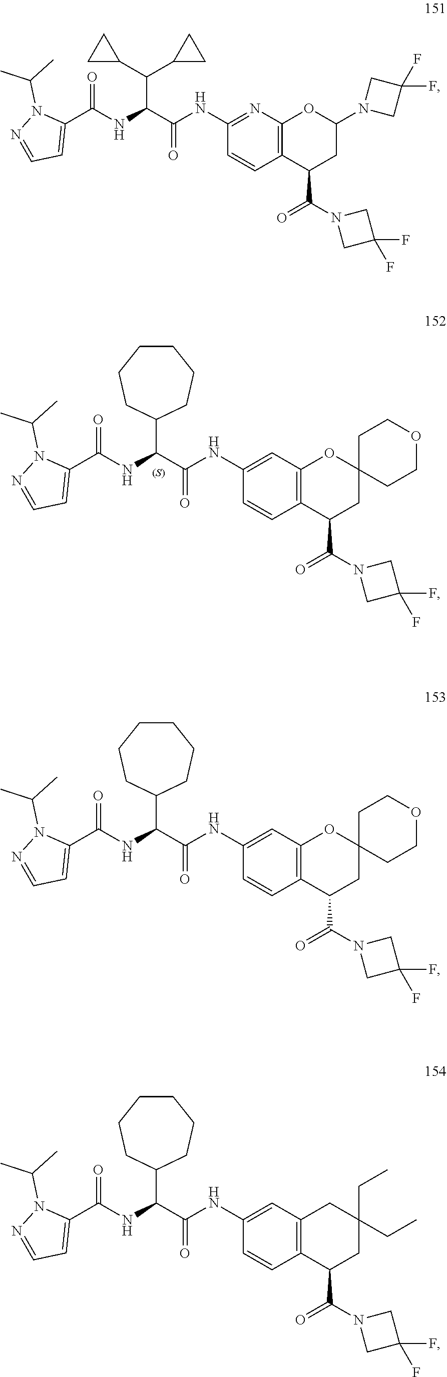

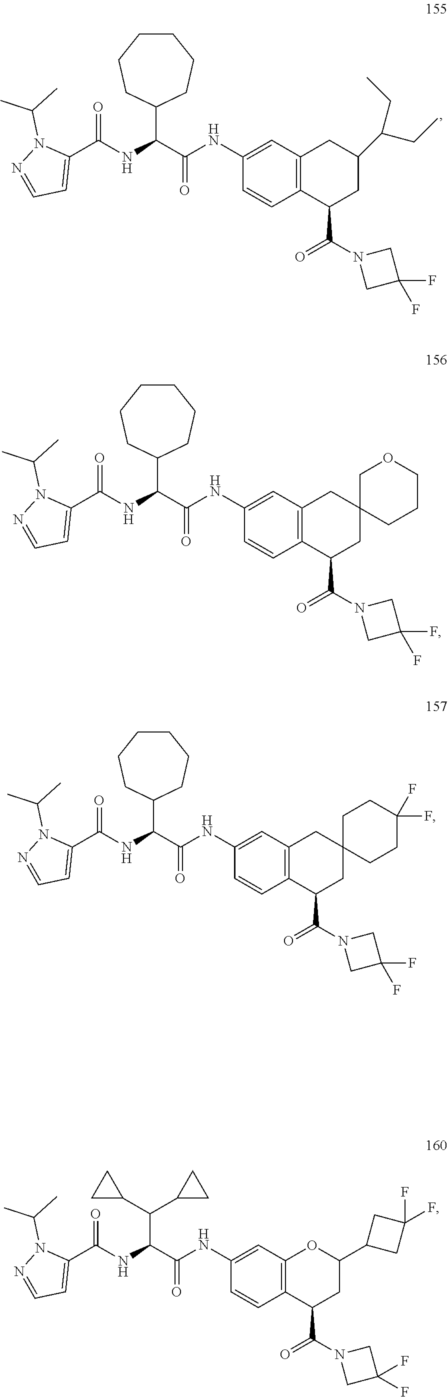

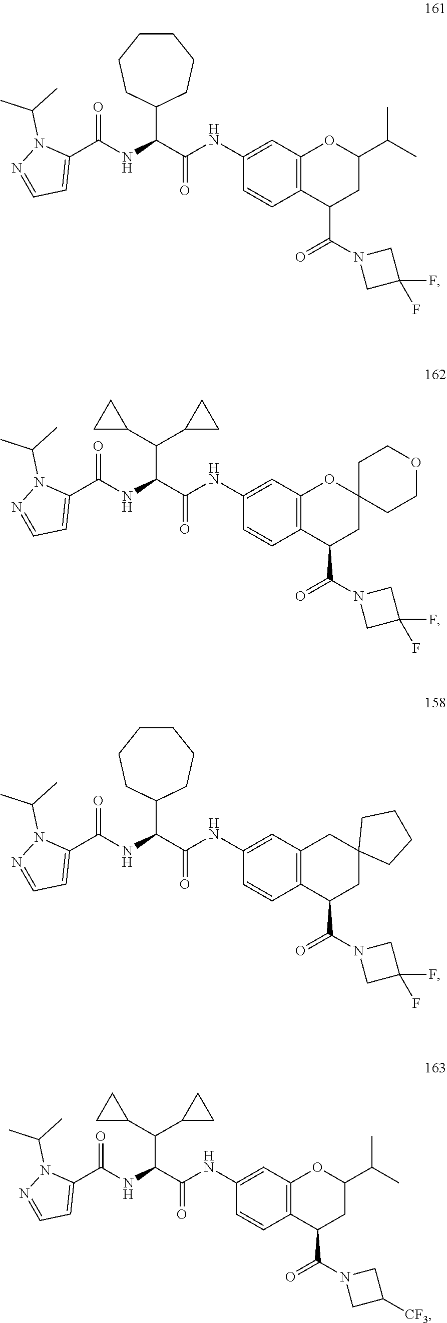 [SMILES: CC(C)C1CC(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCOCC1)C[C@H]2C(=O)N1CC(F)(F)C1)C(C1CC1)C1CC1 ; CC(C)n1nccc1C(=O)N[C@H](C(=O)Nc1ccc2c(c1)CC1(CCCC1)C[C@H]2C(=O)N1CC(F)(F)C1)C1CCCCCC1 ; CC(C)C1C[C@@H](C(=O)N2CC(C(F)(F)F)C2)c2ccc(NC(=O)[C@@H](NC(=O)c3ccnn3C(C)C)C(C3CC3)C3CC3)cc2O1]

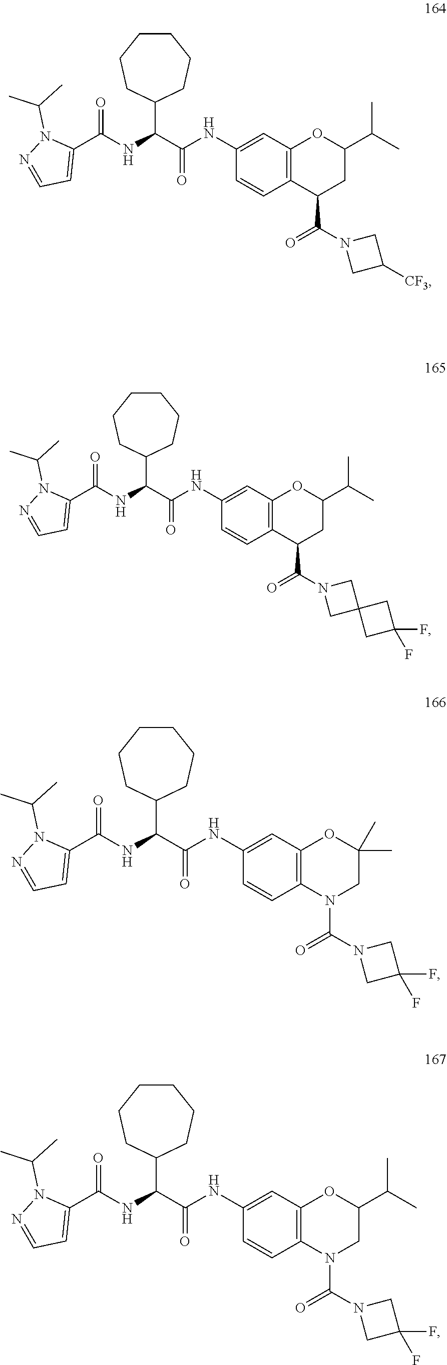 [SMILES: CC(C)C1C[C@@H](C(=O)N2CC(C(F)(F)F)C2)c2ccc(NC(=O)[C@@H](NC(=O)c3ccnn3C(C)C)C3CCCCCC3)cc2O1 ; CC(C)C1C[C@@H](C(=O)N2CC3(C2)CC(F)(F)C3)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC(C)(C)CN2C(=O)N1CC(F)(F)C1)C1CCCCCC1 ; CC(C)C1CN(C(=O)N2CC(F)(F)C2)c2ccc(NC(=O)[C@@H](NC(=O)c3ccnn3C(C)C)C3CCCCCC3)cc2O1]

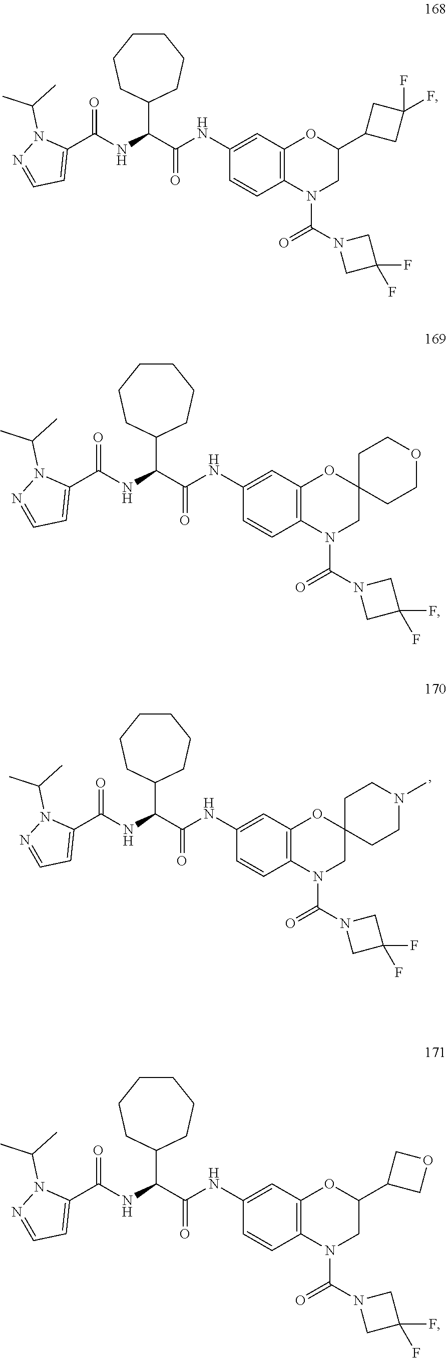

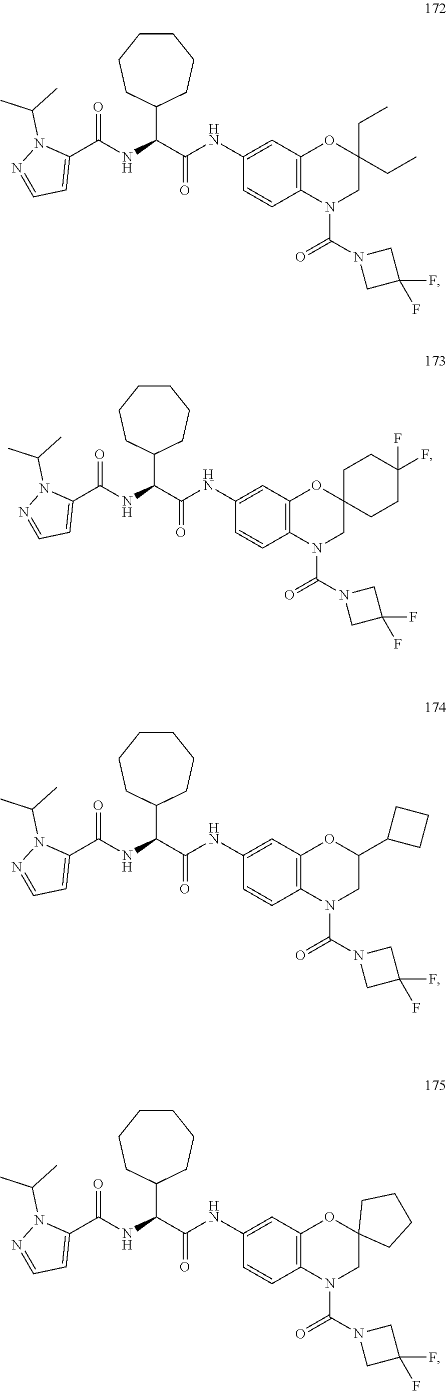 [SMILES: CCC1(CC)CN(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCC(F)(F)CC1)CN2C(=O)N1CC(F)(F)C1)C1CCCCCC1 ; CC(C)n1nccc1C(=O)N[C@H](C(=O)Nc1ccc2c(c1)OC(C1CCC1)CN2C(=O)N1CC(F)(F)C1)C1CCCCCC1 ; CC(C)n1nccc1C(=O)N[C@H](C(=O)Nc1ccc2c(c1)OC1(CCCC1)CN2C(=O)N1CC(F)(F)C1)C1CCCCCC1]

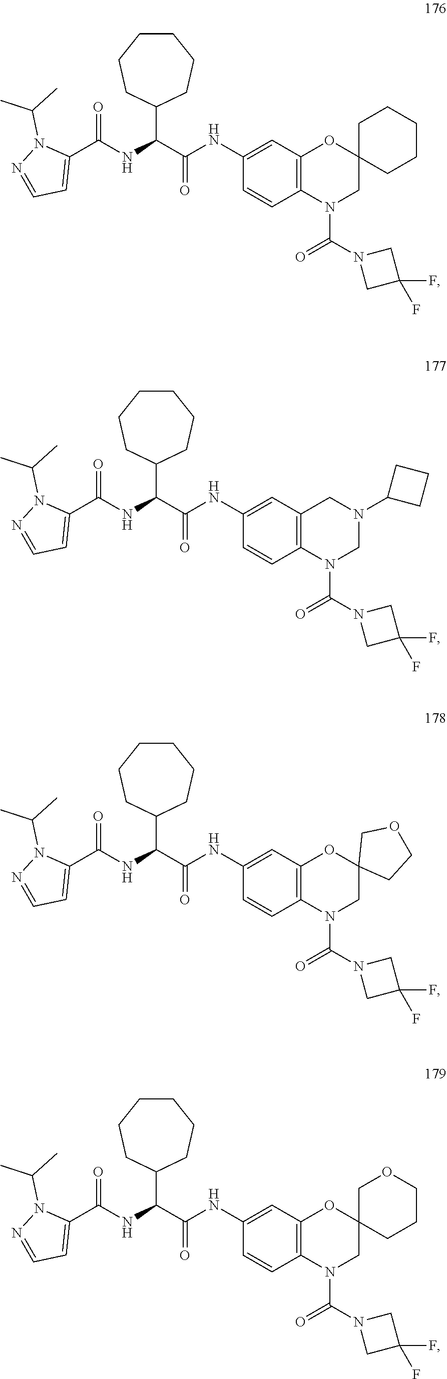 [SMILES: CC(C)n1nccc1C(=O)N[C@H](C(=O)Nc1ccc2c(c1)OC1(CCCCC1)CN2C(=O)N1CC(F)(F)C1)C1CCCCCC1 ; CC(C)n1nccc1C(=O)N[C@H](C(=O)Nc1ccc2c(c1)CN(C1CCC1)CN2C(=O)N1CC(F)(F)C1)C1CCCCCC1 ; CC(C)n1nccc1C(=O)N[C@H](C(=O)Nc1ccc2c(c1)OC1(CCOC1)CN2C(=O)N1CC(F)(F)C1)C1CCCCCC1 ; CC(C)n1nccc1C(=O)N[C@H](C(=O)Nc1ccc2c(c1)OC1(CCCOC1)CN2C(=O)N1CC(F)(F)C1)C1CCCCCC1]

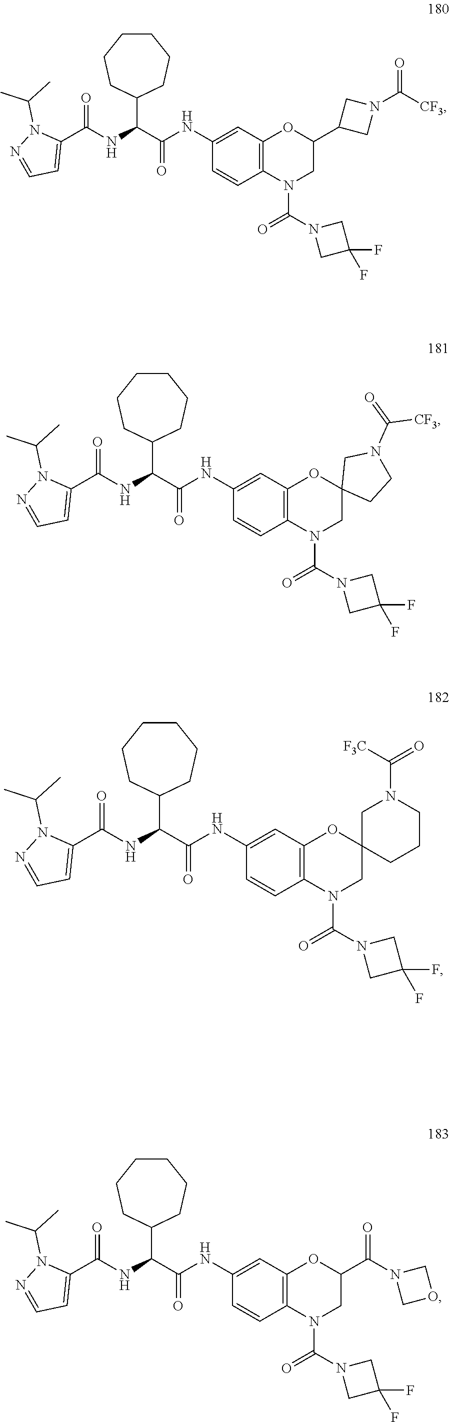 [SMILES: CC(C)n1nccc1C(=O)N[C@H](C(=O)Nc1ccc2c(c1)OC(C1CN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC(C(=O)N1COC1)CN2C(=O)N1CC(F)(F)C1)C1CCCCCC1]

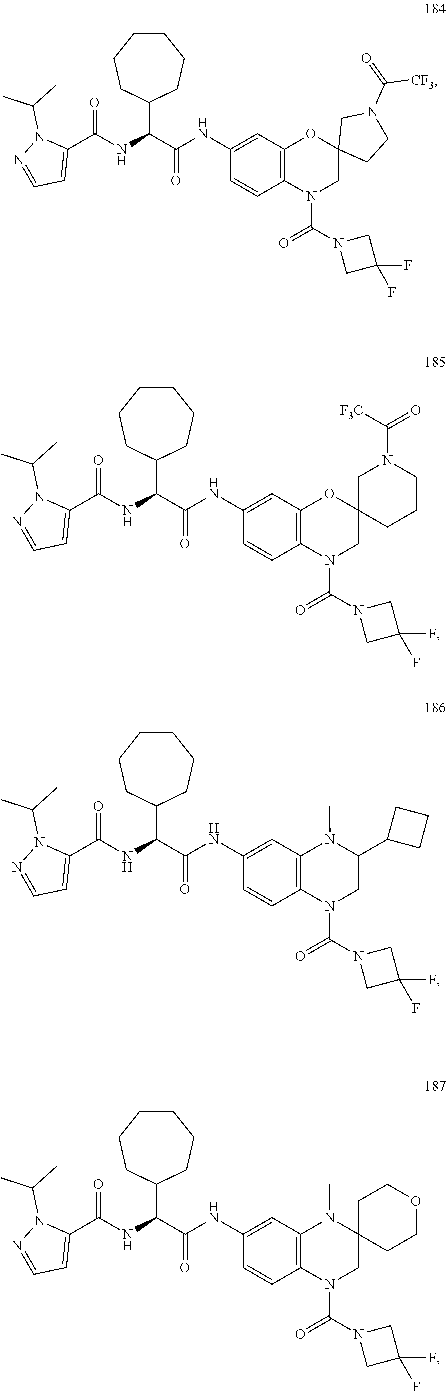 [SMILES: CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)N(C)C(C1CCC1)CN2C(=O)N1CC(F)(F)C1)C1CCCCCC1 ; CC(C)n1nccc1C(=O)N[C@H](C(=O)Nc1ccc2c(c1)N(C)C1(CCOCC1)CN2C(=O)N1CC(F)(F)C1)C1CCCCCC1]

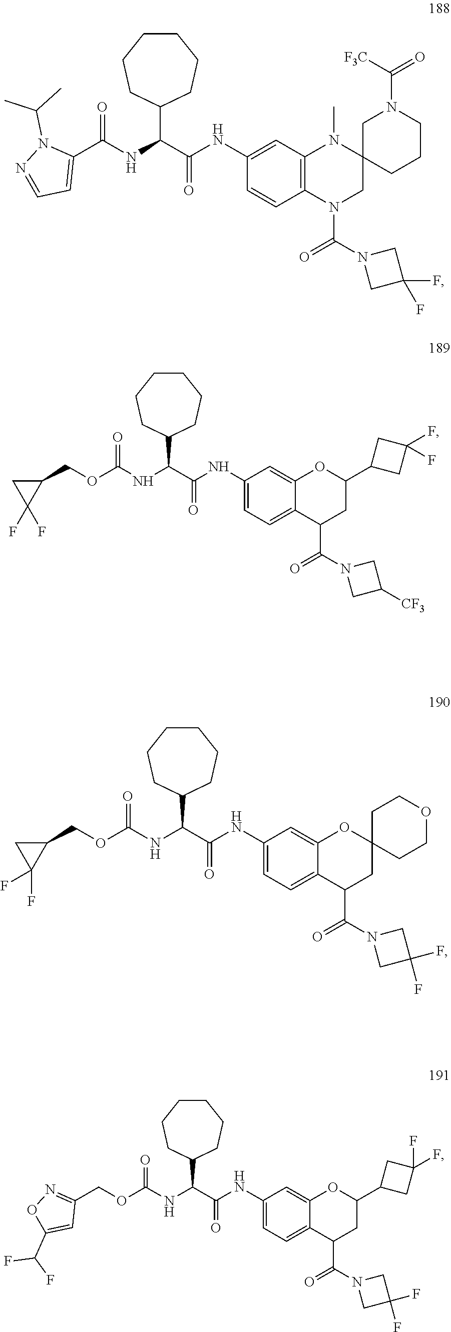 [SMILES: CC(C)n1nccc1C(=O)N[C@H](C(=O)Nc1ccc2c(c1)N(C)C1(CCCN(C(=O)C(F)(F)F)C1)CN2C(=O)N1CC(F)(F)C1)C1CCCCCC1 ; O=C(N[C@H](C(=O)Nc1ccc2c(c1)OC(C1CC(F)(F)C1)CC2C(=O)N1CC(C(F)(F)F)C1)C1CCCCCC1)OC[C@@H]1CC1(F)F ; O=C(N[C@H](C(=O)Nc1ccc2c(c1)OC1(CCOCC1)CC2C(=O)N1CC(F)(F)C1)C1CCCCCC1)OC[C@@H]1CC1(F)F ; O=C(N[C@H](C(=O)Nc1ccc2c(c1)OC(C1CC(F)(F)C1)CC2C(=O)N1CC(F)(F)C1)C1CCCCCC1)OCc1cc(C(F)F)on1]

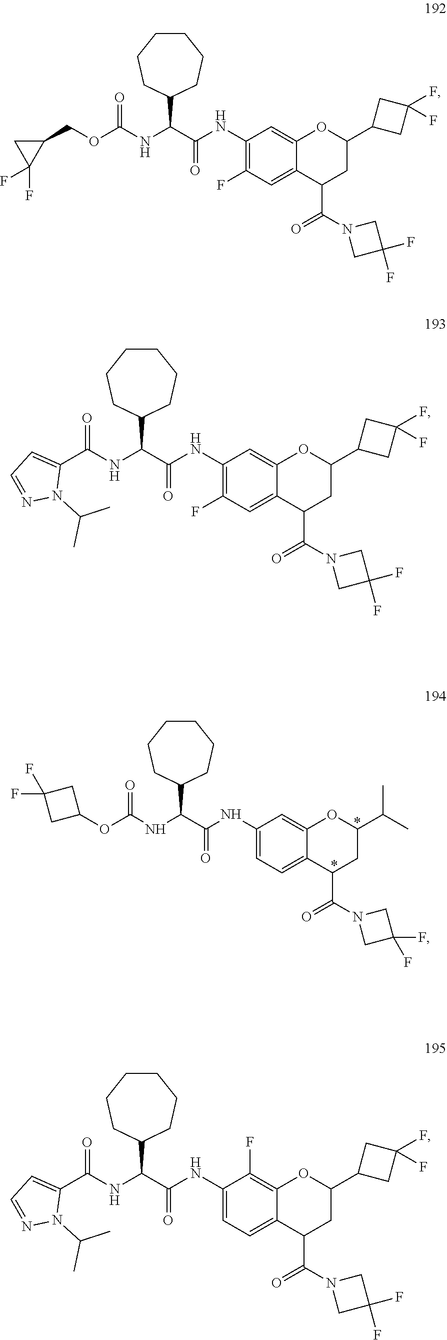 [SMILES: O=C(N[C@H](C(=O)Nc1cc2c(cc1F)C(C(=O)N1CC(F)(F)C1)CC(C1CC(F)(F)C1)O2)C1CCCCCC1)OC[C@@H]1CC1(F)F ; CC(C)n1nccc1C(=O)N[C@H](C(=O)Nc1cc2c(cc1F)C(C(=O)N1CC(F)(F)C1)CC(C1CC(F)(F)C1)O2)C1CCCCCC1 ; CC(C)C1CC(C(=O)N2CC(F)(F)C2)c2ccc(NC(=O)[C@@H](NC(=O)OC3CC(F)(F)C3)C3CCCCCC3)cc2O1 ; CC(C)n1nccc1C(=O)N[C@H](C(=O)Nc1ccc2c(c1F)OC(C1CC(F)(F)C1)CC2C(=O)N1CC(F)(F)C1)C1CCCCCC1]

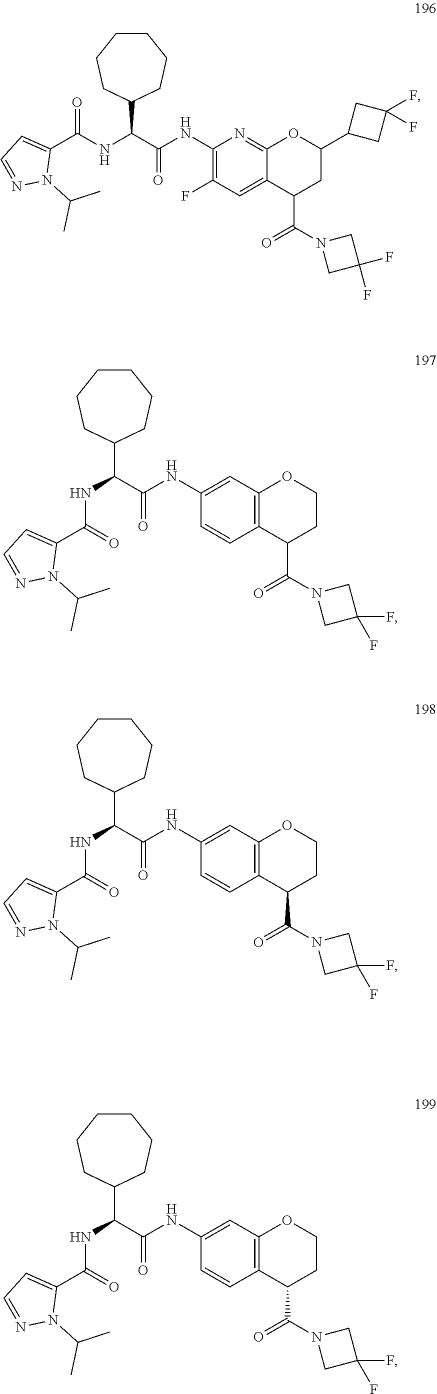 [SMILES: CC(C)n1nccc1C(=O)N[C@H](C(=O)Nc1nc2c(cc1F)C(C(=O)N1CC(F)(F)C1)CC(C1CC(F)(F)C1)O2)C1CCCCCC1 ; CC(C)n1nccc1C(=O)N[C@H](C(=O)Nc1ccc2c(c1)OCCC2C(=O)N1CC(F)(F)C1)C1CCCCCC1 ; CC(C)n1nccc1C(=O)N[C@H](C(=O)Nc1ccc2c(c1)OCC[C@H]2C(=O)N1CC(F)(F)C1)C1CCCCCC1 ; CC(C)n1nccc1C(=O)N[C@H](C(=O)Nc1ccc2c(c1)OCC[C@@H]2C(=O)N1CC(F)(F)C1)C1CCCCCC1]

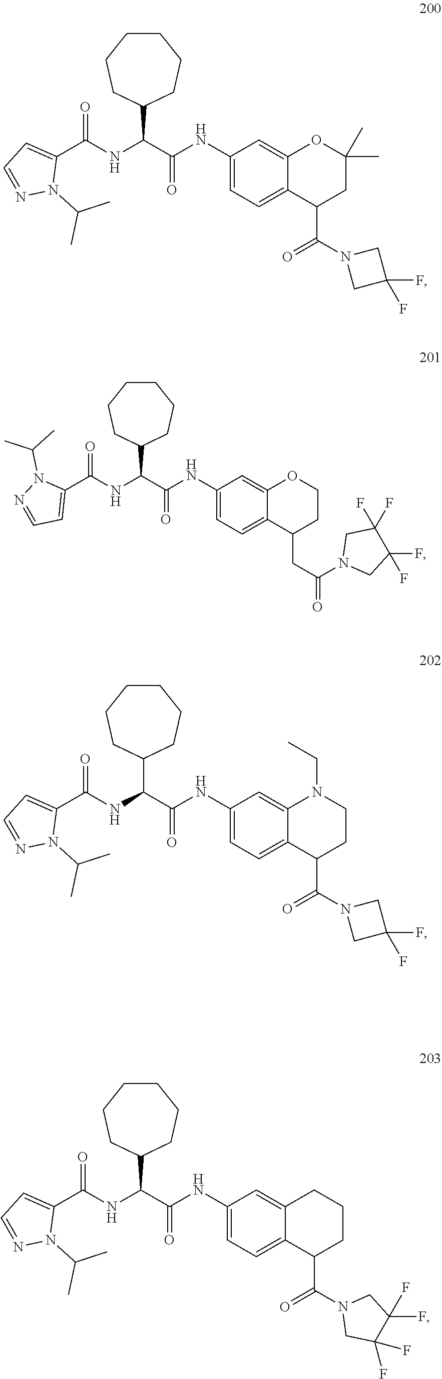 [SMILES: CC(C)n1nccc1C(=O)N[C@H](C(=O)Nc1ccc2c(c1)OC(C)(C)CC2C(=O)N1CC(F)(F)C1)C1CCCCCC1 ; CC(C)n1nccc1C(=O)N[C@H](C(=O)Nc1ccc2c(c1)OCCC2CC(=O)N1CC(F)(F)C(F)(F)C1)C1CCCCCC1 ; CCN1CCC(C(=O)N2CC(F)(F)C2)c2ccc(NC(=O)[C@@H](NC(=O)c3ccnn3C(C)C)C3CCCCCC3)cc21 ; CC(C)n1nccc1C(=O)N[C@H](C(=O)Nc1ccc2c(c1)CCCC2C(=O)N1CC(F)(F)C(F)(F)C1)C1CCCCCC1]

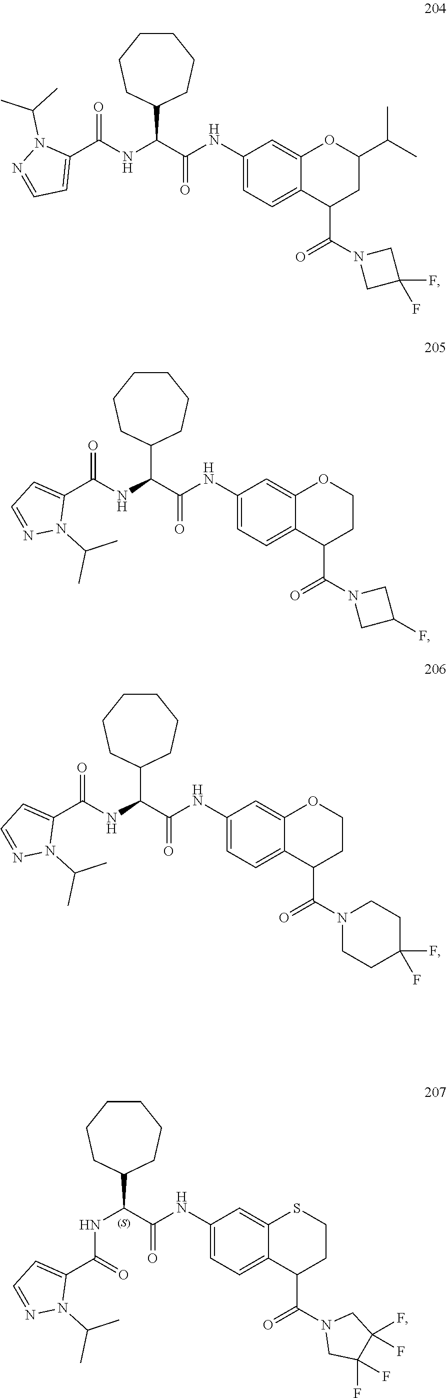

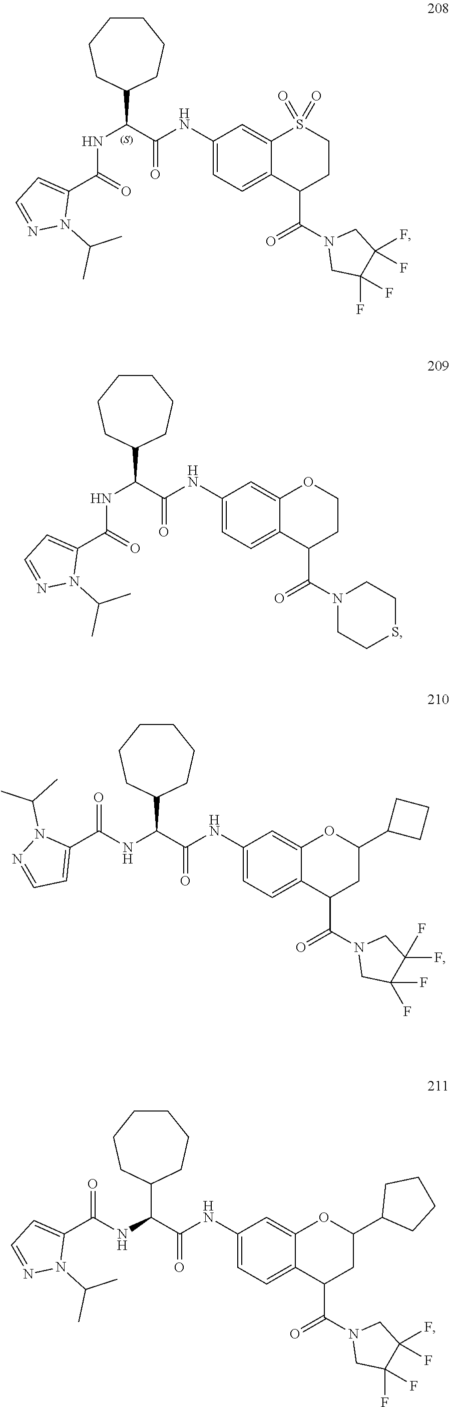

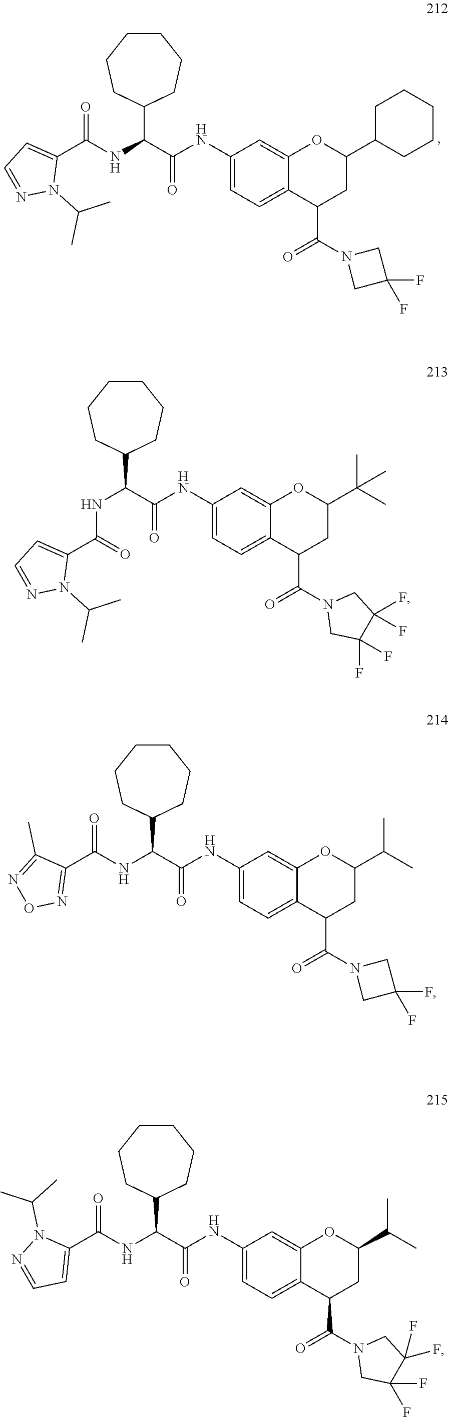 [SMILES: CC(C)n1nccc1C(=O)N[C@H](C(=O)Nc1ccc2c(c1)OC(C1CCCCC1)CC2C(=O)N1CC(F)(F)C1)C1CCCCCC1 ; CC(C)n1nccc1C(=O)N[C@H](C(=O)Nc1ccc2c(c1)OC(C(C)(C)C)CC2C(=O)N1CC(F)(F)C(F)(F)C1)C1CCCCCC1 ; Cc1nonc1C(=O)N[C@H](C(=O)Nc1ccc2c(c1)OC(C(C)C)CC2C(=O)N1CC(F)(F)C1)C1CCCCCC1 ; CC(C)[C@H]1C[C@@H](C(=O)N2CC(F)(F)C(F)(F)C2)c2ccc(NC(=O)[C@@H](NC(=O)c3ccnn3C(C)C)C3CCCCCC3)cc2O1]

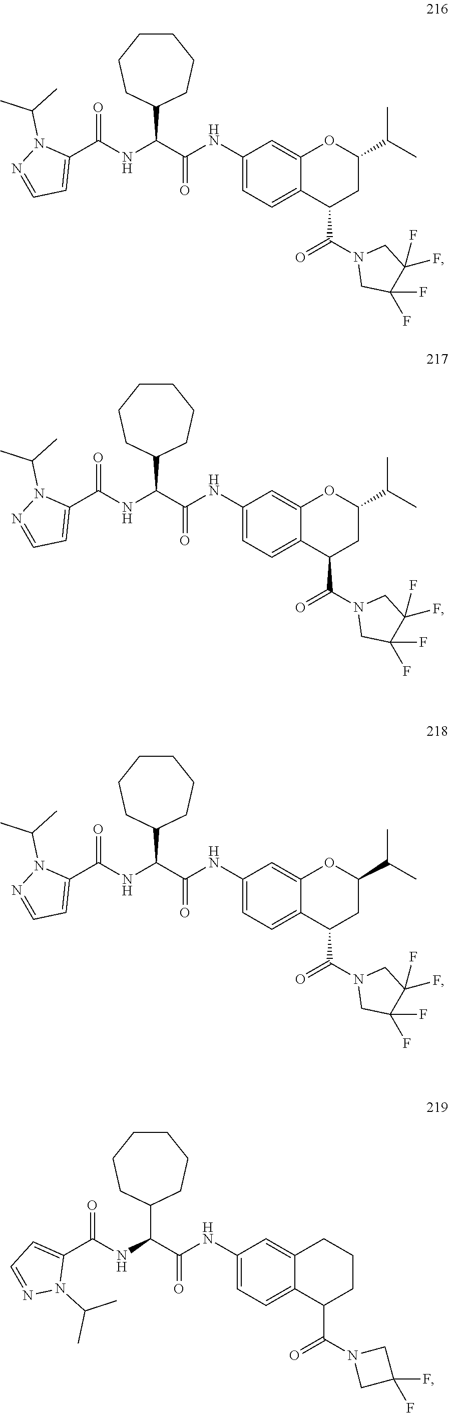 [SMILES: CC(C)[C@@H]1C[C@H](C(=O)N2CC(F)(F)C(F)(F)C2)c2ccc(NC(=O)[C@@H](NC(=O)c3ccnn3C(C)C)C3CCCCCC3)cc2O1 ; CC(C)[C@@H]1C[C@@H](C(=O)N2CC(F)(F)C(F)(F)C2)c2ccc(NC(=O)[C@@H](NC(=O)c3ccnn3C(C)C)C3CCCCCC3)cc2O1 ; CC(C)[C@H]1C[C@H](C(=O)N2CC(F)(F)C(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)CCCC2C(=O)N1CC(F)(F)C1)C1CCCCCC1]

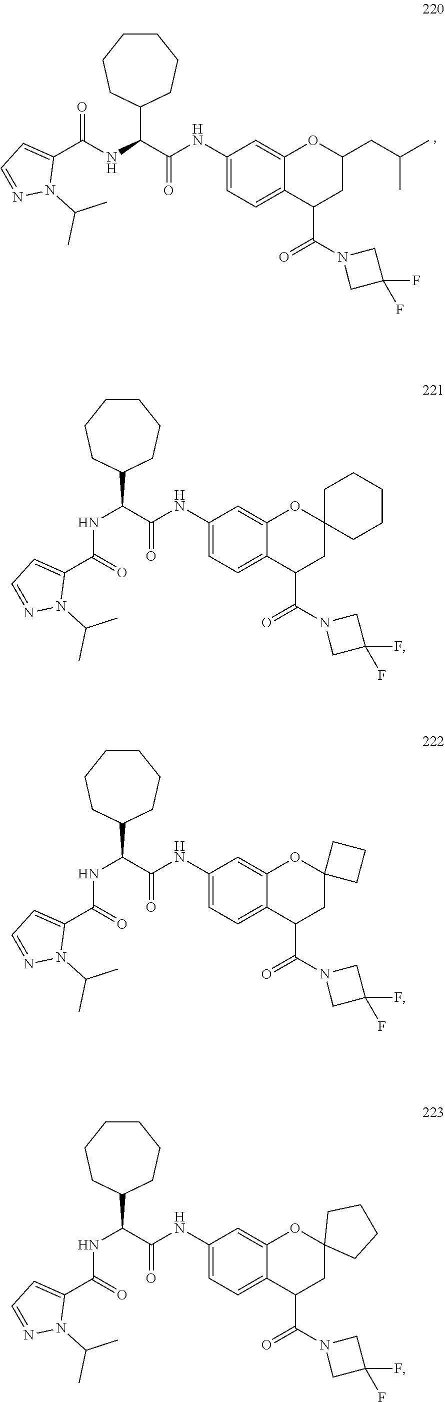 [SMILES: CC(C)CC1CC(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCCCC1)CC2C(=O)N1CC(F)(F)C1)C1CCCCCC1 ; CC(C)n1nccc1C(=O)N[C@H](C(=O)Nc1ccc2c(c1)OC1(CCC1)CC2C(=O)N1CC(F)(F)C1)C1CCCCCC1 ; CC(C)n1nccc1C(=O)N[C@H](C(=O)Nc1ccc2c(c1)OC1(CCCC1)CC2C(=O)N1CC(F)(F)C1)C1CCCCCC1]

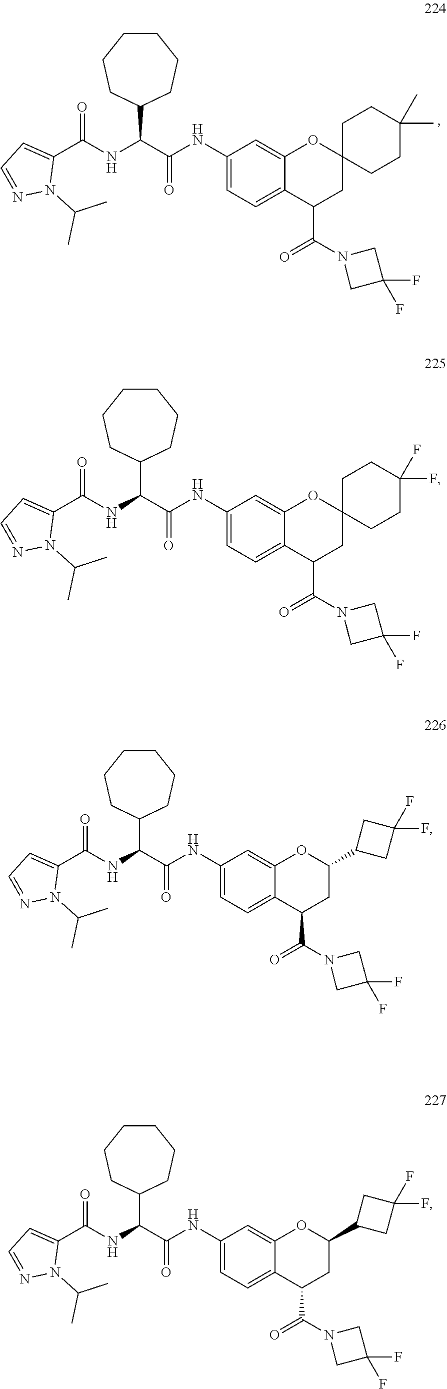 [SMILES: CC(C)n1nccc1C(=O)N[C@H](C(=O)Nc1ccc2c(c1)OC1(CCC(C)(C)CC1)CC2C(=O)N1CC(F)(F)C1)C1CCCCCC1 ; CC(C)n1nccc1C(=O)N[C@H](C(=O)Nc1ccc2c(c1)OC1(CCC(F)(F)CC1)CC2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1]

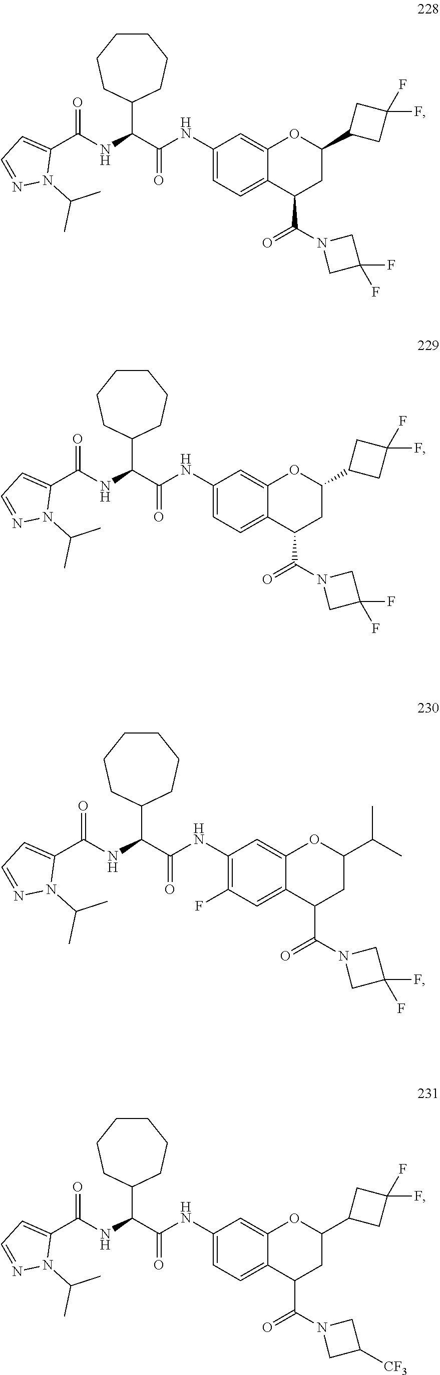 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1 ; CC(C)C1CC(C(=O)N2CC(F)(F)C2)c2cc(F)c(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC(C1CC(F)(F)C1)CC2C(=O)N1CC(C(F)(F)F)C1)C1CCCCCC1]

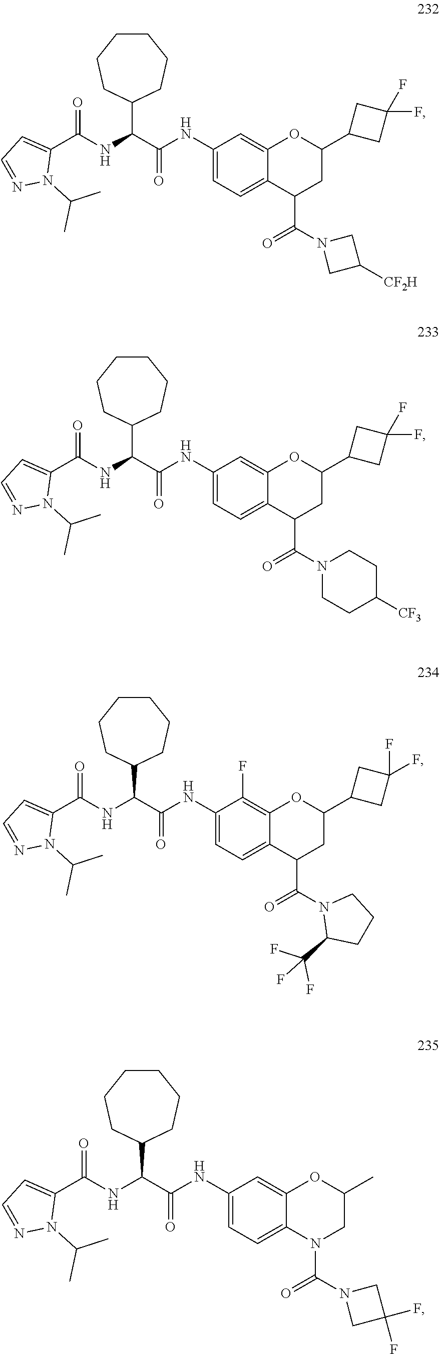 [SMILES: CC(C)n1nccc1C(=O)N[C@H](C(=O)Nc1ccc2c(c1)OC(C1CC(F)(F)C1)CC2C(=O)N1CC(C(F)F)C1)C1CCCCCC1 ; CC(C)n1nccc1C(=O)N[C@H](C(=O)Nc1ccc2c(c1)OC(C1CC(F)(F)C1)CC2C(=O)N1CCC(C(F)(F)F)CC1)C1CCCCCC1 ; CC(C)n1nccc1C(=O)N[C@H](C(=O)Nc1ccc2c(c1F)OC(C1CC(F)(F)C1)CC2C(=O)N1CCC[C@H]1C(F)(F)F)C1CCCCCC1 ; CC1CN(C(=O)N2CC(F)(F)C2)c2ccc(NC(=O)[C@@H](NC(=O)c3ccnn3C(C)C)C3CCCCCC3)cc2O1]

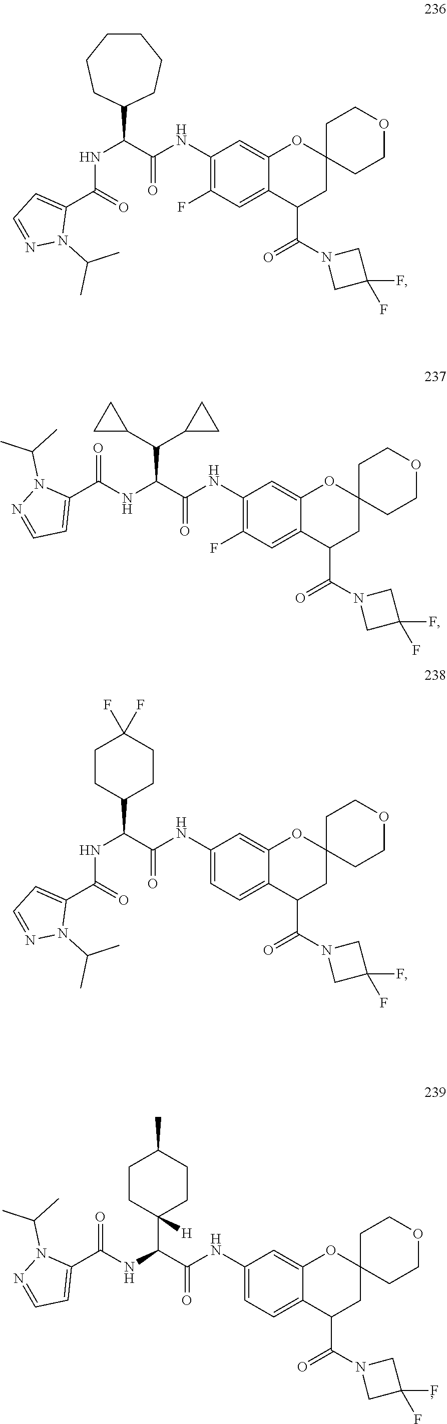 [SMILES: CC(C)n1nccc1C(=O)N[C@H](C(=O)Nc1cc2c(cc1F)C(C(=O)N1CC(F)(F)C1)CC1(CCOCC1)O2)C1CCCCCC1 ; CC(C)n1nccc1C(=O)N[C@H](C(=O)Nc1cc2c(cc1F)C(C(=O)N1CC(F)(F)C1)CC1(CCOCC1)O2)C(C1CC1)C1CC1 ; CC(C)n1nccc1C(=O)N[C@H](C(=O)Nc1ccc2c(c1)OC1(CCOCC1)CC2C(=O)N1CC(F)(F)C1)C1CCC(F)(F)CC1 ; CC(C)n1nccc1C(=O)N[C@H](C(=O)Nc1ccc2c(c1)OC1(CCOCC1)CC2C(=O)N1CC(F)(F)C1)[C@H]1CC[C@H](C)CC1]

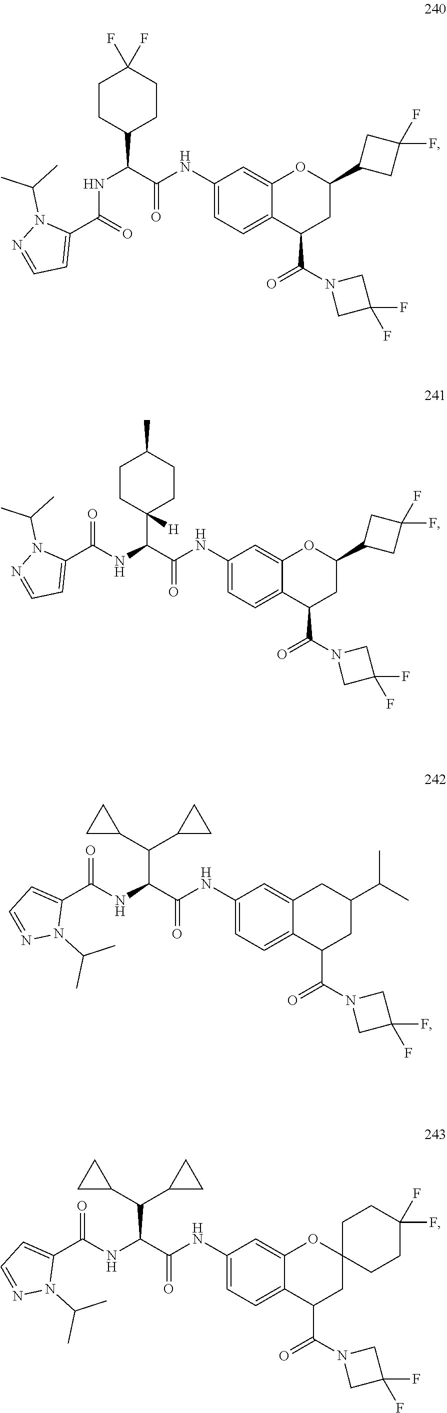 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCC(F)(F)CC1 ; CC(C)n1nccc1C(=O)N[C@H](C(=O)Nc1ccc2c(c1)O[C@@H](C1CC(F)(F)C1)C[C@H]2C(=O)N1CC(F)(F)C1)[C@H]1CC[C@H](C)CC1 ; CC(C)C1Cc2cc(NC(=O)[C@@H](NC(=O)c3ccnn3C(C)C)C(C3CC3)C3CC3)ccc2C(C(=O)N2CC(F)(F)C2)C1 ; CC(C)n1nccc1C(=O)N[C@H](C(=O)Nc1ccc2c(c1)OC1(CCC(F)(F)CC1)CC2C(=O)N1CC(F)(F)C1)C(C1CC1)C1CC1]

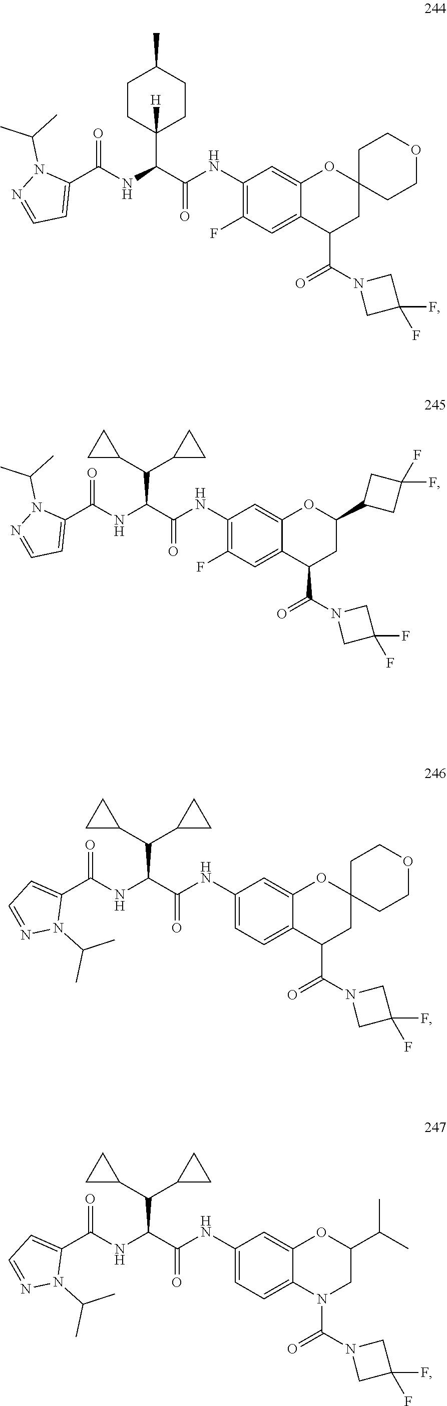

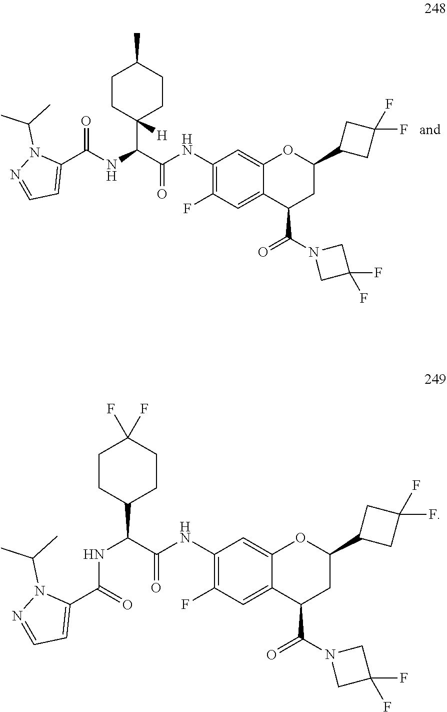 [SMILES: CC(C)n1nccc1C(=O)N[C@H](C(=O)Nc1cc2c(cc1F)[C@H](C(=O)N1CC(F)(F)C1)C[C@H](C1CC(F)(F)C1)O2)[C@H]1CC[C@H](C)CC1 ; CC(C)n1nccc1C(=O)N[C@H](C(=O)Nc1cc2c(cc1F)[C@H](C(=O)N1CC(F)(F)C1)C[C@H](C1CC(F)(F)C1)O2)C1CCC(F)(F)CC1]

### III. Pharmaceutical Compositions

Another aspect of the present application relates to a pharmaceutical composition that contains a compound of the present application. In some embodiments, the present application provides a pharmaceutical composition comprising a compound of the present application, and one or more pharmaceutically acceptable excipients.

In some embodiments, the pharmaceutical composition comprises a compound of Formula I or a pharmaceutically acceptable salt thereof.

In some embodiments, the pharmaceutical composition of the present application further comprises one or more additional therapeutic agents.

Pharmaceutical compositions of the present application may be in any form suitable for the intended method of administration. In some embodiments, the pharmaceutical composition is in the form of tablets, sachets, troches, lozenges, aqueous or oil suspensions, dispersible powders or granules, emulsions, hard or soft capsules, syrups or elixirs for oral administration.

In some embodiments, the pharmaceutical compositions of the present application are presented in unit dosage form, including but not limited to, capsules, sachets or tablets each containing a predetermined amount of the active ingredient. In one embodiment, the pharmaceutical composition is a tablet.

The pharmaceutical composition may be prepared by any of the methods well known in the art of pharmacy. Such methods include the step of bringing into association the active ingredient (e.g., a compound of the present application or a pharmaceutical salt thereof) with the one or more pharmaceutically acceptable excipients. In some embodiments, the pharmaceutical composition is prepared by uniformly and intimately bringing into association the active ingredient with one or more liquid excipients or one or more finely divided solid excipients or both to form a mixture, and then, if desired, shaping the mixture into a final product. Techniques and formulations generally are found in Remington: The Science and Practice of Pharmacy, 21st Edition, Lippincott Wiliams and Wilkins, Philadelphia, Pa., 2006.

Examples of the one or more excipients include, but not limited to, fillers, binders, bulking agent, glidands, sweetening agents, flavoring agents, coloring agents and preserving agents. In some embodiments, the pharmaceutical composition is in tablet form and comprises one or more pharmaceutically acceptable excipients which are suitable for manufacture of tablets. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the mode of administration. For example, in some embodiments, a dosage form for oral administration to humans may contain approximately 1 to 1000 mg of active material formulated with an appropriate and convenient amount of a pharmaceutically acceptable excipient. In some embodiments, the pharmaceutically acceptable excipient varies from about 5% to about 95% wt/wt of the total compositions.

In some embodiments, the pharmaceutical composition of the present application does not contain an agent that affects the rate at which the active ingredient is metabolized. In some embodiments, the pharmaceutical composition of the present application does not comprise an agent that would negatively affect (e.g., slow, hinder or retard) the metabolism of a compound of the present application or any other active ingredient administered separately, sequentially or simultaneously with a compound of the present application. It is also understood that any of the methods, kits, articles of manufacture and the like detailed herein do not comprise an agent that would negatively affect (e.g., slow, hinder or retard) the metabolism of a compound of the present application or any other active ingredient administered separately, sequentially or simultaneously with a compound of the present application.

### IV. Methods of Treatment

Another aspect of the present application relates to a method for preventing, treating or ameliorating a symptom of an IL-17A mediated inflammatory syndrome, disorder, or disease with the compound of the present application. In some embodiments, the method comprises the step of administering to a subject in need of such treatment an effective amount of a compound of Formula I, a pharmaceutically acceptable salt thereof, a deuterium substitute thereof, or an isomer thereof.

### Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of Formula I, or a pharmaceutically acceptable salt thereof, e.g., compounds having structures described by one or more of Formula I, or other formulas or compounds disclosed herein, may be accomplished as described in the following examples.

### General Syntheses

Typical embodiments of compounds in accordance with the present application may be synthesized using the general reaction schemes and/or examples described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Starting materials are typically obtained from commercial sources or synthesized using published methods for synthesizing compounds which are embodiments of the present application, inspection of the structure of the compound to be synthesized will provide the identity of each substituent group The identity of the final product will generally render apparent the identity of the necessary starting materials by a simple process of inspection, given the examples herein. Group labels (e.g., R1, R2) used in the reaction schemes herein are for illustrative purposes only and unless otherwise specified do not necessarily match by name or function the labels used elsewhere to describe compounds of Formula I or aspects or fragments thereof.

### Synthetic Reaction Parameters

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of the present application may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), N, N-dimethylformamide (“DMF”), chloroform, methylene chloride (or dichloromethane), diethyl ether, methanol, pyridine and the like). Unless specified to the contrary, the solvents used in the reactions of the present application are inert organic solvents, and the reactions are carried out under an inert gas, preferably nitrogen.

The term “q.s.” means adding a quantity sufficient to achieve a stated function, e.g., to bring a solution to the desired volume (i.e., 100%).

Compounds as provided herein may be synthesized according to the general schemes provided below. In the Schemes below, it should be appreciated that each of the compounds shown therein may have protecting groups as required present at any step. Standard protecting groups are well within the purview of one skilled in the art.

### EXAMPLES

### Example 1: Preparation of Compounds

In another aspect, the present invention provides a method for preparing a compound of Formula I, a pharmaceutically acceptable salt, an ester or a stereoisomer thereof, the method comprising nine general routes (Route 1, Route 2, Route 3, Route 4, Route 5, Route 6, Route 7, Route 8, Route 9, Route 10, Route 11, Route 12, Route 13, Route 14 and Route 15):

### Route 1. Chiral Alpha-Methylbenzylamine Assisted Asymmetric Synthesis of Compound 1

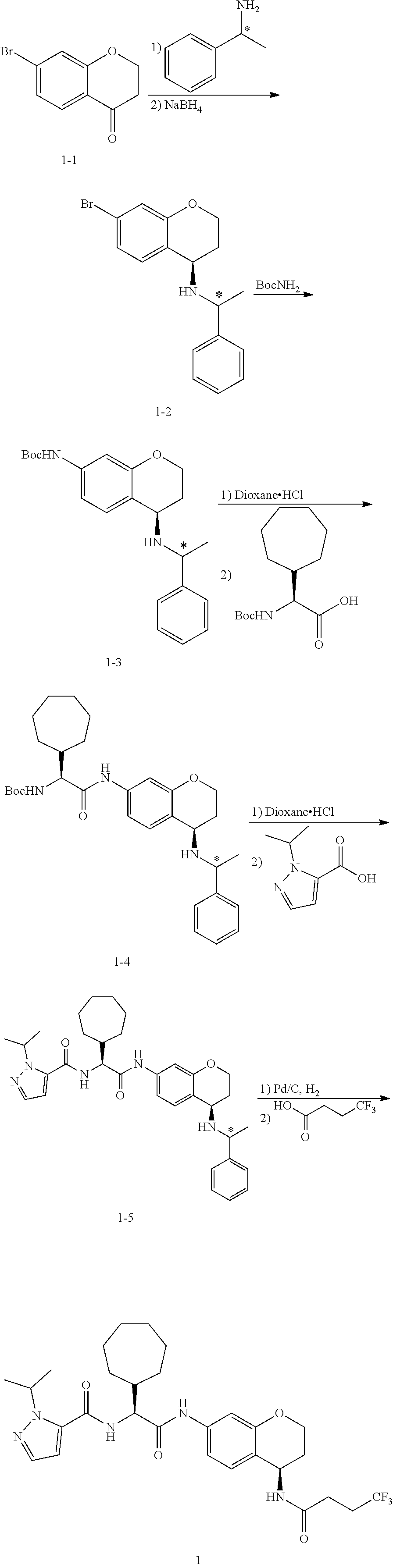

### Synthesis of Compound 1-2

A THF solution of compound 1-1 (1.0 equiv.) and alpha-methylbenzylamine (1.2 equiv.) were stirred with Ti(OEt)₄ (3.0 equiv.) at 85° C. to reach a full conversion. Then NaBH₄ (1.2 equiv.) was added into the abovementioned solution at −40° C., and stirred at 25° C. to reach a full conversion. The reaction was quenched with deionized water and extracted with EtOAc. The organic phase was acidified with HCl and concentrated at reduce pressure to obtain a white crystal, which was washed with EA-MeOH (10:1) solution to give an optional pure compound 1-2.

### Synthesis of Compound 1-3

Compound 1-2 (1.0 equiv.), BocNH₂ (1.5 equiv.), Pd₂(dba)₃ (5 mol %), X-Phos (20 mol %), Cs₂CO₃ (3.0 equiv.) were added into the dioxane under nitrogen, and stirred for 3 hours at 90-100° C. After cooling, EtOAc and water were added into the mixture solution and stirred for 10 mins. Then the organic phase was separated and washed with water and brine, dried over MgSO₄ and concentrated in vacuo. The concentrated product was purified by column chromatography on silica gel to obtain compound 1-3.

### Synthesis of Compound 1-4

Compound 1-3 was dissolved in the least amount of EtOAc, then dioxane-HCl was added and stirred at 25° C. for 2 hours. Then the reaction solvent was removed at reduced pressure to give an ammonia hydrochloride salt with quantitative yield. This salt was used directly without further purification. (2S)-2-{[(tert-butoxy)carbonyl]amino}-2-cycloheptylacetic acid (1.0 equiv.), HATU (1.5 equiv.) and TEA (1.5 equiv.) were added into DCM (1M), and stirred for 1 hours at 20-25° C. the abovementioned salt (1.0 equiv.) was added and stirred for 2 hours. Water was added and stirred for 15 mins. The organic layer was separated, washed with water and brine, dried over MgSO₄ and concentrated in vacuo to produce compound 1-4.

### Synthesis of Compound 1-5

Compound 1-4 was dissolved in the least amount of EtOAc, then dioxane HCl was added and stirred at 25° C. for 2 hours. Then the reaction solvent was removed at reduced pressure to give an ammonia hydrochloride salt with quantitative yield. This salt was used directly without further purification. HATU (1.5 equiv.) and TEA (1.5 equiv.) were added to a solution of 1-isopropyl-1H-pyrazole-5-carboxylic acid (1.0 equiv.) in DCM (1 M) and stirred for 30 mins at room temperature. The abovementioned salt (1.0 equiv.) was added into the mixture reaction and kept stirring for 2 hours. Water was added and stirred for 10 mins. The organic phase was separated, washed with water and brine, dried over MgSO₄ and concentrated in vacuo to produce compound 1-5.

### Synthesis of Compound 1

Compound 1-5 (1.0 equiv.) underwent Pd/C catalyzed hydrolysis to generate a primary ammonia, then it was mixed with 4,4,4-trifluorobutyric acid and HATU (1.5 equiv.) and TEA (1.5 equiv.) in DCM (1 M) and kept stirring for 2 hours at room temperature. Water was added and stirred for 10 mins. The organic phase was separated, washed with water and brine, dried over MgSO₄ and concentrated in vacuo to produce compound 1.

### Route 2. Chiral Sulfinylamide Assisted Asymmetric Synthesis of Compound 1

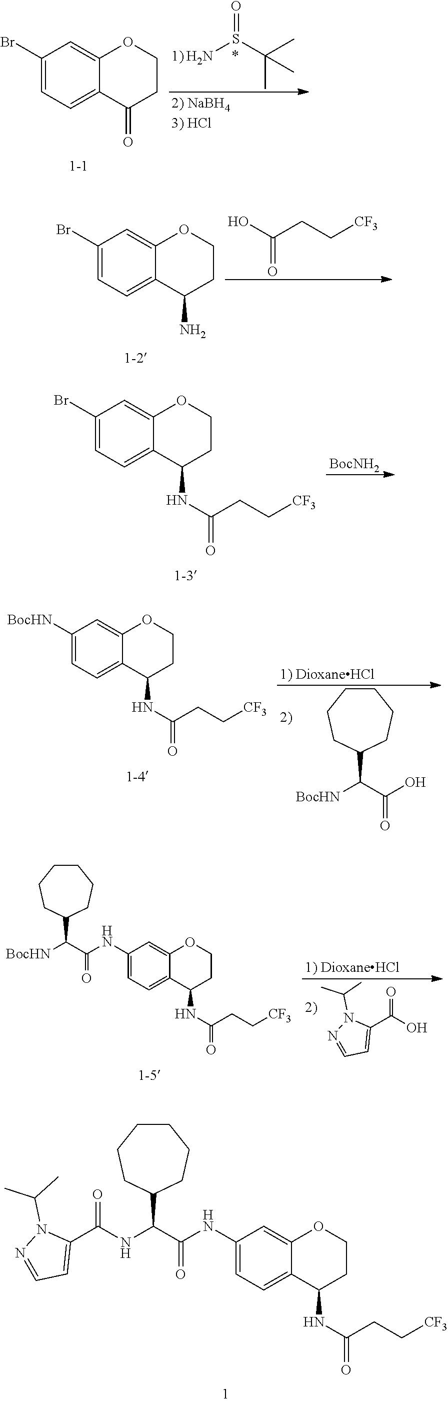

### Synthesis of Compound 1-2′

A THF solution of 1-1 (1.0 equiv.) and chiral sulfinylamide (1.2 equiv.) was stirred with Ti(OEt)₄ (3.0 equiv.) at 85° C. to reach a full conversion. Then NaBH₄ (1.2 equiv.) was added into the above-mentioned solution at −40° C., and stirred at 25° C. to reach a full conversion. The reaction was quenched with deionized water and extracted with EtOAc. The organic phase was acidified with HCl and concentrated at reduced pressure to obtain a white crystal compound 1-2′ (98% yield).

### Synthesis of Compound 1-3′

Compound 1-2′ was mixed with 4,4,4-trifluorobutyric acid and HATU (1.5 equiv.) and TEA (1.5 equiv.) in DCM (1 M) and stirred for 2 hours at room temperature. Water was added and stirred for 10 mins. The organic phase was separated, washed with water and brine, dried over MgSO₄ and concentrated in vacuo to produce compound 1-3′.

### Synthesis of Compound 1-4′

Compound 1-3′ (1.0 equiv.), BocNH₂ (1.5 equiv.), Pd₂(dba)₃ (10 mol %), X-Phos (20 mol %), Cs₂CO₃ (3.0 equiv.) were added into the dioxane under nitrogen, and stirred for 3 hours at 90-100° C. After cooling, EtOAc and water were added into the mixture solution and stirred for 10 mins. Then the organic phase was separated, washed with water and brine, dried over MgSO₄ and concentrated in vacuo. The concentrated product was purified by column chromatography on silica gel to produce compound 1-4′.

### Synthesis of Compound 1-5′

Compound 1-4′ was dissolved in the least amount of EtOAc, then dioxane HCl was added and stirred at 25° C. for 2 hours. Then the reaction solvent was removed at reduced pressure to give an ammonia hydrochloride salt with quantitative yield. This salt was used directly without further purification. (2S)-2-{[(tert-butoxy)carbonyl]amino}-2-cycloheptylacetic acid (1.0 equiv.), HATU (1.5 equiv.) and TEA (1.5 equiv.) were added into DCM (1M), and stirred for 1 hour at 20-25° C. The abovementioned salt (1.0 equiv.) was added and stirred for 2 hours. Water was added and stirred for 15 mins. The organic layer was separated, washed with water and brine, dried over MgSO₄ and concentrated in vacuo to produce compound 1-5′.

### Synthesis of Compound 1

Compound 1-5′ was dissolved in the least amount of EtOAc, then dioxane HCl was added and stirred at 25° C. for 2 hours. Then the reaction solvent was removed at reduced pressure to give an ammonia hydrochloride salt with quantitative yield. This salt was used directly without further purification. HATU (1.5 equiv.) and TEA (1.5 equiv.) were added to a solution of 1-isopropyl-1H-pyrazole-5-carboxylic acid (1.0 equiv.) in DCM (1 M) and stirred for 30 mins at room temperature. The abovementioned salt (1.0 equiv.) was added into the mixture reaction and stirred for 2 hours. Water was added and stirred for 10 mins. The organic phase was separated, washed with water and brine, dried over MgSO₄ and concentrated in vacuo to produce compound 1.

### Route 3. Preparation of Intermediate 2-6

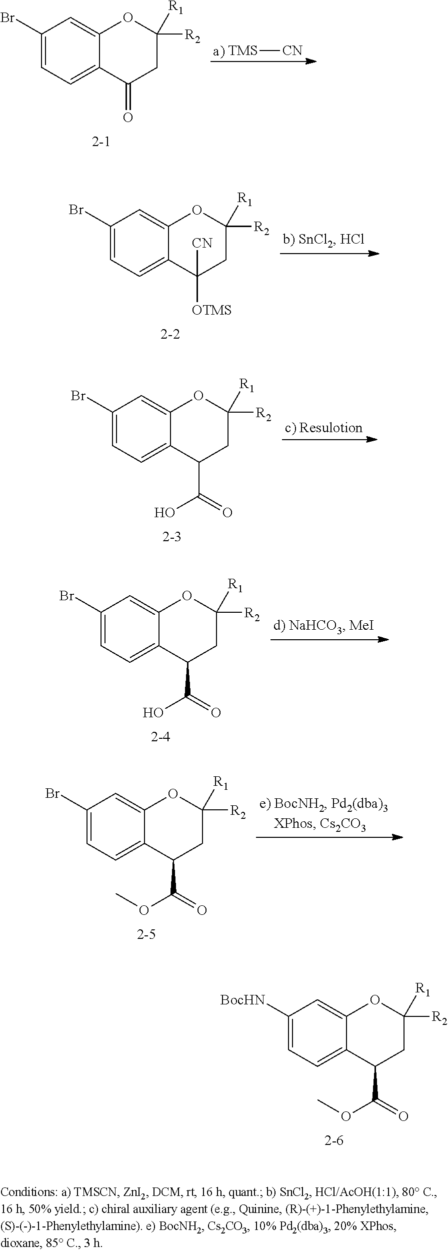

The following intermediates were prepared according to the procedures of intermediate 2-6:

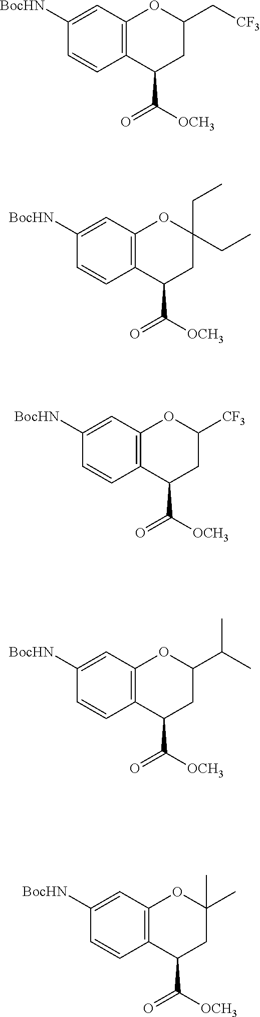 [SMILES: COC(=O)[C@@H]1CC(CC(F)(F)F)Oc2cc(NC(=O)OC(C)(C)C)ccc21 ; CCC1(CC)C[C@@H](C(=O)OC)c2ccc(NC(=O)OC(C)(C)C)cc2O1 ; COC(=O)[C@@H]1CC(C(F)(F)F)Oc2cc(NC(=O)OC(C)(C)C)ccc21 ; COC(=O)[C@@H]1CC(C(C)C)Oc2cc(NC(=O)OC(C)(C)C)ccc21 ; COC(=O)[C@@H]1CC(C)(C)Oc2cc(NC(=O)OC(C)(C)C)ccc21]

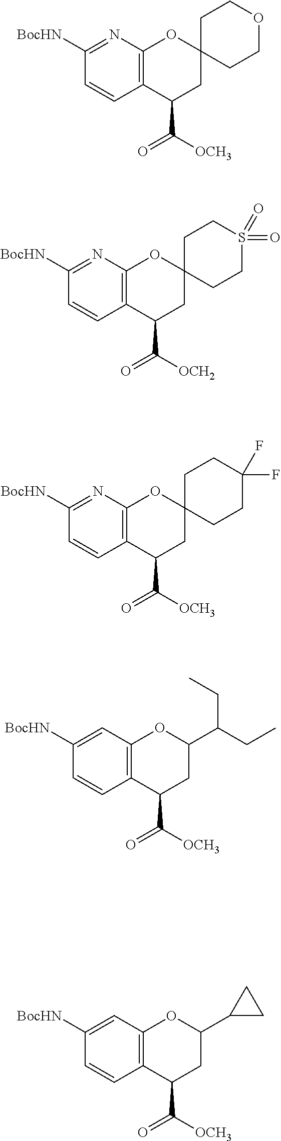

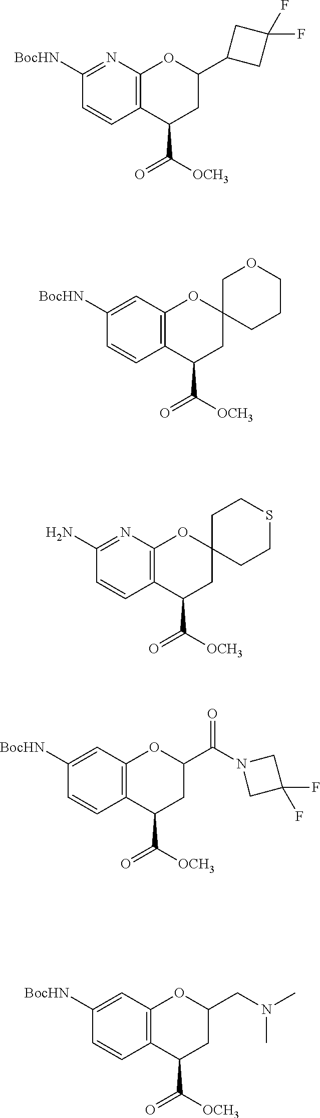 [SMILES: COC(=O)[C@@H]1CC(C2CC(F)(F)C2)Oc2nc(NC(=O)OC(C)(C)C)ccc21 ; COC(=O)[C@@H]1CC2(CCCOC2)Oc2cc(NC(=O)OC(C)(C)C)ccc21 ; COC(=O)[C@@H]1CC2(CCSCC2)Oc2nc(N)ccc21 ; COC(=O)[C@@H]1CC(C(=O)N2CC(F)(F)C2)Oc2cc(NC(=O)OC(C)(C)C)ccc21 ; COC(=O)[C@@H]1CC(CN(C)C)Oc2cc(NC(=O)OC(C)(C)C)ccc21]

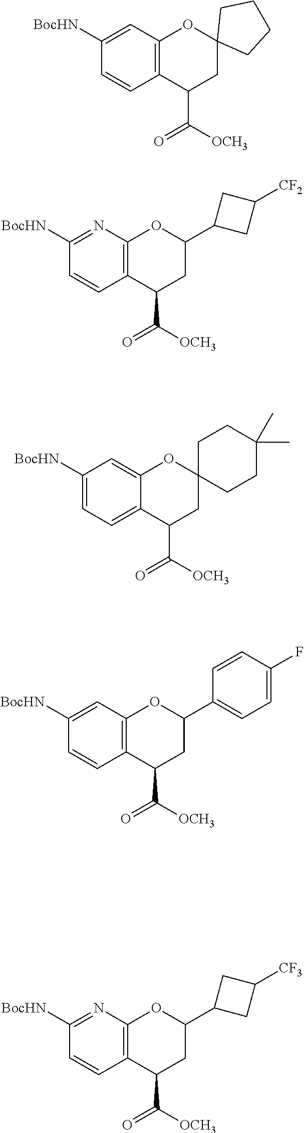

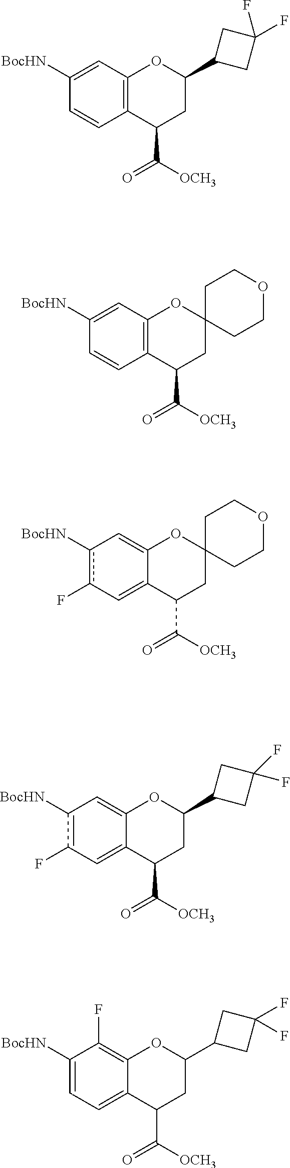

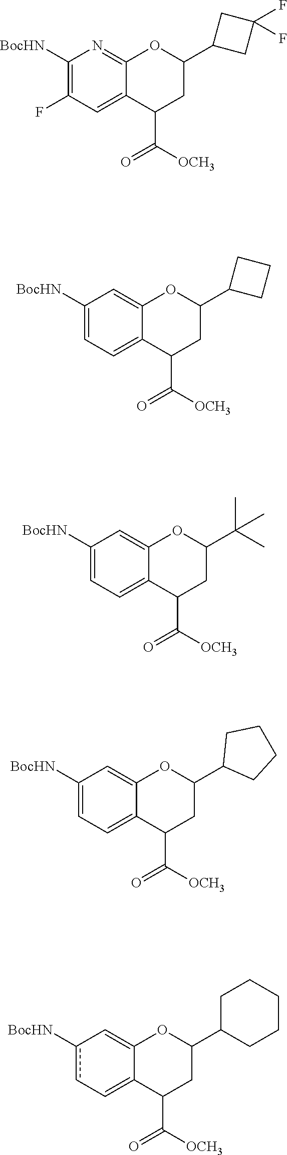

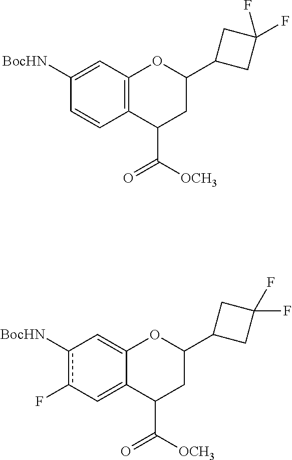

### Synthesis of Compound 2-2

A DCM solution of 2-1 (1.0 equiv.) and TMSCN (1.5 equiv.) was stirred with ZnI₂ (2 mol %) at RT for 16 h to reach a full conversion. The reaction mixture was washed with saturated aqueous sodium bicarbonate solution. The organic layer was dried (MgSO₄), and concentrated in vacuo to produce compound 2-2 (97% yield).

### Synthesis of Compound 2-3

A mixture of compound 2-2 (1.0 equiv) and SnCl₂·2H₂O (4.0 equiv) in a mixture of acetic acid and concentrated HCl solution (1:1) was refluxed overnight. The mixture was extracted with CH₂Cl₂. The combined organic phase was washed with 2N sodium hydroxide solution. The combined basic washes were extracted with ether, and subsequently acidified to pH=2 with 5N HCl solution. The acidic aqueous mixture was extracted with EA, and the combined organic layer was dried and concentrated in vacuo to produce compound 2-3 (85% yield).

### Synthesis of Compound 2-4

Compound 2-3 was treated with oxalyl chloride and DMF in DCM at room temperature, and a solution of Evans' auxiliary and TEA was added into the reaction flask. The resultant diastereomers were separated through silica gel column to produce compound 2-4.

### Synthesis of Compound 2-5

Compound 2-4 was dissolved in DMF, NaHCO₃ (1.2 equiv.) and Mel (1.5 equiv.) were then added to the reaction, and stirred at RT for 4 h to reach a full conversion. EA was added, and the organic phase was washed with NaHCO₃ solution, dried with MgSO₄, and concentrated in reduced pressure To produce compound 2-5 in quantitative yield.

### Synthesis of Compound 2-6

Compound 2-5 (1.0 equiv.), BocNH₂ (1.5 equiv.), Pd₂(dba)₃ (10 mol %), X-Phos (20 mol %), Cs₂CO₃ (3.0 equiv.) were added into dioxane under nitrogen, and stirred for 3 hours at 90˜100° C. After cooling, EtOAc and water were added into the mixture solution and stirred for 10 mins. Then the organic phase was separated, washed with water and brine, dried over MgSO₄ and concentrated in vacuo. The concentrated product was purified by column chromatography on silica gel to produce compound 2-6.

### Route 4. Preparation of Intermediate 3-4

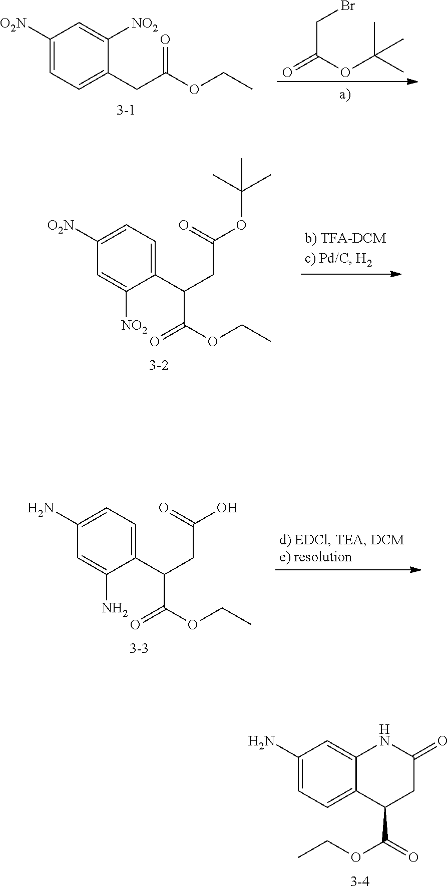

### Synthesis of Compound 3-2

A MeCN solution of compound 3-1 (1.0 equiv.) and tert-Butyl bromoacetate (1.2 equiv.) was stirred with Cs₂CO₃ (1.2 equiv.) at 60° C. for 2 h to reach a full conversion The reaction mixture was washed with 1N aqueous HCl solution. The organic layer was dried (MgSO₄), and concentrated in vacuo to produce compound 3-2 (97% yield).

### Synthesis of Compound 3-3

Compound 3-2 (1.0 equiv) was dissolved in a mixture of trifluoroacetic acid and CH₂Cl₂ (1:1) and stirred at room temperature for 2 hours. The mixture was extracted with CH₂Cl₂. The combined organic phase was washed with 2N sodium hydroxide solution. The combined basic washes were extracted with ether, and subsequently acidified to pH=2 with 5N HCl solution. The acidic aqueous mixture was extracted with EA, and the combined organic layer was added Pd/C (10 wt %, 10 mol %), charged with H₂ balloon and stirred at room temperature for 2 hours. Then the organic layer was filtrated, dried and concentrated in vacuo to produce compound 3-3 (99% yield).

### Synthesis of Compound 3-4

TEA (1.5 equiv.) was added to a solution of 3-3 (1.0 equiv.) in DCM (1 M) and stirred for 30 mins at room temperature. The reaction was quenched by adding EA. The organic phase was washed with 2N sodium hydroxide solution, then washed with water and brine, dried over MgSO₄ and concentrated in vacuo to produce alactam intermediate. The lactam was then separated by chiral HPLC with CHIRALPAK ODH to produce chiral compound 3-4.

### Route 5. Preparation of Intermediate 4-4

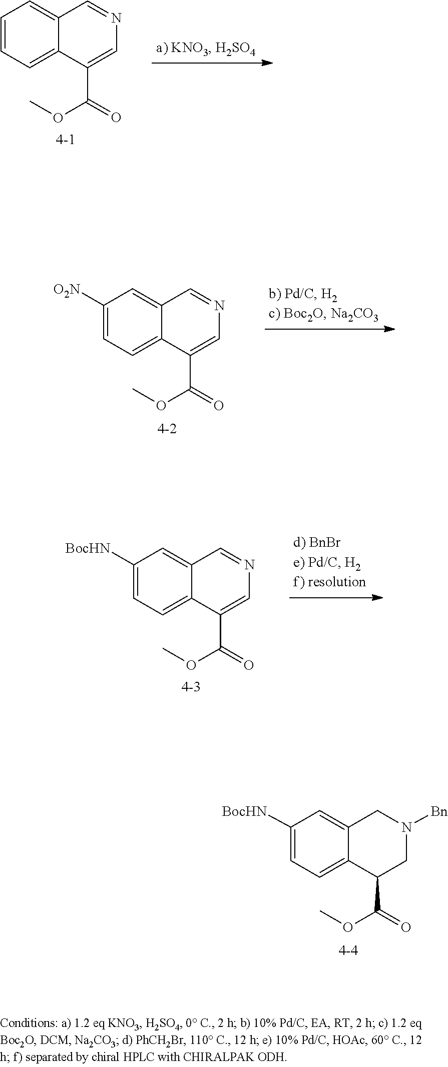 [SMILES: COC(=O)c1cncc2ccccc12 ; COC(=O)c1cncc2cc([N+](=O)[O-])ccc12 ; COC(=O)c1cncc2cc(NC(=O)OC(C)(C)C)ccc12 ; COC(=O)[C@@H]1CN(Cc2ccccc2)Cc2cc(NC(=O)OC(C)(C)C)ccc21]

### Synthesis of Compound 4-2

A H₂SO₄ solution of compound 4-1 (1.0 equiv.) was stirred at 0° C., and KNO₃ was added in portions to the solution for 2 h to reach a full conversion. The reaction was quenched with EA and Brine. The reaction mixture was washed with brine. The organic layer was dried (MgSO₄), and concentrated in vacuo to produce compound 4-2 (97% yield).

### Synthesis of Compound 4-3

Compound 4-2 (1.0 equiv) was dissolved in EA. Pd/C (10 wt %, 10 mol %) was added, charged with H2 balloon and stirred at room temperature for 0.5 hours. Then the organic layer was filtrated, dried and concentrated in vacuo to produce compound 4-3 (99% yield).

### Synthesis of Compound 4-4

A solution of compound 4-3 (1.0 equiv.) in BnBr (1.1 equiv.) was heated at 110° C. for 30 mins. The reaction mixture underwent solidification to reach a full conversion. The solid cake was washed with PE and used directly without further purification. The resultant was subsequently acidified to pH=2 with 5N HCl solution. Pd/C (10 wt %, 10 mol %) was added, charged with H₂ balloon and stirred at 60° C. for 2 hours. The organic layer was filtrated, dried and concentrated in vacuo to produce racemic compound 4-4 (99% yield). Then the lactam was separated by chiral HPLC with CHIRALPAK ODH to produce chiral compound 4-4.

### Route 6. Preparation of Intermediate 5-4

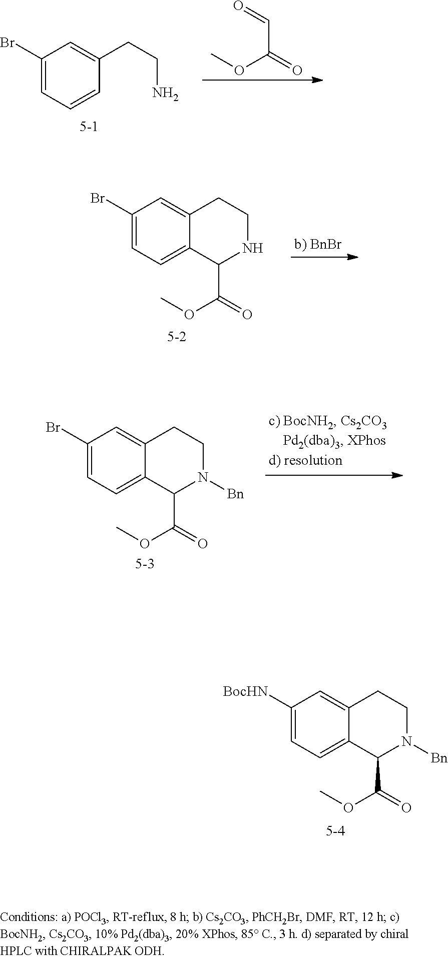

### Synthesis of Compound 5-2

Methyl 2-oxoacetate (1.0 equiv.) was added to a toluene solution of compound 5-1 (1.0 equiv.) and stirred at 80° C. for 2 hours, and POCl₃ (20 equiv.) was added and stirred at 120° C. for 8 hours. The reaction was quenched with EA and NaHCO₃ at 0° C. The reaction mixture was washed with brine. The organic layer was dried (MgSO₄), and concentrated in vacuo to produce compound 5-2 (77% yield).

### Synthesis of Compound 5-3

Compound 5-2 (1.0 equiv.) was dissolved in DMF, BnBr (1.0 equiv.) was added, and stirred with Cs2CO₃ (1.2 equiv.) at room temperature for 6 h to reach a full conversion. The reaction mixture was then quenched and extracted with EA, and washed with brine. The organic layer was dried (MgSO₄), and concentrated in vacuo to produce compound 5-3 (95% yield).

### Synthesis of Compound 5-4

Compound 5-3 (1.0 equiv.), BocNH₂ (1.5 equiv.), Pd₂(dba)₃ (5 mol %), X-Phos (20 mol %), Cs₂CO₃ (3.0 equiv.) were added into dioxane under nitrogen, and stirred for 3 hours at 90˜100° C. After cooling, EtOAc and water were added into the mixture solution and stirred for 10 mins. Then the organic phase was separated, washed with water and brine, dried over MgSO₄ and concentrated in vacuo. The concentrated product was purification by column chromatography on silica gel to produce racemic compound 5-4. The racemic compound 5-4 was separated by chiral HPLC with CHIRALPAK ODH to produce chiral compound 5-4.

### Route 7. Preparation of Compound 2-9, Int 3, Int 4, and Int 5

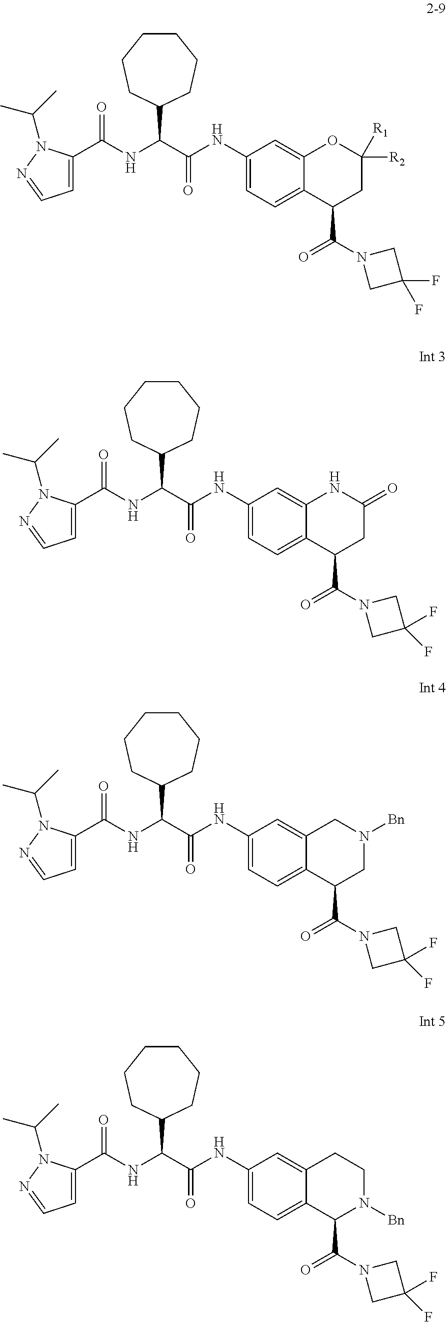

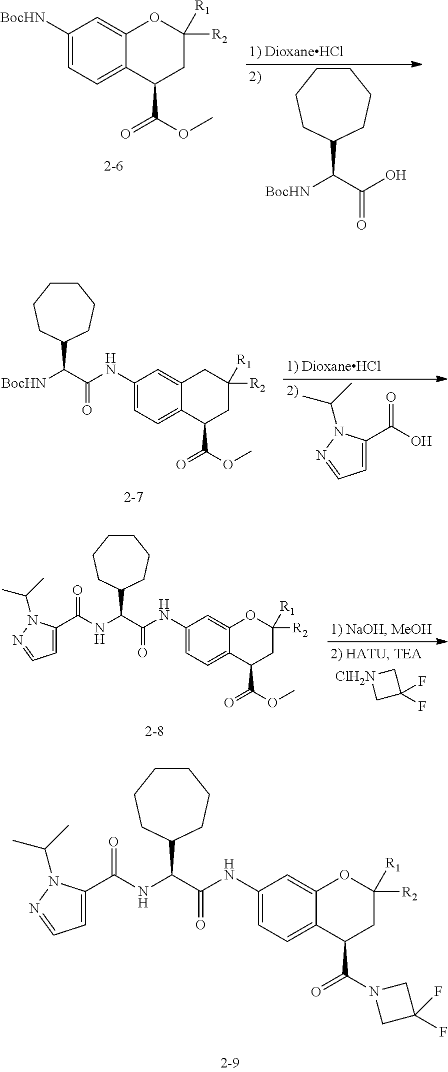

### Synthesis of Compound 2-7 series compound

Corresponding protected aromatic amine (2-6, 3-6, 4-4, 5-4) were individually dissolved in least amount of EtOAc, then dioxane-HCl was added and stirred at 25° C. for 2 hours. Then the reaction solvent and removed at reduce pressure to give ammonia hydrochloride salts with quantitative yield. This salt was used directly without further purification. (2S)-2-{[(tert-butoxy)carbonyl]amino}-2-cycloheptylacetic acid (1.0 equiv.), HATU (1.5 equiv.) and TEA (1.5 equiv.) were added into DCM (1M), and stirred for 1 hours at 20-25° C. The abovementioned salt (1.0 equiv.) was added and stirred for 2 hours. Water was added and stirred for 15 mins. The organic layers was separated, washed with water and brine, dried over MgSO₄ and concentrated in vacuo to produce corresponding N-protected amino amide (for example compound 2-7).

### Synthesis of Compound 2-8

Compound 2-7 was dissolved in the least amount of EtOAc, then dioxane-HCl was added and stirred at 25° C. for 2 hours. Then the reaction solvent and removed at reduced pressure to produce an ammonia hydrochloride salt with quantitative yield. This salt was used directly without further purification. HATU (1.5 equiv.) and TEA (1.5 equiv.) were added to a solution of 1-isopropyl-1H-pyrazole-5-carboxylic acid (1.0 equiv.) in DCM (1 M) and stirred for 30 mins at room temperature. The abovementioned salt (1.0 equiv.) was added into the mixture reaction and stirred for 2 hours. Water was added and stirred for 10 mins. The organic phase was separated, washed with water and brine, dried over MgSO₄ and concentrated in vacuo to produce compound 2-8.

### Synthesis of Compound 2-9, Int 3, Int 4, Int 5

3N NaOH (1 M) was added to a solution of compound 2-8 (1.0 equiv.) in MeOH (1 M) and stirred for 8 hours at room temperature. The reaction was quenched with deionized water. The quenched reaction mixture was extracted with ether, and subsequently acidified to pH=2 with 5N HCl solution. The acidic aqueous mixture was extracted with EA, and the combined organic layer was dried and concentrated in vacuo to produce a carboxyl acid. Then the carboxyl acid (1.0 equiv.), HATU (1.5 equiv.) 3,3-difluoroazetidine hydrochloric salt (1.1 equiv.) and TEA (1.5 equiv.) were added into DCM (1M), and stirred for 1 hours at 20-25° C. Water was added and stirred for 15 mins. The organic layers was separated, washed with water and brine, dried over MgSO₄ and concentrated in vacuo to produce compound 2-9 (85% yield). Int 3, int 4, and int 5 were synthesized in the same way.

### Route 8. Preparation of Int 3-1, and Int 3-2

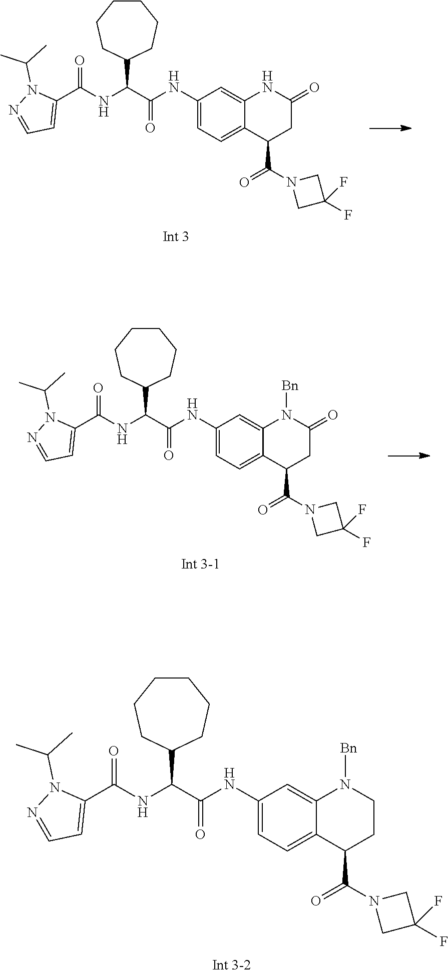 [SMILES: CC(C)n1nccc1C(=O)N[C@H](C(=O)Nc1ccc2c(c1)NC(=O)C[C@H]2C(=O)N1CC(F)(F)C1)C1CCCCCC1 ; CC(C)n1nccc1C(=O)N[C@H](C(=O)Nc1ccc2c(c1)N(Cc1ccccc1)C(=O)C[C@H]2C(=O)N1CC(F)(F)C1)C1CCCCCC1 ; CC(C)n1nccc1C(=O)N[C@H](C(=O)Nc1ccc2c(c1)N(Cc1ccccc1)CC[C@H]2C(=O)N1CC(F)(F)C1)C1CCCCCC1]

Int 3 (1.0 equiv.) was dissolved in DMF, BnBr (1.0 equiv.) was added, and stirred with Cs₂CO₃ (1.2 equiv.) at room temperature for 6 h to reach a full conversion. The reaction mixture was then quenched and extracted with EA, then washed with brine. The organic layer was dried over MgSO₄, and concentrated in vacuo to produce int 3-1 (95% yield).

Int 3-1 (1.0 equiv.) was dissolved in HOAc, Pyridine·BH₃ (10 equiv.) was added, and stirred at room temperature for 12 h to reach a full conversion. The reaction mixture was then quenched and extracted with EA, then washed with brine. The organic layer was dried over MgSO₄, and concentrated in vacuo to produce int 3-2 (95% yield).

### Route 9. Preparation of Compound 67, Compound 89, and Compound 92

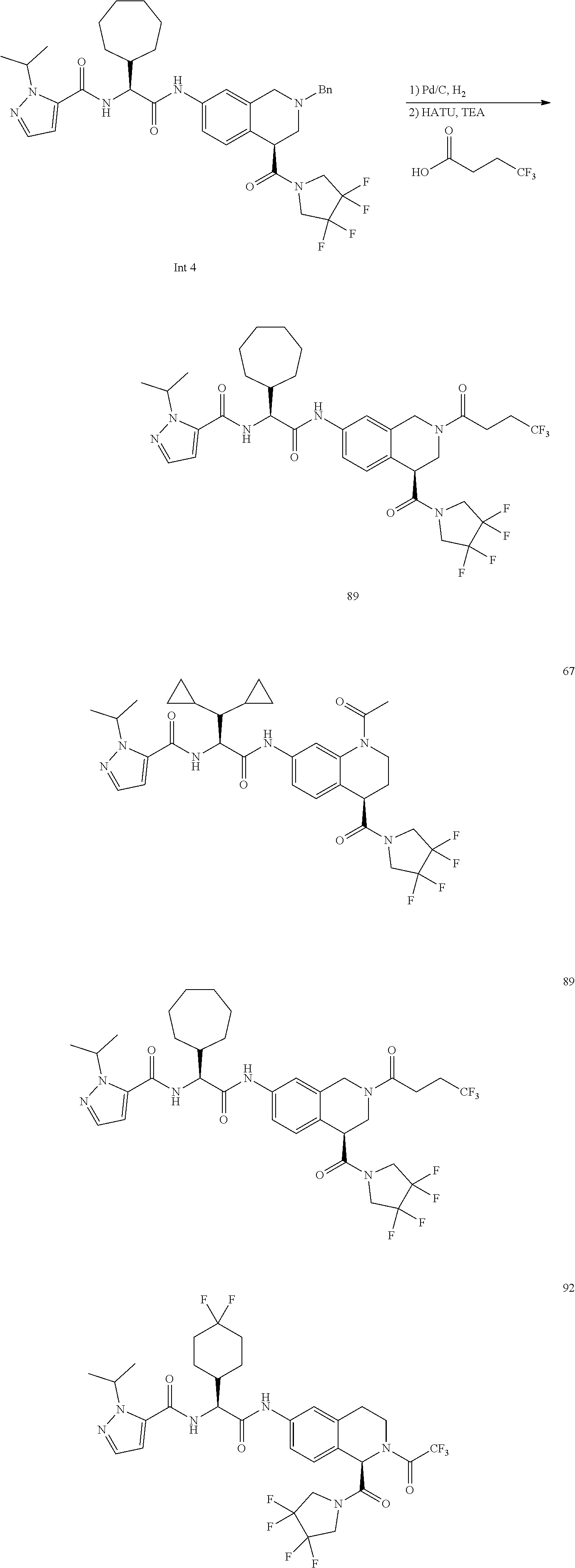

Int 4 underwent Pd/C catalyzed hydrolysis to generate a secondary ammonia, then it was mixed with 4,4,4-trifluorobutyric acid and HATU (1.5 equiv.) and TEA (1.5 equiv.) in DCM (1 M) and stirred for 2 hours at room temperature. Water was added and stirred for 10 mins. The organic phase was separated, washed with water and brine, dried over MgSO₄ and concentrated in vacuo to produce compound 89. Compound 67 and compound 92 were synthesized in the same way.

### Route 10. Preparation of Int 6

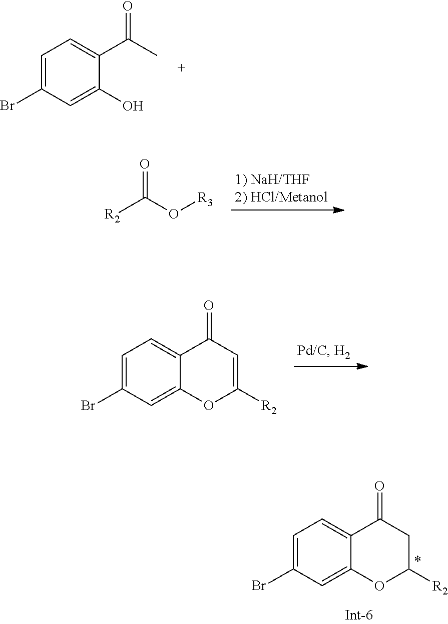

Sodium hydride (60% dispersion in mineral oil, 3.53 g, 88.2 mmol) was rinsed three times with hexanes, then suspended in THF (9 mL). A mixture of 2′-hydroxyacetophenone (22.0 mmol) and corresponding ester (55.1 mmol) in THF (2.5 mL) was added dropwise to the above suspension at room temperature. A vigorous reaction was observed, and the temperature rose to reflux. After complete addition, the reaction mixture was stirred for a further 5 min, then quenched by pouring onto ice and further acidified to pH 6 with 6 M aq HCl. The solution was extracted with ethyl acetate and the combined organic layers were washed with brine and dried over magnesium sulfate. The solvent was evaporated to yield the crude 1,3-dione. Without further purification, a solution of the crude 1,3-dione product (2.50 g, 14.0 mmol) in methanol (30 mL) was treated by use of conc. HCl (1 mL), and the mixture was stirred at room temperature for 14 h. The mixture was concentrated under reduced pressure and the residue diluted with ethyl acetate (50 mL), then washed successively with solutions of saturated aq NaHCO₃, water, and brine. The organic layer was then dried over MgSO₄ and concentrated under reduced pressure. The residue was purified via flash column chromatography (silica gel, ethyl acetate/hexanes 1:3 as eluent) to produce 2-substituted chromen-4-one. 2-substituted chromen-4-one was hydrogenated by Pd/C and H₂ in Methanol to produce Int-6.

### Route 11. Preparation of Int 7

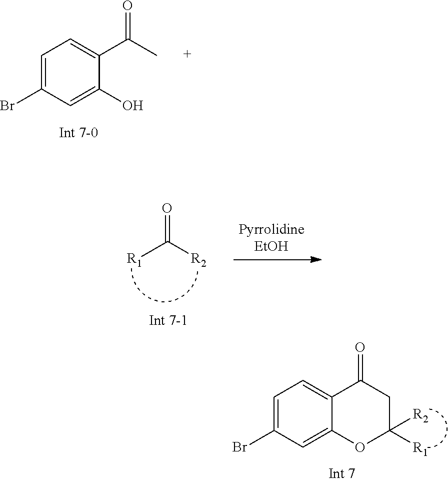

A round bottom flask equipped with magnetic stirrer, were charged with 1-(4-bromo-2-hydroxyphenyl)ethan-1-one (2 g, 9.30 mmol), Int 7-1 (1.16 g, 10.23 mmol), pyrrolidine (0.99 g, 13.95 mmol) and METHANOL (20 mL). The reaction mixture was stirred at 60° C. for 16 h. The progress of the reaction was monitored by TLC (PE/EtOAc 10:1). Upon completion of the reaction, the reaction mixture or solution was concentrated and dried. The dried mixture was partitioned between EA (100 mL) and 3N NaOH aq. (50 mL). The organic layer was separated, washed with water and brine (50 ml), dried over Na₂SO₄ and evaporated to dryness. After removal of solvent evaporation, Int 7 (Yield 60.66%) was obtained as a brown solid.

### Route 12. Preparation of Int 8

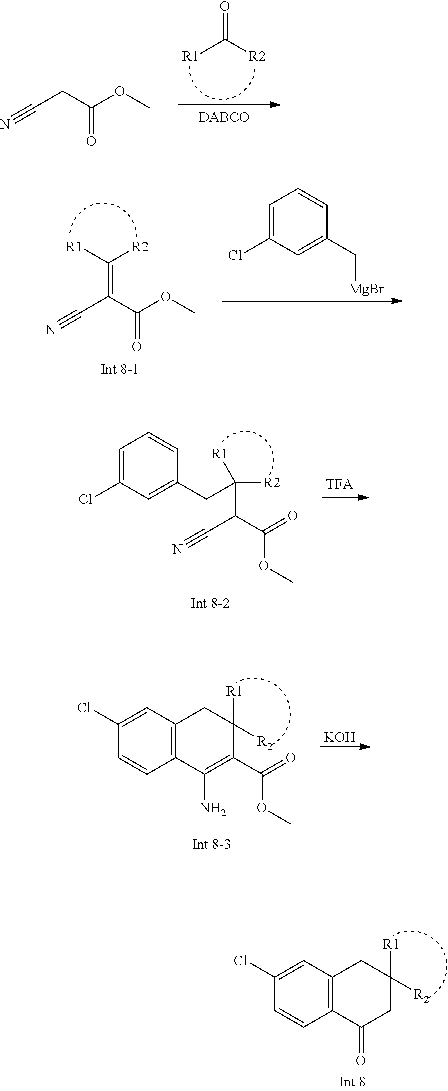

### Synthesis of Int 8-2

Int 8-1 was synthesized by Knoevenagel condensation with ketone and Methyl cyanoacetate. Then Int 8-1 was added dropwise into a THF solution of Grignard reagent (3-chlorobenzyl)magnesium bromide, then stirred, heated for 2 h at 42-45° C., cooled in ice water, treated dropwise with H₂SO₄ (20/O), and stirred at room temperature until the complex was completely decomposed. The organic layer was separated. The aqueous layer was extracted with Et₂O. The organic fractions were combined, washed with H₂O (2 times), and dried over MgSO₄. The solvent was vacuum distilled using a 10-cm fractionating column to produce Int 8-2.

### Synthesis of Int 8-3

Int 8-2 (16 mmol) was placed into a reaction flask, stirred, treated from a dropping funnel with conc. H₂SO₄ (8 mL) (with cooling by H₂O to maintain the mixture at 25-30° C.), stirred at room temperature for 3 h, and poured onto ice (120 g). The resulting crystals were filtered off, rinsed with H₂O, treated with H₂O (60 mL) and aqueous NH₄OH (6 mL), and extracted with Et₂O. The solvent was distilled off. The compound was recrystallized from EtOH—H₂O (2:1) to produce Int 8-3.

### Synthesis of Int 8

A mixture of Int 8-3 (20 mmol), the appropriate acid chloride (20 mmol), and anhydrous toluene (30 mL) was refluxed for 4 h. The solvent was distilled off. The residue was recrystallized from anhydrous EtOH to produce Int 8.

### Route 13. Preparation of Compound 167, Compound 176 and Compound 247

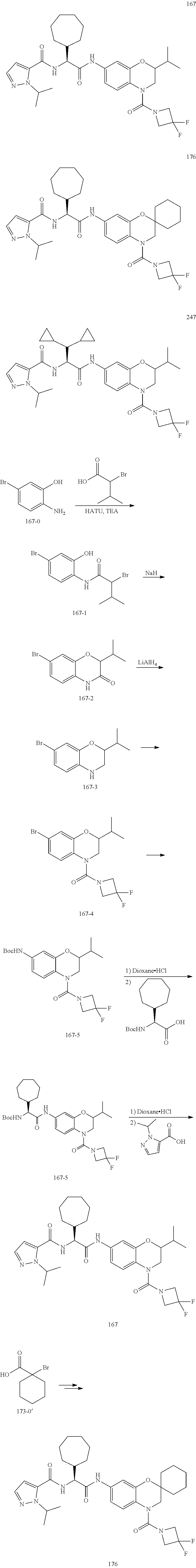

### Synthesis of Compound 167-1

2-bromo-3-methylbutanoic acid (1.1 equiv.), HATU (1.5 equiv.), compound 167-0 (1.0 equiv.) and TEA (1.5 equiv.) were added into DCM (1M), and stirred for 1 hours at 20-25° C. Water was added and stirred for 15 min. The organic layer was separated, washed with HCl (aq, 1 M) and brine, dried over MgSO₄ and concentrated in vacuo to produce compound 167-1.

### Synthesis of Compound 167-2

Compound 167-1 was dissolved in THF (0.5 M), NaH (2.0 equiv.) was added portionwise. Then the reaction was stirred at 20-25° C. for 8 hours until it reached a full conversion. Water (5 equiv.) and EA (0.1 M) was added and stirred for 15 min. The organic layer was separated, washed with water and brine, dried over MgSO₄ and concentrated in vacuo to produce compound 167-2.

### Synthesis of Compound 167-3

Compound 167-2 was dissolved in THF (0.5 M), and added slowly to the solution of lithium aluminum hydride (2 equiv.) in tetrahydrofuran (1 M) at 0° C. The mixture was stirred for 16 hours at room temperature. After completion of reaction, the reaction was quenched with wet sodium sulfate. The reaction mixture was filtered through high-low bed and washed with tetrahydrofuran (25 ml). The filtrate was distilled out completely to produce compound 167-3.

### Synthesis of Compound 167-4

Compound 167-3 (1.0 equiv.), DCM (0.3 M), Ethyl diisopropylamine (10 equiv.), and bis(trichloromethyl) carbonate (1.1 equiv.) were added into a round bottom flask equipped with a magnetic stirrer. Water was added and stirred for 15 min. The organic layer was separated, washed with HCl (aq, 1 M) and brine, dried over MgSO₄ and concentrated in vacuo to produce compound 167-4.

### Synthesis of Compound 167-5

Compound 167-4, (1.0 equiv.), tert-Butyl carbamate (1.5 equiv.), Pd₂(dba)₃ (0.1 equiv.) and Xantphos were added to a round bottom flask equipped with a magnetic stirrer, and pretreated by stir in least amount of 1,4-dioxane, 1,4-dioxane (0.01 M) was then added to the abovementioned mixture. The mixture was stirred under N₂ balloon at 85° C. for 3 hours. Samples were taken from the reaction mixture by dropping tube and checked for reaction progress via TLC (PE/EtOAc 1:1), LC-MS. After completion of the reaction, the reaction mixture was concentrated under reduced pressure or evaporated to remove dioxane. The concentrated reaction mixture was diluted with solvent and poured into water. The aqueous layer was extracted with organic solvent (20 mL) twice. The combined organic layers were washed with (30 mL) water and dried over Na₂SO₄. An oily crude product was obtained after removal of solvent by concentration. The crude product was purified by silica gel chromatography, eluted with PE:EtOAc=1:1 to produce compound 167-5 (90% yield) as yellow solid.

### Synthesis of Compound 167-6

Compound 167-5 was dissolved in the least amount of EtOAc, then dioxane-HCl was added and stirred at 25° C. for 2 hours. Then the reaction solvent was removed at reduced pressure to produce an ammonia hydrochloride salt with quantitative yield. This salt was used directly without further purification. (2S)-2-{[(tert-butoxy)carbonyl]amino}-2-cycloheptylacetic acid (1.0 equiv.), HATU (1.5 equiv.) and TEA (1.5 equiv.) were added into DCM (1M), and stirred for 1 hours at 20-25° C. The abovementioned salt (1.0 equiv.) was added and stirred for 2 hours. Water was added and stirred for 15 mins. The organic layer was separated, washed with water and brine, dried over MgSO₄ and concentrated in vacuo to produce compound 167-6.

### Synthesis of Compound 167

Compound 167-6 was dissolved in the least amount of EtOAc, then dioxane-HCl was added and stirred at 25° C. for 2 hours. Then the reaction solvent was removed at reduced pressure to produce an ammonia hydrochloride salt with quantitative yield. This salt was used directly without further purification. 1-isopropyl-1H-pyrazole-5-carboxylic acid (1.0 equiv.), HATU (1.5 equiv.) and TEA (1.5 equiv.) were added into DCM (1M), and stirred for 1 hours at 20-25° C. The abovementioned salt (1.0 equiv.) was added and stirred for 2 hours. Water was added and stirred for 15 mins. The organic layer was separated, washed with water and brine, dried over MgSO₄ and concentrated in vacuo to produce compound 167.

### Route 14. Preparation of Compound 169 and Compound 246

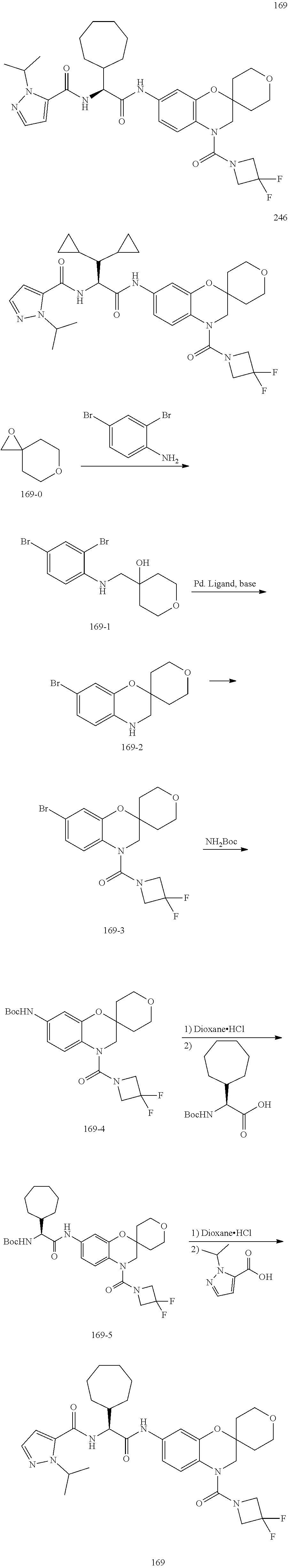

### Synthesis of Compound 169-2

Compound 169-0 (1.0 equiv.) and 2,4-dibromoaniline (1.0 equiv.) were dissolved in toluene (1 M). Activated silica gel (100 mg/mmol) was added and the mixture was stirred at 100° C. for hours until full conversion was reached. Pd(OAc)₂ (10 mol %), and rac-BINAP (10 mol %) were then added to the abovementioned mixture and stirred at 100° C. for another 2 hours to produce compound 169-2.

### Synthesis of Compound 169-3

Compound 169-2 (1.0 equiv.), DCM (0.3 M), Ethyl diisopropylamine (10 equiv.), and bis(trichloromethyl) carbonate (1.1 equiv.) were added to a round bottom flask equipped with a magnetic stirrer. Water was added and stirred for 15 min. The organic layer was separated, washed with HCl (aq, 1 M) and brine, dried over MgSO₄ and concentrated in vacuo to produce compound 169-3.

### Synthesis of Compound 169-4

Compound 169-3, (1.0 equiv.), tert-Butyl carbamate (1.5 equiv.), Pd₂(dba)₃ (0.1 equiv.) and Xantphos were added to a round bottom flask equipped with a magnetic stirrer and pretreated by stirring in the least amount of 1,4-dioxane. 1,4-dioxane (0.01 M) was then added to the abovementioned mixture. The mixture was stirred under N₂ balloon at 85° C. for 3 hours. Samples were taken from the reaction mixture by dropping tube and checked for reaction progress via TLC (PE/EtOAc 1:1), LC-MS. After completion of the reaction, the reaction mixture was concentrated under reduced pressure or evaporated to remove dioxane. The concentrated reaction mixture was diluted with solvent and poured into water. The aqueous layer was extracted with organic solvent (20 mL) twice. The combined organic layers were washed with (30 mL) water and dried over Na₂SO₄. An oily crude product was obtained after removal of solvent by concentration. The crude product was purified by silica gel chromatography, eluted with PE:EtOAc=1:1 to produce compound 169-4 (90% yield) as yellow solid.

### Synthesis of Compound 169-5

Compound 169-4 was dissolved in the least amount of EtOAc, then dioxane-HCl was added and stirred at 25° C. for 2 hours. Then the reaction solvent and removed at reduced pressure to produce an ammonia hydrochloride salt with quantitative yield. This salt was used directly without further purification. (2S)-2-{[(tert-butoxy)carbonyl]amino}-2-cycloheptylacetic acid (1.0 equiv.), HATU (1.5 equiv.) and TEA (1.5 equiv.) were added into DCM (1M), and stirred for 1 hours at 20-25° C. The abovementioned salt (1.0 equiv.) was added and stirred for 2 hours. Water was added and stirred for 15 mins. The organic layer was separated, washed with water and brine, dried over MgSO₄ and concentrated in vacuo to produce compound 169-5.

### Synthesis of Compound 169

Compound 169-5 was dissolved in the least amount of EtOAc, then dioxane-HCl was added and stirred at 25° C. for 2 hours. Then the reaction solvent was removed at reduced pressure to produce an ammonia hydrochloride salt with quantitative yield. This salt was used directly without further purification. 1-isopropyl-1H-pyrazole-5-carboxylic acid (1.0 equiv.), HATU (1.5 equiv.) and TEA (1.5 equiv.) were added into DCM (1M), and stirred for 1 hours at 20-25° C. The abovementioned salt (1.0 equiv.) was added and stirred for 2 hours. Water was added and stirred for 15 mins. The organic layer was separated, washed with water and brine, dried over MgSO₄ and concentrated in vacuo to produce compound 169.

### Route 15. Preparation of Compound 189, 190, 191, 192, and 194

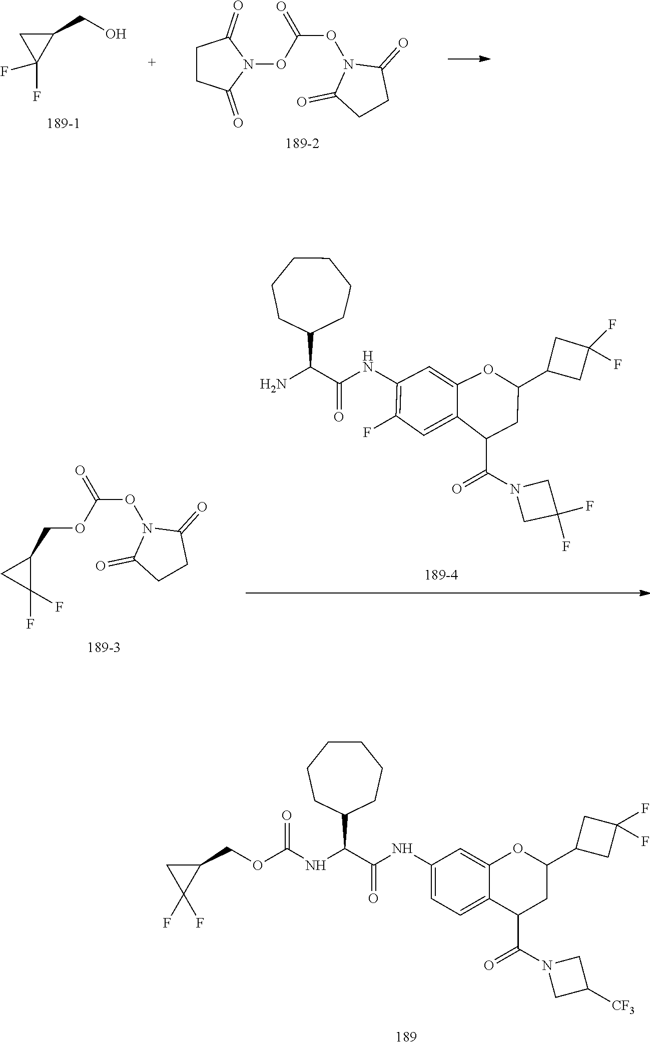 [SMILES: OC[C@@H]1CC1(F)F ; O=C(ON1C(=O)CCC1=O)ON1C(=O)CCC1=O ; O=C(OC[C@@H]1CC1(F)F)ON1C(=O)CCC1=O ; N[C@H](C(=O)Nc1cc2c(cc1F)C(C(=O)N1CC(F)(F)C1)CC(C1CC(F)(F)C1)O2)C1CCCCCCCC1 ; O=C(N[C@H](C(=O)Nc1ccc2c(c1)OC(C1CC(F)(F)C1)CC2C(=O)N1CC(C(F)(F)F)C1)C1CCCCCC1)OC[C@@H]1CC1(F)F]

N,N′-Disuccinimidyl carbonate (0.51 g, 2 mmol) and pyridine (0.19 g, 2.4 mmol) were added to a solution of 2,2-Difluorocyclopropanemethanol (216 mg, 2.00 mmol) in acetonitrile (4 mL). The mixture was stirred overnight at room temperature to produce a clear solution. Sat. aq. NaHCO₃ (5 mL) and brine (20 mL) were added and the phases were separated. The aqueous phase was extracted with EtOAc (2×50 ml) and the combined organic phases were washed with brine (20 ml), dried over MgSO₄, and concentrated in vacuo to produce (2,2-difluorocyclopropyl)methyl 2,5-dioxopyrrolidin-1-yl carbonate (313 mg, Yield 62.86%) without purification for next step.

A mixture of 189-4 (25 mg, 0.046 mmol), 189-3 (0.013 g, 0.055 mmol), TEA, and Triethylamine (0.014 g, 0.14 mmol) in acetonitrile (1.5 mL) was heated at RT overnight. The reaction mixture was washed with water and brine, then extracted with EtOAc (3×5 mL). The combined organic extracts were dried over Na₂SO₄ and concentrated in vacuo. The concentrated product was purified by column chromatography (silica gel, eluting with DCM/CH₃₀H) to produce compound 189: (2,2-difluorocyclopropyl)methyl N—[(S)-cycloheptyl[(2-(3,3-difluorocyclobutyl)-4-(3-(trifluoromethyl)azetidine-1-carbonyl)-3,4-dihydro-2H-1-benzopyran-7-yl)carbamoyl]methyl]carbamate (3.6 mg, Yield 11.55%).

Compound 190, 191, 192 and 194 were synthesized in the same way.

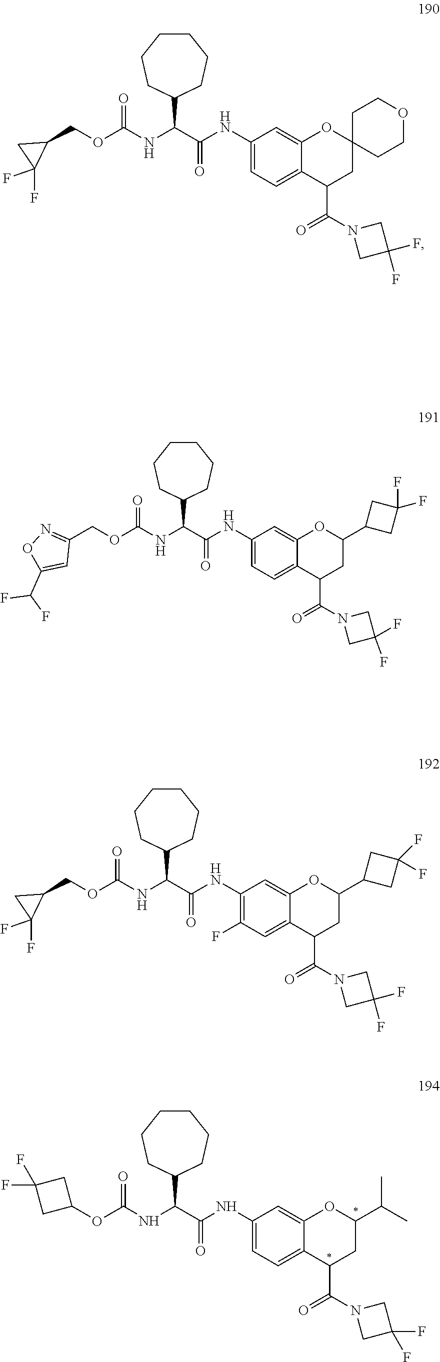 [SMILES: O=C(N[C@H](C(=O)Nc1ccc2c(c1)OC1(CCOCC1)CC2C(=O)N1CC(F)(F)C1)C1CCCCCC1)OC[C@@H]1CC1(F)F ; O=C(N[C@H](C(=O)Nc1ccc2c(c1)OC(C1CC(F)(F)C1)CC2C(=O)N1CC(F)(F)C1)C1CCCCCC1)OCc1cc(C(F)F)on1 ; O=C(N[C@H](C(=O)Nc1cc2c(cc1F)C(C(=O)N1CC(F)(F)C1)CC(C1CC(F)(F)C1)O2)C1CCCCCC1)OC[C@@H]1CC1(F)F ; CC(C)C1CC(C(=O)N2CC(F)(F)C2)c2ccc(NC(=O)[C@@H](NC(=O)OC3CC(F)(F)C3)C3CCCCCC3)cc2O1]

### Chiral Resolution

The compounds of formula I were chiral separated by pre-HPLC: chromatographic column: CHIRALPAK® IA, 10 μm, 30*250 mm; mobile phase A: HEX; mobile phase B: IPC; UV: 254 Nm; Isocratic elution program: mobile phase A: mobile phase B=60:40 (V/V).

The following compounds were prepared according to the procedures described herein using the appropriate starting material(s) and intermediate(s) and appropriate protecting group chemistry as needed, and certified by ¹HNMR and MS. Table 1. List of Compounds.

| | | ES/ | |
|---|---|---|---|
| | | MS | |
| | | (m/z, | |
| | | M + | |
| No. | Structure | H⁺) | ¹H NMR |
| 1 | 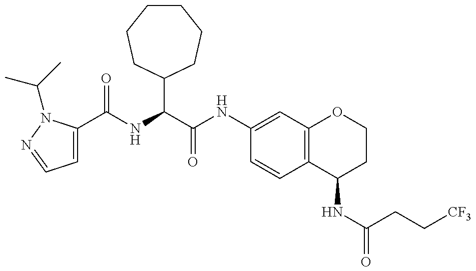 [SMILES: CC(C)n1nccc1C(=O)N[C@H](C(=O)Nc1ccc2c(c1)OCC[C@H]2NC(=O)CCC(F)(F)F)C1CCCCCC1] | 578.2 | ¹H NMR (400 MHz, Chloroform-d) δ 8.55 (d, 1H), 7.41 − 7.45 (m, 1H), 7.13 − 6.83 (m, 4H), 6.61 − 6.63 (m, 1H), 6.26 − 6.31 (m, 1H), 5.43 − 5.47 (m, 1H), 5.06 − 5.11 (m, 1H), 4.56 − 5.57 (m, 1H), 4.01 − 4.19 (m, 2H), 2.45 − 2.47 (m, 4H), 2.15 − 2.17 (m, 2H), 2.05 − 1.94 (m, 1H), 1.90-1.82 (m, 2H), 1.35 − 1.74 (m, 16H). |
| 2 | 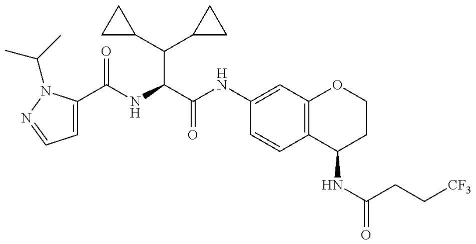 [SMILES: CC(C)n1nccc1C(=O)N[C@H](C(=O)Nc1ccc2c(c1)OCC[C@H]2NC(=O)CCC(F)(F)F)C(C1CC1)C1CC1] | 576.3 | ¹H NMR (400 MHz, Chloroform-d) δ 8.57 (d, 1H), 7.42 − 7.45 (m, 1H), 7.14 − 6.84 (m, 4H), 6.61 − 6.63 (m, 1H), 6.26 − 6.30 (m, 1H), 5.43 − 5.45 (m, 1H), 5.04 − 5.06 (m, 1H), 4.56 − 5.57 (m, 1H), 402 − 4.29 (m, 2H), 2.45 − 2.47 (m, 4H), 2.15 − 2.17 (m, 2H), 2.05 − 1.94 (m, 1H), 1.34 − 1.86 (m, 16H). |
| 3 | 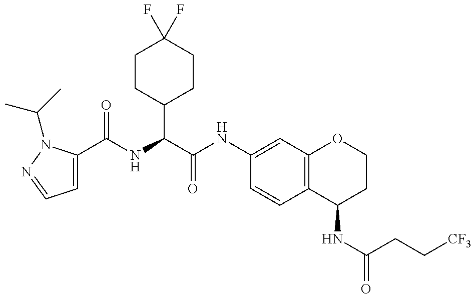 [SMILES: CC(C)n1nccc1C(=O)N[C@H](C(=O)Nc1ccc2c(c1)OCC[C@H]2NC(=O)CCC(F)(F)F)C1CCC(F)(F)CC1] | 600.3 | ¹H NMR (400 MHz, Chloroform-d) δ 8.59 (d, 1H), 7.42 − 7.45 (m, 1H), 7.12 − 6.82 (m, 4H), 6.61 − 6.63 (m, 1H), 6.26 − 6.31 (m, 1H), 5.43 − 5.45 (m, 1H), 5.04 − 5.07 (m, 1H), 4.56 − 5.57 (m, 1H), 4.01 − 4.27 (m, 2H), 2.45 − 2.47 (m, 4H), 2.15 − 2.14 (m, 2H), 2.05 − 1.94 (m, 1H), 1.42 − 1.86 (m, 14H). |
| 4 | 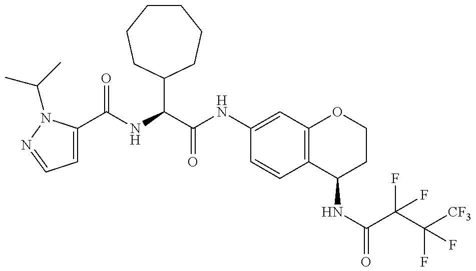 [SMILES: CC(C)n1nccc1C(=O)N[C@H](C(=O)Nc1ccc2c(c1)OCC[C@H]2NC(=O)C(F)(F)C(F)(F)C(F)(F)F)C1CCCCCC1] | 650.2 | ¹H NMR (400 MHz, Chloroform-d) δ 8.55 (d, 1H), 7.41 − 7.45 (m, 1H), 7.13 − 6.83 (m, 4H), 6.61 − 6.63 (m, 1H), 6.26 − 6.31 (m, 1H), 5.43 − 5.45 (m, 1H), 5.04 − 5.08 (m, 1H), 4.56 − 5.57 (m, 1H), 4.01 − 4.26 (m, 2H), 2.15 − 2.17 (m, 2H), 2.05 − 1.94 (m, 1H), 1.35 − 1.86 (m, 18H). |
| 5 | 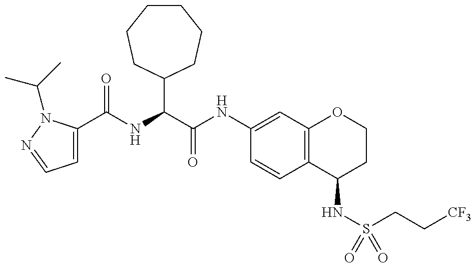 [SMILES: CC(C)n1nccc1C(=O)N[C@H](C(=O)Nc1ccc2c(c1)OCC[C@H]2NS(=O)(=O)CCC(F)(F)F)C1CCCCCC1] | 614.3 | ¹H NMR (400 MHz, Chloroform-d) δ 8.59 (d, 1H), 7.42 − 7.45 (m, 1H), 7.12 − 6.82 (m, 4H), 6.61 − 6.63 (m, 1H), 6.26 − 6.31 (m, 1H), 5.43 − 5.45 (m, 1H), 5.04 − 5.07 (m, 1H), 4.56 − 5.57 (m, 1H), 4.01 − 4.27 (m, 2H), 3.10 − 3.12 (m, 2H), 2.45 − 2.47 (m, 2H), 2.15 − 2.14 (m, 2H), 2.05 − 1.94 (m, 1H), 1.42 − 1.86 (m, 18H). |
| 6 | 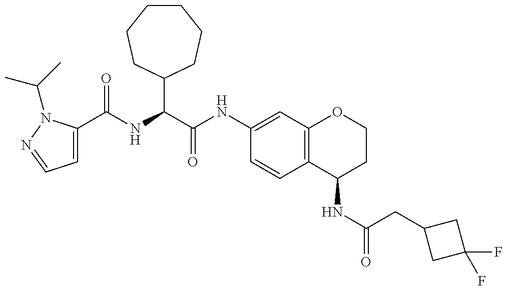 [SMILES: CC(C)n1nccc1C(=O)N[C@H](C(=O)Nc1ccc2c(c1)OCC[C@H]2NC(=O)CC1CC(F)(F)C1)C1CCCCCC1] | 586.3 | ¹H NMR (400 MHz, Chloroform-d) δ 8.55 (d, 1H), 7.41 − 7.45 (m, 1H), 7.13 − 6.83 (m, 4H), 6.61 − 6.63 (m, 1H), 6.26 − 6.31 (m, 1H), 5.43 − 5.45 (m, 1H), 5.04 − 5.08 (m, 1H), 4.56 − 5.57 (m, 1H), 4.01 − 4.26 (m, 2H), 2.42 − 2.50 (m, 7H), 2.15 − 2.17 (m, 2H), 2.05 − 1.94 (m, 1H), 1.35 − 1.86 (m, 18H). |
| 7 | 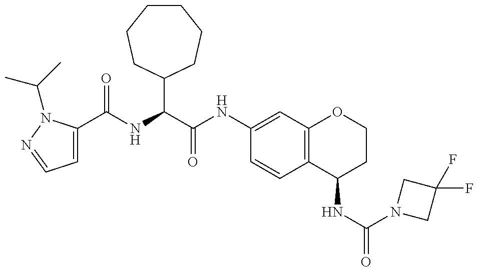 [SMILES: CC(C)n1nccc1C(=O)N[C@H](C(=O)Nc1ccc2c(c1)OCC[C@H]2NC(=O)N1CC(F)(F)C1)C1CCCCCC1] | 573.3 | ¹H NMR (400 MHz, Chloroform-d) δ 8.55 (d, 1H), 7.41 − 7.45 (m, 1H), 7.13 − 6.83 (m, 4H), 6.61 − 6.63 (m, 1H), 6.26 − 6.31 (m, 1H), 5.43 − 5.45 (m, 1H), 5.04 − 5.08 (m, 1H), 4.56 − 5.57 (m, 1H), 4.01 − 4.26 (m, 6H), 2.15 − 2.17 (m, 2H), 2.05 − 1.94 (m, 1H), 1.35 − 1.86 (m, 18H). |
| 8 | 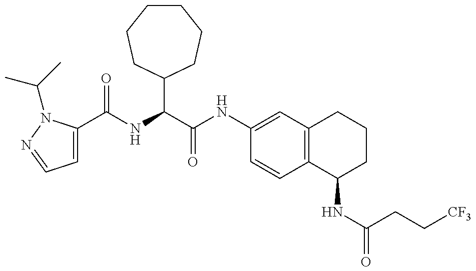 [SMILES: CC(C)n1nccc1C(=O)N[C@H](C(=O)Nc1ccc2c(c1)CCC[C@H]2NC(=O)CCC(F)(F)F)C1CCCCCC1] | 576.3 | ¹H NMR (400 MHz, Chloroform-d) δ 8.55 (d, 1H), 7.41 − 7.45 (m, 1H), 7.13 − 6.83 (m, 4H), 6.61 − 6.63 (m, 1H), 6.26 − 6.31 (m, 1H), 5.43 − 5.45 (m, 1H), 5.04 − 5.08 (m, 1H), 4.56 − 5.57 (m, 1H), 2.59 − 2.64 (m, 2H), 2.45 − 2.47 (m, 4H), 2.15 − 2.17 (m, 2H), 2.05 − 1.94 (m, 3H), 1.35 − 1.86 (m, 18H). |
| 9 | 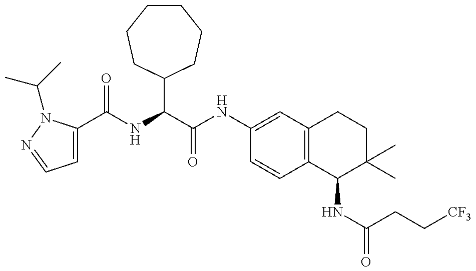 [SMILES: CC(C)n1nccc1C(=O)N[C@H](C(=O)Nc1ccc2c(c1)CCC(C)(C)[C@H]2NC(=O)CCC(F)(F)F)C1CCCCCC1] | 603.3 | ¹H NMR (400 MHz, Chloroform-d) δ 8.55 (d, 1H), 7.47 − 7.45 (m, 1H), 7.13 − 6.83 (m, 4H), 6.67 − 6.63 (m, 1H), 6.36 − 6.21 (m, 1H), 5.49 − 5.45 (m, 1H), 5.14 − 5.08 (m, 1H), 4.58 − 4.54 (m, 1H), 2.87 − 2.83 (m, 2H), 2.55 − 2.47 (m, 4H), 2.25 − 2.17 (m, 2H), 2.05 − 1.94 (m, 1H), 1.95 − 1.36 (m, 16H), 1.08 − 0.93 (d, 6H). |
| 10 | 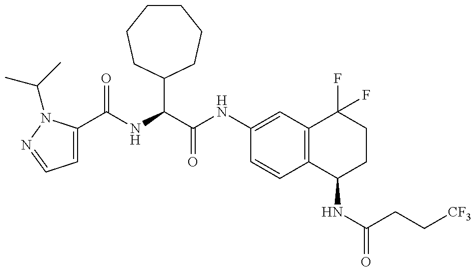 [SMILES: CC(C)n1nccc1C(=O)N[C@H](C(=O)Nc1ccc2c(c1)C(F)(F)CC[C@H]2NC(=O)CCC(F)(F)F)C1CCCCCC1] | 612.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.28 − 4.01 (m, 2H), 2.47 − 2.45 (m, 4H), 2.25 − 1.94 (m, 3H), 1.86 − 1.56 (m, 18H). |
| 11 | 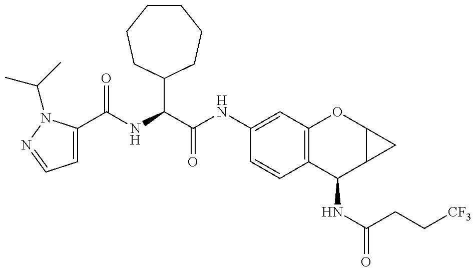 [SMILES: CC(C)n1nccc1C(=O)N[C@H](C(=O)Nc1ccc2c(c1)OC1CC1[C@H]2NC(=O)CCC(F)(F)F)C1CCCCCC1] | 590.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.41 − 6.38 (m, 1H), 6.33 − 6.31 (m, 1H), 5.47 − 5.43 (m, 2H), 5.10 − 5.08 (m, 1H), 4.58 − 4.57 (m, 1H), 2.47 − 2.45 (m, 4H), 2.05 − 1.94 (m, 1H), 1.86 − 1.56 (m, 18H). |
| 12 | 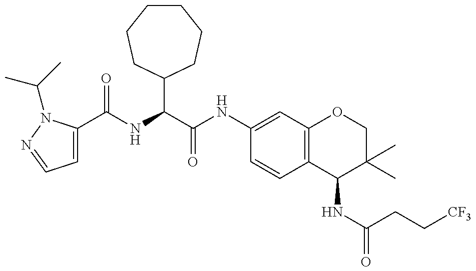 [SMILES: CC(C)n1nccc1C(=O)N[C@H](C(=O)Nc1ccc2c(c1)OCC(C)(C)[C@H]2NC(=O)CCC(F)(F)F)C1CCCCCC1] | 606.3 | ¹H NMR (400 MHz. Chloroform-d) δ 8.55 (d, 1H), 7.47 − 7.45 (m, 1H), 7.13 − 6.83 (m, 4H), 6.67 − 6.63 (m, 1H), 6.36 − 6.21 (m, 1H), 5.49 − 5.45 (m, 1H), 5.14 − 5.08 (m, 1H), 4.58 − 4.54 (m, 1H), 2.69 − 2.64 (m, 2H), 2.55 − 2.47 (m, 4H), 2.25 − 2.17 (m, 2H), 2.05 − 1.94 (m, 1H), 1.95 − 1.36 (m, 16H), 1.08 − 0.93 (d, 6H). |
| 13 | 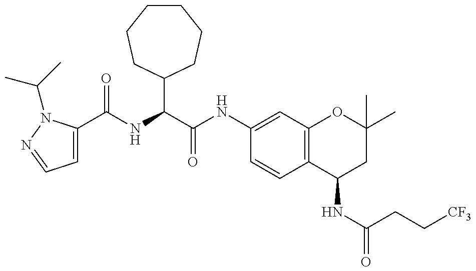 [SMILES: CC(C)n1nccc1C(=O)N[C@H](C(=O)Nc1ccc2c(c1)OC(C)(C)C[C@H]2NC(=O)CCC(F)(F)F)C1CCCCCC1] | 607.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 2.47 − 2.45 (m, 4H), 2.19 − 2.17 (m, 2H), 2.05 − 1.94 (m, 1H), 1.86 − 1.56 (m, 18H), 1.08 − 0.93 (d, 6H). |
| 14 | 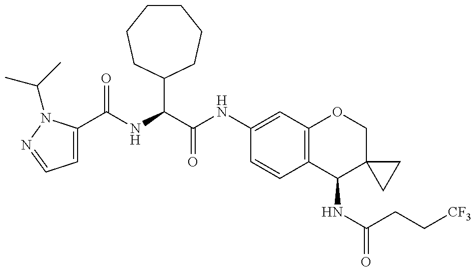 [SMILES: CC(C)n1nccc1C(=O)N[C@H](C(=O)Nc1ccc2c(c1)OCC1(CC1)[C@H]2NC(=O)CCC(F)(F)F)C1CCCCCC1] | 604.3 | ¹H NMR (400 MHz, Chloroform-d) δ 8.55 (d, 1H), 7.47 − 7.45 (m, 1H), 7.13 − 6.83 (m, 4H), 6.67 − 6.63 (m, 1H), 6.36 − 6.21 (m, 1H), 5.49 − 5.45 (m, 1H), 5.14 − 5.08 (m, 1H), 4.58 − 4.54 (m, 1H), 2.69 − 2.64 (m, 2H), 2.55 − 2.47 (m, 4H), 2.25 − 2.17 (m, 2H), 2.05 − 1.94 (m, 1H), 1.95 − 1.36 (m, 16H), 0.43 − 0.16 (m, 4H). |
| 15 | 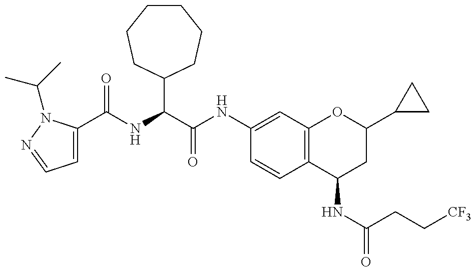 [SMILES: CC(C)n1nccc1C(=O)N[C@H](C(=O)Nc1ccc2c(c1)OC(C1CC1)C[C@H]2NC(=O)CCC(F)(F)F)C1CCCCCC1] | 618.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 3.00 − 2.95 (m, 1H), 2.47 − 2.45 (m, 4H), 2.19 − 2.17 (m, 2H), 2.05 − 1.94 (m, 1H), 1.86 − 1.56 (m, 18H), 0.93 − 0.90 (m, 1H), 0.43 − 0.16 (m, 4H). |
| 16 | 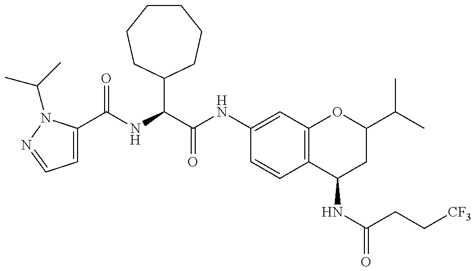 [SMILES: CC(C)C1C[C@@H](NC(=O)CCC(F)(F)F)c2ccc(NC(=O)[C@@H](NC(=O)c3ccnn3C(C)C)C3CCCCCC3)cc2O1] | 620.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 3.00 − 2.95 (m, 1H), 2.47 − 2.45 (m, 4H), 2.19 − 2.17 (m, 2H), 2.05 − 1.94 (m, 1H), 1.86 − 1.56 (m, 18H), 0.93 − 0.77 (m, 7H). |
| 17 | 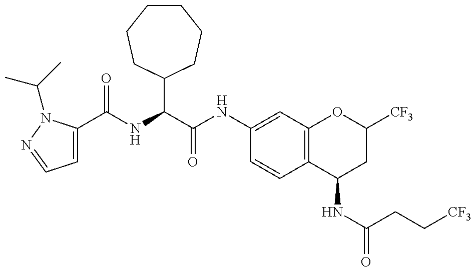 [SMILES: CC(C)n1nccc1C(=O)N[C@H](C(=O)Nc1ccc2c(c1)OC(C(F)(F)F)C[C@H]2NC(=O)CCC(F)(F)F)C1CCCCCC1] | 646.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 3.00 − 2.95 (m, 1H), 2.47 − 2.45 (m, 4H), 2.19 − 2.17 (m, 2H), 2.05 − 1.94 (m, 1H), 1.86 − 1.56 (m, 18H). |
| 18 | 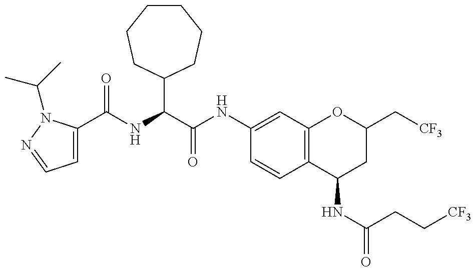 [SMILES: CC(C)n1nccc1C(=O)N[C@H](C(=O)Nc1ccc2c(c1)OC(CC(F)(F)F)C[C@H]2NC(=O)CCC(F)(F)F)C1CCCCCC1] | 660.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 3.00 − 2.95 (m, 1H), 2.47 − 2.45 (m, 4H), 2.38 − 2.17 (m, 4H), 2.05 − 1.94 (m, 1H), 1.86 − 1.56 (m, 18H). |
| 19 | 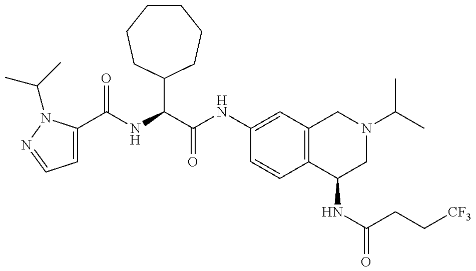 [SMILES: CC(C)N1Cc2cc(NC(=O)[C@@H](NC(=O)c3ccnn3C(C)C)C3CCCCCC3)ccc2[C@H](NC(=O)CCC(F)(F)F)C1] | 619.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.47 − 2.45 (m, 5H), 2.19 − 2.17 (m, 2H), 2.05 − 1.94 (m, 1H), 1.86 − 1.56 (m, 18H), 1.22 − 1.02 (m, 6H). |
| 20 | 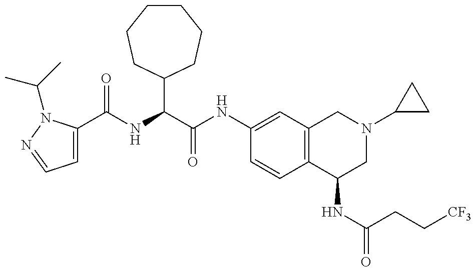 [SMILES: CC(C)n1nccc1C(=O)N[C@H](C(=O)Nc1ccc2c(c1)CN(C1CC1)C[C@H]2NC(=O)CCC(F)(F)F)C1CCCCCC1] | 617.3 | ¹H NMR (400 MHz. Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.47 − 2.45 (m, 5H), 2.19 − 2.17 (m, 2H), 2.05 − 1.94 (m, 1H), 1.86 − 1.56 (m, 18H), 0.87 − 0.54 (m, 4H). |
| 21 | 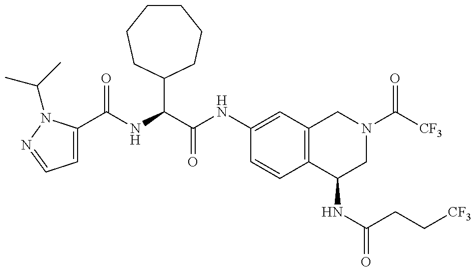 [SMILES: CC(C)n1nccc1C(=O)N[C@H](C(=O)Nc1ccc2c(c1)CN(C(=O)C(F)(F)F)C[C@H]2NC(=O)CCC(F)(F)F)C1CCCCCC1] | 673.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.47 − 2.45 (m, 5H), 2.19 − 2.17 (m, 1H), 2.05 − 1.94 (m, 1H), 1.86 − 1.56 (m, 18H). |
| 22 | 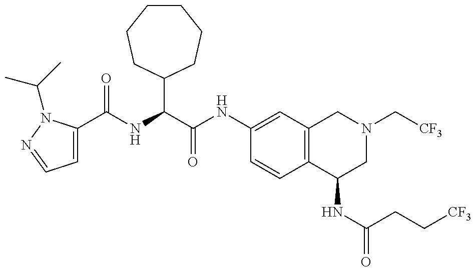 [SMILES: CC(C)n1nccc1C(=O)N[C@H](C(=O)Nc1ccc2c(c1)CN(CC(F)(F)F)C[C@H]2NC(=O)CCC(F)(F)F)C1CCCCCC1] | 659.3 | ¹H NMR (400 MHz. Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 2H), 2.47 − 2.45 (m, 6H), 2.19 − 2.17 (m, 1H), 2.05 − 1.94 (m, 1H), 1.86 − 1.56 (m, 18H). |
| 23 | 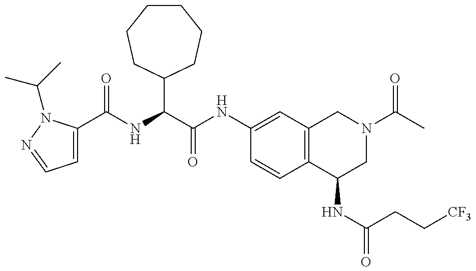 [SMILES: CC(=O)N1Cc2cc(NC(=O)[C@@H](NC(=O)c3ccnn3C(C)C)C3CCCCCC3)ccc2[C@H](NC(=O)CCC(F)(F)F)C1] | 619.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.47 − 2.45 (m, 5H), 2.19 − 2.17 (m, 1H), 2.05 − 1.94 (m, 4H), 1.86 1.56 (m, 18H). |
| 24 | 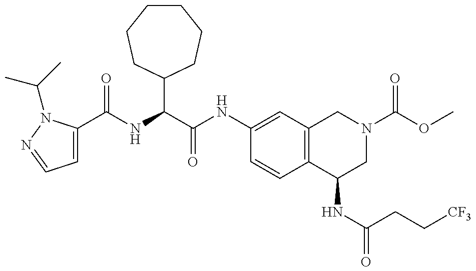 [SMILES: COC(=O)N1Cc2cc(NC(=O)[C@@H](NC(=O)c3ccnn3C(C)C)C3CCCCCC3)ccc2[C@H](NC(=O)CCC(F)(F)F)C1] | 635.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 4H), 2.47 − 2.45 (m, 5H), 2.19 − 2.17 (m, 1H), 2.05 − 1.94 (m, 1H), 1.86 − 1.56 (m, 18H). |
| 25 | 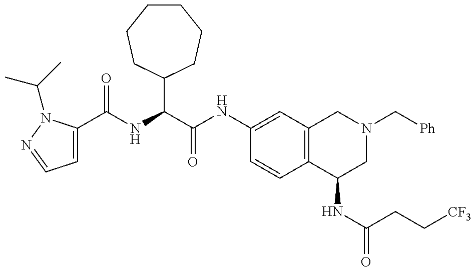 [SMILES: CC(C)n1nccc1C(=O)N[C@H](C(=O)Nc1ccc2c(c1)CN(Cc1ccccc1)C[C@H]2NC(=O)CCC(F)(F)F)C1CCCCCC1] | 667.3 | ¹H NMR (400 MHz, Chloroform-d) δ 8.55 (d, 1H), 7.47 − 7.19 (m, 6H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 2H), 2.47 − 2.45 (m, 6H), 2.19 − 2.17 (m, 1H), 2.05 − 1.94 (m, 1H), 1.86 − 1.56 (m, 18H), |
| 26 | 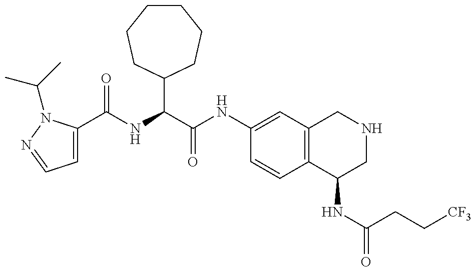 [SMILES: CC(C)n1nccc1C(=O)N[C@H](C(=O)Nc1ccc2c(c1)CNC[C@H]2NC(=O)CCC(F)(F)F)C1CCCCCC1] | 577.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.47 − 2.45 (m, 5H), 2.19 − 2.17 (m, 2H), 2.05 − 1.94 (m, 1H), 1.86 − 1.56 (m, 18H). |
| 27 | 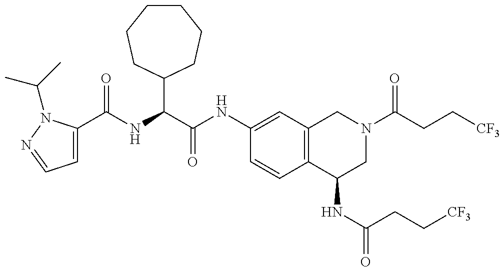 [SMILES: CC(C)n1nccc1C(=O)N[C@H](C(=O)Nc1ccc2c(c1)CN(C(=O)CCC(F)(F)F)C[C@H]2NC(=O)CCC(F)(F)F)C1CCCCCC1] | 701.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 2.90 − 2.70 (m, 2H), 2.63 − 2.45 (m, 6H), 2.40 − 2.30 (m, 1H), 2.19 − 2.17 (m, 1H), 2.05 − 1.94 (m, 1H), 1.86 − 1.56 (m, 18H). |
| 28 | 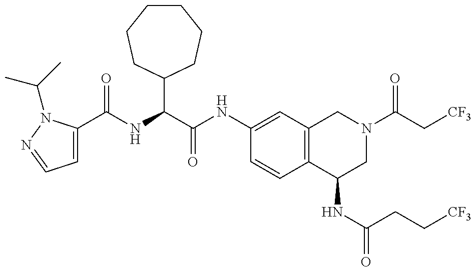 [SMILES: CC(C)n1nccc1C(=O)N[C@H](C(=O)Nc1ccc2c(c1)CN(C(=O)CC(F)(F)F)C[C@H]2NC(=O)CCC(F)(F)F)C1CCCCCC1] | 687.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 4.23 − 4.18 (m, 1H), 3.61 − 3.57 (m, 1H), 3.15 − 2.92 (m, 2H), 2.47 − 2.45 (m, 5H), 2.19 − 2.17 (m, 1H), 2.05 − 1.94 (m, 1H), 1.86 − 1.56 (m, 18H). |
| 29 | 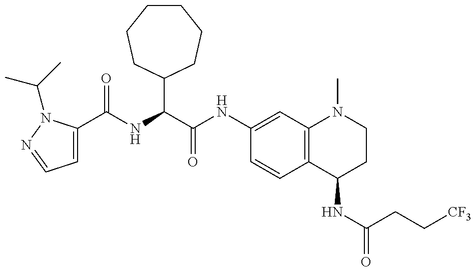 [SMILES: CC(C)n1nccc1C(=O)N[C@H](C(=O)Nc1ccc2c(c1)N(C)CC[C@H]2NC(=O)CCC(F)(F)F)C1CCCCCC1] | 591.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 3.29 − 3.22 (m, 1H), 3.04 − 2.93 (m, 1H), 2.83 (s, 3H), 2.47 − 2.45 (m, 5H), 2.19 − 2.17 (m, 1H), 2.05 − 1.94 (m, 1H), 1.86 − 1.56 (m, 18H). |
| 30 | 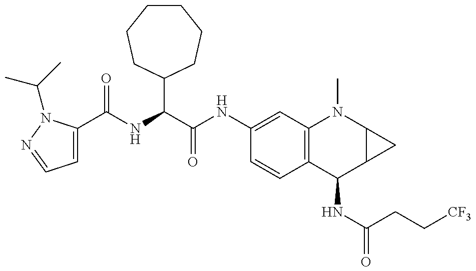 [SMILES: CC(C)n1nccc1C(=O)N[C@H](C(=O)Nc1ccc2c(c1)N(C)C1CC1[C@H]2NC(=O)CCC(F)(F)F)C1CCCCCC1] | 603.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 6.31 (m, 1H), 5.47 − 5.45 (m, 1H), 5.10 − 5.08 (m, 1H), 4.58 − 4.57 (m, 1H), 2.83 (s, 3H), 2.47 − 2.45 (m, 5H), 2.25 − 2.17 (m, 1H), 1.86 − 1.56 (m, 19H), 0.56 − 0.32 (m, 2H). |
| 31 | 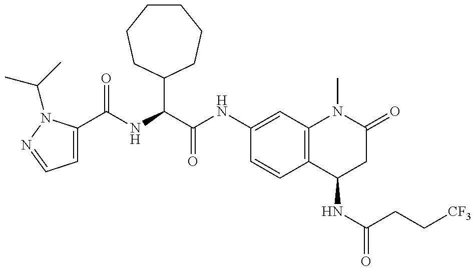 [SMILES: CC(C)n1nccc1C(=O)N[C@H](C(=O)Nc1ccc2c(c1)N(C)C(=O)C[C@H]2NC(=O)CCC(F)(F)F)C1CCCCCC1] | 605.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 2.89 − 2.86 (m, 1H), 2.83 (s, 3H), 2.53 − 2.45 (m, 6H), 1.86 − 1.56 (m, 18H). |
| 32 | 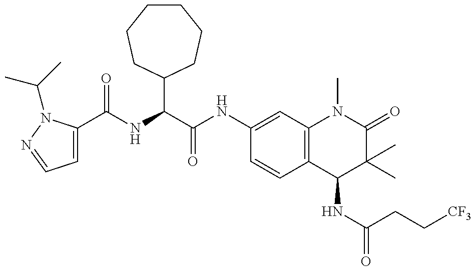 [SMILES: CC(C)n1nccc1C(=O)N[C@H](C(=O)Nc1ccc2c(c1)N(C)C(=O)C(C)(C)[C@H]2NC(=O)CCC(F)(F)F)C1CCCCCC1] | 633.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 5.10 − 5.08 (m, 1H), 4.58 − 4.57 (m, 1H), 2.83 (s, 3H), 2.53 − 2.45 (m, 5H), 1.86 − 1.06 (m, 24H). |
| 33 | 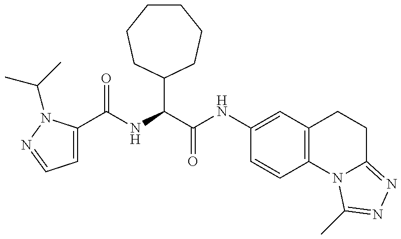 [SMILES: Cc1nnc2n1-c1ccc(NC(=O)[C@@H](NC(=O)c3ccnn3C(C)C)C3CCCCCC3)cc1CC2] | 490.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 3.59-3.36 (m, 2H), 3.25-3.12 (m, 2H), 2.23 (s, 3H), 2.06-1.96 (m, 1H), 1.75- 1.37 (m, 16H), 1.21 − 1.07 (m, 2H). |
| 34 | 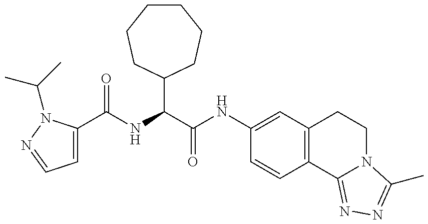 [SMILES: Cc1nnc2n1CCc1cc(NC(=O)[C@@H](NC(=O)c3ccnn3C(C)C)C3CCCCCC3)ccc1-2] | 490.3 | ¹H NMR (400 MHz, Chloroform-d) δ 8.55 (d, 1H), 7.47 − 7.45 (m, 1H), 7.13 − 6.83 (m, 4H), 6.64 − 6.62 (m, 1H), 6.33 − 6.31 (m, 1H), 5.47 − 5.45 (m, 1H), 3.30-3.25 (m, 1H), 3.04-3.96 (m, 1H), 2.48 (s, 3H), 2.25-2.17 (m, 1H), 1.72- 1.33 (m, 18H), 1.21 − 1.07 (m, 2H). |
| 35 | 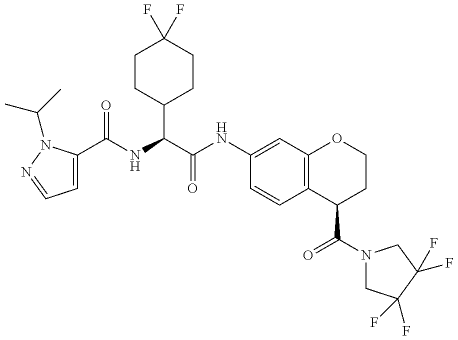 [SMILES: CC(C)n1nccc1C(=O)N[C@H](C(=O)Nc1ccc2c(c1)OCC[C@H]2C(=O)N1CC(F)(F)C(F)(F)C1)C1CCC(F)(F)CC1] | 631.2 | ¹H NMR (400 MHz, Chloroform-d) δ 8.24 (d, 1H), 7.47 (d, 1H), 7.13 − 6.91 (m, 2H), 6.84 − 6.82 (m, 2H), 6.56 (s, 1H), 5.41 − 5.39 (m, 1H), 4.57 − 4.27 (m, 5H), 4.25 − 4.05 (m, 2H), 3.77 − 3.70 (m, 1H), 2.28 − 2.01 (m, 3H), 1.91 − 1.77 (m, 2H), 1.74 − 1.18 (m, 14H). |
| 36 | 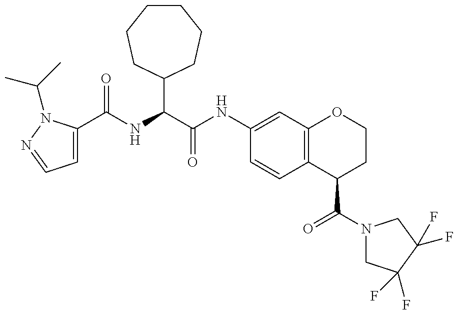 [SMILES: CC(C)n1nccc1C(=O)N[C@H](C(=O)Nc1ccc2c(c1)OCC[C@H]2C(=O)N1CC(F)(F)C(F)(F)C1)C1CCCCCC1] | 609.3 | ¹H NMR (400 MHz, Chloroform-d) δ 8.23 (d, 1H), 7.49 (d, 1H), 7.13 − 6.91 (m, 2H), 6.84 − 6.82 (m, 2H), 6.56 (s, 1H), 5.41 − 5.39 (m, 1H), 4.57 − 4.27 (m, 5H), 4.25 − 4.05 (m, 2H), 3.77 − 3.70 (m, 1H), 2.28 − 2.01 (m, 3H), 1.91 − 1.77 (m, 2H), 1.74 − 1.18 (m, 18H). |
| 37 | 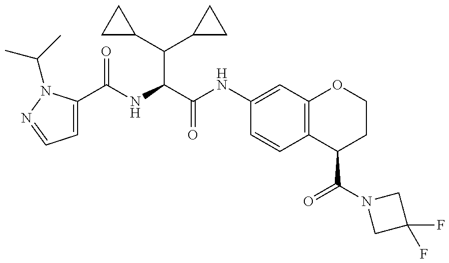 [SMILES: CC(C)n1nccc1C(=O)N[C@H](C(=O)Nc1ccc2c(c1)OCC[C@H]2C(=O)N1CC(F)(F)C1)C(C1CC1)C1CC1] | 557.3 | ¹H NMR (400 MHz, Chloroform-d) δ 8.24 (d, 1H), 7.46 (d, 1H), 7.13 − 6.91 (m, 2H), 6.84 − 6.82 (m, 2H), 6.56 (s, 1H), 5.41 − 5.39 (m, 1H), 4.57 − 4.27 (m, SH), 4.25 − 4.05 (m, 2H), 3.77 − 3.70 (m, 1H), 2.28 − 2.01 (m, 3H), 1.91 − 1.77 (m, 2H), 1.74 − 1.18 (m, 16H). |
| 38 | 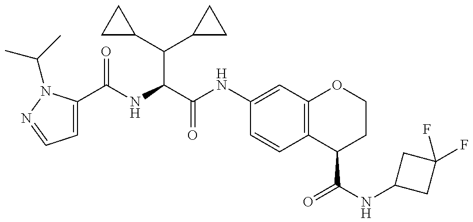 [SMILES: CC(C)n1nccc1C(=O)N[C@H](C(=O)Nc1ccc2c(c1)OCC[C@H]2C(=O)NC1CC(F)(F)C1)C(C1CC1)C1CC1] | 570.3 | ¹H NMR (400 MHz, Chloroform-d) δ 9.50 (s, 1H), 7.67 − 7.60 (m, 1H), 7.50 (d, J = 7.4 Hz, 1H), 7.18 − 7.11 (m, 1H), 6.98 (d, J = 1.9 Hz, 1H), 6.80 − 6.70 (m, 2H), 6.28 (s, 1H), 5.56 − 5.41 (m, 1H), 4.95 (d, J = 6.8 Hz, 1H), 4.51 − 4.39 (m, 1H), 4.34 − 4.22 (m, 1H), 4.17 − 3.98 (m, 2H), 2.91 − 2.69 (m, 2H), 2.74 − 2.61 (m, 2H), 2.66 − 2.46 (m, 2H), 2.07 − 1.94 (m, 2H), 1.55 − 1.43 (m, 6H), 1.20 − 1.01 (m, 2H), 0.71 − 0.54 (m, 2H), 0.58 − 0.30 (m, 5H), 0.26 − 0.12 (m, 1H). |
| 39 | 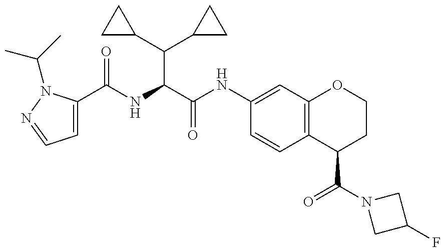 [SMILES: CC(C)n1nccc1C(=O)N[C@H](C(=O)Nc1ccc2c(c1)OCC[C@H]2C(=O)N1CC(F)C1)C(C1CC1)C1CC1] | 538.3 | ¹H NMR (400 MHz, Chloroform-d) δ 9.50 (s, 1H), 7.76 − 7.68 (m, 1H), 7.53 (d, J = 7.6 Hz, 1H), 7.28 − 7.21 (m, 1H), 7.03 (d, 1H), 6.82 − 6.70 (m, 2H), 5.38 − 5.23 (m, 1H), 5.12 − 4.95 (m, 2H), 4.35 − 4.12 (m, 4H), 3.89 − 3.69 (m, 3H), 2.45 − 2.31 (m, 1H), 2.11 − 1.97 (m, 2H), 1.55 − 1.43 (m, 6H), 1.33 − 1.22 (m, 2H), 0.71 − 0.58 (m, 4H), 0.43 − 0.30 (m, 4H). |
| 40 | 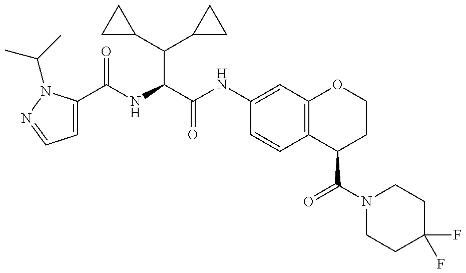 [SMILES: CC(C)n1nccc1C(=O)N[C@H](C(=O)Nc1ccc2c(c1)OCC[C@H]2C(=O)N1CCC(F)(F)CC1)C(C1CC1)C1CC1] | 584.3 | ¹H NMR (400 MHz, Chloroform-d) δ 9.50 (s, 1H), 7.99 (s, 1H), 7.81 − 7.73 (m, 1H), 7.52 (d, J = 7.4 Hz, 1H), 7.31 − 7.24 (m, 1H), 6.88 − 6.81 (m, 2H), 5.43 − 5.28 (m, 1H), 5.10 (d, J = 7.0 Hz, 1H), 4.34 − 4.23 (m, 1H), 4.15 − 4.04 (m, 1H), 3.76 − 3.67 (m, 1H), 3.39 (t, J = 5.8 Hz, 4H), 2.75 − 2.62 (m, 1H), 2.11 − 1.97 (m, 3H), 2.01 − 1.88 (m, 3H), 1.55 − 1.43 (m, 6H), 1.27 − 1.13 (m, 1H), 1.12 − 0.98 (m, 1H), 0.69 − 0.57 (m, 1H), 0.55 − 0.30 (m, 6H), 0.02 − −0.11 (m, 1H), |
| 41 | 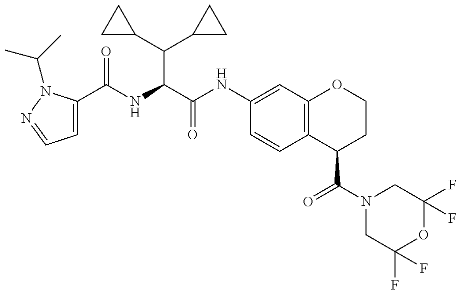 [SMILES: CC(C)n1nccc1C(=O)N[C@H](C(=O)Nc1ccc2c(c1)OCC[C@H]2C(=O)N1CC(F)(F)OC(F)(F)C1)C(C1CC1)C1CC1] | 622.3 | ¹H NMR (400 MHz, Chloroform-d) δ 9.50 (s, 1H), 7.97 (s, 1H), 7.82 − 7.74 (m, 1H), 7.52 (d, J = 7.6 Hz, 1H), 7.32 − 7.25 (m, 1H), 6.88 − 6.81 (m, 2H), 5.42 − 5.27 (m, 1H), 5.09 − 4.85 (m, 2H), 4.55 − 4.36 (m, 1H), 4.35 − 4.23 (m, 1H), 4.16 − 3.73 (m, 4H), 2.77 − 2.64 (m, 1H), 2.09 − 1.97 (m, 2H), 1.55 − 1.43 (m, 6H), 1.11 − 1.00 (m, 2H), 0.61 − 0.35 (m, 6H), 0.40 − 0.24 (m, 2H), 0.23 − 0.11 (m, 1H). |
| 42 | 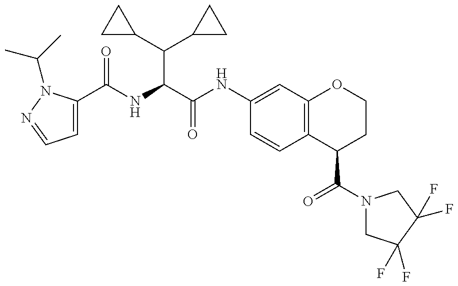 [SMILES: CC(C)n1nccc1C(=O)N[C@H](C(=O)Nc1ccc2c(c1)OCC[C@H]2C(=O)N1CC(F)(F)C(F)(F)C1)C(C1CC1)C1CC1] | 606.3 | ¹H NMR (400 MHz, Chloroform-d) δ 9.50 (s, 1H), 7.69 − 7.61 (m, 1H), 7.50 (d, J = 7.6 Hz, 1H), 7.17 − 7.10 (m, 1H), 6.99 (d, 1H), 6.74 (d, J = 7.6 Hz, 1H), 6.29 (s, 1H), 5.57 − 5.41 (m, 1H), 4.95 (d, J = 6.8 Hz, 1H), 4.58 − 4.32 (m, 2H), 4.34 − 4.22 (m, 2H), 4.19 − 3.82 (m, 4H), 2.49 − 2.36 (m, 1H), 2.09 − 1.97 (m, 2H), 1.55 − 1.43 (m, 6H), 1.20 − 1.01 (m, 2H), 0.72 − 0.55 (m, 2H), 0.60 − 0.39 (m, 5H), 0.43 − 0.30 (m, 2H), 0.26 − 0.12 (m, 1H). |
| 43 | 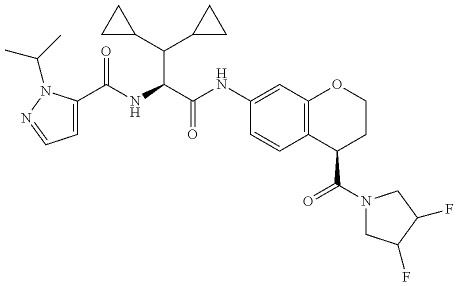 [SMILES: CC(C)n1nccc1C(=O)N[C@H](C(=O)Nc1ccc2c(c1)OCC[C@H]2C(=O)N1CC(F)C(F)C1)C(C1CC1)C1CC1] | 570.3 | ¹H NMR (400 MHz, Chloroform-d) δ 9.50 (s, 1H), 8.07 (s, 1H), 7.78 − 7.71 (m, 1H), 7.52 (d, J = 7.4 Hz, 1H), 7.18 − 7.11 (m, 1H), 6.88 − 6.79 (m, 2H), 5.44 − 5.29 (m, 1H), 5.11 (d, J = 7.0 Hz, 1H), 4.34 − 4.22 (m, 1H), 4.22 − 3.90 (m, 5H), 3.94 − 3.82 (m, 2H), 3.77 − 3.52 (m, 2H), 2.68 − 2.55 (m, 1H), 2.07 − 1.91 (m, 2H), 1.55 − 1.43 (m, 6H), 1.22 − 1.12 (m, 1H), 1.10 − 1.00 (m, 1H), 0.67 − 0.56 (m, 1H), 0.55 − 0.29 (m, 6H), 0.03 − −0.10 (m, 1H). |
| 44 | 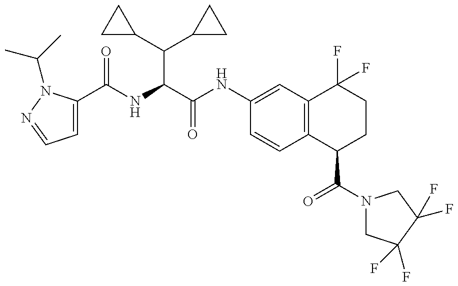 [SMILES: CC(C)n1nccc1C(=O)N[C@H](C(=O)Nc1ccc2c(c1)C(F)(F)CC[C@H]2C(=O)N1CC(F)(F)C(F)(F)C1)C(C1CC1)C1CC1] | 640.3 | ¹H NMR (400 MHz, Chloroform-d) δ 9.50 (s, 1H), 8.29 − 8.22 (m, 1H), 7.50 (d, J = 7.6 Hz, 1H), 7.09 (d, J = 2.1 Hz, 1H), 7.00 − 6.93 (m, 1H), 6.76 (d, J = 7.4 Hz, 1H), 6.32 (s, 1H), 5.28 − 5.13 (m, 1H), 4.94 (d, J = 7.0 Hz, 1H), 4.57 − 4.30 (m, 2H), 4.09 − 3.88 (m, 3H), 2.32 − 1.87 (m, 6H), 1.55 − 1.43 (m, 6H), 1.22 − 1.00 (m, 2H), 0.74 − 0.32 (m, 8H), 0.27 − 0.12 (m, 1H). |
| 45 | 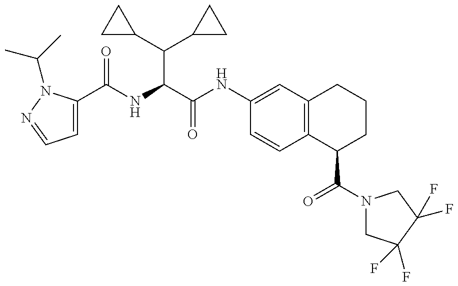 [SMILES: CC(C)n1nccc1C(=O)N[C@H](C(=O)Nc1ccc2c(c1)CCC[C@H]2C(=O)N1CC(F)(F)C(F)(F)C1)C(C1CC1)C1CC1] | 604.3 | ¹H NMR (400 MHz, Chloroform-d) δ 9.50 (s, 1H), 8.20 − 8.12 (m, 1H), 7.52 (d, J = 7.4 Hz, 1H), 7.05 − 6.97 (m, 2H), 6.76 (d, J = 7.4 Hz, 1H), 6.51 (s, 1H), 5.61 − 5.45 (m, 1H), 5.03 (d, J = 7.0 Hz, 1H), 4.56 − 4.33 (m, 2H), 4.08 − 3.85 (m, 3H), 3.00 − 2.76 (m, 2H), 2.12 − 1.80 (m, 5H), 1.55 − 1.43 (m, 6H), 1.17 − 1.02 (m, 2H), 0.80 − 0.69 (m, 1H), 0.66 − 0.21 (m, 8H). |
| 46 | 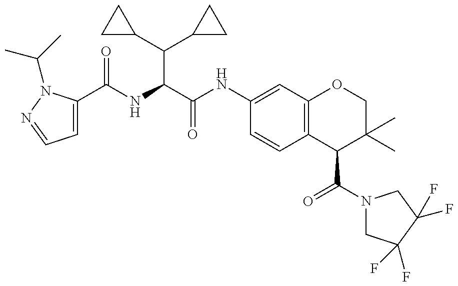 [SMILES: CC(C)n1nccc1C(=O)N[C@H](C(=O)Nc1ccc2c(c1)OCC(C)(C)[C@H]2C(=O)N1CC(F)(F)C(F)(F)C1)C(C1CC1)C1CC1] | 634.3 | ¹H NMR (400 MHz, Chloroform-d) δ 9.50 (s, 1H), 7.68 − 7.61 (m, 1H), 7.52 (d, J = 7.4 Hz, 1H), 7.09 − 7.02 (m, 1H), 6.98 (d, J = 1.9 Hz, 1H), 6.75 (d, J = 7.6 Hz, 1H), 6.54 (s, 1H), 5.59 − 5.44 (m, 1H), 5.04 (d, J = 7.0 Hz, 1H), 4.65 − 4.31 (m, 2H), 4.06 − 3.81 (m, 2H), 3.81 − 3.69 (m, 2H), 3.60 (d, J = 11.5 Hz, 1H), 2.07 − 1.97 (m, 1H), 1.55 − 1.43 (m, 6H), 1.15 − 1.03 (m, 2H), 0.98 (d, J = 10.0 Hz, 6H), 0.79 − 0.67 (m, 1H), 0.65 − 0.21 (m, 8H). |
| 47 | 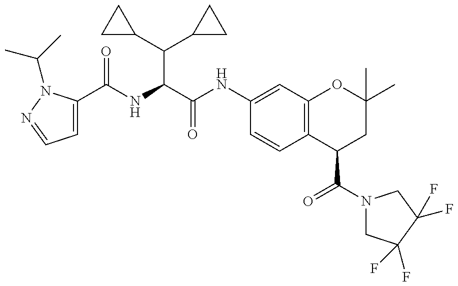 [SMILES: CC(C)n1nccc1C(=O)N[C@H](C(=O)Nc1ccc2c(c1)OC(C)(C)C[C@H]2C(=O)N1CC(F)(F)C(F)(F)C1)C(C1CC1)C1CC1] | 634.3 | ¹H NMR (400 MHz, Chloroform-d) δ 9.50 (s, 1H), 7.68 − 7.61 (m, 1H), 7.52 (d, J = 7.4 Hz, 1H), 7.16 − 7.09 (m, 1H), 6.96 (d, 1H), 6.76 (d, J = 7.6 Hz, 1H), 6.60 (s, 1H), 5.60 − 5.45 (m, 1H), 5.06 (d, J = 7.0 Hz, 1H), 4.57 − 4.29 (m, 2H), 4.09 − 3.99 (m, 2H), 4.03 − 3.90 (m, 2H), 2.35 − 2.25 (m, 1H), 2.07 − 1.97 (m, 1H), 1.93 − 1.83 (m, 1H), 1.55 − 1.43 (m, 9H), 1.41 (s, 3H), 1.15 − 1.02 (m, 2H), 0.84 − 0.72 (m, 1H), 0.64 − 0.23 (m, 8H). |
| 48 | 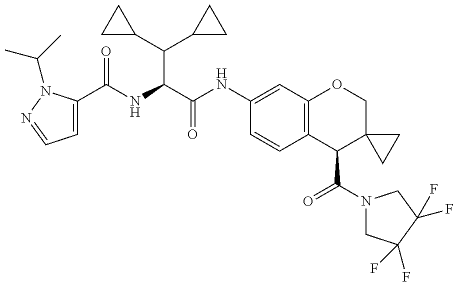 [SMILES: CC(C)n1nccc1C(=O)N[C@H](C(=O)Nc1ccc2c(c1)OCC1(CC1)[C@H]2C(=O)N1CC(F)(F)C(F)(F)C1)C(C1CC1)C1CC1] | 632.3 | ¹H NMR (400 MHz, Chloroform-d) δ 9.50 (s, 1H), 7.78 − 7.71 (m, 1H), 7.67 (s, 1H), 7.51 (d, J = 7.4 Hz, 1H), 7.11 − 7.04 (m, 1H), 6.90 (d, J = 1.9 Hz, 1H), 6.81 (d, J = 7.6 Hz, 1H), 5.42 − 5.27 (m, 1H), 5.06 (d, J = 7.0 Hz, 1H), 4.88 − 4.70 (m, 1H), 4.56 − 4.38 (m, 1H), 4.13 − 3.85 (m, 3H), 3.81 − 3.73 (m, 2H), 2.07 − 1.97 (m, 1H), 1.55 − 1.30 (m, 7H), 1.15 − 1.04 (m, 2H), 0.90 − 0.78 (m, 1H), 0.54 − 0.10 (m, 10H). |
| 49 | 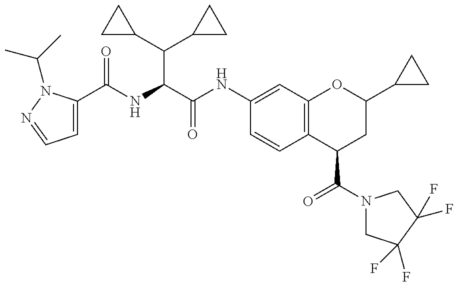 [SMILES: CC(C)n1nccc1C(=O)N[C@H](C(=O)Nc1ccc2c(c1)OC(C1CC1)C[C@H]2C(=O)N1CC(F)(F)C(F)(F)C1)C(C1CC1)C1CC1] | 646.3 | ¹H NMR (400 MHz, Chloroform-d) δ 9.50 (s, 1H), 8.02 (s, 1H), 7.79 − 7.72 (m, 1H), 7.52 (d, J = 7.4 Hz, 1H), 7.22 − 7.15 (m, 1H), 6.88 − 6.82 (m, 2H), 5.44 − 5.29 (m, 1H), 5.10 (d, J = 7.0 Hz, 1H), 4.60 − 4.26 (m, 3H), 4.21 − 3.96 (m, 2H), 3.22 − 3.12 (m, 1H), 2.75 − 2.63 (m, 1H), 2.08 − 1.97 (m, 2H), 1.55 − 1.43 (m, 6H), 1.26 − 1.13 (m, 1H), 1.10 − 0.98 (m, 2H), 0.70 − 0.59 (m, 1H), 0.55 − 0.32 (m, 8H), 0.36 − 0.26 (m, 2H), 0.00 − −0.13 (m, 1H). |
| 50 | 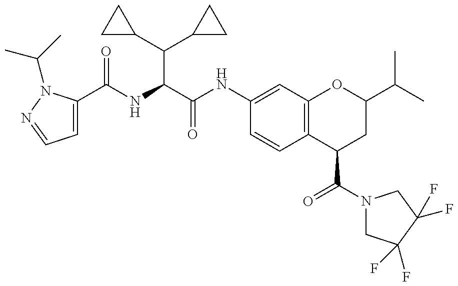 [SMILES: CC(C)C1C[C@@H](C(=O)N2CC(F)(F)C(F)(F)C2)c2ccc(NC(=O)[C@@H](NC(=O)c3ccnn3C(C)C)C(C3CC3)C3CC3)cc2O1] | 648.3 | ¹H NMR (400 MHz, Chloroform-d) δ 9.50 (s, 1H), 7.77 (s, 1H), 7.52 (d, J = 7.6 Hz, 1H), 7.22 − 7.14 (m, 1H), 6.88 − 6.77 (m, 2H), 5.39 − 5.24 (m, 1H), 5.18 (d, ) = 7.0 Hz, 1H), 4.58 − 4.40 (m, 1H), 4.34 − 3.86 (m, 4H), 3.79 − 3.69 (m, 1H), 2.40 − 2.29 (m, 1H), 2.07 − 1.88 (m, 3H), 1.55 − 1.43 (m, 6H), 1.20 − 1.03 (m, 2H), 0.91 (d, J = 6.8 Hz, 3H), 0.81 (d, J = 6.8 Hz, 3H), 0.65 − 0.47 (m, 2H), 0.51 − 0.35 (m, 4H), 0.40 − 0.31 (m, 2H). 0.01 − −0.13 (m, 1H). |
| 51 | 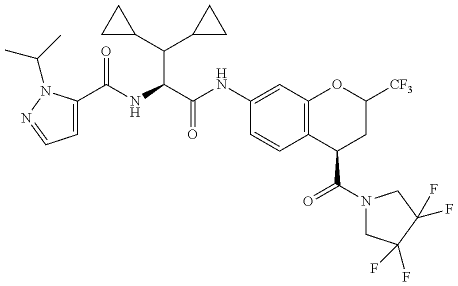 [SMILES: CC(C)n1nccc1C(=O)N[C@H](C(=O)Nc1ccc2c(c1)OC(C(F)(F)F)C[C@H]2C(=O)N1CC(F)(F)C(F)(F)C1)C(C1CC1)C1CC1] | 674.2 | ¹H NMR (400 MHz, Chloroform-d) δ 9.50 (s, 1H), 7.71 − 7.64 (m, 1H), 7.51 (d, .J = 7.4 Hz, 1H), 7.18 − 7.11 (m, 1H), 7.03 (d, J = 2.1 Hz, 1H), 6.76 (d, J = 7.4 Hz, 1H), 6.32 (s, 1H), 4.97 − 4.84 (m, 2H), 4.57 − 4.43 (m, 2H), 4.47 − 4.31 (m, 2H), 4.09 − 3.96 (m, 2H), 3.99 − 3.89 (m, 2H), 2.48 − 2.37 (m, 1H), 2.27 − 2.16 (m, 1H), 2.07 − 1.97 (m, 1H), 1.55 − 1.43 (m, 6H), 1.20 − 1.01 (m, 2H), 0.73 − 0.60 (m, 1H), 0.61 − 0.30 (m, 7H). 0.25 − 0.13 (m, 1H). |
| 52 | 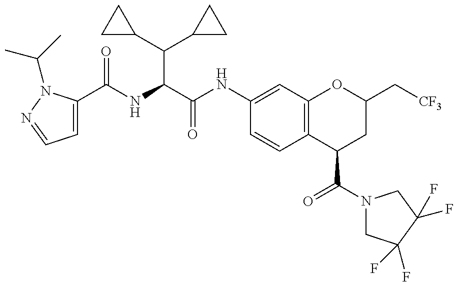 [SMILES: CC(C)n1nccc1C(=O)N[C@H](C(=O)Nc1ccc2c(c1)OC(CC(F)(F)F)C[C@H]2C(=O)N1CC(F)(F)C(F)(F)C1)C(C1CC1)C1CC1] | 688.3 | ¹H NMR (400 MHz, Chloroform-d) δ 9.50 (s, 1H), 7.76 − 7.68 (m, 1H), 7.52 (d, J = 7.4 Hz, 1H), 7.28 − 7.21 (m, 1H), 7.19 (s, 1H), 6.88 (d, J = 1.9 Hz, 1H), 6.78 (d, J = 7.4 Hz, 1H), 5.32 − 5.15 (m, 2H), 4.57 − 4.34 (m, 2H), 4.18 − 3.87 (m, 4H), 2.71 − 2.53 (m, 1H), 2.48 − 2.32 (m, 2H), 2.24 − 2.12 (m, 1H), 2.07 − 1.97 (m, 1H), 1.55 − 1.43 (m, 6H), 1.19 − 1.07 (m, 2H), 0.61 − 0.37 (m, 6H), 0.41 − 0.32 (m, 2H), 0.10 − −0.04 (m, 1H). |
| 53 | 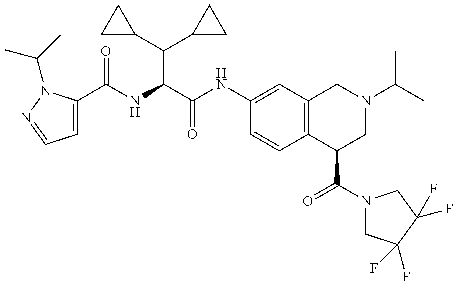 [SMILES: CC(C)N1Cc2cc(NC(=O)[C@@H](NC(=O)c3ccnn3C(C)C)C(C3CC3)C3CC3)ccc2[C@H](C(=O)N2CC(F)(F)C(F)(F)C2)C1] | 647.3 | ¹H NMR (400 MHz, Chloroform-d) δ 9.50 (s, 1H), 8.12 (s, 1H), 8.04 − 7.97 (m, 1H), 7.52 (d, J = 7.4 Hz, 1H), 7.21 − 7.14 (m, 1H), 7.13 − 7.07 (m, 1H), 6.82 (d, J = 7.4 Hz, 1H), 5.44 − 5.28 (m, 1H), 5.12 (d, J = 7.0 Hz, 1H), 4.59 − 4.29 (m, 2H), 4.26 − 3.94 (m, 4H), 3.60 − 3.51 (m, 1H), 3.15 − 3.05 (m, 1H), 2.93 − 2.83 (m, 1H), 2.69 − 2.53 (m, 1H), 2.07 − 1.97 (m, 1H), 1.55 − 1.43 (m, 6H), 1.24 − 1.10 (m, 4H), 1.10 − 0.97 (m, 4H), 0.69 − 0.58 (m, 1H), 0.55 − 0.30 (m, 6H), 0.01 − −0.13 (m, 1H). |
| 54 | 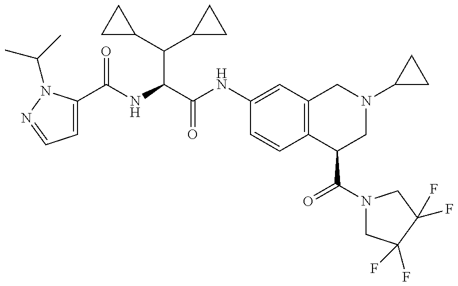 [SMILES: CC(C)n1nccc1C(=O)N[C@H](C(=O)Nc1ccc2c(c1)CN(C1CC1)C[C@H]2C(=O)N1CC(F)(F)C(F)(F)C1)C(C1CC1)C1CC1] | 645.3 | ¹H NMR (400 MHz. Chloroform-d) δ 9.50 (s, 1H), 8.03 − 7.96 (m, 1H), 7.75 (s, 1H), 7.52 (d, J = 7.4 Hz, 1H), 7.19 − 7.09 (m, 2H), 6.82 (d, J = 7.4 Hz, 1H), 5.40 − 5.24 (m, 1H), 5.17 (d, J = 7.0 Hz, 1H), 4.58 − 4.40 (m, 1H), 4.36 − 3.91 (m, 6H), 3.52 − 3.42 (m, 1H), 3.06 − 2.96 (m, 1H), 2.36 − 2.24 (m, 1H), 2.07 − 1.97 (m, 1H), 1.55 − 1.43 (m, 6H), 1.23 − 1.01 (m, 2H), 0.66 − 0.23 (m, 12H), 0.01 − −0.13 (m, 1H). |
| 55 | 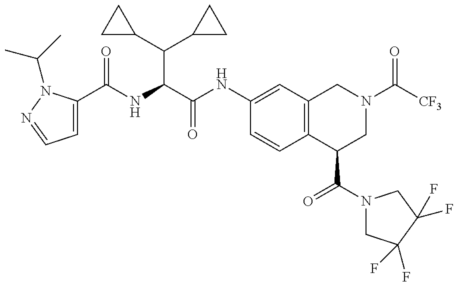 [SMILES: CC(C)n1nccc1C(=O)N[C@H](C(=O)Nc1ccc2c(c1)CN(C(=O)C(F)(F)F)C[C@H]2C(=O)N1CC(F)(F)C(F)(F)C1)C(C1CC1)C1CC1] | 701.3 | ¹H NMR (400 MHz, Chloroform-d) δ 9.50 (s, 1H), 7.95 − 7.87 (m, 1H), 7.50 (d, J = 7.4 Hz, 1H), 7.37 − 7.31 (m, 1H), 7.21 − 7.14 (m, 1H), 6.73 (d, J = 7.4 Hz, 1H), 6.29 (s, 1H), 5.57 − 5.41 (m, 1H), 5.06 − 4.91 (m, 2H), 4.64 − 4.47 (m, 2H), 4.38 − 4.30 (m, 1H), 4.28 − 4.17 (m, 2H), 4.19 − 4.03 (m, 1H), 4.05 − 3.90 (m, 1H), 2.07 − 1.97 (m, 1H), 1.55 − 1.43 (m, 6H), 1.20 − 1.02 (m, 2H), 0.72 − 0.54 (m, 2H), 0.59 − 0.48 (m, 2H), 0.53 − 0.39 (m, 2H), 0.43 − 0.31 (m, 2H). 0.26 − 0.13 (m, 1H). |
| 56 | 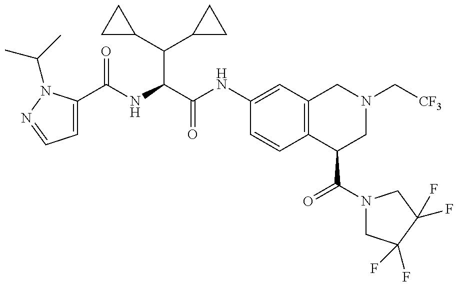 [SMILES: CC(C)n1nccc1C(=O)N[C@H](C(=O)Nc1ccc2c(c1)CN(CC(F)(F)F)C[C@H]2C(=O)N1CC(F)(F)C(F)(F)C1)C(C1CC1)C1CC1] | 687.3 | ¹H NMR (400 MHz, Chloroform-d) δ 9.50 (s, 1H), 8.05 − 7.97 (m, 1H), 7.95 (s, 1H), 7.52 (d, J = 7.6 Hz, 1H), 7.22 − 7.15 (m, 1H), 7.14 − 7.08 (m, 1H), 6.84 (d, J = 7.4 Hz, 1H), 5.43 − 5.28 (m, 1H), 5.08 (d, J = 7.0 Hz, 1H), 4.59 − 4.28 (m, 2H), 4.20 − 3.94 (m, 4H), 3.83 − 3.63 (m, 2H), 3.30 − 3.20 (m, 1H), 3.19 − 2.96 (m, 2H), 2.07 − 1.97 (m, 1H), 1.55 − 1.43 (m, 6H), 1.25 − 1.14 (m, 1H), 1.12 − 1.02 (m, 1H), 0.67 − 0.56 (m, 1H), 0.55 − 0.27 (m, 6H), 0.05 − −0.09 (m, 1H). |
| 57 | 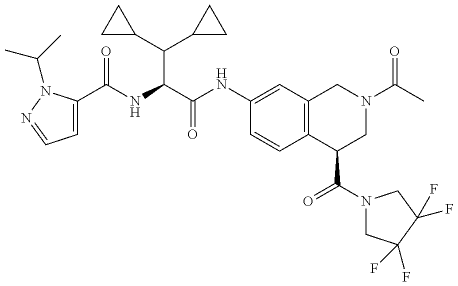 [SMILES: CC(=O)N1Cc2cc(NC(=O)[C@@H](NC(=O)c3ccnn3C(C)C)C(C3CC3)C3CC3)ccc2[C@H](C(=O)N2CC(F)(F)C(F)(F)C2)C1] | 647.3 | ¹H NMR (400 MHz, Chloroform-d) δ 9.50 (s, 1H), 8.08 − 8.01 (m, 1H), 7.97 (s, 1H), 7.52 (d, J = 7.4 Hz, 1H), 7.24 − 7.17 (m, 2H), 6.84 (d, J = 7.4 Hz, 1H), 5.42 − 5.27 (m, 1H), 5.07 (d, J = 6.8 Hz, 1H), 5.00 − 4.90 (m, 1H), 4.65 − 4.30 (m, 4H), 4.18 − 4.05 (m, 2H), 4.09 − 3.98 (m, 2H), 3.92 − 3.82 (m, 1H), 2.18 (s, 3H), 2.07 − 1.97 (m, 1H), 1.55 − 1.43 (m, 6H), 1.10 − 0.99 (m, 2H), 0.61 − 0.48 (m, 2H), 0.53 − 0.30 (m, 5H), 0.33 − 0.13 (m, 2H). |
| 58 | 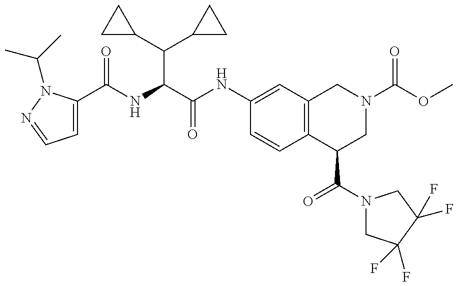 [SMILES: COC(=O)N1Cc2cc(NC(=O)[C@@H](NC(=O)c3ccnn3C(C)C)C(C3CC3)C3CC3)ccc2[C@H](C(=O)N2CC(F)(F)C(F)(F)C2)C1] | 663.3 | ¹H NMR (400 MHz, Chloroform-d) δ 9.50 (s, 1H), 7.95 − 7.87 (m, 1H), 7.50 (d, J = 7.4 Hz, 1H), 7.39 − 7.32 (m, 1H), 7.20 − 7.13 (m, 1H), 6.73 (d, J = 7.4 Hz, 1H), 6.29 (s, 1H), 5.57 − 5.42 (m, 1H), 5.08 − 4.92 (m, 2H), 4.66 − 4.48 (m, 2H), 4.38 − 4.04 (m, 5H), 4.07 − 3.89 (m, 2H), 3.61 (s, 3H), 2.07 − 1.97 (m, 1H), 1.55 − 1.43 (m, 6H), 1.20 − 1.02 (m, 2H), 0.72 − 0.31 (m, 8H), 0.26 − 0.12 (m, 1H). |
| 59 | 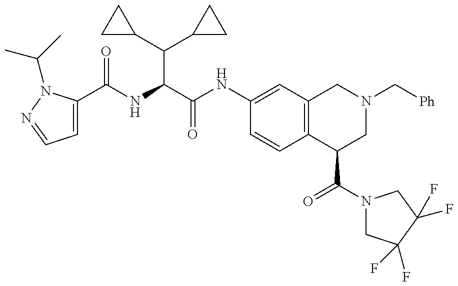 [SMILES: CC(C)n1nccc1C(=O)N[C@H](C(=O)Nc1ccc2c(c1)CN(Cc1ccccc1)C[C@H]2C(=O)N1CC(F)(F)C(F)(F)C1)C(C1CC1)C1CC1] | 695.3 | ¹H NMR (400 MHz, Chloroform-d) δ 9.50 (s, 1H), 8.14 (s, 1H), 8.04 − 7.97 (m, 1H), 7.52 (d, J = 7.6 Hz, 1H), 7.31 − 7.10 (m, 7H), 6.83 (d, J = 7.4 Hz, 1H), 5.44 − 5.29 (m, 1H), 5.13 (d, J = 7.0 Hz, 1H), 4.53 − 4.35 (m, 1H), 4.24 − 3.95 (m, 5H), 3.98 − 3.80 (m, 2H), 3.74 − 3.64 (m, 1H), 3.52 − 3.43 (m, 1H), 3.18 − 3.08 (m, 1H), 2.80 − 2.71 (m, 1H), 2.07 − 1.97 (m, 1H), 1.55 − 1.43 (m, 6H), 1.25 − 1.11 (m, 1H), 1.11 − 0.97 (m, 1H), 0.70 − 0.58 (m, 1H), 0.55 − 0.31 (m, 6H), 0.02 − −0.12 (m, 1H). |
| 60 | 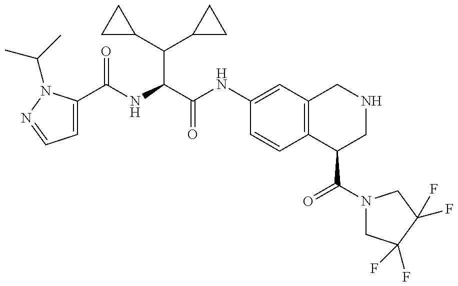 [SMILES: CC(C)n1nccc1C(=O)N[C@H](C(=O)Nc1ccc2c(c1)CNC[C@H]2C(=O)N1CC(F)(F)C(F)(F)C1)C(C1CC1)C1CC1] | 605.3 | ¹H NMR (400 MHz, Chloroform-d) δ 9.50 (s, 1H), 7.92 − 7.84 (m, 1H), 7.51 (d, J = 7.4 Hz, 1H), 7.27 − 7.21 (m, 1H), 7.17 − 7.10 (m, 1H), 6.74 (d, J = 7.4 Hz, 1H), 6.48 (s, 1H), 5.58 − 5.43 (m, 1H), 5.02 (d, J = 6.9 Hz, 1H), 4.57 − 4.39 (m, 1H), 4.30 − 3.91 (m, 6H), 3.63 − 3.53 (m, 1H), 3.17 − 3.07 (m, 1H), 2.07 − 1.97 (m, 1H), 1.55 − 1.43 (m, 7H), 1.18 − 1.00 (m, 2H), 0.79 − 0.67 (m, 1H), 0.66 − 0.56 (m, 1H), 0.60 − 0.20 (m, 6H). |
| 61 | 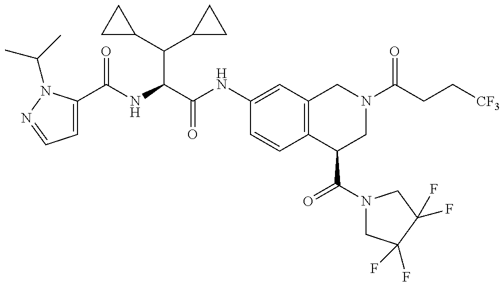 [SMILES: CC(C)n1nccc1C(=O)N[C@H](C(=O)Nc1ccc2c(c1)CN(C(=O)CCC(F)(F)F)C[C@H]2C(=O)N1CC(F)(F)C(F)(F)C1)C(C1CC1)C1CC1] | 729.3 | ¹H NMR (400 MHz. Chloroform-d) δ 9.50 (s, 1H), 7.94 − 7.87 (m, 1H), 7.50 (d, J = 7.6 Hz, 1H), 7.37 − 7.31 (m, 1H), 7.21 − 7.14 (m, 1H), 6.75 (d, J = 7.4 Hz, 1H), 6.31 (s, 1H), 5.30 − 5.15 (m, 1H), 5.03 − 4.91 (m, 2H), 4.64 − 4.46 (m, 2H), 4.38 − 4.30 (m, 1H), 4.30 − 3.97 (m, 4H), 3.91 − 3.81 (m, 1H), 3.41 − 3.23 (m, 1H), 3.02 − 2.88 (m, 2H), 2.52 − 2.35 (m, 1H), 2.07 − 1.97 (m, 1H), 1.55 − 1.43 (m, 6H), 1.20 − 1.02 (m, 2H), 0.72 − 0.53 (m, 2H), 0.58 − 0.31 (m, 6H), 0.26 − 0.12 (m, 1H). |
| 62 | 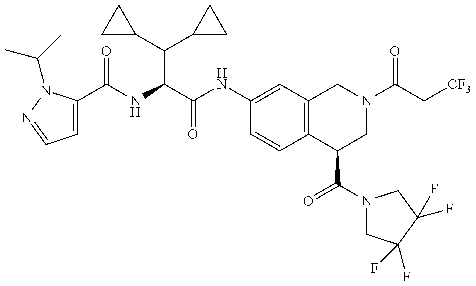 [SMILES: CC(C)n1nccc1C(=O)N[C@H](C(=O)Nc1ccc2c(c1)CN(C(=O)CC(F)(F)F)C[C@H]2C(=O)N1CC(F)(F)C(F)(F)C1)C(C1CC1)C1CC1] | 715.3 | ¹H NMR (400 MHz, Chloroform-d) δ 9.50 (s, 1H), 8.09 − 8.01 (m, 1H), 8.00 (s, 1H), 7.52 (d, J = 7.4 Hz, 1H), 7.25 − 7.18 (m, 2H), 6.85 (d, J = 7.4 Hz, 1H), 5.44 − 5.29 (m, 1H), 5.11 (d, J = 7.0 Hz, 1H), 5.02 − 4.92 (m, 1H), 4.65 − 4.30 (m, 4H), 4.19 − 4.03 (m, 2H), 4.06 − 3.96 (m, 1H), 3.93 − 3.83 (m, 1H), 3.46 − 3.31 (m, 1H), 3.26 − 3.11 (m, 1H), 2.07 − 1.97 (m, 1H), 1.55 − 1.43 (m, 6H), 1.26 − 1.12 (m, 1H), 1.11 − 0.97 (m, 1H), 0.71 − 0.59 (m, 1H), 0.55 − 0.31 (m, 6H), 0.01 − −0.13 (m, 1H). |
| 63 | 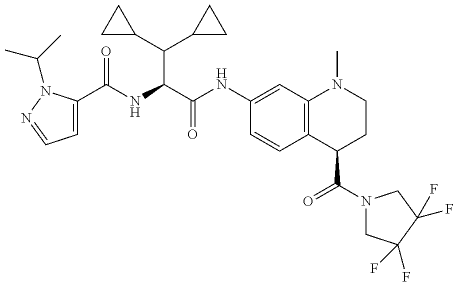 [SMILES: CC(C)n1nccc1C(=O)N[C@H](C(=O)Nc1ccc2c(c1)N(C)CC[C@H]2C(=O)N1CC(F)(F)C(F)(F)C1)C(C1CC1)C1CC1] | 619.3 | ¹H NMR (400 MHz, Chloroform-d) δ 9.50 (s, 1H), 7.50 (d, J = 7.6 Hz, 1H), 7.36 − 7.29 (m, 1H), 7.02 − 6.95 (m, 1H), 6.91 (d, J = 1.9 Hz, 1H), 6.77 (d, J = 7.4 Hz, 1H), 6.60 (s, 1H), 5.25 − 5.09 (m, 1H), 4.96 (d, J = 6.8 Hz, 1H), 4.58 − 4.26 (m, 2H), 4.16 − 3.93 (m, 2H), 3.78 − 3.69 (m, 1H), 3.33 − 3.21 (m, 1H), 3.12 − 3.00 (m, 1H), 3.00 (s, 3H), 2.62 − 2.48 (m, 1H), 2.07 − 1.97 (m, 1H), 1.85 − 1.72 (m, 1H), 1.55 − 1.43 (m, 6H), 1.18 − 1.01 (m, 2H), 0.68 − 0.32 (m, 7H), 0.27 − 0.15 (m, 1H). |
| 64 | 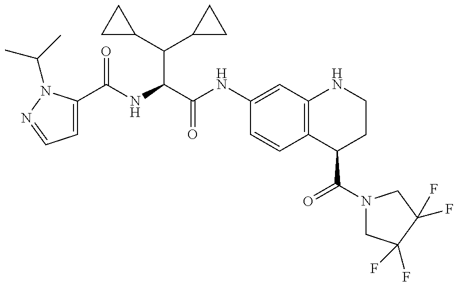 [SMILES: CC(C)n1nccc1C(=O)N[C@H](C(=O)Nc1ccc2c(c1)NCC[C@H]2C(=O)N1CC(F)(F)C(F)(F)C1)C(C1CC1)C1CC1] | 605.3 | ¹H NMR (400 MHz, Chloroform-d) δ 9.50 (s, 1H), 7.52 (d, J = 7.4 Hz, 1H), 7.40 − 7.32 (m, 1H), 7.14 − 7.07 (m, 1H), 6.75 (d, J = 7.4 Hz, 1H), 6.51 − 6.42 (m, 2H), 5.59 − 5.44 (m, 1H), 5.03 (d, J = 7.0 Hz, 1H), 4.56 − 4.31 (m, 2H), 4.06 − 3.85 (m, 3H), 3.51 − 3.39 (m, 1H), 3.20 − 3.09 (m, 1H), 2.27 − 2.14 (m, 1H), 2.07 − 1.84 (m, 2H), 1.55 − 1.43 (m, 6H), 1.17 − 1.02 (m, 2H), 0.79 − 0.67 (m, 1H), 0.66 − 0.20 (m, 8H). |
| 65 | 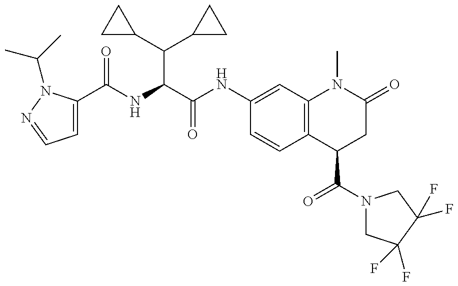 [SMILES: CC(C)n1nccc1C(=O)N[C@H](C(=O)Nc1ccc2c(c1)N(C)C(=O)C[C@H]2C(=O)N1CC(F)(F)C(F)(F)C1)C(C1CC1)C1CC1] | 633.3 | ¹H NMR (400 MHz, Chloroform-d) δ 9.50 (s, 1H), 7.95 − 7.88 (m, 1H), 7.51 (d, J = 7.6 Hz, 1H), 7.46 − 7.39 (m, 1H), 6.95 (d, 1H), 6.75 (d, J = 7.4 Hz, 1H), 6.33 (s, 1H), 5.57 − 5.42 (m, 1H), 4.95 (d, J = 7.0 Hz, 1H), 4.62 − 4.44 (m, 1H), 4.44 − 4.26 (m, 2H), 4.05 − 3.82 (m, 2H), 3.55 (s, 3H), 3.16 − 3.06 (m, 1H), 2.92 − 2.82 (m, 1H), 2.07 − 1.97 (m, 1H), 1.55 − 1.43 (m, 6H), 1.21 − 1.02 (m, 2H), 0.72 − 0.60 (m, 1H), 0.63 − 0.31 (m, 7H), 0.27 − 0.13 (m, 1H). |
| 66 | 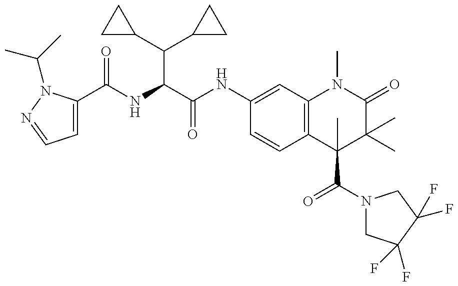 | 675.3 | ¹H NMR (400 MHz, Chloroform-d) δ 9.50 (s, 1H), 7.99 (s, 1H), 8.03 − 7.96 (m, 1H), 7.53 (d, J = 7.4 Hz, 1H), 7.32 (d, J = 7.4 Hz, 1H), 6.85 (d, J = 7.4 Hz, 1H), 6.79 (d, 1H), 5.44 − 5.29 (m, 1H), 5.11 (d, J = 7.0 Hz, 1H), 4.65 − 4.47 (m, 1H), 4.18 − 3.90 (m, 3H), 3.52 (s, 3H), 2.07 − 1.97 (m, 1H), 1.58 (s, 3H), 1.55 − 1.43 (m, 6H), 1.26 (s, 3H), 1.12 − 1.00 (m, 1H), 0.91 (s, 3H), 0.69 − 0.58 (m, 1H), 0.56 − 0.29 (m, 7H), 0.05 − −0.07 (m, 1H). |
| 67 | 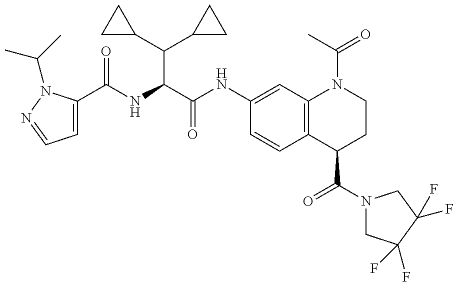 [SMILES: CC(=O)N1CC[C@@H](C(=O)N2CC(F)(F)C(F)(F)C2)c2ccc(NC(=O)[C@@H](NC(=O)c3ccnn3C(C)C)C(C3CC3)C3CC3)cc21] | 647.3 | ¹H NMR (400 MHz, Chloroform-d) δ 9.50 (s, 1H), 8.07 − 8.00 (m, 1H), 7.60 (s, 1H), 7.54 (d, J = 7.4 Hz, 1H), 7.38 − 7.31 (m, 1H), 6.83 − 6.76 (m, 2H), 5.43 − 5.28 (m, 1H), 5.11 (d, J = 7.0 Hz, 1H), 4.51 − 4.33 (m, 1H), 4.25 − 4.10 (m, 2H), 4.12 − 4.00 (m, 2H), 4.02 − 3.86 (m, 2H), 3.50 − 3.38 (m, 1H), 2.22 − 2.09 (m, 1H), 2.09 (s, 3H), 2.07 − 1.88 (m, 2H), 1.55 − 1.43 (m, 7H), 1.36 − 1.25 (m, 1H), 1.22 − 1.12 (m, 1H), 0.56 − 0.47 (m, 2H), 0.48 (s, 1H), 0.49 − 0.38 (m, 2H), 0.43 − 0.32 (m, 2H), 0.31 − 0.21 (m, 2H). |
| 68 | 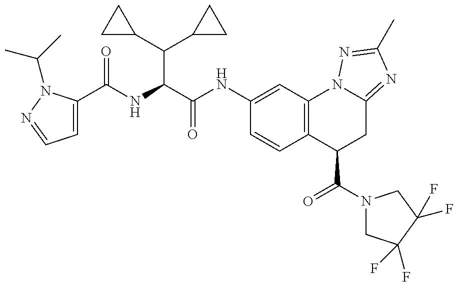 [SMILES: Cc1nc2n(n1)-c1cc(NC(=O)[C@@H](NC(=O)c3ccnn3C(C)C)C(C3CC3)C3CC3)ccc1[C@H](C(=O)N1CC(F)(F)C(F)(F)C1)C2] | 657.3 | ¹H NMR (400 MHz, Chloroform-d) δ 9.50 (s, 1H), 8.06 − 7.98 (m, 1H), 7.88 (d, J = 2.1 Hz, 1H), 7.54 (d, J = 7.6 Hz, 1H), 7.25 − 7.18 (m, 1H), 6.96 (s, 1H), 6.77 (d, J = 7.4 Hz, 1H), 5.07 − 4.89 (m, 2H), 4.68 − 4.50 (m, 1H), 4.45 − 4.37 (m, 1H), 4.30 − 4.14 (m, 2H), 4.13 − 3.96 (m, 1H), 3.82 − 3.71 (m, 1H), 3.25 − 3.14 (m, 1H), 2.58 (s, 3H), 2.07 − 1.97 (m, 1H), 1.55 − 1.43 (m, 6H), 1.21 − 1.10 (m, 2H), 1.03 − 0.91 (m, 1H), 0.91 − 0.79 (m, 1H), 0.61 − 0.21 (m, 7H). |
| 69 | 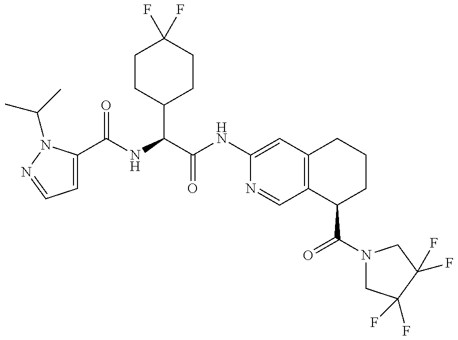 [SMILES: CC(C)n1nccc1C(=O)N[C@H](C(=O)Nc1cc2c(cn1)[C@H](C(=O)N1CC(F)(F)C(F)(F)C1)CCC2)C1CCC(F)(F)CC1] | 629.3 | ¹H NMR (400 MHz, Chloroform-d) δ 8.02 (d, J = 17.5 Hz, 2H), 7.51 (d, J = 7.6 Hz, 1H), 6.50 (d, J = 7.4 Hz, 1H), 6.25 (s, 1H), 5.56 − 5.41 (m, 1H), 4.56 − 4.38 (m, 2H), 4.15 − 3.91 (m, 3H), 3.77 (t, J = 6.9 Hz, 1H), 3.48 − 3.25 (m, 2H), 2.40 − 1.94 (m, 6H), 1.95 − 1.43 (m, 14H), 1.36 − 1.22 (m, 1H). |
| 70 | 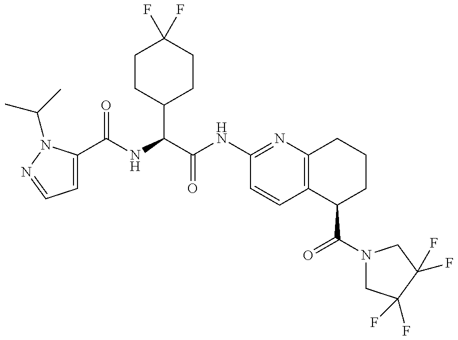 [SMILES: CC(C)n1nccc1C(=O)N[C@H](C(=O)Nc1ccc2c(n1)CCC[C@H]2C(=O)N1CC(F)(F)C(F)(F)C1)C1CCC(F)(F)CC1] | 629.3 | ¹H NMR (400 MHz, Chloroform-d) δ 7.94 (d, J = 8.0 Hz, 1H), 7.49 (d, J = 7.4 Hz, 1H), 7.29 (d, J = 8.0 Hz, 1H), 6.49 (d, J = 7.6 Hz, 1H), 6.22 (s, 1H), 5.60 − 5.45 (m, 1H), 4.61 (d, J = 7.0 Hz, 1H), 4.56 − 4.38 (m, 1H), 4.35 − 3.91 (m, 3H), 3.81 (t, J = 6.9 Hz, 1H), 3.27 − 3.14 (m, 1H), 3.11 − 2.98 (m, 1H), 2.40 − 1.98 (m, 5H), 1.94 − 1.43 (m, 14H), 1.36 − 1.22 (m, 1H). |
| 71 | 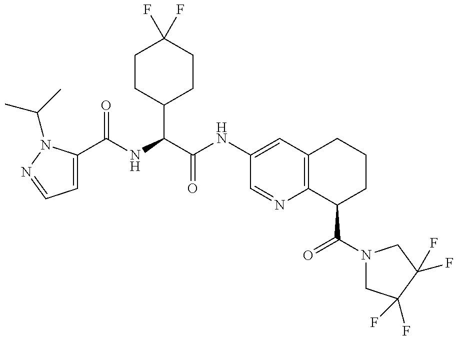 [SMILES: CC(C)n1nccc1C(=O)N[C@H](C(=O)Nc1cnc2c(c1)CCC[C@H]2C(=O)N1CC(F)(F)C(F)(F)C1)C1CCC(F)(F)CC1] | 629.3 | ¹H NMR (400 MHz, Chloroform-d) δ 9.80 (s, 1H), 9.58 (d, J = 1.1 Hz, 1H), 7.58 − 7.47 (m, 2H), 6.50 (d, J = 7.4 Hz, 1H), 6.26 (s, 1H), 5.29 − 5.13 (m, 1H). 4.88 − 4.70 (m, 1H), 4.54 (d, J = 7.0 Hz, 1H), 4.51 − 4.34 (m, 1H), 4.09 − 3.89 (m, 3H), 3.08 − 2.95 (m, 1H), 2.95 − 2.82 (m, 1H), 2.41 − 2.28 (m, 2H), 2.32 − 2.16 (m, 2H), 2.20 − 1.99 (m, 2H), 1.94 − 1.80 (m, 1H), 1.84 − 1.67 (m, 4H), 1.72 − 1.58 (m, 2H), 1.63 − 1.43 (m, 8H). 1.36 − 1.22 (m, 1H). |
| 72 | 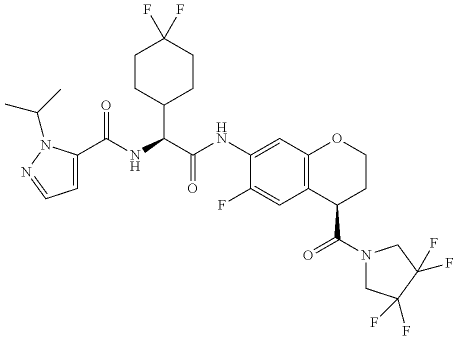 [SMILES: CC(C)n1nccc1C(=O)N[C@H](C(=O)Nc1cc2c(cc1F)[C@H](C(=O)N1CC(F)(F)C(F)(F)C1)CCO2)C1CCC(F)(F)CC1] | 648.2 | ¹H NMR (400 MHz, Chloroform-d) δ 7.68 (d, J = 5.7 Hz, 1H), 7.50 (d, J = 7.4 Hz, 1H), 6.92 − 6.84 (m, 1H), 6.71 (d, J = 7.4 Hz, 1H), 6.21 (s, 1H), 5.60 − 5.44 (m, 1H), 4.70 (d, J = 6.9 Hz, 1H), 4.59 − 4.22 (m, 3H), 4.19 − 4.01 (m, 1H), 4.03 − 3.85 (m, 3H), 2.41 − 1.99 (m, 6H), 1.85 − 1.71 (m, 1H), 1.76 − 1.67 (m, 1H), 1.72 − 1.43 (m, 8H), 1.36 − 1.22 (m, 1H). |
| 73 | 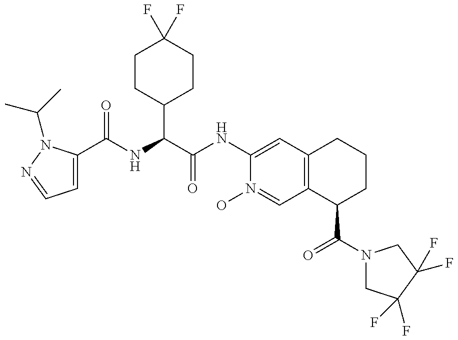 | 645.3 | ¹H NMR (400 MHz, Chloroform-d) δ 9.20 (s, 1H), 9.14 (s, 1H), 8.48 (s, 1H), 7.71 (s, 1H), 7.29 (d, J = 7.4 Hz, 1H), 6.53 (d, J = 7.4 Hz, 1H), 5.06 − 4.90 (m, 1H), 4.34 (d, J = 7.0 Hz, 1H), 3.87 − 3.46 (m, 7H), 2.43 − 2.29 (m, 1H), 2.15 − 1.90 (m, 3H), 1.85 − 1.34 (m, 16H), 1.20 (s, 1H). |
| 74 | 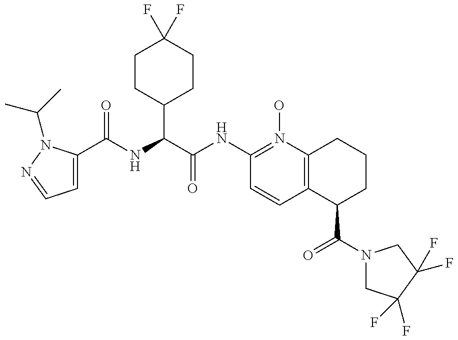 | 645.3 | ¹H NMR (400 MHz, Chloroform-d) δ 9.20 (s, 1H), 7.97 (d, J = 7.4 Hz, 1H), 7.71 (s, 1H), 7.60 (d, J = 7.6 Hz, 1H), 7.29 (d, J = 7.4 Hz, 1H), 6.53 (d, J = 7.4 Hz, 1H), 5.06 − 4.90 (m, 1H), 4.34 (d, J = 7.0 Hz, 1H), 3.87 − 3.69 (m, 4H), 3.62 (t, J = 7.0 Hz, 1H), 2.96 − 2.77 (m, 2H), 2.43 − 2.29 (m, 1H), 2.13 − 1.90 (m, 3H), 1.85 − 1.57 (m, 6H), 1.61 − 1.47 (m, 4H), 1.50 − 1.34 (m, 5H), 1.20 (s, 1H). |
| 75 | 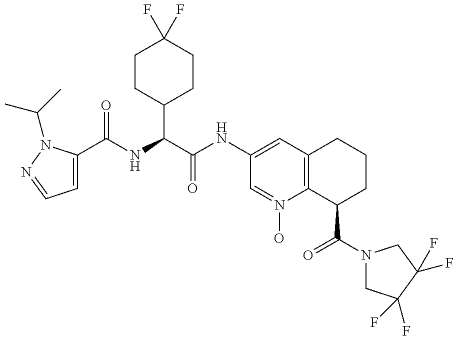 | 645.3 | ¹H NMR (400 MHz, Chloroform-d) δ 9.80 (s, 1H), 9.20 (s, 1H), 8.97 (d, J = 1.3 Hz, 1H), 8.11 (d, J = 1.5 Hz, 1H), 7.29 (d, J = 7.4 Hz, 1H), 6.53 (d, J = 7.4 Hz, 1H), 5.06 − 4.90 (m, 1H), 4.34 (d, J = 7.0 Hz, 1H), 3.87 − 3.69 (m, 4H), 3.62 (t, J = 7.0 Hz. 1H), 2.99 − 2.86 (m, 1H), 2.81 − 2.68 (m, 1H), 2.43 − 2.29 (m, 1H), 2.13 − 1.90 (m, 3H), 1.85 − 1.57 (m, 6H), 1.61 − 1.47 (m, 4H), 1.50 − 1.34 (m, 5H), 1.20 (s, 1H). |
| 76 | 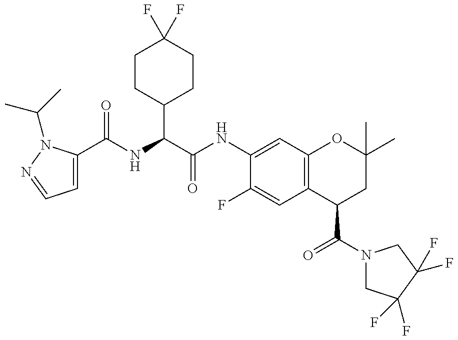 [SMILES: CC(C)n1nccc1C(=O)N[C@H](C(=O)Nc1cc2c(cc1F)[C@H](C(=O)N1CC(F)(F)C(F)(F)C1)CC(C)(C)O2)C1CCC(F)(F)CC1] | 676.3 | ¹H NMR (400 MHz, Chloroform-d) δ 7.50 (d, J = 7.6 Hz, 1H), 6.96 (d, J = 5.7 Hz, 1H), 6.91 − 6.84 (m, 1H), 6.72 (d, J = 7.4 Hz, 1H), 6.23 (s, 1H), 5.56 − 5.41 (m, 1H), 4.62 (d, J = 7.0 Hz, 1H), 4.58 − 4.36 (m, 2H), 4.10 − 3.84 (m, 3H), 2.41 − 2.01 (m, 5H), 1.96 − 1.86 (m, 1H), 1.85 − 1.71 (m, 1H), 1.76 − 1.62 (m, 2H), 1.63 − 1.40 (m, 12H), 1.36 − 1.22 (m, 1H). |
| 77 | 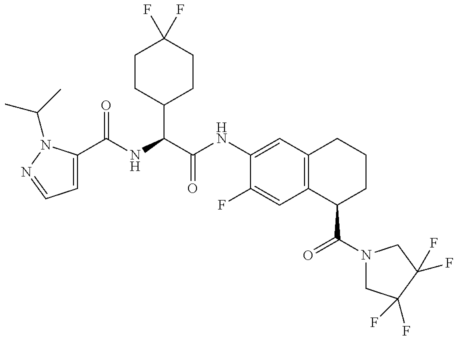 [SMILES: CC(C)n1nccc1C(=O)N[C@H](C(=O)Nc1cc2c(cc1F)[C@H](C(=O)N1CC(F)(F)C(F)(F)C1)CCC2)C1CCC(F)(F)CC1] | 646.3 | ¹H NMR (400 MHz, Chloroform-d) δ 7.52 (d, J = 7.6 Hz, 1H), 7.05 (s, 1H), 6.98 − 6.91 (m, 1H), 6.82 − 6.70 (m, 2H), 5.38 − 5.23 (m, 1H), 4.75 (d, J = 7.0 Hz, 1H), 4.51 − 4.33 (m, 1H), 4.27 − 4.09 (m, 1H), 4.08 − 3.86 (m, 4H), 2.99 − 2.82 (m, 2H), 2.42 − 2.29 (m, 2H), 2.34 − 2.18 (m, 2H), 2.21 − 2.07 (m, 2H), 2.12 − 2.00 (m, 2H), 2.02 − 1.61 (m, SH), 1.66 − 1.53 (m, 2H), 1.55 − 1.43 (m, 7H), 1.36 − 1.22 (m, 1H). |
| 78 | 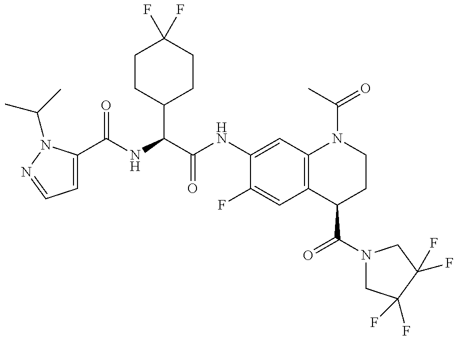 [SMILES: CC(=O)N1CC[C@@H](C(=O)N2CC(F)(F)C(F)(F)C2)c2cc(F)c(NC(=O)[C@@H](NC(=O)c3ccnn3C(C)C)C3CCC(F)(F)CC3)cc21] | 689.3 | ¹H NMR (400 MHz, Chloroform-d) δ 7.49 (d, J = 7.4 Hz, 1H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 3H), 3.74 − 3.63 (m, 1H), 2.41 − 2.15 (m, 2H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H). 1.36 − 1.22 (m, 1H). |
| 79 | 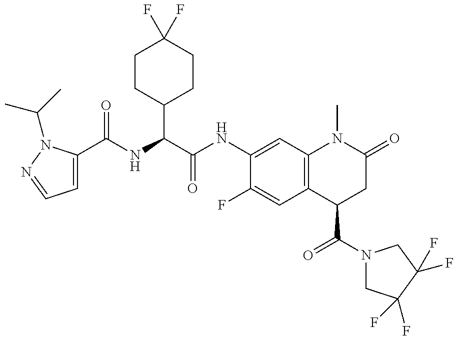 [SMILES: CC(C)n1nccc1C(=O)N[C@H](C(=O)Nc1cc2c(cc1F)[C@H](C(=O)N1CC(F)(F)C(F)(F)C1)CC(=O)N2C)C1CCC(F)(F)CC1] | 675.2 | ¹H NMR (400 MHz, Chloroform-d) δ 7.86 (s, 1H), 7.74 (s, 1H), 7.52 (d, J = 7.4 Hz, 1H), 7.23 − 7.15 (m, 1H), 6.87 − 6.78 (m, 2H), 5.44 − 5.29 (m, 1H), 4.81 (d, J = 6.9 Hz, 1H), 4.62 − 4.27 (m, 3H), 4.05 − 3.82 (m, 2H), 3.52 (s. 3H), 3.19 − 3.08 (m, 1H), 2.93 − 2.83 (m, 1H), 2.40 − 2.09 (m, 3H), 2.09 − 1.92 (m, 1H), 1.77 − 1.43 (m, 11H), 1.36 − 1.22 (m, 1H). |
| 80 | 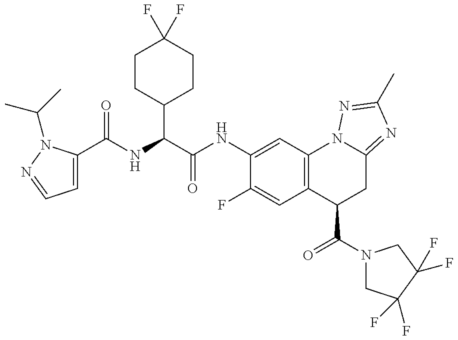 [SMILES: Cc1nc2n(n1)-c1cc(NC(=O)[C@@H](NC(=O)c3ccnn3C(C)C)C3CCC(F)(F)CC3)c(F)cc1[C@H](C(=O)N1CC(F)(F)C(F)(F)C1)C2] | 699.3 | ¹H NMR (400 MHz, Chloroform-d) δ 7.86 (s, 1H), 7.74 (s, 1H), 7.52 (d, J = 7.4 Hz, 1H), 7.23 − 7.15 (m, 1H), 6.87 − 6.78 (m, 2H), 5.44 − 5.29 (m, 1H), 4.81 (d, J = 6.9 Hz, 1H), 4.62 − 4.27 (m, 3H), 4.05 − 3.82 (m, 2H), 2.42 (s, 3H), 3.19 − 3.08 (m, 1H), 2.93 − 2.83 (m, 1H), 2.40 − 2.09 (m, 3H), 2.09 − 1.92 (m, 1H), 1.77 − 1.43 (m, 11H), 1.36 − 1.22 (m, 1H). |
| 81 | 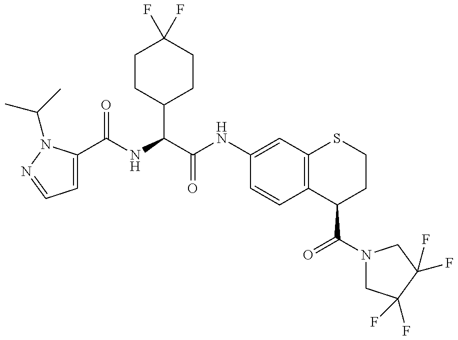 [SMILES: CC(C)n1nccc1C(=O)N[C@H](C(=O)Nc1ccc2c(c1)SCC[C@H]2C(=O)N1CC(F)(F)C(F)(F)C1)C1CCC(F)(F)CC1] | 646.2 | ¹H NMR (400 MHz, Chloroform-d) δ 8.24 (d, 1H), 7.47 (d, 1H), 7.13 − 6.91 (m, 2H), 6.84 − 6.82 (m, 2H), 6.56 (s, 1H), 5.41 − 5.39 (m, 1H), 4.25 − 4.05 (m, 2H), 3.77 − 3.70 (m, 1H), 2.87 − 2.72 (m, 5H), 2.28 − 2.01 (m, 3H), 1.91 − 1.77 (m, 2H), 1.74 − 1.18 (m, 14H). |
| 82 | 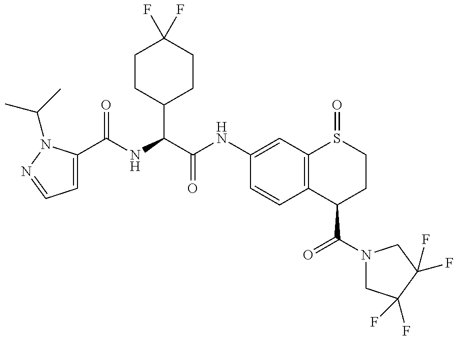 [SMILES: CC(C)n1nccc1C(=O)N[C@H](C(=O)Nc1ccc2c(c1)S(=O)CC[C@H]2C(=O)N1CC(F)(F)C(F)(F)C1)C1CCC(F)(F)CC1] | 662.2 | ¹H NMR (400 MHz, Chloroform-d) δ 8.24 (d, 1H), 7.47 (d, 1H), 7.13 − 6.91 (m, 2H), 6.84 − 6.82 (m, 2H), 6.56 (s, 1H), 5.41 − 5.39 (m, 1H), 4.25 − 4.05 (m, 2H), 3.77 − 3.70 (m, 1H), 2.87 − 2.72 (m, 5H), 2.28 − 2.01 (m, 3H), 1.91 − 1.77 (m, 2H), 1.74 − 1.18 (m, 14H). |
| 83 | 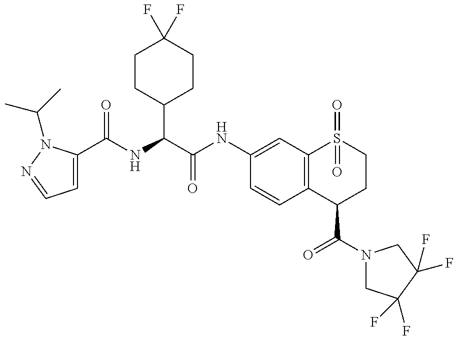 | 678.2 | ¹H NMR (400 MHz, Chloroform-d) δ 8.24 (d, 1H), 7.47 (d, 1H), 7.13 − 6.91 (m, 2H), 6.84 − 6.82 (m, 2H), 6.56 (s, 1H), 5.41 − 5.39 (m, 1H), 4.25 − 4.05 (m, 2H), 3.77 − 3.70 (m, 1H), 3.47 − 3.42 (m, 5H), 2.28 − 2.01 (m, 3H), 1.91 − 1.77 (m, 2H), 1.74 − 1.18 (m, 14H). |
| 84 | 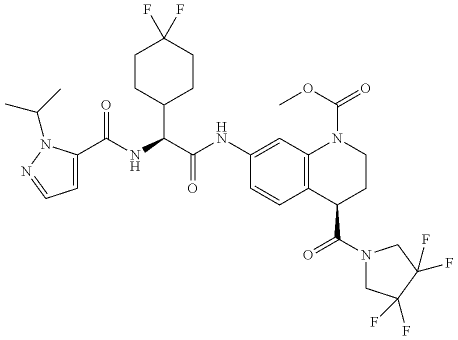 [SMILES: COC(=O)N1CC[C@@H](C(=O)N2CC(F)(F)C(F)(F)C2)c2ccc(NC(=O)[C@@H](NC(=O)c3ccnn3C(C)C)C3CCC(F)(F)CC3)cc21] | 687.3 | ¹H NMR (400 MHz, Chloroform-d) δ 7.49 (d, J = 7.4 Hz, 1H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 6H), 3.74 − 3.63 (m, 1H), 2.41 − 2.15 (m, 2H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H), 1.36 − 1.22 (m, 1H). |
| 85 | 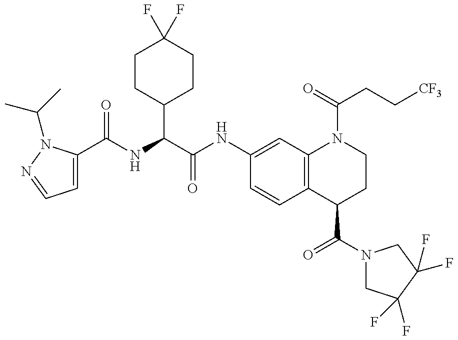 [SMILES: CC(C)n1nccc1C(=O)N[C@H](C(=O)Nc1ccc2c(c1)N(C(=O)CCC(F)(F)F)CC[C@H]2C(=O)N1CC(F)(F)C(F)(F)C1)C1CCC(F)(F)CC1] | 753.3 | ¹H NMR (400 MHz, Chloroform-d) δ 7.49 (d, J = 7.4 Hz, 1H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 3H), 3.74 − 3.63 (m, 1H), 2.41 − 2.15 (m, 6H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H), 1.36 − 1.22 (m, 1H). |
| 86 | 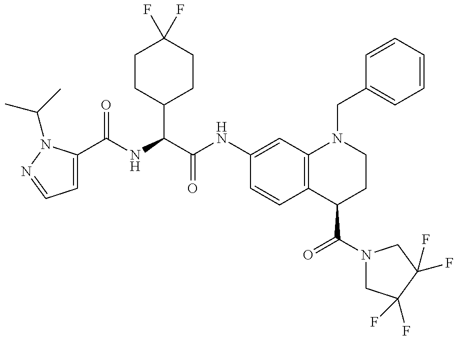 [SMILES: CC(C)n1nccc1C(=O)N[C@H](C(=O)Nc1ccc2c(c1)N(Cc1ccccc1)CC[C@H]2C(=O)N1CC(F)(F)C(F)(F)C1)C1CCC(F)(F)CC1] | 719.3 | ¹H NMR (400 MHz. Chloroform-d) δ 7.49 (d, J = 7.4 Hz, 1H), 7.31 − 7.23 (m, 5H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 3H), 3.74 − 3.63 (m, 1H), 2.41 − 2.15 (m, 2H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H), 1.36 − 1.22 (m, 1H). |
| 87 | 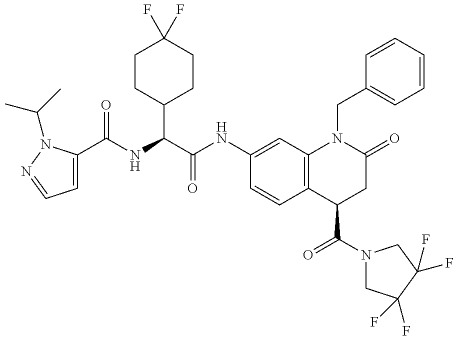 [SMILES: CC(C)n1nccc1C(=O)N[C@H](C(=O)Nc1ccc2c(c1)N(Cc1ccccc1)C(=O)C[C@H]2C(=O)N1CC(F)(F)C(F)(F)C1)C1CCC(F)(F)CC1] | 733.3 | ¹H NMR (400 MHz, Chloroform-d) δ 7.49 (d, J = 7.4 Hz, 1H), 7.31 − 7.23 (m, 5H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 3H), 2.41 − 2.15 (m, 2H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H), 1.36 − 1.22 (m, 1H). |
| 88 | 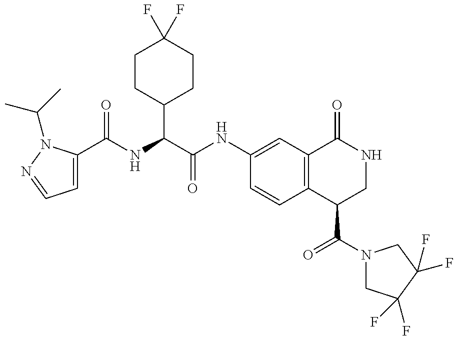 [SMILES: CC(C)n1nccc1C(=O)N[C@H](C(=O)Nc1ccc2c(c1)C(=O)NC[C@H]2C(=O)N1CC(F)(F)C(F)(F)C1)C1CCC(F)(F)CC1] | 743.2 | ¹H NMR (400 MHz, Chloroform-d) δ 7.49 (d, J = 7.4 Hz, 1H), 7.31 − 7.23 (m, 5H), 7.11 − 7.03 (m, 1H), 6.91 (d, J = 5.6 Hz, 1H), 6.72 (d, J = 7.6 Hz, 1H), 6.24 (s, 1H), 5.15 − 5.00 (m, 1H), 4.62 (d, J = 6.9 Hz, 1H), 4.58 − 4.35 (m, 2H), 4.11 − 4.01 (m, 1H), 4.04 − 3.88 (m, 3H), 3.41 − 3.25 (m, 2H), 2.20 − 2.11 (m, 1H), 2.13 (s, 3H), 2.15 − 2.03 (m, 1H), 2.07 − 1.91 (m, 1H), 1.85 − 1.65 (m, 3H), 1.70 − 1.52 (m, 1H), 1.55 − 1.43 (m, 6H), 1.36 − 1.22 (m, 1H). |
| 89 | 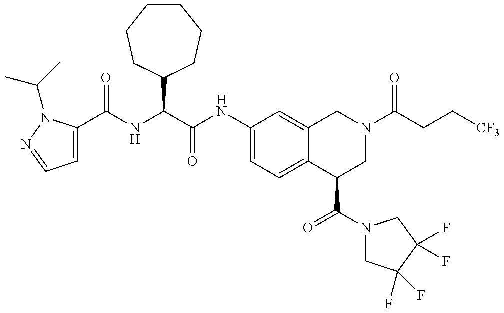 [SMILES: CC(C)n1nccc1C(=O)N[C@H](C(=O)Nc1ccc2c(c1)CN(C(=O)CCC(F)(F)F)C[C@H]2C(=O)N1CC(F)(F)C(F)(F)C1)C1CCCCCC1] | 731.3 | ¹H NMR (400 MHz, Chloroform-d) δ 8.24 (d, 1H), 7.47 (d, 1H), 7.13 − 6.91 (m, 2H), 6.84 − 6.82 (m, 2H), 6.56 (s, 1H), 5.41 − 5.39 (m, 1H), 4.25 − 4.05 (m, 2H), 3.77 − 3.70 (m, 1H), 2.87 − 2.72 (m, 5H), 2.28 − 2.01 (m, 3H), 1.91 − 1.77 (m, 2H), 1.74 − 1.18 (m, 17H). |
| 90 | 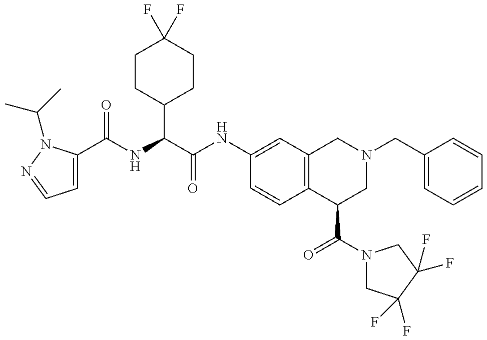 [SMILES: CC(C)n1nccc1C(=O)N[C@H](C(=O)Nc1ccc2c(c1)CN(Cc1ccccc1)C[C@H]2C(=O)N1CC(F)(F)C(F)(F)C1)C1CCC(F)(F)CC1] | 719.3 | ¹H NMR (400 MHz, Chloroform-d) δ 9.50 (s, 1H), 8.14 (s, 1H), 8.04 − 7.97 (m, 1H), 7.52 (d, J = 7.6 Hz, 1H), 7.31 − 7.10 (m, 7H), 6.83 (d, J = 7.4 Hz, 1H), 5.44 − 5.29 (m, 1H), 5.13 (d, J = 7.0 Hz. 1H), 4.53 − 4.35 (m, 1H), 4.24 − 3.95 (m, 5H), 3.98 − 3.80 (m, 2H), 3.74 − 3.64 (m, 1H), 3.52 − 3.43 (m, 1H), 3.18 − 3.08 (m, 1H), 2.80 − 2.71 (m, 1H), 2.07 − 1.97 (m, 1H), 1.76 − 1.62 (m, 2H), 1.63 − 1.40 (m, 12H), 1.36 − 1.22 (m, 1H). |
| 91 | 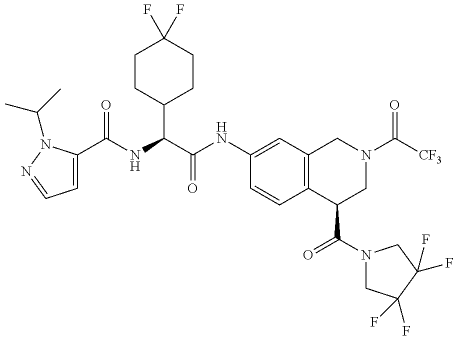 [SMILES: CC(C)n1nccc1C(=O)N[C@H](C(=O)Nc1ccc2c(c1)CN(C(=O)C(F)(F)F)C[C@H]2C(=O)N1CC(F)(F)C(F)(F)C1)C1CCC(F)(F)CC1] | 725.2 | ¹H NMR (400 MHz, Chloroform-d) δ 9.50 (s, 1H), 8.14 (s, 1H), 8.04 − 7.97 (m, 1H), 7.52 (d, J = 7.6 Hz, 1H), 7.31 − 7.10 (m, 2H), 6.83 (d, J = 7.4 Hz, 1H), 5.44 − 5.29 (m, 1H), 5.13 (d, J = 7.0 Hz, 1H), 4.53 − 4.35 (m, 1H), 4.24 − 3.95 (m, 3H), 3.98 − 3.80 (m, 2H), 3.74 − 3.64 (m, 1H), 3.52 − 3.43 (m, 1H), 3.18 − 3.08 (m, 1H), 2.80 − 2.71 (m, 1H), 2.07 − 1.97 (m, 1H), 1.76 − 1.62 (m, 2H), 1.63 − 1.40 (m, 12H), 1.36 − 1.22 (m, 1H). |
| 92 | 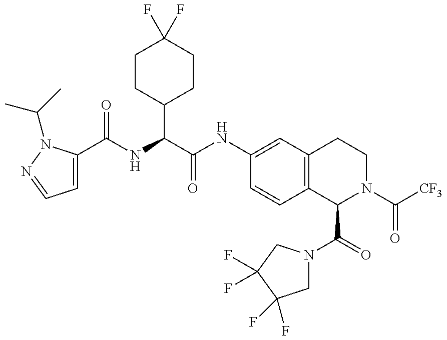 [SMILES: CC(C)n1nccc1C(=O)N[C@H](C(=O)Nc1ccc2c(c1)CCN(C(=O)C(F)(F)F)[C@H]2C(=O)N1CC(F)(F)C(F)(F)C1)C1CCC(F)(F)CC1] | 725.2 | ¹H NMR (400 MHz, Chloroform-d) δ 9.50 (s, 1H), 8.14 (s, 1H), 8.04 − 7.97 (m, 1H), 7.52 (d, J = 7.6 Hz, 1H), 7.31 − 7.10 (m, 2H), 6.83 (d, J = 7.4 Hz, 1H), 5.44 − 5.29 (m, 2H), 5.13 (d, J = 7.0 Hz, 1H), 4.53 − 4.35 (m, 1H), 4.24 − 3.95 (m, 1H), 3.98 − 3.80 (m, 4H), 3.74 − 3.64 (m, 1H), 3.52 − 3.43 (m, 1H), 3.18 − 3.08 (m, 1H), 2.80 − 2.71 (m, 1H), 2.07 − 1.97 (m, 1H), 1.76 − 1.62 (m, 2H), 1.63 − 1.40 (m, 12H), 1.36 − 1.22 (m, 1H). |
| 93 | 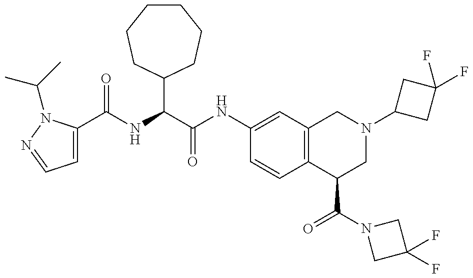 [SMILES: CC(C)n1nccc1C(=O)N[C@H](C(=O)Nc1ccc2c(c1)CN(C1CC(F)(F)C1)C[C@H]2C(=O)N1CC(F)(F)C1)C1CCCCCC1] | 647.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.99 − 2.95 (m, 4H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H) |
| 94 | 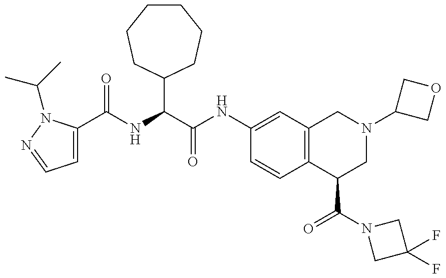 [SMILES: CC(C)n1nccc1C(=O)N[C@H](C(=O)Nc1ccc2c(c1)CN(C1COC1)C[C@H]2C(=O)N1CC(F)(F)C1)C1CCCCCC1] | 613.3 | ¹H NMR (400 MHz, Chloroform-d) δ 8.57 (s, 1H), 7.43 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 5H), 3.89 − 3.85 (m, 2H), 2.99 − 2.95 (m, 4H), 2.77 − 2.46 (m, 4H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 9H). |
| 95 | 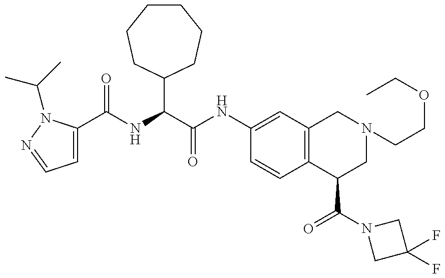 [SMILES: CCOCCN1Cc2cc(NC(=O)[C@@H](NC(=O)c3ccnn3C(C)C)C3CCCCCC3)ccc2[C@H](C(=O)N2CC(F)(F)C2)C1] | 629.4 | ¹H NMR (400 MHz, Chloroform-d) δ 8.27 (s, 1H), 7.47 − 7.42 (m, 1H), 7.11 (s, 1H), 7.05 − 7.03 (m, 1H), 6.90 − 6.88 (m, 1H), 6.85 − 6.82 (m, 1H), 6.56 − 6.54 (m, 1H), 5.40 − 5.36 (m, 1H), 4.53 − 4.50 (m, 1H), 4.43 − 4.25 (m, 4H), 3.97 − 3.95 (m, 1H), 3.82 − 3.80 (m, 1H), 2.93 − 2.83 (m, 2H), 2.21 − 2.15 (m, 3H), 1.99 (m, 2H), 1.87 − 1.78 (m, 5H), 1.78 − 1.71 (m, 3H), 1.66 (m, 5H), 1.57 (m, 2H), 1.48 − 1.41 (m, 12H). |
| 96 | 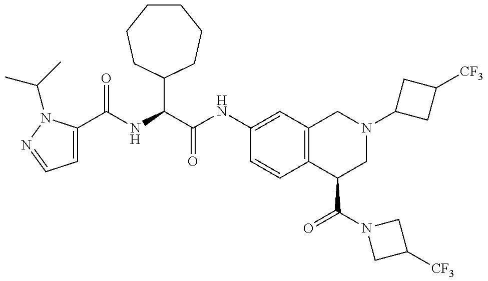 [SMILES: CC(C)n1nccc1C(=O)N[C@H](C(=O)Nc1ccc2c(c1)CN(C1CC(C(F)(F)F)C1)C[C@H]2C(=O)N1CC(C(F)(F)F)C1)C1CCCCCC1] | 711.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.99 − 2.95 (m, 4H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H) |
| 97 | 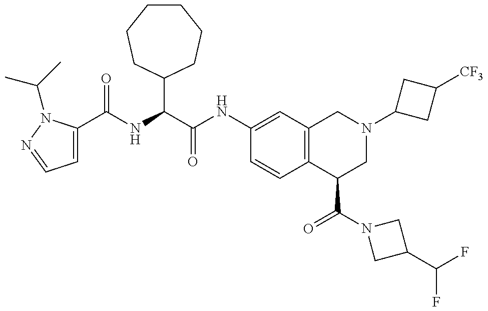 [SMILES: CC(C)n1nccc1C(=O)N[C@H](C(=O)Nc1ccc2c(c1)CN(C1CC(C(F)(F)F)C1)C[C@H]2C(=O)N1CC(C(F)F)C1)C1CCCCCC1] | 693.3 | ¹H NMR (400 MHz, Chloroform-d) δ 8.54 (s, 1H), 7.43 − 7.39 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 3H), 4.43 − 4.35 (m, 3H), 4.13 − 4.09 (m, 1H), 3.89 − 3.85 (m, 2H), 2.99 − 2.95 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H). |
| 98 | 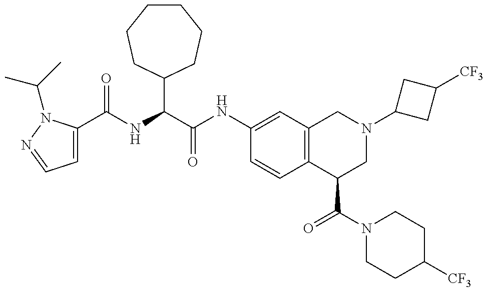 [SMILES: CC(C)n1nccc1C(=O)N[C@H](C(=O)Nc1ccc2c(c1)CN(C1CC(C(F)(F)F)C1)C[C@H]2C(=O)N1CCC(C(F)(F)F)CC1)C1CCCCCC1] | 739.4 | ¹H NMR (400 MHz, Chloroform-d) δ 8.56 (s, 1H), 7.46 − 7.42 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.99 − 2.95 (m, 2H), 2.77 − 2.46 (m, 4H), 2.34 (s, 1H), 2.23 − 2.03 (m, 2H), 2.01 − 1.76 (m, 7H), 1.75 − 1.64 (m, 4H), 1.64 − 1.54 (m, 1H), 1.54 − 1.31 (m, 9H). |
| 99 | 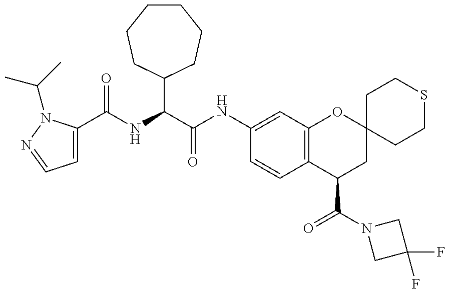 [SMILES: CC(C)n1nccc1C(=O)N[C@H](C(=O)Nc1ccc2c(c1)OC1(CCSCC1)C[C@H]2C(=O)N1CC(F)(F)C1)C1CCCCCC1] | 644.3 | ¹H NMR (400 MHz, Chloroform-d) δ 8.27 (s, 1H), 7.45 − 7.41 (m, 1H), 7.11 (s, 1H), 7.05 − 7.01 (m, 1H), 6.90 − 6.86 (m, 1H), 6.86 − 6.83 (m, 1H), 6.56 (m, 1H), 5.40 − 5.35 (m, 1H), 4.53 − 4.50 (m, 1H), 4.43 − 4.25 (m, 4H), 3.97 (m, 1H), 3.82 (m, 1H), 2.21 − 2.15 (m, 3H), 1.99 (m, 2H), 1.87 − 1.78 (m, 5H), 1.78 − 1.71 (m, 3H), 1.66 (m, 5H), 1.57 (m, 2H), 1.48 − 1.41 (m, 12H). |
| 100 | 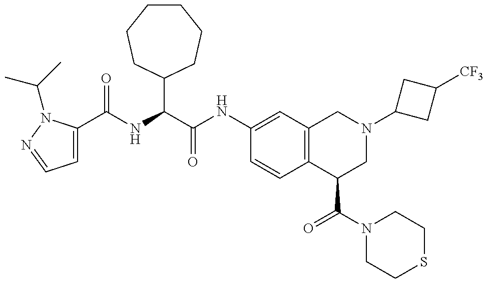 [SMILES: CC(C)n1nccc1C(=O)N[C@H](C(=O)Nc1ccc2c(c1)CN(C1CC(C(F)(F)F)C1)C[C@H]2C(=O)N1CCSCC1)C1CCCCCC1] | 689.3 | ¹H NMR (400 MHz, Chloroform-d) δ 8.58 (s, 1H), 7.43 − 7.39 (m, 1H), 7.25 − 7.23 (m, 1H), 6.99 − 6.95 (m, 1H), 6.91 − 6.78 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.99 − 2.95 (m, 2H), 2.77 − 2.46 (m, 4H), 2.34 (s, 1H), 2.23 − 2.03 (m, 4H), 2.01 − 1.76 (m, 7H), 1.75 − 1.64 (m, 2H), 1.64 − 1.54 (m, 1H), 1.54 − 1.31 (m, 9H). |
| 101 | 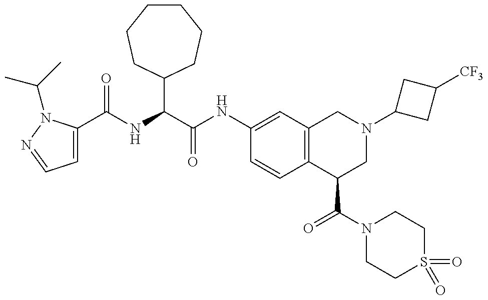 [SMILES: CC(C)n1nccc1C(=O)N[C@H](C(=O)Nc1ccc2c(c1)CN(C1CC(C(F)(F)F)C1)C[C@H]2C(=O)N1CCS(=O)(=O)CC1)C1CCCCCC1] | 721.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 4H), 2.99 − 2.95 (m, 2H), 2.77 − 2.46 (m, 2H), 2.34 (s, 1H), 2.23 − 2.03 (m, 4H), 2.01 − 1.76 (m, 7H), 1.75 − 1.64 (m, 2H), 1.64 − 1.54 (m, 1H), 1.54 − 1.31 (m, 9H). |
| 102 | 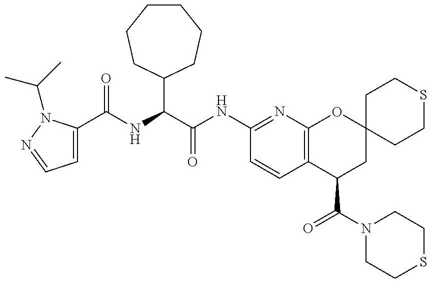 [SMILES: CC(C)n1nccc1C(=O)N[C@H](C(=O)Nc1ccc2c(n1)OC1(CCSCC1)C[C@H]2C(=O)N1CCSCC1)C1CCCCCC1] | 655.3 | ¹H NMR (400 MHz, Chloroform-d) δ 8.56 (s, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.99 − 2.95 (m, 2H), 2.77 − 2.46 (m, 8H), 2.34 (s, 1H), 2.23 − 2.03 (m, 4H), 2.01 − 1.76 (m, 7H), 1.75 − 1.64 (m, 2H), 1.64 − 1.54 (m, 1H), 1.54 − 1.31 (m, 9H). |
| 103 | 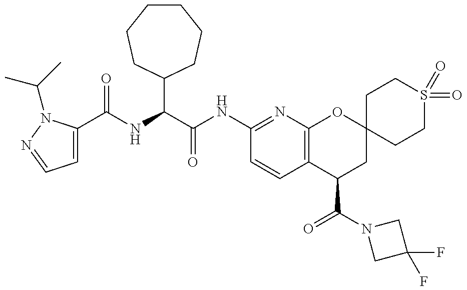 [SMILES: CC(C)n1nccc1C(=O)N[C@H](C(=O)Nc1ccc2c(n1)OC1(CCS(=O)(=O)CC1)C[C@H]2C(=O)N1CC(F)(F)C1)C1CCCCCC1] | 677.3 | ¹H NMR (400 MHz, Chloroform-d) δ 8.60 (s, 1H), 7.28 − 7.25 (m, 1H), 6.98 − 6.94 (m, 1H), 6.90 − 6.79 (m, 2H), 6.60 − 6.55 (m, 1H), 5.42 − 5.38 (m, 1H), 4.60 − 4.55 (m, 1H), 4.43 − 4.35 (m, 3H), 4.13 − 4.09 (m, 1H), 3.89 − 3.85 (m, 6H), 2.99 − 2.95 (m, 2H), 2.77 − 2.46 (m, 2H), 2.34 (s, 1H), 2.23 − 2.03 (m, 4H), 2.01 − 1.76 (m, 9H), 1.75 − 1.64 (m, 8H), 1.64 − 1.54 (m, 1H), 1.54 − 1.31 (m, 9H). |
| 104 | 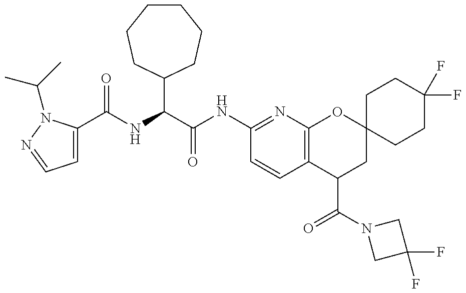 [SMILES: CC(C)n1nccc1C(=O)N[C@H](C(=O)Nc1ccc2c(n1)OC1(CCC(F)(F)CC1)CC2C(=O)N1CC(F)(F)C1)C1CCCCCC1] | 663.3 | ¹H NMR (400 MHz, Chloroform-d) δ 8.55 (s, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.99 − 2.95 (m, 2H), 2.77 − 2.46 (m, 2H), 2.34 (s, 1H), 2.23 − 2.03 (m, 4H), 2.01 − 1.76 (m, 11H), 1.75 − 1.64 (m, 10H), 1.64 − 1.54 (m, 1H), 1.54 − 1.31 (m, 9H). |
| 105 | 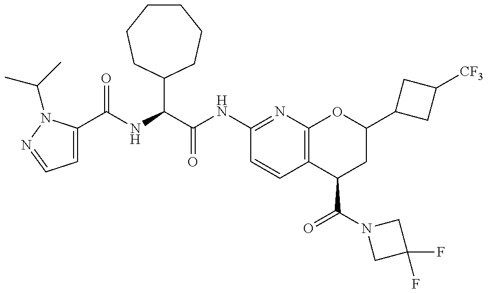 [SMILES: CC(C)n1nccc1C(=O)N[C@H](C(=O)Nc1ccc2c(n1)OC(C1CC(C(F)(F)F)C1)C[C@H]2C(=O)N1CC(F)(F)C1)C1CCCCCC1] | 681.3 | ¹H NMR (400 MHz, Chloroform-d) δ 8.56 (s, 1H), 7.27 − 7.23 (m, 1H), 6.99 − 6.95 (m, 1H), 6.93 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H). |
| 106 | 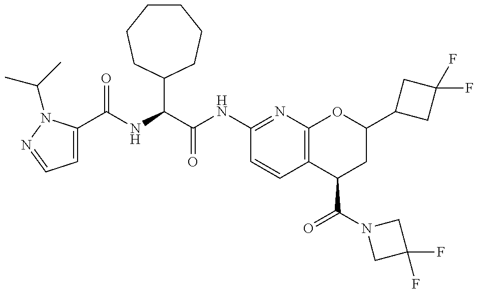 [SMILES: CC(C)n1nccc1C(=O)N[C@H](C(=O)Nc1ccc2c(n1)OC(C1CC(F)(F)C1)C[C@H]2C(=O)N1CC(F)(F)C1)C1CCCCCC1] | 667.3 | ¹H NMR (400 MHz, Chloroform-d) δ 8.57 (s, 1H), 7.24 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H). |
| 107 | 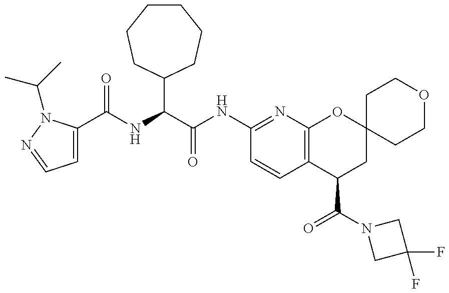 [SMILES: CC(C)n1nccc1C(=O)N[C@H](C(=O)Nc1ccc2c(n1)OC1(CCOCC1)C[C@H]2C(=O)N1CC(F)(F)C1)C1CCCCCC1] | 629.3 | ¹H NMR (400 MHz, Chloroform-d) δ 8.07 (s, 1H), 7.53 − 7.46 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H). |
| 108 | 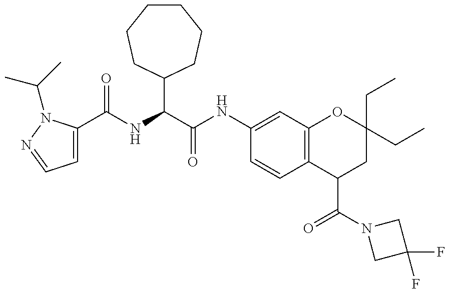 [SMILES: CCC1(CC)CC(C(=O)N2CC(F)(F)C2)c2ccc(NC(=O)[C@@H](NC(=O)c3ccnn3C(C)C)C3CCCCCC3)cc2O1] | 614.3 | ¹H NMR (400 MHz, Chloroform-d) δ 8.27 (s, 1H), 7.47 − 7.42 (m, 1H), 7.11 (s, 1H), 7.05 − 7.00 (m, 1H), 6.90 − 6.85 (m, 1H), 6.85 − 6.81 (m, 1H), 6.56 − 6.52 (m, 1H), 5.40 − 5.36 (m, 1H), 4.53 − 4.50 (m, 1H), 4.43 − 4.25 (m, 4H), 3.97 − 3.93 (m, 1H), 3.82 − 3.78 (m, 1H), 2.21 − 2.15 (m, 1H), 1.99 −1.93 (m, 2H), 1.87 − 1.78 (m, 5H), 1.78 − 1.71 (m, 3H), 1.66 − 1.62 (m, 5H), 1.57 − 1.49 (m, 2H), 1.48 − 1.41 (m, 14H). |
| 109 | 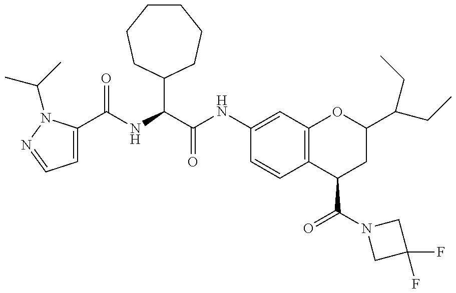 [SMILES: CCC(CC)C1C[C@@H](C(=O)N2CC(F)(F)C2)c2ccc(NC(=O)[C@@H](NC(=O)c3ccnn3C(C)C)C3CCCCCC3)cc2O1] | 628.4 | ¹H NMR (400 MHz, Chloroform-d) δ 8.27 (s, 1H), 7.49 − 7.46 (m, 1H), 7.11 (s, 1H), 7.02 − 6.98 (m, 1H), 6.90 − 6.86 (m, 1H), 6.85 − 6.82 (m, 1H), 6.56 − 6.52 (m, 1H), 5.40 − 5.33 (m, 1H), 4.53 − 4.50 (m, 1H), 4.43 − 4.25 (m, 5H), 3.97 − 3.92 (m, 1H), 3.82 − 3.74 (m, 1H), 2.21 − 2.15 (m, 1H), 1.99 − 1.95 (m, 2H), 1.87 − 1.78 (m, 5H), 1.78 − 1.71 (m, 3H), 1.66 − 1.58 (m, 5H), 1.57 − 1.48(m, 3H), 1.48 − 1.41 (m, 14H). |
| 110 | 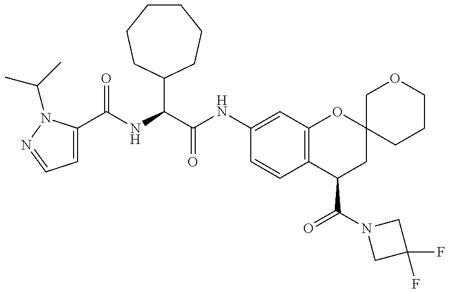 [SMILES: CC(C)n1nccc1C(=O)N[C@H](C(=O)Nc1ccc2c(c1)OC1(CCCOC1)C[C@H]2C(=O)N1CC(F)(F)C1)C1CCCCCC1] | 628.3 | ¹H NMR (400 MHz, Chloroform-d) δ 8.27 (s, 1H), 7.47 − 7.44 (m, 1H), 7.11 (s, 1H), 7.05 − 6.97 (m, 1H), 6.90 − 6.86 (m, 1H), 6.85 − 6.82 (m, 1H), 6.56 − 6.52 (m, 1H), 5.40 − 5.33 (m, 1H), 4.53 − 4.50 (m, 1H), 4.43 − 4.25 (m, 4H), 3.97 − 3.94 (m, 1H), 3.82 − 3.79 (m, 5H), 2.21 − 2.15 (m, 1H), 1.99 − 1.94 (m, 2H), 1.87 − 1.78 (m, 5H), 1.78 − 1.71 (m, 3H), 1.66 − 1.62 (m, 5H), 1.57 − 1.52 (m, 2H), 1.48 − 1.41 (m, 8H). |
| 111 | 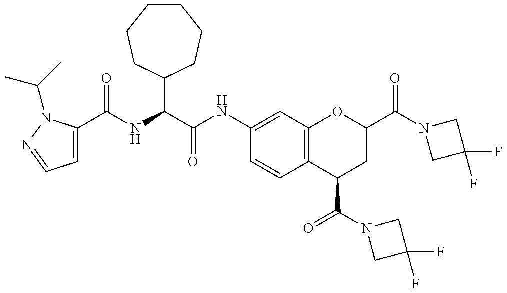 [SMILES: CC(C)n1nccc1C(=O)N[C@H](C(=O)Nc1ccc2c(c1)OC(C(=O)N1CC(F)(F)C1)C[C@H]2C(=O)N1CC(F)(F)C1)C1CCCCCC1] | 677.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 8H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 112 | 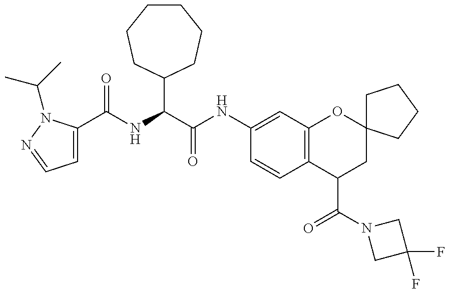 [SMILES: CC(C)n1nccc1C(=O)N[C@H](C(=O)Nc1ccc2c(c1)OC1(CCCC1)CC2C(=O)N1CC(F)(F)C1)C1CCCCCC1] | 612.3 | ¹H NMR (400 MHz, Chloroform-d) δ 8.27 (s, 1H), 7.49 − 7.44 (m, 1H), 7.11 (s, 1H), 7.05 − 7.02 (m, 1H), 6.92 − 6.90 (m, 1H), 6.85 − 6.82 (m, 1H), 6.56 − 6.52 (m, 1H), 5.40 − 5.33 (m, 1H), 4.51 − 4.44 (m, 1H), 4.43 − 4.25 (m, 4H), 3.97 − 3.94 (m, 1H), 3.82 − 3.79 (m, 1H), 2.21 − 2.15 (m, 1H), 1.99 − 1.93 (m, 2H), 1.87 − 1.78 (m, 5H), 1.78 − 1.71 (m, 3H), 1.66 − 1.62 (m, 5H), 1.57 − 1.52 (m, 2H), 1.48 − 1.41 (m, 12H). |
| 113 | 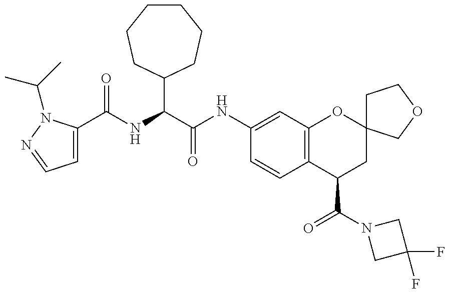 [SMILES: CC(C)n1nccc1C(=O)N[C@H](C(=O)Nc1ccc2c(c1)OC1(CCOC1)C[C@H]2C(=O)N1CC(F)(F)C1)C1CCCCCC1] | 614.3 | ¹H NMR (400 MHz, Chloroform-d) δ 8.27 (s, 1H), 7.49 − 7.44 (m, 1H), 7.11 (s, 1H), 7.05 − 7.02 (m, 1H), 6.92 − 6.90 (m, 1H), 6.85 − 6.82 (m, 1H), 6.56 − 6.52 (m, 1H), 5.40 − 5.33 (m, 1H), 4.51 − 4.44 (m, 1H), 4.43 − 4.25 (m, 4H), 3.97 − 3.94 (m, 3H), 3.82 − 3.79 (m, 3H), 2.21 − 2.15 (m, 1H), 1.99 − 1.93 (m, 2H), 1.87 − 1.78 (m, 5H), 1.78 − 1.71 (m, 3H), 1.66 − 1.62 (m, 5H), 1.57 − 1.52 (m, 2H), 1.48 − 1.41 (m, 8H). |
| 114 | 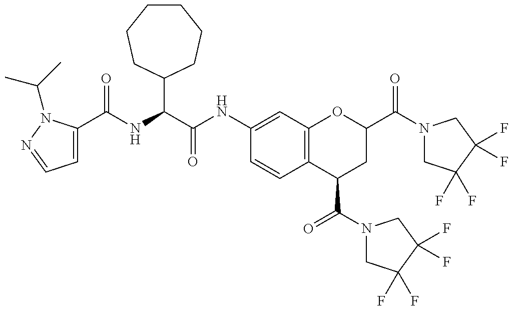 [SMILES: CC(C)n1nccc1C(=O)N[C@H](C(=O)Nc1ccc2c(c1)OC(C(=O)N1CC(F)(F)C(F)(F)C1)C[C@H]2C(=O)N1CC(F)(F)C(F)(F)C1)C1CCCCCC1] | 777.3 | ¹H NMR (400 MHz. Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 8H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 115 | 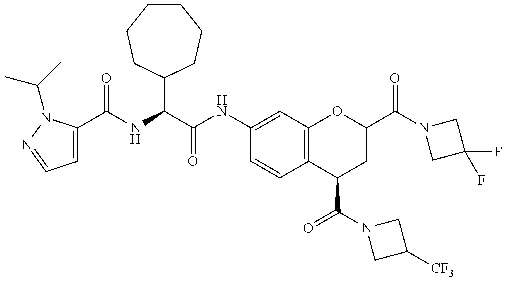 [SMILES: CC(C)n1nccc1C(=O)N[C@H](C(=O)Nc1ccc2c(c1)OC(C(=O)N1CC(F)(F)C1)C[C@H]2C(=O)N1CC(C(F)(F)F)C1)C1CCCCCC1] | 709.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 4H), 4.13 − 4.09 (m, 1H), 3.89 − 3.85 (m, 2H), 2.77 − 2.46 (m, 8H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 116 | 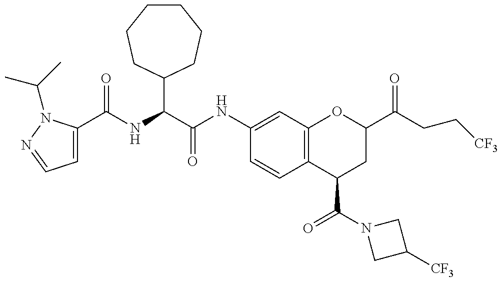 [SMILES: CC(C)n1nccc1C(=O)N[C@H](C(=O)Nc1ccc2c(c1)OC(C(=O)CCC(F)(F)F)C[C@H]2C(=O)N1CC(C(F)(F)F)C1)C1CCCCCC1] | 714.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 6H), 4.13 − 4.09 (m, 1H), 3.89 − 3.85 (m, 2H), 2.77 − 2.46 (m, 6H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 117 | 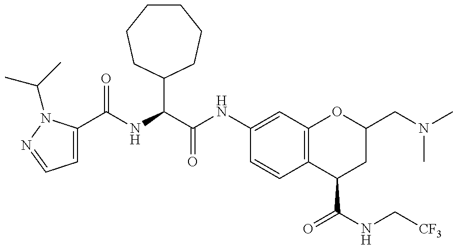 [SMILES: CC(C)n1nccc1C(=O)N[C@H](C(=O)Nc1ccc2c(c1)OC(CN(C)C)C[C@H]2C(=O)NCC(F)(F)F)C1CCCCCC1] | 621.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 3H), 4.59 − 4.55 (m, 1H), 4.43 − 4.35 (m, 4H), 4.13 − 4.09 (m, 1H), 3.89 − 3.85 (m, 2H), 2.77 − 2.46 (m, 2H), 2.36- 2.32 (m, 7H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 118 | 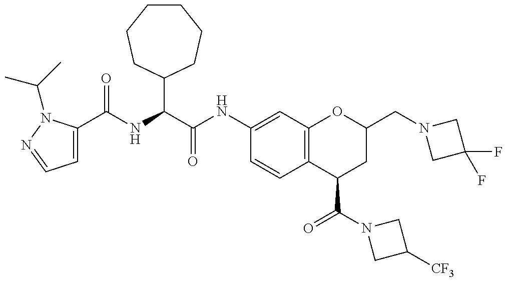 [SMILES: CC(C)n1nccc1C(=O)N[C@H](C(=O)Nc1ccc2c(c1)OC(CN1CC(F)(F)C1)C[C@H]2C(=O)N1CC(C(F)(F)F)C1)C1CCCCCC1] | 695.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 2H), 4.59 − 4.55 (m, 1H), 4.43 − 4.35 (m, 4H), 4.13 − 4.09 (m, 1H), 3.89 − 3.85 (m, 2H), 2.77 − 2.46 (m, 10H), 2.34 (m, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 119 | 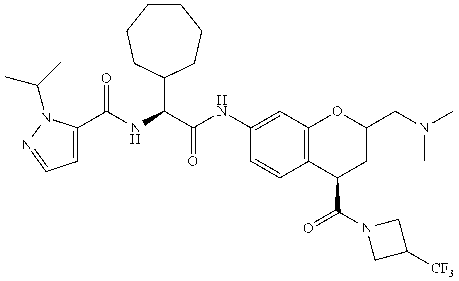 [SMILES: CC(C)n1nccc1C(=O)N[C@H](C(=O)Nc1ccc2c(c1)OC(CN(C)C)C[C@H]2C(=O)N1CC(C(F)(F)F)C1)C1CCCCCC1] | 647.3 | ¹H NMR (400 MHz. Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 2H), 4.59 − 4.55 (m, 1H), 4.43 4.35 (m, 4H), 4.13 4.09 (m, 1H), 3.89- 3.85 (m, 2H), 2.77 − 2.46 (m, 11H), 2.35- 2.33 (m, 2H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 120 | 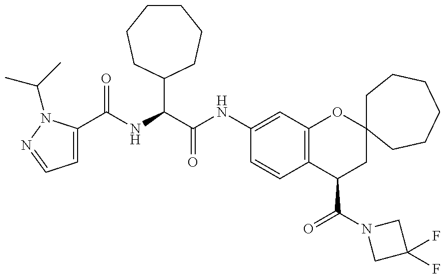 [SMILES: CC(C)n1nccc1C(=O)N[C@H](C(=O)Nc1ccc2c(c1)OC1(CCCCCC1)C[C@H]2C(=O)N1CC(F)(F)C1)C1CCCCCC1] | 640.4 | ¹H NMR (400 MHz, Chloroform-d) δ 8.27 (s, 1H), 7.49 − 7.45 (m, 1H), 7.11 (s, 1H), 7.05 − 7.01 (m, 1H), 6.91 − 6.88 (m, 1H), 6.85 − 6.82 (m, 1H), 6.55 − 6.52 (m, 1H), 5.42 − 5.38 (m, 1H), 4.53 − 4.50 (m, 1H), 4.43 − 4.25 (m, 4H), 3.97 − 3.92 (m, 1H), 3.82 − 3.77 (m, 1H), 2.21 − 2.15 (m, 1H), 1.99 − 194 (m, 2H), 1.87 − 1.78 (m, 5H), 1.78 − 1.71 (m, 3H), 1.68 − 1.62 (m, 5H), 1.57 − 1.55 (m, 2H), 1.48 − 1.41 (m, 16H). |
| 121 | 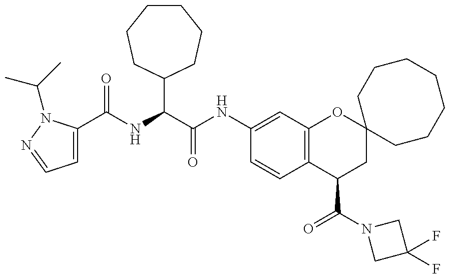 [SMILES: CC(C)n1nccc1C(=O)N[C@H](C(=O)Nc1ccc2c(c1)OC1(CCCCCCC1)C[C@H]2C(=O)N1CC(F)(F)C1)C1CCCCCC1] | 654.4 | ¹H NMR (400 MHz, Chloroform-d) δ 8.27 (s, 1H), 7.47 − 7.44 (m, 1H), 7.11 (s, 1H), 7.09 − 7.07 (m, 1H), 6.90 − 6.85 (m, 1H), 6.85 − 6.82 (m, 1H), 6.56 − 6.53 (m, 1H), 5.40 − 5.36 (m, 1H), 4.53 − 4.50 (m, 1H), 4.43 − 4.25 (m, 4H), 3.97 − 3.92 (m, 1H), 3.82 − 3.80 (m, 1H), 2.21 − 2.15 (m, 1H), 2.02 − 1.96 (m, 2H), 1.87 − 1.78 (m, SH), 1.78 − 1.71 (m, 3H), 1.68 − 1.63 (m, 5H), 1.57 − 1.53 (m, 2H), 1.48 − 1.41 (m, 18H). |
| 122 | 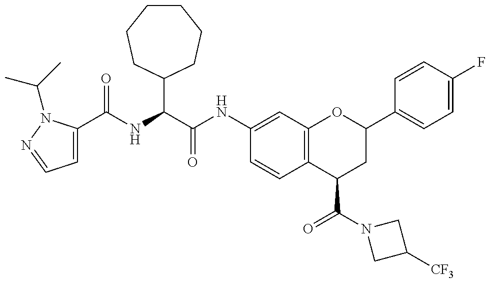 [SMILES: CC(C)n1nccc1C(=O)N[C@H](C(=O)Nc1ccc2c(c1)OC(c1ccc(F)cc1)C[C@H]2C(=O)N1CC(C(F)(F)F)C1)C1CCCCCC1] | 684.3 | ¹H NMR (400 MHz, Chloroform-d) δ 8.56 (s, 1H), 7.49 − 7.45 (m, 3H), 7.25 − 7.23 (m, 3H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 123 | 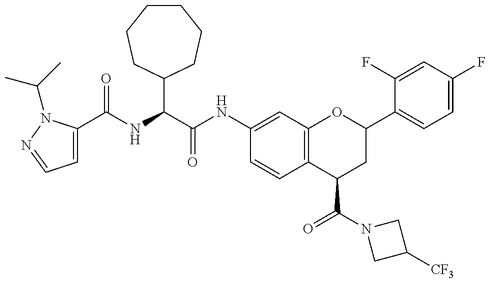 [SMILES: CC(C)n1nccc1C(=O)N[C@H](C(=O)Nc1ccc2c(c1)OC(c1ccc(F)cc1F)C[C@H]2C(=O)N1CC(C(F)(F)F)C1)C1CCCCCC1] | 702.3 | ¹H NMR (400 MHz, Chloroform-d) δ 8.56 (s, 1H), 7.49 − 7.45 (m, 3H), 7.25 − 7.23 (m, 2H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 124 | 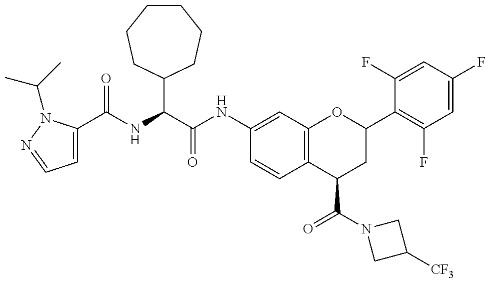 [SMILES: CC(C)n1nccc1C(=O)N[C@H](C(=O)Nc1ccc2c(c1)OC(c1c(F)cc(F)cc1F)C[C@H]2C(=O)N1CC(C(F)(F)F)C1)C1CCCCCC1] | 720.3 | ¹H NMR (400 MHz, Chloroform-d) δ 8.56 (s, 1H), 7.49 − 7.45 (m, 3H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 125 | 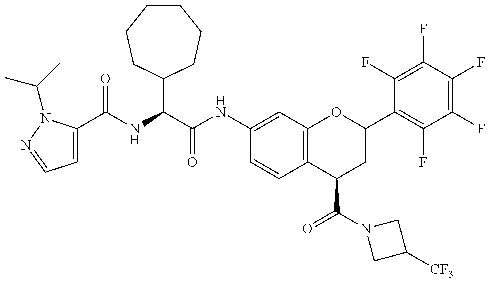 [SMILES: CC(C)n1nccc1C(=O)N[C@H](C(=O)Nc1ccc2c(c1)OC(c1c(F)c(F)c(F)c(F)c1F)C[C@H]2C(=O)N1CC(C(F)(F)F)C1)C1CCCCCC1] | 756.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 126 | 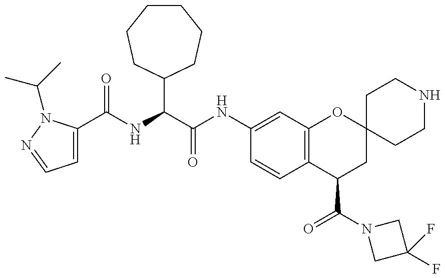 [SMILES: CC(C)n1nccc1C(=O)N[C@H](C(=O)Nc1ccc2c(c1)OC1(CCNCC1)C[C@H]2C(=O)N1CC(F)(F)C1)C1CCCCCC1] | 628.3 | ¹H NMR (400 MHz, Chloroform-d) δ 8.07 (s, 1H), 7.53 − 7.46 (m, 1H), 7.27 − 7.25 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 1H), 3.70 − 3.66 (m, 1H), 2.75 − 2.72 (m, 4H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H). |
| 127 | 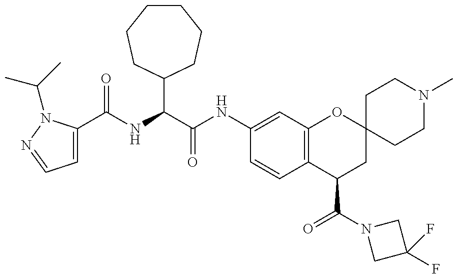 [SMILES: CC(C)n1nccc1C(=O)N[C@H](C(=O)Nc1ccc2c(c1)OC1(CCN(C)CC1)C[C@H]2C(=O)N1CC(F)(F)C1)C1CCCCCC1] | 644.4 | ¹H NMR (400 MHz, Chloroform-d) δ 8.07 (s, 1H), 7.53 − 7.46 (m, 1H), 7.27 − 7.25 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 1H), 3.70 − 3.66 (m, 1H), 2.75 − 2.72 (m, 7H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H), |
| 128 | 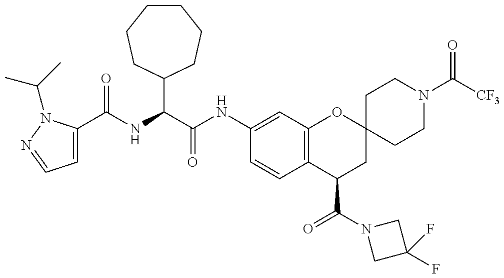 [SMILES: CC(C)n1nccc1C(=O)N[C@H](C(=O)Nc1ccc2c(c1)OC1(CCN(C(=O)C(F)(F)F)CC1)C[C@H]2C(=O)N1CC(F)(F)C1)C1CCCCCC1] | 723.3 | ¹H NMR (400 MHz, Chloroform-d) δ 8.07 (s, 1H), 7.53 − 7.46 (m, 1H), 7.27 − 7.25 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 1H), 3.70 − 3.66 (m, 1H), 2.75 − 2.72 (m, 4H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H). |
| 129 | 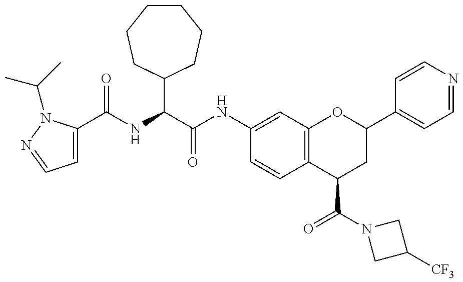 [SMILES: CC(C)n1nccc1C(=O)N[C@H](C(=O)Nc1ccc2c(c1)OC(c1ccncc1)C[C@H]2C(=O)N1CC(C(F)(F)F)C1)C1CCCCCC1] | 667.3 | ¹H NMR (400 MHz, Chloroform-d) δ 8.56 (s, 1H), 7.82 − 7.79 (m, 1H), 7.49 − 7.45 (m, 1H), 7.25 − 7.23 (m, 3H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, SH), 1.75 − 1.64 (m, 1H), 1.64 − 1.54 (m, 1H), 1.54 − 1.31 (m, 7H). |
| 130 | 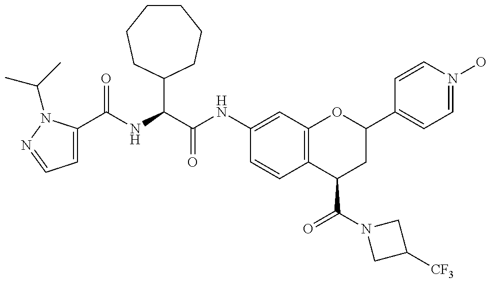 | 683.3 | ¹H NMR (400 MHz, Chloroform-d) δ 8.56 (s, 1H), 7.82 − 7.79 (m, 1H), 7.49 − 7.45 (m, 1H), 7.25 − 7.23 (m, 3H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 131 | 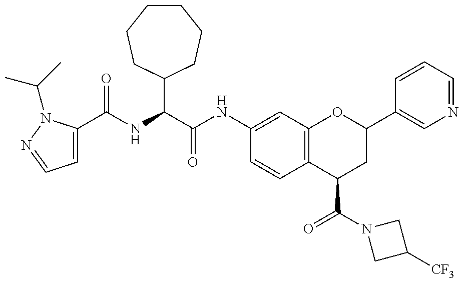 [SMILES: CC(C)n1nccc1C(=O)N[C@H](C(=O)Nc1ccc2c(c1)OC(c1cccnc1)C[C@H]2C(=O)N1CC(C(F)(F)F)C1)C1CCCCCC1] | 667.3 | ¹H NMR (400 MHz, Chloroform-d) δ 8.56 (s, 1H), 7.82 − 7.79 (m, 1H), 7.49 − 7.45 (m, 1H), 7.25 − 7.23 (m, 3H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 − 1.31 (m, 7H). |
| 132 | 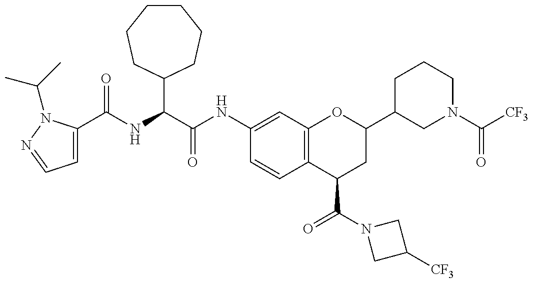 [SMILES: CC(C)n1nccc1C(=O)N[C@H](C(=O)Nc1ccc2c(c1)OC(C1CCCN(C(=O)C(F)(F)F)C1)C[C@H]2C(=O)N1CC(C(F)(F)F)C1)C1CCCCCC1] | 769.3 | ¹H NMR (400 MHz, Chloroform-d) δ 8.06 (s, 1H), 7.51 − 7.46 (m, 1H), 7.27 − 7.25 (m, 1H), 7.03 − 6.90 (m, 2H), 6.58 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 1H), 3.70 − 3.66 (m, 1H), 2.75 − 2.72 (m, 4H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H) |
| 133 | 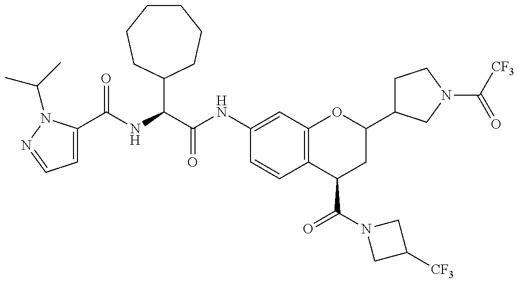 [SMILES: CC(C)n1nccc1C(=O)N[C@H](C(=O)Nc1ccc2c(c1)OC(C1CCN(C(=O)C(F)(F)F)C1)C[C@H]2C(=O)N1CC(C(F)(F)F)C1)C1CCCCCC1] | 755.3 | ¹H NMR (400 MHz, Chloroform-d) δ 8.07 (s, 1H), 7.53 − 7.46 (m, 1H), 7.27 − 7.25 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 1H), 3.70 − 3.66 (m, 1H), 2.75 − 2.72 (m, 4H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 5H). |
| 134 | 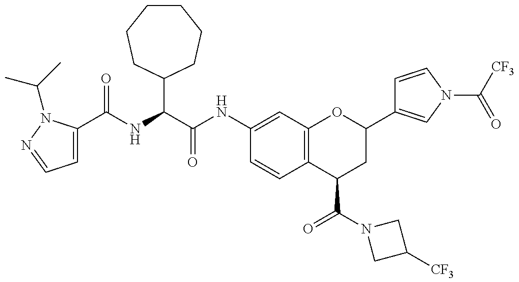 [SMILES: CC(C)n1nccc1C(=O)N[C@H](C(=O)Nc1ccc2c(c1)OC(c1ccn(C(=O)C(F)(F)F)c1)C[C@H]2C(=O)N1CC(C(F)(F)F)C1)C1CCCCCC1] | 751.3 | ¹H NMR (400 MHz, Chloroform-d) δ 8.56 (s, 1H), 7.49 − 7.45 (m, 3H), 7.25 − 7.23 (m, 2H), 6.99 − 6.95 (m, 1H), 6.90 − 6.79 (m, 2H), 6.61 − 6.57 (m, 1H), 5.42 − 5.38 (m, 1H), 4.59 − 4.55 (m, 2H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 1H), 1.64 − 1.54 (m, 1H), 1.54 1.31 (m, 7H) |
| 135 | 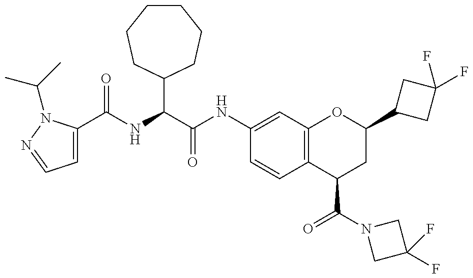 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCCCCC1] | 648.3 | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.43 − 4.35 (m, 4H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 2H), 1.64 − 1.54 (m, 2H), 1.54 − 1.31 (m, 8H). |
| 136 | 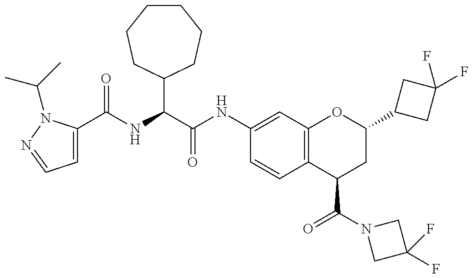 [SMILES: CC(C)n1nccc1C(=O)N[C@H](C(=O)Nc1ccc2c(c1)O[C@H](C1CC(F)(F)C1)C[C@H]2C(=O)N1CC(F)(F)C1)C1CCCCCC1] | | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H). 5.45 − 5.35 (m, 1H), 4.43 − 4.35 (m, 4H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 2H), 1.64 − 1.54 (m, 2H), 1.54 − 1.31 (m, 8H). |
| 137 | 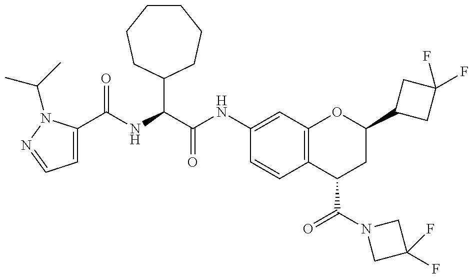 [SMILES: CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1] | | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.43 − 4.35 (m, 4H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 2H), 1.64 − 1.54 (m, 2H), 1.54 − 1.31 (m, 8H). |
| 138 | 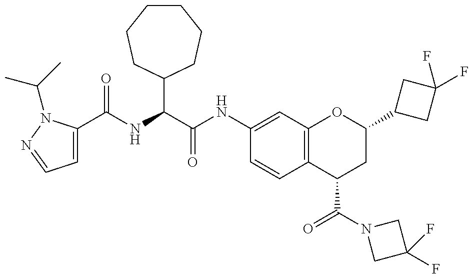 [SMILES: CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1] | | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.43 − 4.35 (m, 4H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 2H), 1.64 − 1.54 (m, 2H), 1.54 − 1.31 (m, 8H). |
| 139 | 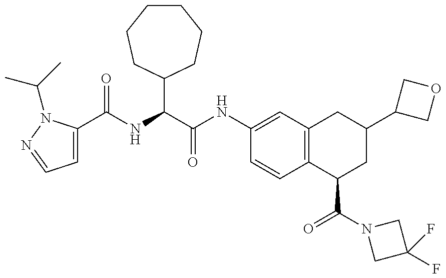 [SMILES: CC(C)n1nccc1C(=O)N[C@H](C(=O)Nc1ccc2c(c1)CC(C1COC1)C[C@H]2C(=O)N1CC(F)(F)C1)C1CCCCCC1] | 612.3 | ¹H NMR (400 MHz. Chloroform-d) o 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 1H), 4.13. 4.09 (m, 1H), 3.89 − 3.85 (m, 2H), 2.77 − 2.46 (m, 6H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 9H) |
| 140 | 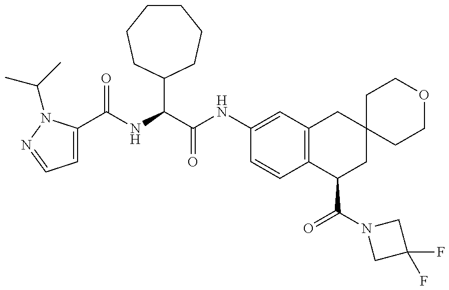 [SMILES: CC(C)n1nccc1C(=O)N[C@H](C(=O)Nc1ccc2c(c1)CC1(CCOCC1)C[C@H]2C(=O)N1CC(F)(F)C1)C1CCCCCC1] | 626.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 1H), 4.13 − 4.09 (m, 1H), 3.89 − 3.85 (m, 2H), 3.77 − 3.46 (m, 6H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 13H). |
| 141 | 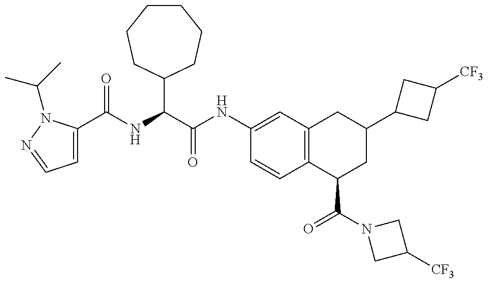 [SMILES: CC(C)n1nccc1C(=O)N[C@H](C(=O)Nc1ccc2c(c1)CC(C1CC(C(F)(F)F)C1)C[C@H]2C(=O)N1CC(C(F)(F)F)C1)C1CCCCCC1] | 710.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 2H), 4.13 − 4.09 (m, 1H), 3.89 − 3.85 (m, 2H), 2.77 − 2.46 (m, 2H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 9H). |
| 142 | 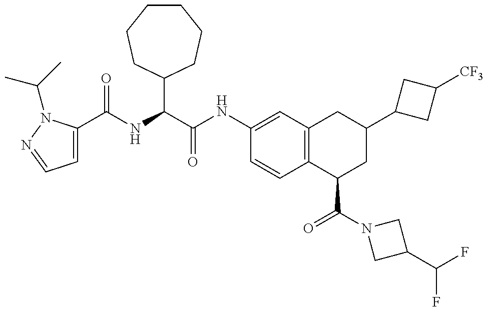 [SMILES: CC(C)n1nccc1C(=O)N[C@H](C(=O)Nc1ccc2c(c1)CC(C1CC(C(F)(F)F)C1)C[C@H]2C(=O)N1CC(C(F)F)C1)C1CCCCCC1] | 692.4 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 3H), 4.13 − 4.09 (m, 1H), 3.89 − 3.85 (m, 2H), 2.77 − 2.46 (m, 2H), 2.34 (s, 1H), 2.23 − 2.03 (m, 2H), 2.01 − 1.76 (m, 1H), 1.75 − 1.64 (m, 2H), 1.64 − 1.54 (m, 1H). 1.54 − 1.31 (m, 9H). |
| 143 | 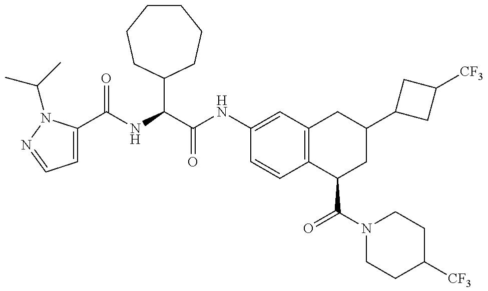 [SMILES: CC(C)n1nccc1C(=O)N[C@H](C(=O)Nc1ccc2c(c1)CC(C1CC(C(F)(F)F)C1)C[C@H]2C(=O)N1CCC(C(F)(F)F)CC1)C1CCCCCC1] | 738.4 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 3H), 4.13 − 4.09 (m, 1H), 3.89 − 3.85 (m, 2H), 2.77 − 2.46 (m, 2H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 13H). |
| 144 | 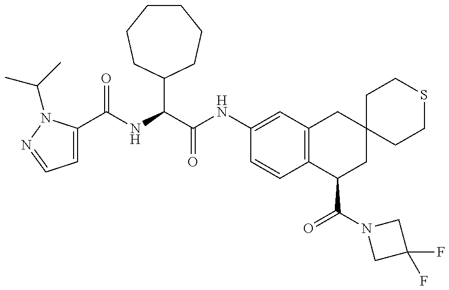 [SMILES: CC(C)n1nccc1C(=O)N[C@H](C(=O)Nc1ccc2c(c1)CC1(CCSCC1)C[C@H]2C(=O)N1CC(F)(F)C1)C1CCCCCC1] | 642.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 1H), 4.13 − 4.09 (m, 1H), 3.89 − 3.85 (m, 2H), 2.77 − 2.46 (m, 6H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 13H). |
| 145 | 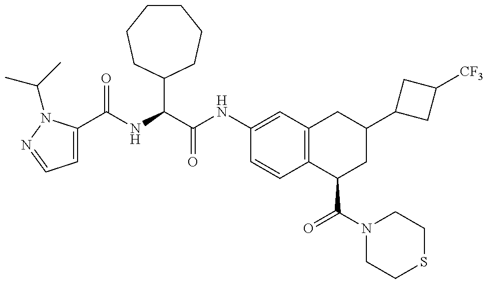 [SMILES: CC(C)n1nccc1C(=O)N[C@H](C(=O)Nc1ccc2c(c1)CC(C1CC(C(F)(F)F)C1)C[C@H]2C(=O)N1CCSCC1)C1CCCCCC1] | 688.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 2H), 4.13 − 4.09 (m, 1H), 3.89 − 3.85 (m, 2H), 2.77 − 2.46 (m, 6H), 2.34 (S, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 13H). |
| 146 | 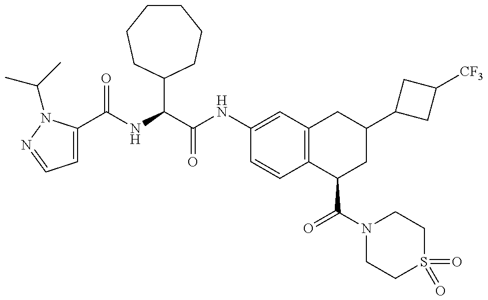 [SMILES: CC(C)n1nccc1C(=O)N[C@H](C(=O)Nc1ccc2c(c1)CC(C1CC(C(F)(F)F)C1)C[C@H]2C(=O)N1CCS(=O)(=O)CC1)C1CCCCCC1] | 720.3 | ¹H NMR (400 MHz, Chloroform-d) δ 8.56 (s, 1H), 7.42 − 7.38 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.43 − 4.35 (m, 1H), 4.13 − 4.09 (m, 1H), 3.89 − 3.85 (m, 2H), 3.77 − 3.46 (m, 6H), 2.34 (s, 1H), 2.23 − 2.03 (m, 2H), 2.01 − 1.76 (m, 1H), 1.75 − 1.64 (m, 2H), 1.64 − 1.54 (m, 1H), 1.54 − 1.31 (m, 13H). |
| 148 | 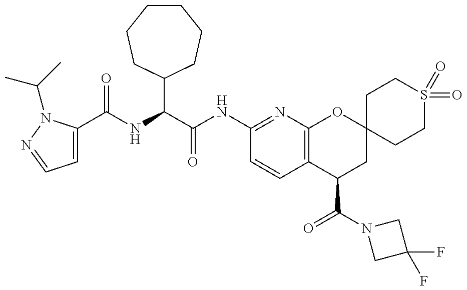 [SMILES: CC(C)n1nccc1C(=O)N[C@H](C(=O)Nc1ccc2c(n1)OC1(CCS(=O)(=O)CC1)C[C@H]2C(=O)N1CC(F)(F)C1)C1CCCCCC1] | 687.3 | ¹H NMR (400 MHz, Chloroform-d) δ 8.07 (s, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 6H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H) |
| 149 | 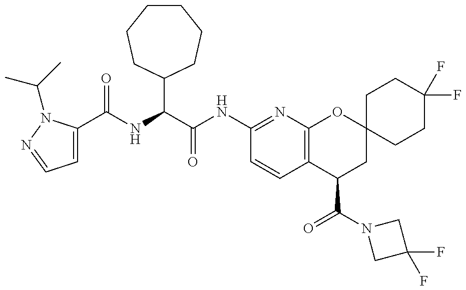 [SMILES: CC(C)n1nccc1C(=O)N[C@H](C(=O)Nc1ccc2c(n1)OC1(CCC(F)(F)CC1)C[C@H]2C(=O)N1CC(F)(F)C1)C1CCCCCC1] | 663.3 | ¹H NMR (400 MHz, Chloroform-d) δ 8.07 (s, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 1H), 2.07 − 1.93 (m, 3H), 1.85-1.81 (m, 7H), 1.72 (m, 2H), 1.64 − 1.55 (m, 1H), 1.52 − 1.44 (m, 7H). |
| 151 | 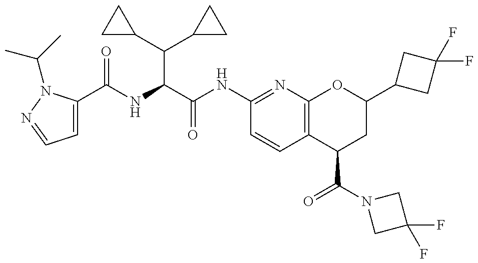 [SMILES: CC(C)n1nccc1C(=O)N[C@H](C(=O)Nc1ccc2c(n1)OC(C1CC(F)(F)C1)C[C@H]2C(=O)N1CC(F)(F)C1)C(C1CC1)C1CC1] | 647.3 | ¹H NMR (400 MHz, Chloroform-d) δ 8.56 (s, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, SH), 1.75 − 1.64 (m, 2H), 1.64 − 1.54 (m, 1H), 1.54 − 1.31 (m, 7H). |
| 152 | 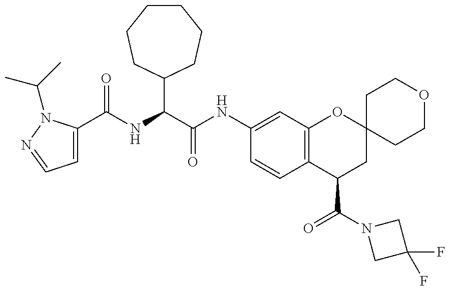 [SMILES: CC(C)n1nccc1C(=O)N[C@H](C(=O)Nc1ccc2c(c1)OC1(CCOCC1)C[C@H]2C(=O)N1CC(F)(F)C1)C1CCCCCC1] | 628.3 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H). |
| 153 | 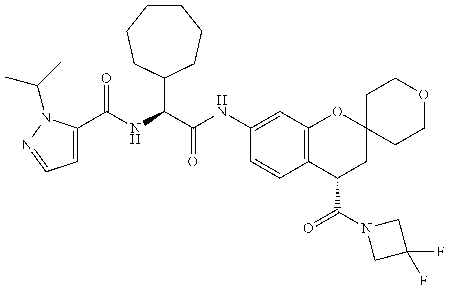 [SMILES: CC(C)n1nccc1C(=O)N[C@H](C(=O)Nc1ccc2c(c1)OC1(CCOCC1)C[C@@H]2C(=O)N1CC(F)(F)C1)C1CCCCCC1] | 628.3 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 7H), |
| 154 | 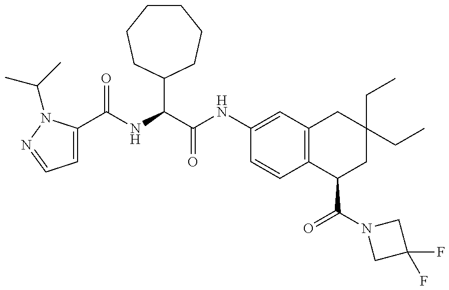 [SMILES: CCC1(CC)Cc2cc(NC(=O)[C@@H](NC(=O)c3ccnn3C(C)C)C3CCCCCC3)ccc2[C@H](C(=O)N2CC(F)(F)C2)C1] | 612.4 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 2.35 − 2.19 (m, 5H), 2.07 − 1.93 (m, 4H), 1.83 (m, 2H), 1.72 (m, 2H), 1.64 − 1.55 (m, 1H), 1.52 − 1.44 (m, 11H). |
| 155 | 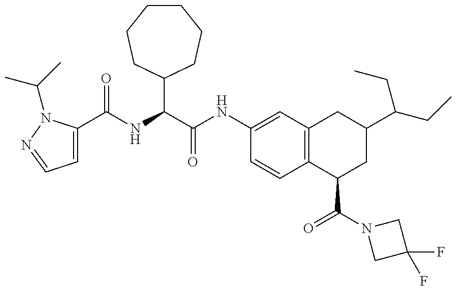 [SMILES: CCC(CC)C1Cc2cc(NC(=O)[C@@H](NC(=O)c3ccnn3C(C)C)C3CCCCCC3)ccc2[C@H](C(=O)N2CC(F)(F)C2)C1] | 626.4 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 2.35 − 2.19 (m, 5H), 2.07 − 1.93 (m, 4H), 1.83 (m, 2H), 1.72 (m, 2H), 1.64 − 1.55 (m, 1H), 1.52 − 1.44 (m, 12H). |
| 156 | 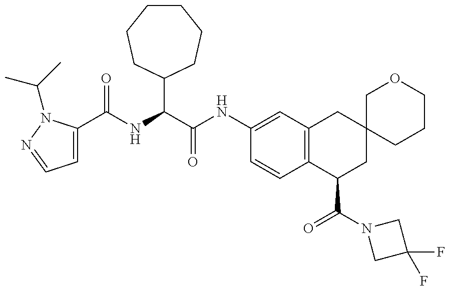 [SMILES: CC(C)n1nccc1C(=O)N[C@H](C(=O)Nc1ccc2c(c1)CC1(CCCOC1)C[C@H]2C(=O)N1CC(F)(F)C1)C1CCCCCC1] | 626.3 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 3H), 2.07 − 1.93 (m, 2H), 1.83 (m, 2H), 1.72 (m, 2H), 1.64 − 1.55 (m, 3H), 1.52 − 1.44 (m, 7H). |
| 157 | 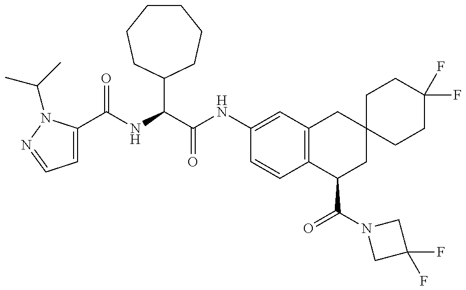 [SMILES: CC(C)n1nccc1C(=O)N[C@H](C(=O)Nc1ccc2c(c1)CC1(CCC(F)(F)CC1)C[C@H]2C(=O)N1CC(F)(F)C1)C1CCCCCC1] | 660.3 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 2.35 − 2.19 (m, 5H), 2.07 − 1.93 (m, 4H), 1.83 (m, 2H), 1.72 (m, 2H), 1.64 − 1.55 (m, 1H), 1.52 − 1.44 (m, 7H). |
| 158 | 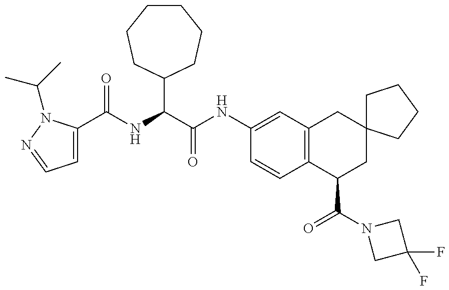 [SMILES: CC(C)n1nccc1C(=O)N[C@H](C(=O)Nc1ccc2c(c1)CC1(CCCC1)C[C@H]2C(=O)N1CC(F)(F)C1)C1CCCCCC1] | 610.3 | ¹H NMR (400 MHz, Chloroform-d) 6 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 2.35 − 2.19 (m, 5H), 2.07 − 1.93 (m, 4H), 1.83 (m, 2H), 1.72 (m, 2H), 1.64 − 1.55 (m, 1H), 1.52 − 1.44 (m, 7H). |
| 159 | 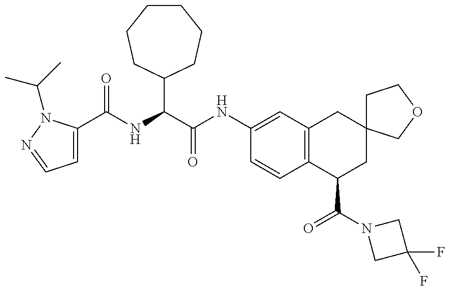 [SMILES: CC(C)n1nccc1C(=O)N[C@H](C(=O)Nc1ccc2c(c1)CC1(CCOC1)C[C@H]2C(=O)N1CC(F)(F)C1)C1CCCCCC1] | 612.3 | ¹H NMR (400 MHz, Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 3H), 2.07 − 1.93 (m, 2H), 1.83 (m, 2H), 1.72 (m, 2H), 1.64 − 1.55 (m, 1H), 1.52 − 1.44 (m, 7H). |
| 160 | 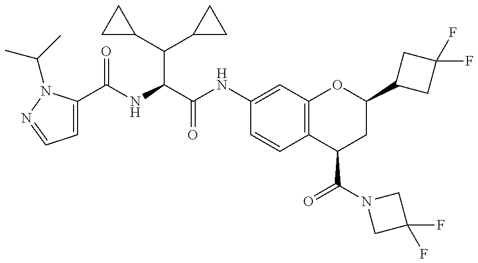 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C(C1CC1)C1CC1] | 646.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 7H). |
| 161 | 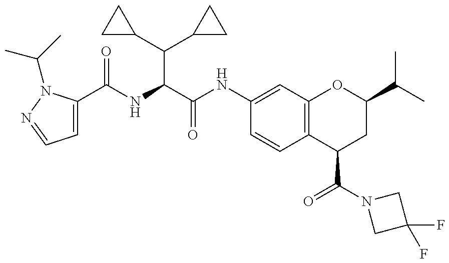 [SMILES: CC(C)[C@H]1C[C@@H](C(=O)N2CC(F)(F)C2)c2ccc(NC(=O)[C@@H](NC(=O)c3ccnn3C(C)C)C(C3CC3)C3CC3)cc2O1] | 598.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 7H). |
| 162 | 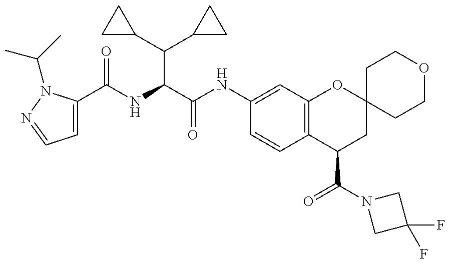 [SMILES: CC(C)n1nccc1C(=O)N[C@H](C(=O)Nc1ccc2c(c1)OC1(CCOCC1)C[C@H]2C(=O)N1CC(F)(F)C1)C(C1CC1)C1CC1] | 626.3 | ¹H NMR (400 MHz. Chloroform-d) δ 8.07 (s, 1H), 7.53 − 7.46 (m, 1H), 7.30 − 7.26 (m, 1H), 7.02 − 6.90 (m, 2H), 6.59 − 6.55 (m, 1H), 5.45 − 5.41 (m, 1H), 4.50 − 4.38 (m, 1H), 4.36 − 4.33 (m, 3H), 3.96 − 3.92 (m, 1H), 3.85 − 3.81 (m, 2H), 3.70 − 3.66 (m, 2H), 2.35 − 2.19 (m, 1H), 2.07 − 1.93 (m, 3H), 1.83 (m, 3H), 1.72 (m, 2H), 1.64 − 1.55 (m, 1H), 1.52 − 1.44 (m, 5H). |
| 163 | 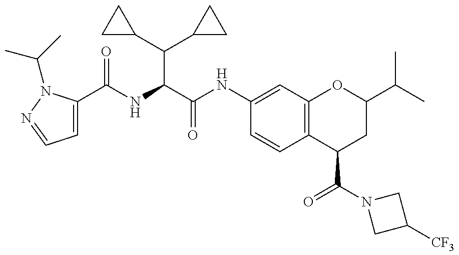 [SMILES: CC(C)C1C[C@@H](C(=O)N2CC(C(F)(F)F)C2)c2ccc(NC(=O)[C@@H](NC(=O)c3ccnn3C(C)C)C(C3CC3)C3CC3)cc2O1] | 630.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 164 | 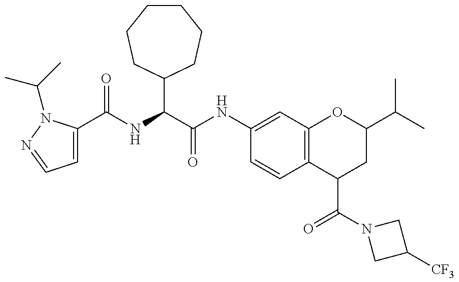 [SMILES: CC(C)C1CC(C(=O)N2CC(C(F)(F)F)C2)c2ccc(NC(=O)[C@@H](NC(=O)c3ccnn3C(C)C)C3CCCCCC3)cc2O1] | 632.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 165 | 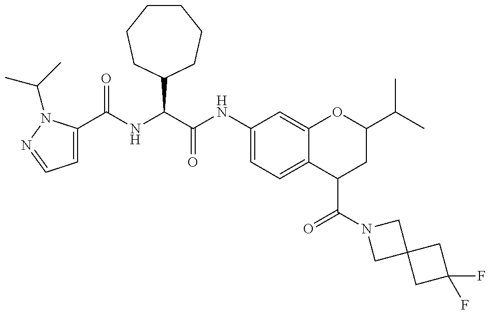 [SMILES: CC(C)C1CC(C(=O)N2CC3(C2)CC(F)(F)C3)c2ccc(NC(=O)[C@@H](NC(=O)c3ccnn3C(C)C)C3CCCCCC3)cc2O1] | 640.4 | ¹H NMR (400 MHz, Chloroform-d) δ 8.46 − 8.30 (m, 1H), 7.49 − 7.47 (m, 1H), 7.16 − 7.04 (m, 1H), 6.91 − 6.75 (m, 3H), 6.61 − 6.58 (m, 1H), 5.46 − 5.40 (m, 1H), 4.56 − 4.53 (m, 1H), 4.41 − 4.06 (m, 5H), 3.70 − 3.62 (m, 1H), 2.84 − 2.74 (m, 4H), 1.86 − 1.80 (m, 6H), 1.72 − 1.32 (m, 20H), 0.86 − 0.78 (m, 2H). |
| 166 | 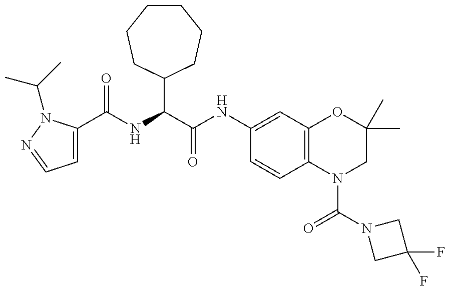 [SMILES: CC(C)n1nccc1C(=O)N[C@H](C(=O)Nc1ccc2c(c1)OC(C)(C)CN2C(=O)N1CC(F)(F)C1)C1CCCCCC1] | 587.3 | ¹H NMR (400 MHz, Chloroform-d) δ 8.52 (s, 1H), 7.45 (d, J = 2.0 Hz, 1H), 7.22 (d, J = 8.8 Hz, 1H), 7.14 (d, J = 2.4 Hz, 1H), 6.98 (d, J = 8.7 Hz, 1H), 6.93 (dd, J = 8.8. 2.4 Hz, 1H), 5.37 (h, J = 6.7 Hz, 1H), 4.57 (t, J = 8.3 Hz, 1H), 4.18 (t, J = 12.2 Hz, 4H), 3.53 − 3.43 (m, 2H), 2.20 − 2.09 (m, 1H), 1.99 (s, 1H), 1.84 (dp, J = 7.5. 5.1. 2.9 Hz, 2H), 1.73 − 1.63 (m, 2H), 1.61 − 1.53 (m, 2H), 1.42 (dd, J = 8.6. 6.6 Hz, 10H), 1.29 (d, J = 3.3 Hz, 7H). |
| 167 | 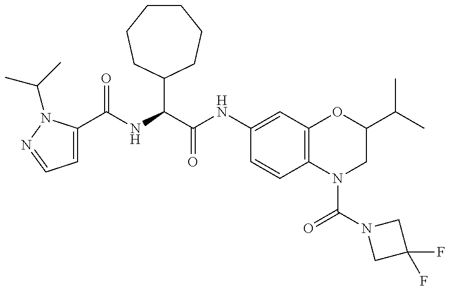 [SMILES: CC(C)C1CN(C(=O)N2CC(F)(F)C2)c2ccc(NC(=O)[C@@H](NC(=O)c3ccnn3C(C)C)C3CCCCCC3)cc2O1] | 601.3 | ¹H NMR (400 MHz, Chloroform-d) δ 8.52 (s, 1H), 7.45 (d, J = 2.0 Hz, 1H), 7.22 (d, J = 8.8 Hz, 1H), 7.14 (d, J = 2.4 Hz, 1H), 6.98 (d, J = 8.7 Hz, 1H), 6.93 (dd, J = 8.8. 2.4 Hz, 1H), 5.37 (h. ) = 6.7 Hz, 1H), 4.57-4.53 (m, 2H), 4.18 (t, J = 12.2 Hz, 4H), 3.53 − 3.43 (m, 2H), 2.20 − 2.09 (m, 1H), 1.99 (s, 1H), 1.84 (dp, J = 7.5. 5.1. 2.9 Hz. 2H), 1.73 − 1.63 (m, 2H), 1.61 − 1.53 (m, 2H), 1.42 (dd, J = 8.6. 6.6 Hz, 10H), 1.29 (d, J = 3.3 Hz, 7H). |
| 168 | 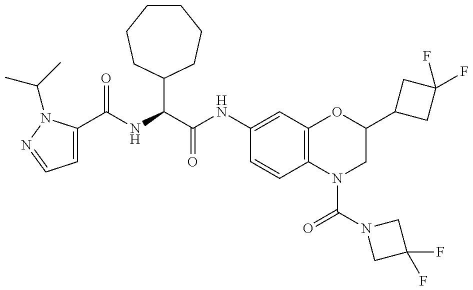 [SMILES: CC(C)n1nccc1C(=O)N[C@H](C(=O)Nc1ccc2c(c1)OC(C1CC(F)(F)C1)CN2C(=O)N1CC(F)(F)C1)C1CCCCCC1] | 649.3 | ¹H NMR (400 MHz, Chloroform-d) δ 8.52 (s, 1H), 7.45 (d, J = 2.0 Hz, 1H), 7.22 (d, J = 8.8 Hz, 1H), 7.14 (d, J = 2.4 Hz, 1H), 6.98 (d, J = 8.7 Hz, 1H), 6.93 (dd, J = 8.8. 2.4 Hz, 1H), 5.37 (h, J = 6.7 Hz, 1H), 4.57-4.53 (m, 2H), 4.18 (t, J = 12.2 Hz, 4H), 3.53 − 3.43 (m, 2H), 2.20 − 2.09 (m, 1H), 1.99 (s, 1H), 1.84 (dp, J = 7.5. 5.1. 2.9 Hz, 2H), 1.73 − 1.63 (m, 2H), 1.61 − 1.53 (m, 2H), 1.42 (dd, J = 8.6. 6.6 Hz, 10H), 1.29 (d, J = 3.3 Hz, 7H). |
| 169 | 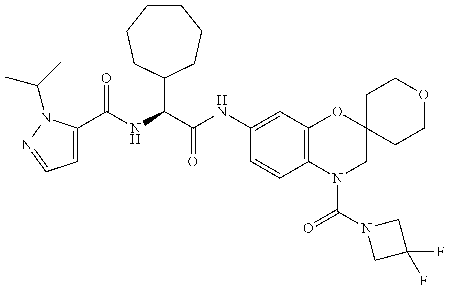 [SMILES: CC(C)n1nccc1C(=O)N[C@H](C(=O)Nc1ccc2c(c1)OC1(CCOCC1)CN2C(=O)N1CC(F)(F)C1)C1CCCCCC1] | 629.3 | ¹H NMR (400 MHz, Chloroform-d) δ 8.52 (s, 1H), 7.45 (d, J = 2.0 Hz, 1H), 7.22 (d, J = 8.8 Hz, 1H), 7.14 (d, J = 2.4 Hz, 1H), 6.98 (d, J = 8.7 Hz, 1H), 6.93 (dd, J = 8.8. 2.4 Hz, 1H), 5.37 (h, J = 6.7 Hz, 1H), 4.57-4.53 (m, 2H), 4.22- 4.18 (m, 8H), 3.53 − 3.43 (m, 2H), 2.20 − 2.09 (m, 1H), 1.99 (s, 1H), 1.84 (dp, J = 7.5. 5.1. 2.9 Hz, 2H), 1.73 − 1.63 (m, 2H), 1.61 − 1.53 (m, 2H), 1.42 (dd, J = 8.6. 6.6 Hz, 10H), 1.29 (d, J = 3.3 Hz, 7H). |
| 170 | 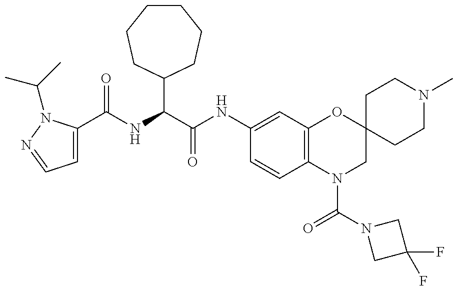 [SMILES: CC(C)n1nccc1C(=O)N[C@H](C(=O)Nc1ccc2c(c1)OC1(CCN(C)CC1)CN2C(=O)N1CC(F)(F)C1)C1CCCCCC1] | 642.4 | ¹H NMR (400 MHz, Chloroform-d) δ 8.46 − 8.30 (m, 1H), 7.49 − 7.47 (m, 1H), 7.16 − 7.04 (m, 1H), 6.91 − 6.75 (m, 3H), 6.61 − 6.58 (m, 1H), 5.46 − 5.40 (m, 1H), 4.56 − 4.53 (m, 1H), 4.41 − 4.06 (m, 5H), 3.70 − 3.62 (m, 1H), 2.84 − 2.74 (m, 4H), 1.86 − 1.80 (m, 6H), 1.72 − 1.32 (m, 20H), 0.86 − 0.78 (m, 2H), |
| 171 | 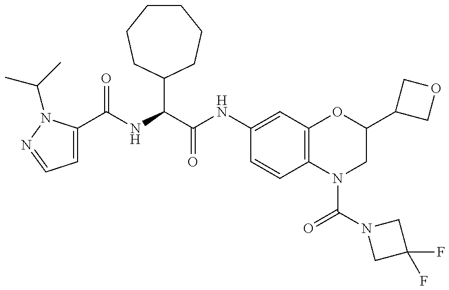 [SMILES: CC(C)n1nccc1C(=O)N[C@H](C(=O)Nc1ccc2c(c1)OC(C1COC1)CN2C(=O)N1CC(F)(F)C1)C1CCCCCC1] | 615.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 172 | 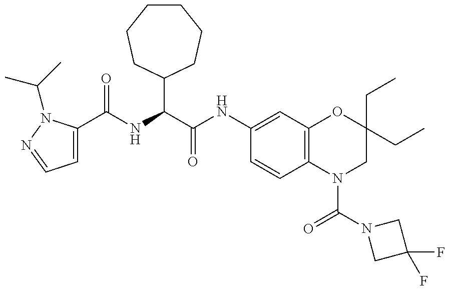 [SMILES: CCC1(CC)CN(C(=O)N2CC(F)(F)C2)c2ccc(NC(=O)[C@@H](NC(=O)c3ccnn3C(C)C)C3CCCCCC3)cc2O1] | 615.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H) |
| 173 | 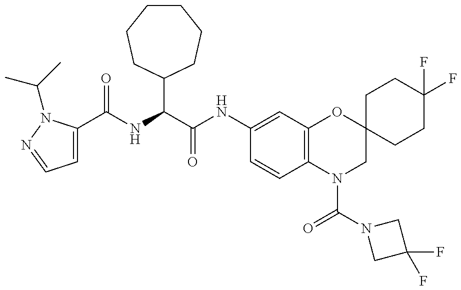 [SMILES: CC(C)n1nccc1C(=O)N[C@H](C(=O)Nc1ccc2c(c1)OC1(CCC(F)(F)CC1)CN2C(=O)N1CC(F)(F)C1)C1CCCCCC1] | 663.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H) |
| 174 | 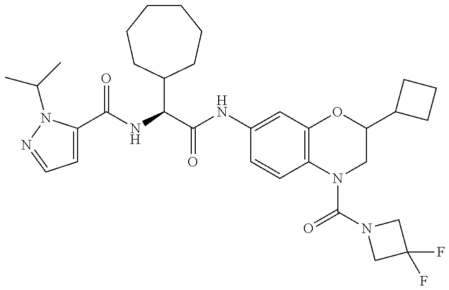 [SMILES: CC(C)n1nccc1C(=O)N[C@H](C(=O)Nc1ccc2c(c1)OC(C1CCC1)CN2C(=O)N1CC(F)(F)C1)C1CCCCCC1] | 613.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 175 | 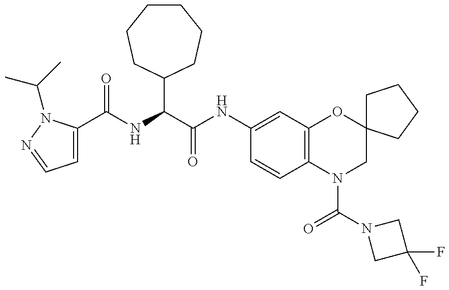 [SMILES: CC(C)n1nccc1C(=O)N[C@H](C(=O)Nc1ccc2c(c1)OC1(CCCC1)CN2C(=O)N1CC(F)(F)C1)C1CCCCCC1] | 613.3 | ¹H NMR (400 MHz, Chloroform-d) δ 8.46 − 8.30 (m, 1H), 7.49 − 7.47 (m, 1H), 7.16 − 7.04 (m, 1H), 6.91 − 6.75 (m, 3H), 6.61 − 6.58 (m, 1H), 5.46 − 5.40 (m, 1H), 4.56 − 4.53 (m, 1H), 4.41 − 4.06 (m, 5H), 3.70 − 3.62 (m, 1H), 2.84 − 2.74 (m, 4H), 1.86 − 1.80 (m, 6H), 1.72 − 1.32 (m, 20H), 0.86 − 0.78 (m, 2H). |
| 176 | 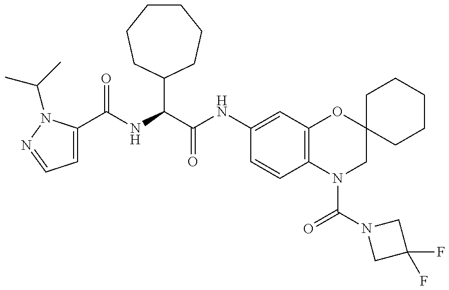 [SMILES: CC(C)n1nccc1C(=O)N[C@H](C(=O)Nc1ccc2c(c1)OC1(CCCCC1)CN2C(=O)N1CC(F)(F)C1)C1CCCCCC1] | 627.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 177 | 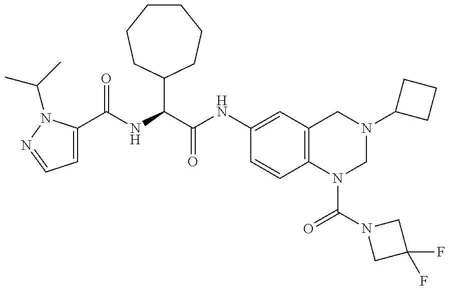 [SMILES: CC(C)n1nccc1C(=O)N[C@H](C(=O)Nc1ccc2c(c1)CN(C1CCC1)CN2C(=O)N1CC(F)(F)C1)C1CCCCCC1] | 612.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 178 | 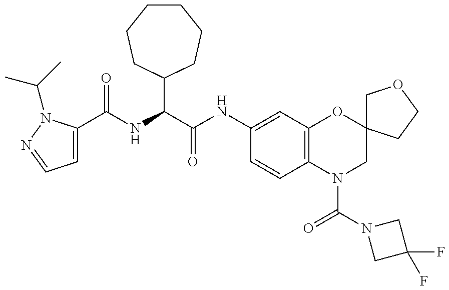 [SMILES: CC(C)n1nccc1C(=O)N[C@H](C(=O)Nc1ccc2c(c1)OC1(CCOC1)CN2C(=O)N1CC(F)(F)C1)C1CCCCCC1] | 615.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 179 | 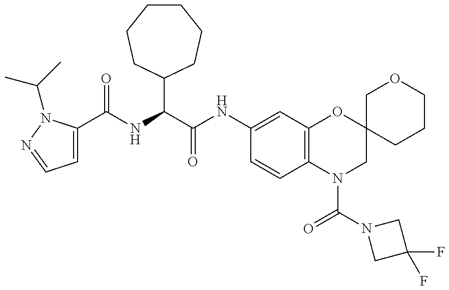 [SMILES: CC(C)n1nccc1C(=O)N[C@H](C(=O)Nc1ccc2c(c1)OC1(CCCOC1)CN2C(=O)N1CC(F)(F)C1)C1CCCCCC1] | 629.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 180 | 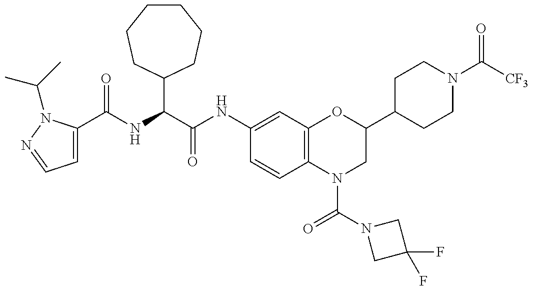 [SMILES: CC(C)n1nccc1C(=O)N[C@H](C(=O)Nc1ccc2c(c1)OC(C1CCN(C(=O)C(F)(F)F)CC1)CN2C(=O)N1CC(F)(F)C1)C1CCCCCC1] | 710.3 | ¹H NMR (400 MHz, Chloroform-d) δ 8.46 − 8.30 (m, 1H), 7.49 − 7.47 (m, 1H), 7.16 − 7.04 (m, 1H), 6.91 − 6.75 (m, 3H), 6.61 − 6.58 (m, 1H), 5.46 − 5.40 (m, 1H), 4.56 − 4.53 (m, 1H), 4.41 − 4.06 (m, 5H), 3.70 − 3.62 (m, 1H), 2.84 − 2.74 (m, 4H), 1.86 − 1.80 (m, 6H), 1.72 − 1.32 (m, 20H), 0.86 − 0.78 (m, 2H). |
| 181 | 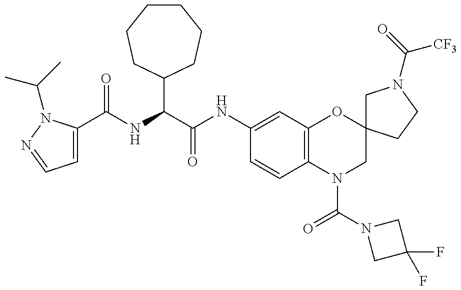 [SMILES: CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1] | 710.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 182 | 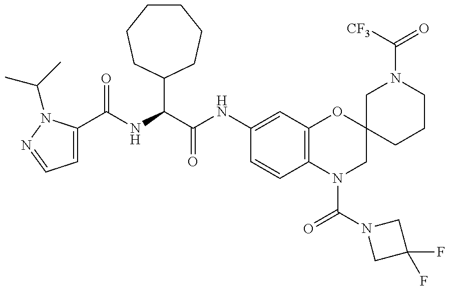 [SMILES: CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1] | 724.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 183 | 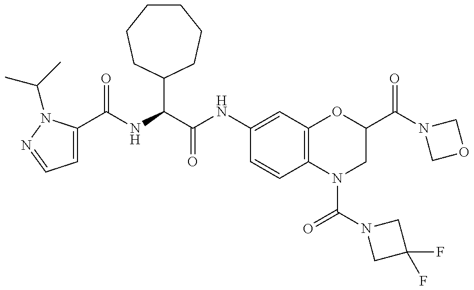 [SMILES: CC(C)n1nccc1C(=O)N[C@H](C(=O)Nc1ccc2c(c1)OC(C(=O)N1COC1)CN2C(=O)N1CC(F)(F)C1)C1CCCCCC1] | 644.3 | ¹H NMR (400 MHz. Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 184 | 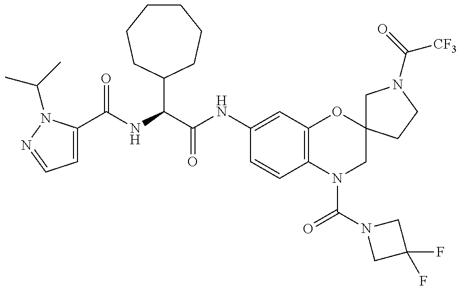 [SMILES: CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1] | 710.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 185 | 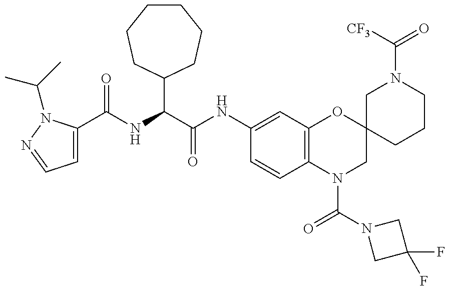 [SMILES: CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1] | 724.3 | ¹H NMR (400 MHz, Chloroform-d) δ 8.46 − 8.30 (m, 1H), 7.49 − 7.47 (m, 1H), 7.16 − 7.04 (m, 1H), 6.91 − 6.75 (m, 3H), 6.61 − 6.58 (m, 1H), 5.46 − 5.40 (m, 1H), 4.56 − 4.53 (m, 1H), 4.41 − 4.06 (m, 5H), 3.70 − 3.62 (m, 1H), 2.84 − 2.74 (m, 4H), 1.86 − 1.80 (m, 6H), 1.72 − 1.32 (m, 20H), 0.86 − 0.78 (m, 2H), |
| 186 | 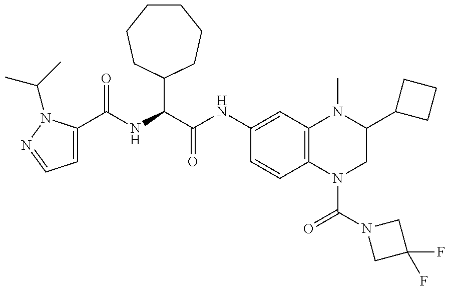 [SMILES: CC(C)n1nccc1C(=O)N[C@H](C(=O)Nc1ccc2c(c1)N(C)C(C1CCC1)CN2C(=O)N1CC(F)(F)C1)C1CCCCCC1] | 626.4 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H). |
| 187 | 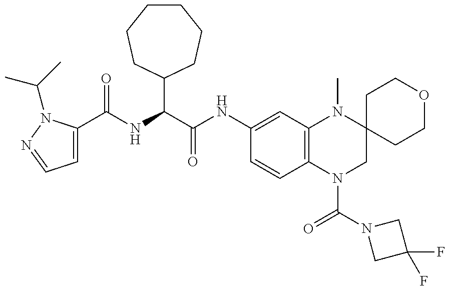 [SMILES: CC(C)n1nccc1C(=O)N[C@H](C(=O)Nc1ccc2c(c1)N(C)C1(CCOCC1)CN2C(=O)N1CC(F)(F)C1)C1CCCCCC1] | 642.4 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 20H), 1.06 − 0.98 (m, 6H). |
| 188 | 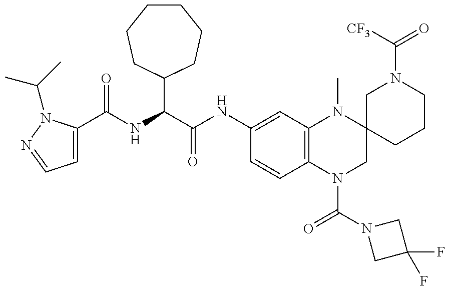 [SMILES: CC(C)n1nccc1C(=O)N[C@H](C(=O)Nc1ccc2c(c1)N(C)C1(CCCN(C(=O)C(F)(F)F)C1)CN2C(=O)N1CC(F)(F)C1)C1CCCCCC1] | 648.3 | ¹H NMR (400 MHz, Chloroform-d) δ 8.11 − 8.04 (m, 1H), 7.50 − 7.48 (m, 1H), 7.20 − 7.14 (m, 1H), 6.91 − 6.86 (m, 2H), 6.78 − 6.74 (m, 1H), 6.58 − 6.54 (m, 1H), 5.46 − 5.40 (m, 1H), 4.56 − 3.64 (m, 7H), 2.18 − 2.06 (m, 2H), 1.72 − 1.24 (m, 18H), 1.06 − 0.98 (m, 6H) |
| 189 | 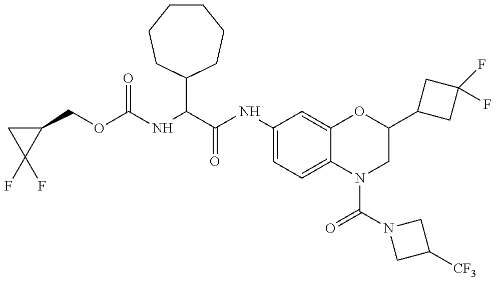 [SMILES: O=C(NC(C(=O)Nc1ccc2c(c1)OC(C1CC(F)(F)C1)CN2C(=O)N1CC(C(F)(F)F)C1)C1CCCCCC1)OC[C@@H]1CC1(F)F] | 678.3 | ¹H NMR (400 MHz, Chloroform-d) δ 7.98-7.87(m, 1H), 7.31-7.27(m, 1H), 7.23-7.12(m, 1H), 7.17-7.06(m, 2H), 5.52-5.48(m, 1H), 5.25-5.17(m, 2H), 4.42-4.34(m, 4H), 4.16-4.10(m, 1H), 3.89-3.84(m, 1H), 2.62-2.51(m, 4H), 2.35-2.33(m, 1H), 2.12-1.86(m,3H), 1.79-1.57(m, 12H), 1.51-1.44(m, 2H), |
| 190 | 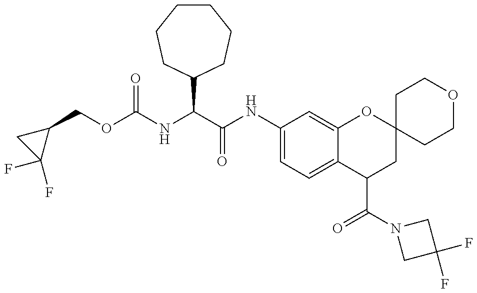 [SMILES: O=C(N[C@H](C(=O)Nc1ccc2c(c1)OC1(CCOCC1)CC2C(=O)N1CC(F)(F)C1)C1CCCCCC1)OC[C@@H]1CC1(F)F] | 626.3 | ¹H NMR (400 MHz, Chloroform-d) δ 7.98-7.87(m, 1H), 7.31-7.27(m, 1H), 7.23-7.12(m, 1H), 7.17-7.06(m, 2H), 5.46 − 5.40 (m, 1H), 4.56 − 3.64 (m, 7H), 4.16-4.10(m, 1H), 2.18 − 2.06 (m, 2H), 1.72 − 1.24 (m, 23H) |
| 191 | 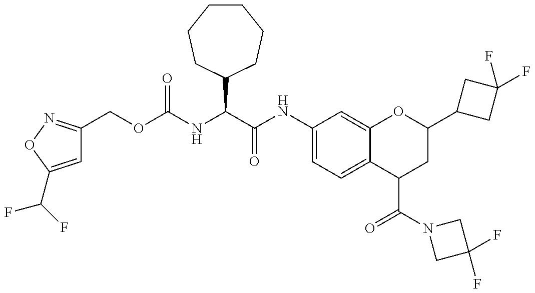 [SMILES: O=C(N[C@H](C(=O)Nc1ccc2c(c1)OC(C1CC(F)(F)C1)CC2C(=O)N1CC(F)(F)C1)C1CCCCCC1)OCc1cc(C(F)F)on1] | 687.3 | ¹H NMR (400 MHz, Chloroform-d) δ 7.80-7.77(m, 1H), 7.29-7.25(m, 1H), 7.23-7.12(m, 1H), 7.02-7.00(m, 1H), 6.95-6.88(m, 1H), 6.65-6.58(m, 1H), 5.52-5.48(m, 1H), 5.25-5.17(m, 2H), 4.42-4.34(m, 4H), 4.16-4.10(m, 1H), 3.89-3.84(m, 1H), 2.62-2.51(m, 4H), 2.35-2.33(m, 1H), 2.12-2.00(m, 2H), 1.76-1.57(m, 6H), 1.51-1.44(m, 2H), 1.32-1.24(m, 6H) |
| 192 | 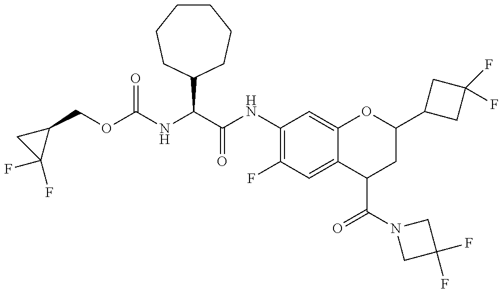 [SMILES: O=C(N[C@H](C(=O)Nc1cc2c(cc1F)C(C(=O)N1CC(F)(F)C1)CC(C1CC(F)(F)C1)O2)C1CCCCCC1)OC[C@@H]1CC1(F)F] | 664.3 | ¹H NMR (400 MHz, Chloroform-d) δ 7.98-7.87(m, 1H), 7.31-7.27(m, 1H), 7.23-7.12(m, 1H), 7.12-7.06(m, 1H), 5.52-5.48(m, 1H), 5.25-5.17(m, 2H), 4.42-4.34(m, 4H), 4.16-4.10(m, 1H), 3.89-3.84(m, 1H), 2.62-2.51(m, 4H), 2.35-2.33(m, 1H), 2.12-2.00(m, 2H), 1.76-1.57(m, 12H), 1.51-1.44(m, 2H), |
| 193 | 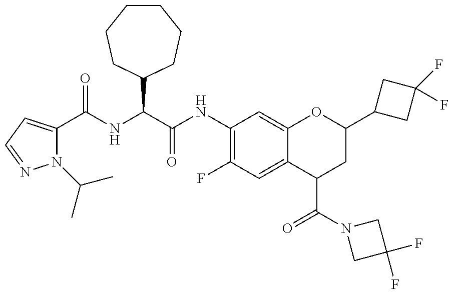 [SMILES: CC(C)n1nccc1C(=O)N[C@H](C(=O)Nc1cc2c(cc1F)C(C(=O)N1CC(F)(F)C1)CC(C1CC(F)(F)C1)O2)C1CCCCCC1] | 666.3 | ¹H NMR (400 MHz, Chloroform-d) δ 8.56 (s, 1H), 7.35 − 7.30 (m, 1H), 6.97 − 6.91 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H). 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H). 1.54 − 1.31 (m, 9H). |
| 194 | 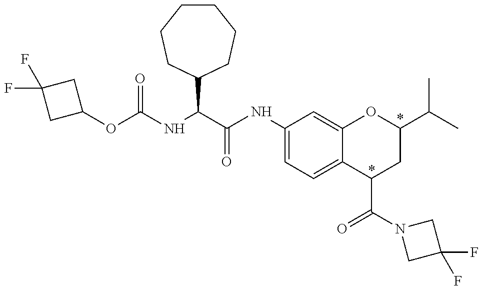 [SMILES: CC(C)C1CC(C(=O)N2CC(F)(F)C2)c2ccc(NC(=O)[C@@H](NC(=O)OC3CC(F)(F)C3)C3CCCCCC3)cc2O1] | 598.3 | ¹H NMR (400 MHz, Chloroform-d) δ 7.98-7.87(m, 1H), 7.31-7.27(m, 1H), 7.23-7.12(m, 1H), 7.17-7.06(m, 2H), 5.42 − 5.38 (m, 1H), 4.57-4.53 (m, 2H), 4.13-4.01(m, 2H), 3.53 − 3.43 (m, 2H), 2.20 − 2.09 (m, 5H), 1.73 − 1.63 (m, 4H), 1.61 − 1.33 (m, 12H), 0.87- 0.83 (m, 6H). |
| 195 | 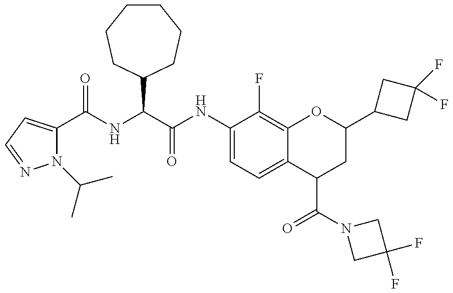 [SMILES: CC(C)n1nccc1C(=O)N[C@H](C(=O)Nc1ccc2c(c1F)OC(C1CC(F)(F)C1)CC2C(=O)N1CC(F)(F)C1)C1CCCCCC1] | 666.3 | ¹H NMR (400 MHz, Chloroform-d) δ 8.54 (s, 1H), 7.47 − 7.44 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, SH), 1.75 − 1.64 (m, 2H), 1.64 − 1.54 (m, 1H), 1.54 − 1.31 (m, 9H). |
| 196 | 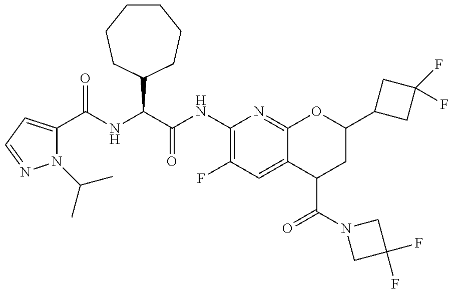 [SMILES: CC(C)n1nccc1C(=O)N[C@H](C(=O)Nc1nc2c(cc1F)C(C(=O)N1CC(F)(F)C1)CC(C1CC(F)(F)C1)O2)C1CCCCCC1] | 667.3 | ¹H NMR (400 MHz, Chloroform-d) δ 8.56 (s, 1H), 7.31 − 7.26 (m, 1H) 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 4.13 − 4.09 (m, 1H), 3.89 − 3.85 (m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H). |
| 197 | 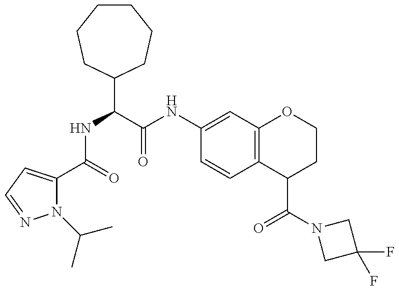 [SMILES: CC(C)n1nccc1C(=O)N[C@H](C(=O)Nc1ccc2c(c1)OCCC2C(=O)N1CC(F)(F)C1)C1CCCCCC1] | 558.3 | ¹H NMR (400 MHz, Chloroform-d) δ 8.25(d, J = 24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 4.13-4.06(m, 2H), 3.74(t, J = 8.0 Hz, 1H), 2.18-2.10(m, 3H), 1.85-1.81(m, 2H), 1.72-1.28(m, 11H) |
| 198 | 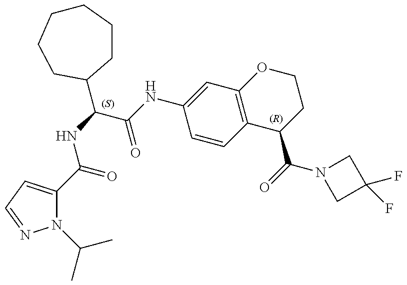 | 558.3 | ¹H NMR (400 MHz, Chloroform-d) δ 8.25(d, J = 24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 4.13-4.06(m, 2H), 3.74(t, J = 8.0 Hz, 1H), 2.18-2.10(m, 3H), 1.85-1.81(m, 2H), 1.72-1.28(m, 11H) |
| 199 | 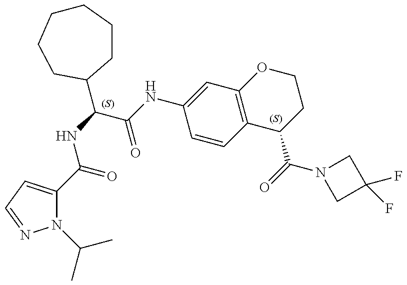 | 558.3 | ¹H NMR (400 MHz, Chloroform-d) δ 8.25(d, J = 24 Hz, 1H), 7.47(d, J = 1.2 Hz, 1H), 7.10(d, J = 4.8 Hz,1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 4.13-4.06(m, 2H), 3.74(t, J = 8.0 Hz, 1H), 2.18-2.10(m, 3H), 1.85-1.81(m, 2H), 1.72-1.28(m, 11H) |
| 200 | 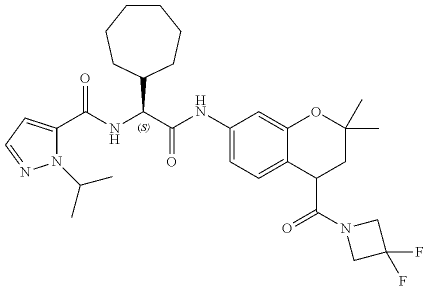 | 586.3 | ¹H NMR (400 MHz, Chloroform-d) δ 8.25(d, J = 24 Hz, 1H), 7.47(d, J = 1.2 Hz, 1H), 7.10(d, J = 4.8 Hz,1H), 7.02-6.99(m, 1H), 6.97-6.93(m, 1H), 6.88-6.82(m, 2H), 6.56(d, .J = 4.8 Hz, 1H), 5.42-5.38(m, 1H), 4.54-4.30(m, 3H), 4.13-4.06(m, 2H), 3.74(t, J = 8.0 Hz, 1H), 2.18-2.10(m, 3H), 1.85-1.81(m, 2H), 1.72-1.28(m, 21H) |
| 201 | 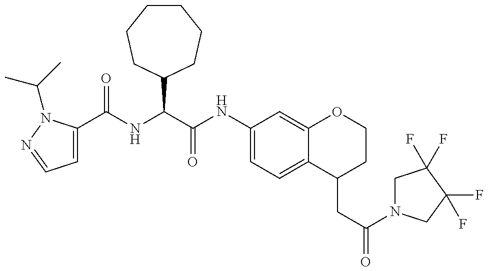 [SMILES: CC(C)n1nccc1C(=O)N[C@H](C(=O)Nc1ccc2c(c1)OCCC2CC(=O)N1CC(F)(F)C(F)(F)C1)C1CCCCCC1] | 622.3 | ¹H NMR (400 MHz, Chloroform-d) δ 8.25(d, J-24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.34-4.26(m, 5H), 4.13-4.06(m, 2H), 3.39(t, J = 8.0 Hz, 1H), 2.57-2.53(m, 2H), 2.18-2.10(m, 3H), 1.85-1.81(m, 2H), 1.72-1.28(m, 15H) |
| 202 | 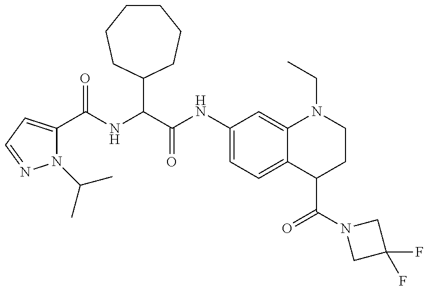 [SMILES: CCN1CCC(C(=O)N2CC(F)(F)C2)c2ccc(NC(=O)C(NC(=O)c3ccnn3C(C)C)C3CCCCCC3)cc21] | 585.3 | ¹H NMR (400 MHz, Chloroform-d) δ 8.25(d, J = 24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 3.74(t, J = 8.0 Hz, 1H), 3.58-3.50(m, 4H), 2.18-2.10(m, 3H), 1.85-1.81(m, 2H), 1.72-1.28(m, 14H) |
| 203 | 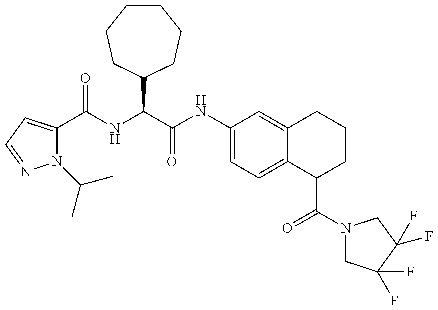 [SMILES: CC(C)n1nccc1C(=O)N[C@H](C(=O)Nc1ccc2c(c1)CCCC2C(=O)N1CC(F)(F)C(F)(F)C1)C1CCCCCC1] | 606.3 | ¹H NMR (400 MHz, Chloroform-d) δ 8.25(d, J = 24 Hz, 1H), 7.47(d, J = 1.2 Hz, 1H), 7.10(d, J = 4.8 Hz,1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.50(m, 1H), 4.30-4.27(m, 4H), 3.74(t, J = 8.0 Hz, 1H), 2.82-2.76(m, 2H), 2.18-2.10(m, 5H), 1.85-1.81(m, 2H), 1.72-1.28(m, 11H) |
| 204 | 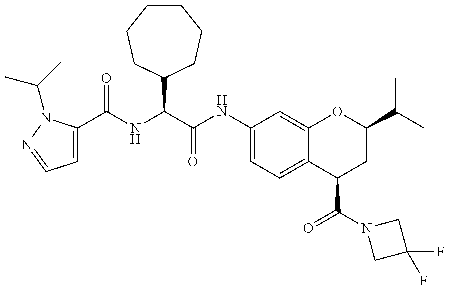 [SMILES: CC(C)[C@H]1C[C@@H](C(=O)N2CC(F)(F)C2)c2ccc(NC(=O)[C@@H](NC(=O)c3ccnn3C(C)C)C3CCCCCC3)cc2O1] | 600.3 | ¹H NMR (400 MHz, Chloroform-d) δ 7.99(dd, J = 16.0 Hz, 4.0 Hz, 1H), 7.51- 7.49(m, 1H), 7.18-7.13(m, 1H), 6.99- 6.87(m, 2H), 6.77-6.72(m, 1H), 6.56- 6.55(m, 1H), 5.45-5.37(m, 1H), 4.52- 4.48(m, 1H), 4.45-4.23(m, 4H), 4.08- 3.97(m, 1H), 3.71-3.65(m, 1H), 2.19- 2.06(m, 2H), 1.94-1.79(m, 7H), 1.68- 1.25(m, 13H), 1.03-0.97(m, 6H) |
| 205 | 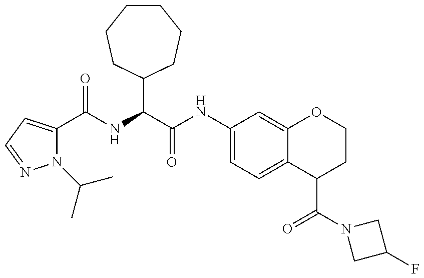 [SMILES: CC(C)n1nccc1C(=O)N[C@H](C(=O)Nc1ccc2c(c1)OCCC2C(=O)N1CC(F)C1)C1CCCCCC1] | 540.3 | ¹H NMR (400 MHz, Chloroform-d) δ 8.25(d, J = 24 Hz, 1H), 7.47(d, J = 1.2 Hz, 1H), 7.10(d, J = 4.8 Hz,1H), 7.02-6.99(m, 1H), 6.97-6.93(m, 1H). 6.88-6.82(m, 2H), 6.56(d, .J = 4.8 Hz, 1H), 5.42-5.38(m, 1H), 4.95-4.90(m, 1H), 4.34-4.26(m, 5H), 4.13-4.06(m, 2H). 3.96(t, J = 8.0 Hz, 1H), 2.18-2.10(m, 3H), 1.85-1.81(m, 2H), 1.72-1.28(m, 15H) |
| 206 | 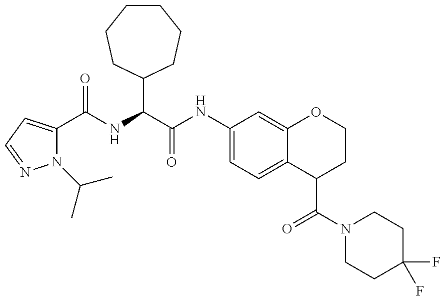 [SMILES: CC(C)n1nccc1C(=O)N[C@H](C(=O)Nc1ccc2c(c1)OCCC2C(=O)N1CCC(F)(F)CC1)C1CCCCCC1] | 586.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H),, 4.34-4.26(m, 5H), 4.13-4.06(m, 2H), 3.96(t, J = 8.0 Hz, 1H), 2.18-2.10(m, 7H), 1.85-1.81(m, 2H), 1.72-1.28(m, 15H) |
| 207 | 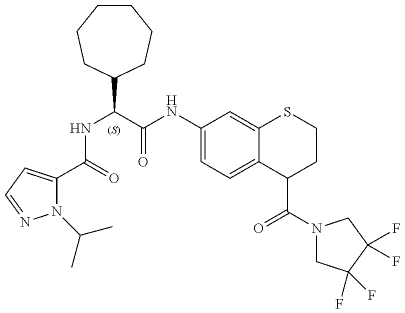 | 624.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.34-4.26(m, 5H), 3.96(t, J = 8.0 Hz, 1H), 3.78-3.70(m, 2H),2.18-2.10(m, 7H), 1.85-1.81(m, 2H), 1.72-1.28(m, 11H) |
| 208 | 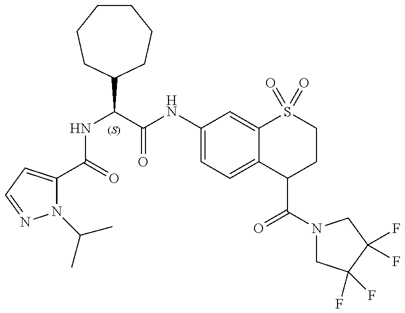 | 656.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H). 6.88-6.82(m, 2H), 6.56(d, J = 4.8 Hz, 1H), 5.42-5.38(m, 1H), 4.34-4.26(m, 5H), 3.96(t, J = 8.0 Hz, 1H), 3.48-3.41(m, 2H), 2.18-2.10(m, 7H), 1.85-1.81(m, 2H), 1.72-1.28(m, 11H) |
| 209 | 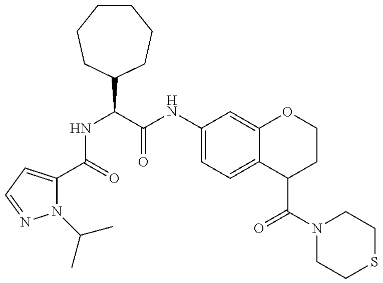 [SMILES: CC(C)n1nccc1C(=O)N[C@H](C(=O)Nc1ccc2c(c1)OCCC2C(=O)N1CCSCC1)C1CCCCCC1] | 568.3 | ¹H NMR (400 MHz, Chloroform-d) δ 8.25(d, J = 24 Hz, 1H), 7.47(d, J = 1.2 Hz, 1H), 7.10(d, J = 4.8 Hz,1H), 7.02-6.99(m, 1H), 6.97-6.93(m, 1H), 6.88-6.82(m, 2H), 6.56(d, J = 4.8 Hz, 1H), 5.42-5.38(m, 1H),, 4.34-4.26(m, 5H), 4.13-4.06(m, 2H), 3.96(t, J = 8.0 Hz, 1H), 3.72-3.68(m, 4H), 2.18-2.10(m, 3H), 1.85-1.81(m, 2H), 1.72-1.28(m, 15H) |
| 210 | 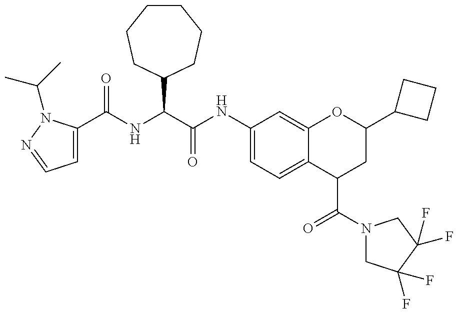 [SMILES: CC(C)n1nccc1C(=O)N[C@H](C(=O)Nc1ccc2c(c1)OC(C1CCC1)CC2C(=O)N1CC(F)(F)C(F)(F)C1)C1CCCCCC1] | 662.3 | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 6H), 1.64 − 1.54 (m, 5H), 1.54 − 1.31 (m, 10H). |
| 211 | 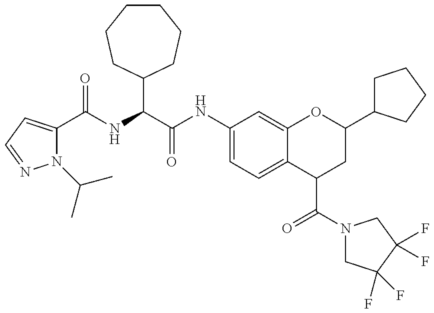 [SMILES: CC(C)n1nccc1C(=O)N[C@H](C(=O)Nc1ccc2c(c1)OC(C1CCCC1)CC2C(=O)N1CC(F)(F)C(F)(F)C1)C1CCCCCC1] | 676.3 | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.22-4.12(m, 1H), 3.95- 3.84 (m, 1H), 2.77 − 2.46 (m, 6H), 2.34 (s, 1H), 2.23 − 2.03 (m, 2H), 2.01 − 1.76 (m, 5H), 1.75 − 1.64 (m, 6H), 1.64 − 1.54 (m, 5H), 1.54 − 1.31 (m, 12H). |
| 212 | 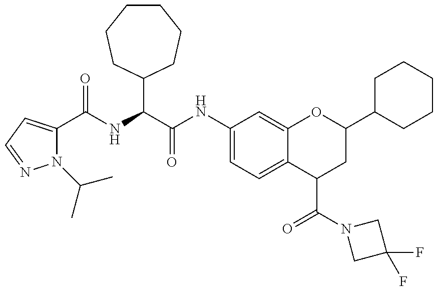 [SMILES: CC(C)n1nccc1C(=O)N[C@H](C(=O)Nc1ccc2c(c1)OC(C1CCCCC1)CC2C(=O)N1CC(F)(F)C1)C1CCCCCC1] | 640.4 | ¹H NMR (400 MHz, Chloroform-d) δ 8.23-27 (m, 1H), 7.24 − 7.15 (m, 1H), 7.07 − 6.81 (m, 3H), 6.58-6.55 (m, 1H), 5.45 − 5.35 (m, 1H), 4.22-4.12(m, 1H), 3.95- 3.84 (m, 3H), 2.77 − 2.46 (m, 6H), 2.34 (s, 1H), 2.23 − 2.03 (m, 2H), 2.01 − 1.76 (m, 3H), 1.75 − 1.64 (m, 6H), 1.64 − 1.54 (m, 5H), 1.54 − 1.31 (m, 14H). |
| 213 | 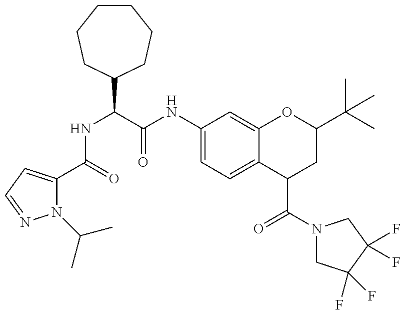 [SMILES: CC(C)n1nccc1C(=O)N[C@H](C(=O)Nc1ccc2c(c1)OC(C(C)(C)C)CC2C(=O)N1CC(F)(F)C(F)(F)C1)C1CCCCCC1] | 664.3 | ¹H NMR (400 MHz, Chloroform-d) δ 8.25(d, J = 24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 4.13-4.06(m, 1H), 3.74(t, J = 8.0 Hz, 1H), 2.18-2.10(m, 3H), 1.85-1.81(m, 2H), 1.72-1.28(m, 11H), 1.10(s, 9H) |
| 214 | 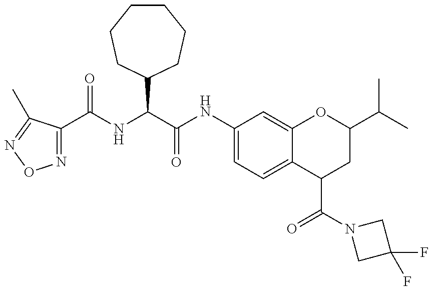 [SMILES: Cc1nonc1C(=O)N[C@H](C(=O)Nc1ccc2c(c1)OC(C(C)C)CC2C(=O)N1CC(F)(F)C1)C1CCCCCC1] | 574.4 | ¹H NMR (400 MHz, Chloroform-d) δ 7.99(dd, J = 16.0 Hz, 4.0 Hz, 1H), 7.51- 7.49(m, 1H), 7.18-7.13(m, 1H), 6.99- 6.87(m, 2H), 6.77-6.72(m, 1H), 6.56- 6.55(m, 1H), 5.45-5.37(m, 1H), 4.52- 4.48(m, 1H), 4.45-4.23(m, 4H), 4.08- 3.97(m, 1H), 3.71-3.65(m, 1H),2.59(s, 3H), 2.19-2.06(m, 2H), 1.94-1.79(m, 7H), 1.68~1.25(m, 7H), 1.03-0.97(m, 6H) |
| 215 | 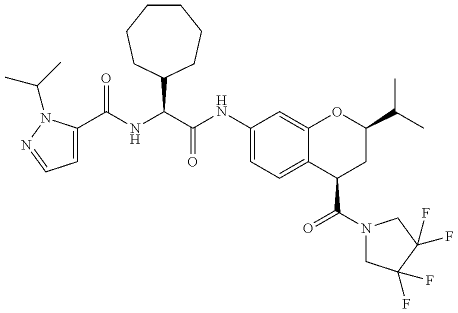 [SMILES: CC(C)[C@H]1C[C@@H](C(=O)N2CC(F)(F)C(F)(F)C2)c2ccc(NC(=O)[C@@H](NC(=O)c3ccnn3C(C)C)C3CCCCCC3)cc2O1] | 650.3 | ¹H NMR (400 MHz, Chloroform-d) δ 8.20-8.14(d, J = 12.0 Hz, 1H), 7.49- 7.47(m, 1H), 7.20-7.18(m, 1H), 6.99- 6.98(m, 1H), 6.89-6.80(m, 2H), 6.56(s, 1H), 5.55-5.37(m, 1H), 4.55-4.50(m, 1H), 4.13-3.97(m, 6H), 2.17-2.07(m, 1H), 1.99-1.81(m, 7H), 1.73-1.25(m, 14H), 1.02-0.98(m, 6H) |
| 216 | 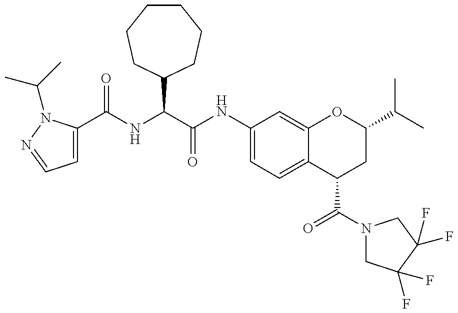 [SMILES: CC(C)[C@@H]1C[C@H](C(=O)N2CC(F)(F)C(F)(F)C2)c2ccc(NC(=O)[C@@H](NC(=O)c3ccnn3C(C)C)C3CCCCCC3)cc2O1] | 650.3 | ¹H NMR (400 MHz, Chloroform-d) δ 8.20-8.14(d, J = 12.0 Hz, 1H), 7.49- 7.47(m, 1H), 7.20-7.18(m, 1H), 6.99- 6.98(m, 1H), 6.89-6.80(m, 2H), 6.56(s, 1H), 5.55-5.37(m, 1H), 4.55-4.50(m, 1H), 4.13-3.97(m, 6H), 2.17-2.07(m, 1H), 1.99-1.81(m, 7H), 1.73-1.25(m, 14H), 1.02-0.98(m, 6H) |
| 217 | 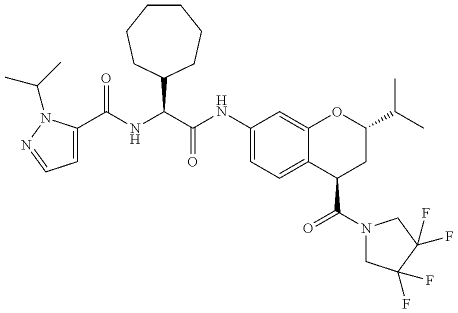 [SMILES: CC(C)[C@@H]1C[C@@H](C(=O)N2CC(F)(F)C(F)(F)C2)c2ccc(NC(=O)[C@@H](NC(=O)c3ccnn3C(C)C)C3CCCCCC3)cc2O1] | 650.3 | ¹H NMR (400 MHz, Chloroform-d) δ 8.20-8.14(d, J = 12.0 Hz, 1H), 7.49- 7.47(m, 1H), 7.20-7.18(m, 1H), 6.99- 6.98(m, 1H), 6.89-6.80(m, 2H), 6.56(s, 1H), 5.55-5.37(m, 1H), 4.55-4.50(m, 1H), 4.13-3.97(m, 6H), 2.17-2.07(m, 1H), 1.99-1.81(m, 7H), 1.73-1.25(m, 14H), 1.02-0.98(m, 6H) |
| 218 | 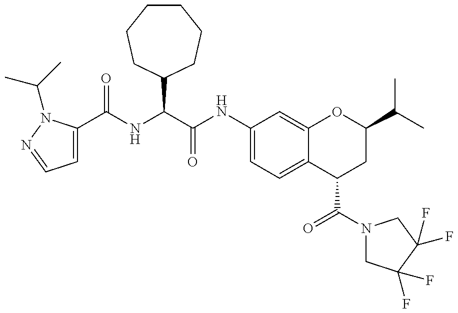 [SMILES: CC(C)[C@H]1C[C@H](C(=O)N2CC(F)(F)C(F)(F)C2)c2ccc(NC(=O)[C@@H](NC(=O)c3ccnn3C(C)C)C3CCCCCC3)cc2O1] | 650.3 | ¹H NMR (400 MHz, Chloroform-d) δ 8.20-8.14(d, J = 12.0 Hz, 1H), 7.49- 7.47(m, 1H), 7.20-7.18(m, 1H), 6.99- 6.98(m, 1H), 6.89-6.80(m, 2H), 6.56(s, 1H), 5.55-5.37(m, 1H), 4.55-4.50(m, 1H), 4.13-3.97(m, 6H), 2.17-2.07(m, 1H), 1.99-1.81(m, 7H), 1.73-1.25(m, 14H), 1.02-0.98(m, 6H) |
| 219 | 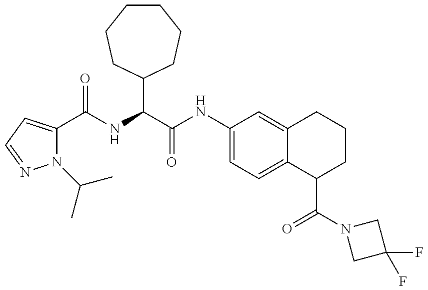 [SMILES: CC(C)n1nccc1C(=O)N[C@H](C(=O)Nc1ccc2c(c1)CCCC2C(=O)N1CC(F)(F)C1)C1CCCCCC1] | 556.3 | ¹H NMR (400 MHz, Chloroform-d) δ 8.25(d, J-24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 3.74(t, J = 8.0 Hz, 1H), 2.82-2.76(m, 2H), 2.18-2.10(m, 5H), 1.85-1.81(m, 2H), 1.72-1.28(m, 11H) |
| 220 | 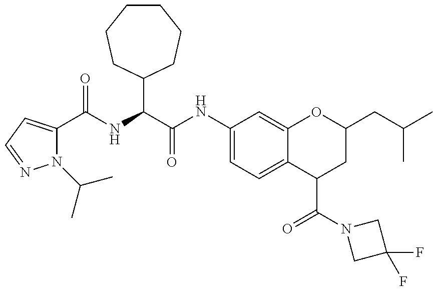 [SMILES: CC(C)CC1CC(C(=O)N2CC(F)(F)C2)c2ccc(NC(=O)[C@@H](NC(=O)c3ccnn3C(C)C)C3CCCCCC3)cc2O1] | 614.3 | ¹H NMR (400 MHz, Chloroform-d) δ 7.99(dd, J = 16.0 Hz, 4.0 Hz, 1H), 7.51- 7.49(m, 1H), 7.18-7.13(m, 1H), 6.99- 6.87(m, 2H), 6.77-6.72(m, 1H), 6.56- 6.55(m, 1H), 5.45-5.37(m, 1H), 4.52- 4.48(m, 1H), 4.45-4.23(m, 4H), 4.08- 3.97(m, 1H), 3.71-3.65(m, 1H), 2.19- 2.06(m, 2H), 1.94-1.79(m, 7H), 1.68- 1.25(m, 15H), 1.03-0.97(m, 6H) |
| 221 | 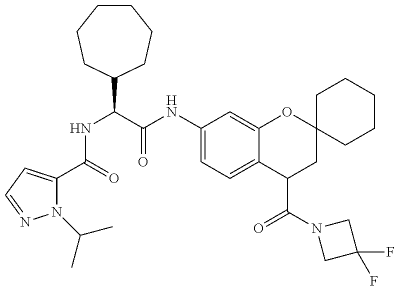 [SMILES: CC(C)n1nccc1C(=O)N[C@H](C(=O)Nc1ccc2c(c1)OC1(CCCCC1)CC2C(=O)N1CC(F)(F)C1)C1CCCCCC1] | 626.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 3.74(t, J = 8.0 Hz, 1H), 2.18-2.10(m, 3H), 1.85-1.81(m, 6H), 1.72-1.28(m, 17H) |
| 222 | 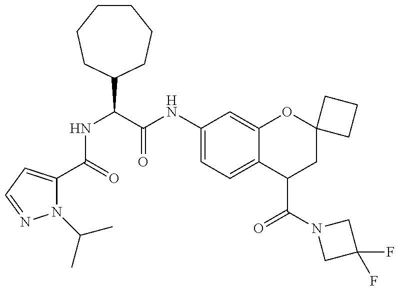 [SMILES: CC(C)n1nccc1C(=O)N[C@H](C(=O)Nc1ccc2c(c1)OC1(CCC1)CC2C(=O)N1CC(F)(F)C1)C1CCCCCC1] | 598.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H), 3.74(t, J = 8.0 Hz, 1H), 2.18-2.10(m, 3H), 1.90-1.81(m, 6H), 1.72-1.28(m, 13H) |
| 223 | 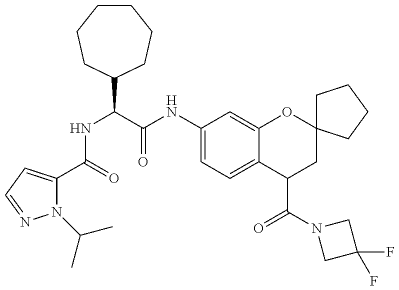 [SMILES: CC(C)n1nccc1C(=O)N[C@H](C(=O)Nc1ccc2c(c1)OC1(CCCC1)CC2C(=O)N1CC(F)(F)C1)C1CCCCCC1] | 612.3 | ¹H NMR (400 MHz, Chloroform-d) δ 8.25(d, J = 24 Hz, 1H), 7.47(d, J = 1.2 Hz,1H), 7.10(d, J = 4.8 Hz, 1H), 7.02-6.99(m, 1H), 6.97-6.93(m, 1H), 6.88-6.82(m, 2H), 6.56(d, .J = 4.8 Hz, 1H), 5.42-5.38(m, 1H), 4.54-4.30(m, 5H), 3.74(t, J = 8.0 Hz, 1H), 2.18-2.10(m, 3H), 1.90-1.81(m, 8H), 1.72-1.28(m, 11H) |
| 224 | 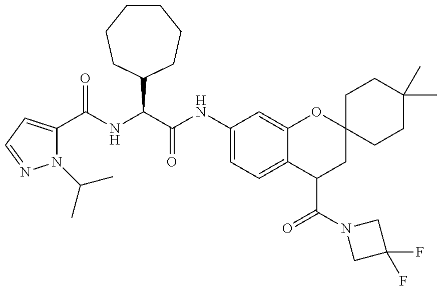 [SMILES: CC(C)n1nccc1C(=O)N[C@H](C(=O)Nc1ccc2c(c1)OC1(CCC(C)(C)CC1)CC2C(=O)N1CC(F)(F)C1)C1CCCCCC1] | 654.4 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d,,J = 4.8 Hz, 1H), 5.42-5.38(m, 1H), 4.54-4.30(m, 5H), 3.74(t, J = 8.0 Hz, 1H), 2.18-2.10(m, 3H), 1.85-1.81(m, 6H), 1.72-1.28(m, 15H), 0.85(s, 6H) |
| 225 | 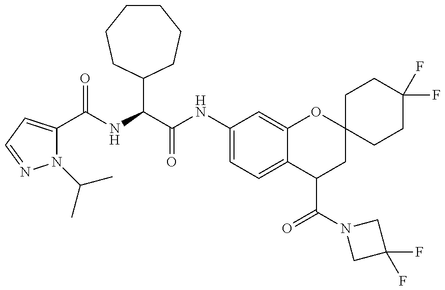 [SMILES: CC(C)n1nccc1C(=O)N[C@H](C(=O)Nc1ccc2c(c1)OC1(CCC(F)(F)CC1)CC2C(=O)N1CC(F)(F)C1)C1CCCCCC1] | 662.3 | ¹H NMR (400 MHz, Chloroform-d) δ 8.25(d, J = 24 Hz, 1H), 7.47(d, J = 1.2 Hz, 1H), 7.10(d, J = 4.8 Hz, 1H), 7.02-6.99(m, 1H), 6.97-6.93(m, 1H), 6.88-6.82(m, 2H), 6.56(d, J = 4.8 Hz, 1H), 5.42-5.38(m, 1H), 4.54-4.30(m, 5H). 3.74(t, J = 8.0 Hz, 1H), 2.18-2.03(m, 7H), 1.85-1.81(m, 6H), 1.72-1.28(m, 11H) |
| 226 | 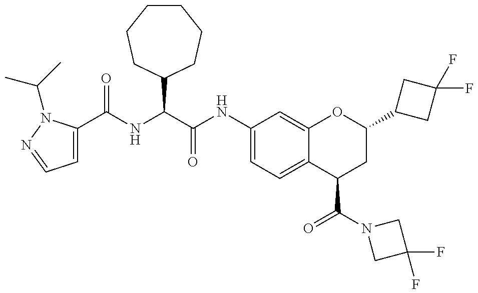 [SMILES: CC(C)n1nccc1C(=O)N[C@H](C(=O)Nc1ccc2c(c1)O[C@H](C1CC(F)(F)C1)C[C@H]2C(=O)N1CC(F)(F)C1)C1CCCCCC1] | 648.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.45-5.38(m, 1H), 4.56-4.52(m, 1H), 4.48-4.32(m, 3H), 4.21-4.07(m, 1H), 3.93-3.82(m, 1H), 2.68-2.54(m, 4H), 1.92-1.78(m, 4H), 1.73-1.36(m, 19H) |
| 227 | 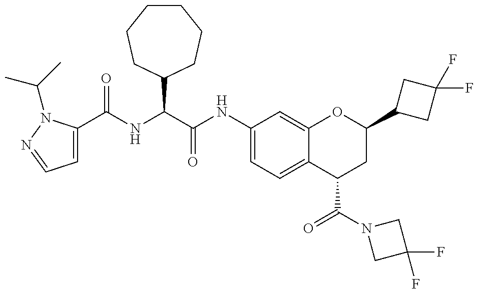 [SMILES: CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1] | 648.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.45-5.38(m, 1H), 4.56-4.52(m, 1H), 4.48-4.32(m, 3H), 4.21-4.07(m, 1H), 3.93-3.82(m, 1H), 2.68-2.54(m, 4H), 1.92-1.78(m, 4H), 1.73-1.36(m, 19H) |
| 228 | 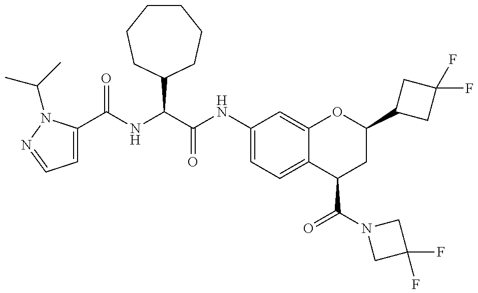 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCCCCC1] | 648.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.45-5.38(m, 1H), 4.56-4.52(m, 1H), 4.48-4.32(m, 3H), 4.21-4.07(m, 1H), 3.93-3.82(m, 1H), 2.68-2.54(m, 4H), 1.92-1.78(m, 4H), 1.73-1.36(m, 19H) |
| 229 | 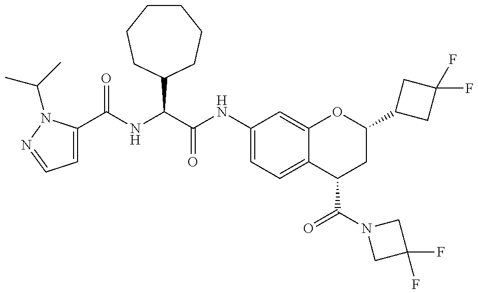 [SMILES: CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1] | 648.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.45-5.38(m, 1H), 4.56-4.52(m, 1H), 4.48-4.32(m, 3H), 4.21-4.07(m, 1H), 3.93-3.82(m, 1H), 2.68-2.54(m, 4H), 1.92-1.78(m, 4H), 1.73-1.36(m, 19H) |
| 230 | 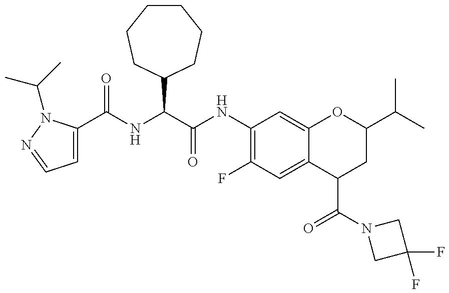 [SMILES: CC(C)C1CC(C(=O)N2CC(F)(F)C2)c2cc(F)c(NC(=O)[C@@H](NC(=O)c3ccnn3C(C)C)C3CCCCCC3)cc2O1] | 618.3 | ¹H NMR (400 MHz, Chloroform-d) δ 7.99(dd, J = 16.0 Hz, 4.0 Hz, 1H), 7.38- 7.33(m, 1H), 6.99-6.87(m, 2H), 6.77- 6.72(m, 1H), 6.56-6.55(m, 1H), 5.45- 5.37(m, 1H), 4.52-4.48(m, 1H), 4.45- 4.23(m, 4H), 4.08-3.97(m, 1H), 3.71- 3.65(m, 1H), 2.19-2.06(m, 2H), 1.94- 1.79(m, 7H), 1.68-1.25(m, 13H), 1.03- 0.97(m, 6H) |
| 231 | 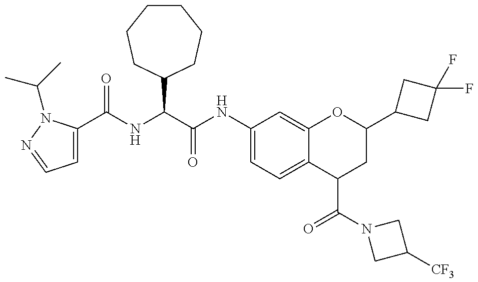 [SMILES: CC(C)n1nccc1C(=O)N[C@H](C(=O)Nc1ccc2c(c1)OC(C1CC(F)(F)C1)CC2C(=O)N1CC(C(F)(F)F)C1)C1CCCCCC1] | 680.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.45-5.38(m, 1H), 4.56-4.52(m, 1H), 4.48-4.32(m, 3H), 4.21-4.07(m, 1H), 3.93-3.82(m, 1H), 2.85-2.76(m, 1H), 2.68-2.54(m, 4H), 1.92-1.78(m, 4H), 1.73-1.36(m, 19H) |
| 232 | 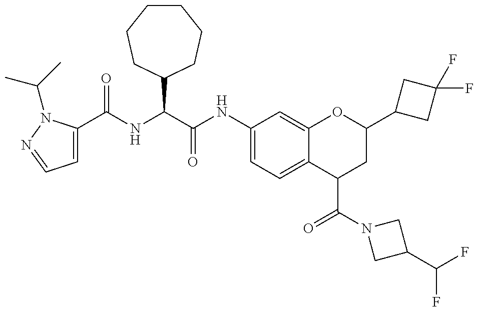 [SMILES: CC(C)n1nccc1C(=O)N[C@H](C(=O)Nc1ccc2c(c1)OC(C1CC(F)(F)C1)CC2C(=O)N1CC(C(F)F)C1)C1CCCCCC1] | 662.3 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.92-5.86(m, 1H), 5.45-5.38(m, 1H), 4.56-4.52(m, 1H), 4.48-4.32(m, 3H), 4.21-4.07(m, 1H), 3.93-3.82(m, 1H), 2.85-2.76(m, 1H), 2.68-2.54(m, 4H), 1.92-1.78(m, 4H), 1.73-1.36(m, 19H) |
| 233 | 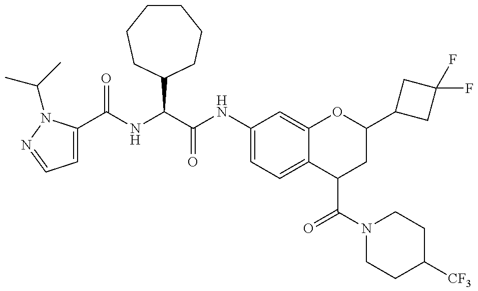 [SMILES: CC(C)n1nccc1C(=O)N[C@H](C(=O)Nc1ccc2c(c1)OC(C1CC(F)(F)C1)CC2C(=O)N1CCC(C(F)(F)F)CC1)C1CCCCCC1] | 708.4 | ¹H NMR (400 MHz, Chloroform-d) δ 8.27(d, J = 12 Hz, 1H), 7.52(s, 1H), 7.23(d, J = 4.0 Hz, 1H), 6.95-6.80(m, 3H), 6.58-5.56(m, 1H), 5.92-5.86(m, 1H), 5.45-5.38(m, 1H), 4.56-4.52(m, 1H), 4.48-4.32(m, 3H), 4.21-4.07(m, 1H). 3.93-3.82(m, 1H), 2.85-2.76(m, 1H), 2.68-2.54(m, 4H), 2.23-2.18(m, 1H), 1.92-1.78(m, 8H), 1.73-1.36(m, 19H) |
| 234 | 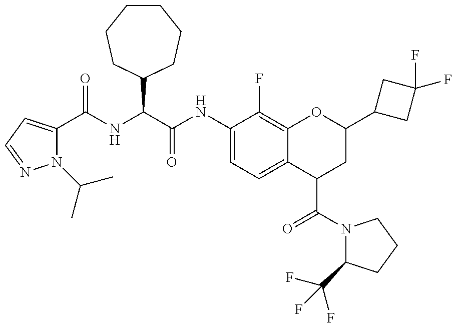 [SMILES: CC(C)n1nccc1C(=O)N[C@H](C(=O)Nc1ccc2c(c1F)OC(C1CC(F)(F)C1)CC2C(=O)N1CCC[C@H]1C(F)(F)F)C1CCCCCC1] | 712.3 | ¹H NMR (400 MHz, Chloroform-d) δ 8.54 (s, 1H), 7.47 − 7.44 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 4H), 4.13 − 4.09 (m, 1H), 3.89 − 3.85 (m, 2H), 3.66-3.51(m, 2H), 2.77 − 2.46 (m, 4H), 2.34 (s, 1H), 2.23 − 2.03 (m, 2H), 2.01 − 1.76 (m, 5H), 1.75 − 1.64 (m, 2H), 1.64 − 1.54 (m, 1H), 1.54 − 1.31 (m, 9H). |
| 235 | 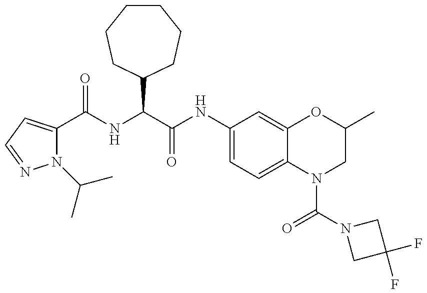 [SMILES: CC1CN(C(=O)N2CC(F)(F)C2)c2ccc(NC(=O)[C@@H](NC(=O)c3ccnn3C(C)C)C3CCCCCC3)cc2O1] | 573.3 | ¹H NMR (400 MHz, Chloroform-d) δ 8.34(s, 1H), 7.47(s. 1H), 7.24-7.18(m, 2H), 6.99-6.88(m, 2H), 6.57-6.56(m, 1H), 5.43-5.36(m, 1H), 4.55-4.52(m, 1H), 4.32-4.22(m, 3H), 4.12-4.01(m, 4H), 3.09-3.03(m, 1H), 1.92-1.82(m, 4H), 1.72-1.67(m, 2H), 1.59-1.30(m, 14H) |
| 236 | 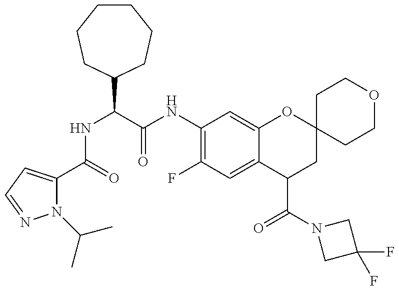 [SMILES: CC(C)n1nccc1C(=O)N[C@H](C(=O)Nc1cc2c(cc1F)C(C(=O)N1CC(F)(F)C1)CC1(CCOCC1)O2)C1CCCCCC1] | 646.3 | ¹H NMR (400 MHz, Chloroform-d) δ 8.25(d, J = 24 Hz, 1H), 7.15(s, 1H), 6.99(s, 1H), 6.97-6.93(m, 1H), 6.88- 6.82(m, 2H), 6.56(d, J = 4.8 Hz, 1H), 5.42-5.38(m, 1H), 4.54- 4.30(m, 5H), 3.74(t, J = 8.0 Hz, 1H), 3.70-3.64(m, 4H), 2.18-2.03(m, 3H), 1.85-1.81(m, 6H), 1.72-1.28(m, 11H) |
| 237 | 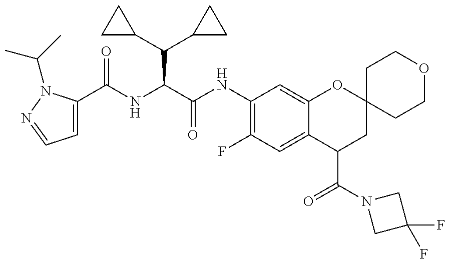 [SMILES: CC(C)n1nccc1C(=O)N[C@H](C(=O)Nc1cc2c(cc1F)C(C(=O)N1CC(F)(F)C1)CC1(CCOCC1)O2)C(C1CC1)C1CC1] | 644.3 | ¹H NMR (400 MHz, Chloroform-d) δ 8.17-8.16(m, 1H), 7.93-7.89(m, 1H), 7.50-7.49(m, 1H),7.09-7.07(m, 1H),6.77-6.73(m, 1H), 6.56-6.55(m, 1H), 5.49-5.41(m, 1H), 4.84-4.81(m, 1H), 4.45-4.36(m, 3H), 4.15-4.06(m, 1H), 3.94-3.88(m, 1H), 3.82-3.78(m, 2.H), 3.70-3.63(m, 2H), 2.00-1.96(m, 2H), 1.88-1.66(m, 5H), 1.49-1.43(m, 6H), 0.89-0.77(m, 6H),0.67-0.50(4H) |
| 238 | 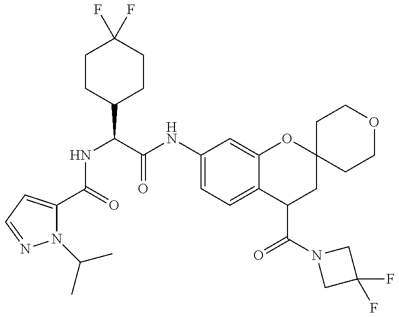 [SMILES: CC(C)n1nccc1C(=O)N[C@H](C(=O)Nc1ccc2c(c1)OC1(CCOCC1)CC2C(=O)N1CC(F)(F)C1)C1CCC(F)(F)CC1] | 650.3 | ¹H NMR (400 MHz, Chloroform-d) δ 8.17-8.16(m, 1H), 7.93-7.89(m, 1H), 7.50-7.49(m, 1H), 7.23(d, J = 4.0 Hz, 1H), 7.05-7.07(m, 1H),6.77-6.73(m, 1H), 6.56-6.55(m, 1H), 5.49-5.41(m, 1H), 4.84-4.81(m, 1H), 4.45-4.36(m, 3H), 4.15-4.06(m, 1H), 3.94-3.88(m, 1H), 3.82-3.78(m, 2H), 3.70-3.63(m, 2H), 2.00-1.90(m, 6H), 1.88-1.66(m, 5H), 1.49-1.43(m, 10H) |
| 239 | 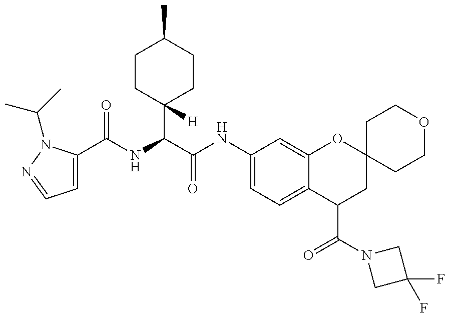 [SMILES: CC(C)n1nccc1C(=O)N[C@H](C(=O)Nc1ccc2c(c1)OC1(CCOCC1)CC2C(=O)N1CC(F)(F)C1)[C@H]1CC[C@H](C)CC1] | 628.3 | ¹H NMR (400 MHz, Chloroform-d) δ 8.17-8.16(m, 1H), 7.93-7.89(m, 1H), 7.50-7.49(m, 1H), 7.09-7.07(m, 1H),6.77-6.73(m, 1H), 6.56-6.55(m, 1H), 5.49-5.41(m, 1H), 4.84-4.81(m, 1H), 4.45-4.36(m, 3H), 4.15-4.06(m, 1H), 3.94-3.88(m, 1H), 3.82-3.78(m, 2H), 3.70-3.63(m, 2H), 2.00-1.96(m, 2H), 1.88-1.66(m, 5H), 1.55-1.24(m, 14H), 0.89-0.87(m, 3H), |
| 240 | 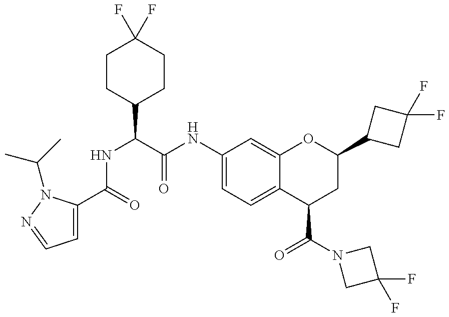 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCC(F)(F)CC1] | 670.3 | ¹H NMR (400 MHz, Chloroform-d) 9.13(d, J = 8.0 Hz, 1H), 7.42(s, 1H), 7.31- 7.24(m, 2H), 6.79-7.76(m, 1H),6.69- 6.66(m, 1H), 6.61(d, J = 1.2 Hz, 1H), 5.36-5.30(m, 1H), 4.69-4.65(m, 1H), 4.45-4.36(m, 4H), 4.17-4.09(m, 1H), 3.87-3.77(m, 2H), 2.63-2.47(m, 5H), 2.33-2.26(m, 3H), 2.09-1.93(m, 4H), 1.82-1.35(m, 9H) |
| 241 | 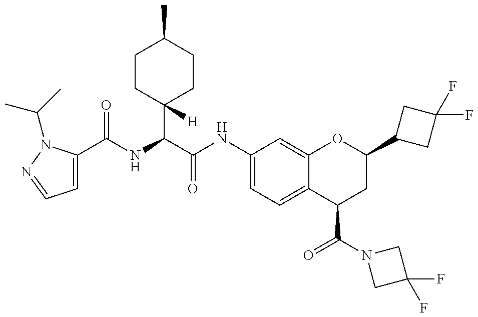 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)[C@H]1CC[C@H](C)CC1] | 648.3 | ¹H NMR (400 MHz, Chloroform-d) 9.15-9.10(m, 1H), 7.41-7.35(m, 2H), 7.25-7.20(m, 1H), 6.83-6.79(m, 2H), 6.64-6.63(m, 1H), 5.40-5.33(m, 1H), 4.58-4.54(m, 1H), 4.41-4.33(m, 4H), 4.12-4.04(m, 1H), 3.84-3.79(m, 2H), 2.61-2.46(m, 5H), 2.33-2.28(m, 3H), 2.06-2.01(m, 1H), 1.90-1.67(m, 4H), 1.44-1.33(m, 4H), 1.27-1.17(m, 4H), 0.83(d, J = 8.0 Hz, 3H) |
| 242 | 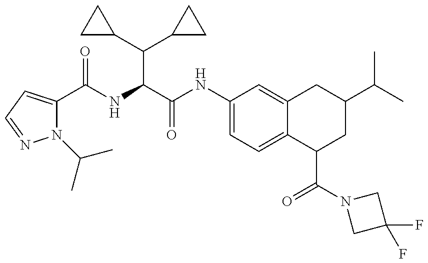 [SMILES: CC(C)C1Cc2cc(NC(=O)[C@@H](NC(=O)c3ccnn3C(C)C)C(C3CC3)C3CC3)ccc2C(C(=O)N2CC(F)(F)C2)C1] | 596.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 2H), 4.13 − 4.09 (m, 1H), 3.89 − 3.85 (m, 2H), 2.77 − 2.46 (m, 6H), 2.34 (s, 1H), 2.23 − 2.03 (m, 3H), 2.01 − 1.76 (m, 5H), 1.75 − 1.64 (m, 2H), 1.64 − 1.54 (m, 1H), 1.54 − 1.31 (m, 7H). |
| 243 | 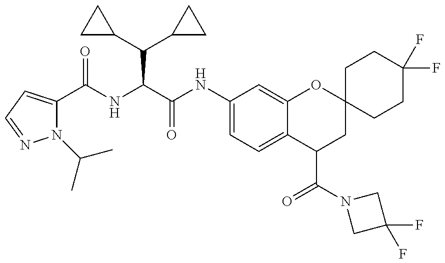 [SMILES: CC(C)n1nccc1C(=O)N[C@H](C(=O)Nc1ccc2c(c1)OC1(CCC(F)(F)CC1)CC2C(=O)N1CC(F)(F)C1)C(C1CC1)C1CC1] | 660.3 | ¹H NMR (400 MHz. Chloroform-d) δ 8.52(s, 1H), 7.48-7.46(m, 1H), 7.20- 7.17(m, 2H), 6.95-6.87(m, 2H), 6.55(dd, J = 8.0 Hz, 2.4 Hz, 1H), 5.44-5.40(m, 1H), 4.87-4.83(m, 1H), 4.47-4.34(m, 3H), 4.15-3.97(m, 1H), 3.80-3.75(m, 1H), 2.30-2.20(m, 2H), 2.05-1.77(m, 3H), 1.57-1.39(m, 8H), 0.56-0.45(m, 5H), 0.38-0.20(m, SH). |
| 244 | 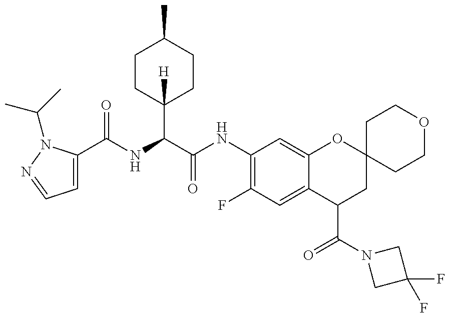 | 646.3 | ¹H NMR (400 MHz, Chloroform-d) 87.97-7.94(m, 1H), 7.91-7.89(m, 1H), 7.09-7.04(m, 1H),6.78-6.74(m, 1H), 6.68-6.65(m, 1H), 6.57-6.56(m, 1H), 5.49-5.41(m, 1H), 4.84-4.81(m, 1H), 4.45-4.36(m, 3H), 4.15-4.06(m, 1H), 3.94-3.88(m, 1H), 3.82-3.78(m, 2H), 3.70-3.63(m, 2H), 2.00-1.96(m, 2H), 1.88-1.66(m, 5H), 1.55-1.24(m, 14H), 0.89-0.87(m, 3H), |
| 245 | 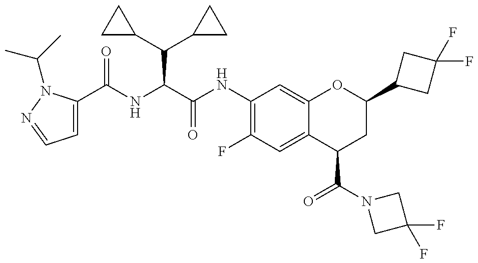 [SMILES: CC(C)n1nccc1C(=O)N[C@H](C(=O)Nc1cc2c(cc1F)[C@H](C(=O)N1CC(F)(F)C1)C[C@H](C1CC(F)(F)C1)O2)C(C1CC1)C1CC1] | 664.3 | ¹H NMR (400 MHz, Chloroform-d) δ 8.13(d, J = 2.4 Hz, 1H), 7.88(d, J = 3.2 Hz, 1H), 7.52(d, J = 2.4 Hz, 1H), 7.05(d, J = 8.0 Hz, 1H), 6.73(d, J = 12 Hz, 1H), 6.56(d, J = 2.0 Hz, 1H), 5.49-5.43(m, 1H), 4.85-4.82(m, 1H), 4.46-4.38(m, 3H), 4.25-4.18(m, 1H), 3.94-3.90(m, 1H), 3.87-3.82(m, 1H), 2.69-2.52(m, 4H), 2.42-2.32(m, 1H), 2.11-2.07(m, 1H), 1.51-1.46(m, 6H), 1.27-1.24(m, 1H), 0.92-0.77(m, 3H), 0.68-0.51(m, 4H), 0.42-0.33(m, 2H), 0.26-0.21(m, 2H) |
| 246 | 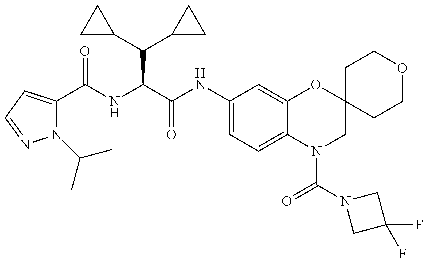 [SMILES: CC(C)n1nccc1C(=O)N[C@H](C(=O)Nc1ccc2c(c1)OC1(CCOCC1)CN2C(=O)N1CC(F)(F)C1)C(C1CC1)C1CC1] | 627.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.84-4.81(m, 1H), 4.45-4.36(m, 3H), 4.15-4.06(m, 2H), 3.82-3.78(m, 2H), 3.70-3.63(m, 3H), 1.88-1.66(m, 5H), 1.49-1.43(m, 6H), 0.89-0.77(m, 6H),0.67-0.50(4H) |
| 247 | 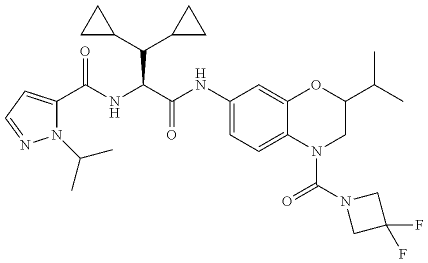 [SMILES: CC(C)C1CN(C(=O)N2CC(F)(F)C2)c2ccc(NC(=O)[C@@H](NC(=O)c3ccnn3C(C)C)C(C3CC3)C3CC3)cc2O1] | 599.3 | ¹H NMR (400 MHz, Chloroform-d) δ 8.56 (s, 1H), 7.49 − 7.45 (m, 1H), 7.25 − 7.23 (m, 1H), 6.99 − 6.95 (m, 1H), 6.90 − 6.79 (m, 2H), 6.61 − 6.57 (m, 1H), 5.42 − 5.38 (m, 1H), 4.59 − 4.55 (m, 1H), 4.43 − 4.35 (m, 3H), 3.89 − 3.85 (m, 3H), 2.77 − 2.46 (m, 2H), 2.34 (s, 1H), 2.23 − 2.03 (m, 3H), 2.01 − 1.76 (m, 5H), 1.75 − 1.64 (m, 2H), 1.64 − 1.54 (m, 1H), 1.54 − 1.31 (m, 7H). |
| 248 | 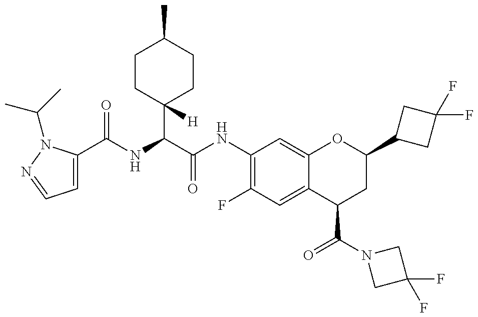 [SMILES: CC(C)n1nccc1C(=O)N[C@H](C(=O)Nc1cc2c(cc1F)[C@H](C(=O)N1CC(F)(F)C1)C[C@H](C1CC(F)(F)C1)O2)[C@H]1CC[C@H](C)CC1] | 666.3 | ¹H NMR (400 MHz, Chloroform-d) δ 8.41-8.38(m, 1H), 7.82-7.76(m, 1H), 7.48-7.44(m, 1H), 6.97-6.80(m, 1H), 6.71-6.65(m, 1H), 6.57(m, 1H), 5.45- 5.37(m, 1H), 4.60-4.52(m, 1H), 4.43- 4.32(m, 4H), 4.21-4.13(m, 1H), 3.90- 3.70(m, 2H), 2.63-2.47(m, 4H), 2.36- 2.29(m, 1H), 2.08-2.02(m, 2H), 1.90- 1.68(m, 4H), 1.46-1.40(m, 4H), 1.30- 1.08(m, 6H), 0.85-0.84(m, 5H) |
| 249 | 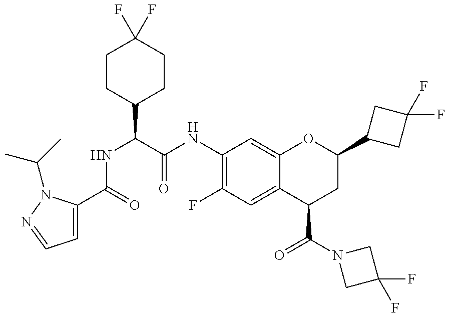 [SMILES: CC(C)n1nccc1C(=O)N[C@H](C(=O)Nc1cc2c(cc1F)[C@H](C(=O)N1CC(F)(F)C1)C[C@H](C1CC(F)(F)C1)O2)C1CCC(F)(F)CC1] | 688.3 | ¹H NMR (400 MHz, Chloroform-d) δ 8.58-8.55(m, 1H), 7.77-7.69(m, 1H), 7.48(s, 1H), 7.25(m, 1H), 6.97-6.89(m, 1H), 6.70-6.67(m, 1H), 6.58(s, 1H), 5.44-5.37(m, 1H), 4.71-4.69(m, 1H), 4.50-4.22(m, 5H), 3.95-3.80(m, 2H), 2.64-2.50(m, 5H), 2.13-1.84(m, 4H), 1.57-1.41(m, 10H), 1.19-1.17(m, 2H) |

### Example 2. Evaluation of the In Vitro Bioactivity

| Materials and Reagents | Vendor | Cat# |
|---|---|---|
| HEK-Blue IL-17 | InvivoGen | hkb-il17 |
| Recombinant human | R&D | 7955-IL |
| IL-17A protein | | |
| Reconstitution | R&D | RB02 |
| Buffer 2 (BSA/PBS) | | |
| DMEM, high glucose | Gibco | 11965-092 |
| DMEM, High Glucose, | Gibco | 21063-029 |
| HEPES, no Phenol Red | | |
| FBS | AusGeneX | FBS500-S |
| Penicillin- | Gibco | 15140-122 |
| Streptomycin(10000 U/ml, 100 ml) | | |
| Normocin | InvivoGen | ant-nr-1 |
| HEK-Blue Selection | InvivoGen | hb-sel |
| DMSO | Sigma | D8418 |
| PBS, pH 7.4 | Gibco | 10010-031 |
| SEAPorter | Novus | NBP2-25285 |
| IL-17A Inhibitor-1 | MCE | HY-139206 |

| Consumables and Instrument | Vendor | Cat# or Model |
|---|---|---|
| CulturPlate-384, White | PerkinElmer | 6007680-50 |
| Opaque 384-well Microplate | | |
| Tissue Culture (TC)-treated surface, | Corning | CLS3764 |
| black polystyrene, flat bottom | | |
| Echo Qualified | LabCtye | PP-0200 |
| 384-Well Polypropylene | | |
| Microplate, Clear, Sterile | | |
| Reagent reservoirs, sterilized | JetBioFil | LTT-001-50 |
| Biological Safety Cabinet(Class II) | Thermo Scientific | 1389 |
| Forma CO₂ Incubator | Thermo Scientific | 371 |
| Countess 3 automated cell counter | Invitrogen | A49891 |
| Plate shaker | Thermo | 4625- |
| | | 1CECN/THZ Q |
| Centrifuge | Eppendorf | 5810R |
| Echo 655 | Labcyte | 655 |
| Envision 2105 multilabel Reader | PerkinElmer | Envision 2105 |
| Votexer | IKA | MS3 digital |

### Compound Management

1. The test Compounds were 3-fold serial diluted from 10 mM for 10 doses in DMSO.
2. Prepare Reference Compounds (IL-17A-Inhibitor-1) were 3-fold serial diluted from 1 mM for 10 doses in DMSO.
3. Prepare 1000× Positive control (1 mM IL-17A-Inhibitor-1) and 1000× vehicle control (100% DMSO).

### Assay Procedure

a) Cells were cultured as the HEK-Blue™ IL-17 technical data sheet recommended. HEK-Blue IL-17 cells were assayed in the exponential growth phase.
b) Aspirate growth medium and rinse cells twice with PBS to remove phenol red.
c) Resuspend cells in test medium to a proper concentration.
d) Only cells with viability greater than 90% were used for assays.
e) Transfer 25 nl of compound dilutions into 384-well assay plates using Echo655;
f) Seed 25 ul of HEK-Blue IL-17 cells at 8,000 cells/well into 384-well plate with rhIL-17A/A.
g) Cells were incubated for 20 hours at 37° C. under 5% CO₂ atmosphere.
h) Transfer 2 ul of cells supernatant into per well of 384-well assay plates.
i) Add 20 ul of PNPP substrate.
j) Measure the Absorbance at 405 nm on Envision 2105 plate reader.

### Data Analysis

RLU signal (LUM_{cmpd}) is calculated for each well.

% Inhibition is calculated as follow:

$$\% inhibition = 100 - ( {Signal}_{cmpd} - {Signal}_{Ave \_ PC} ) / ( {Signal}_{Ave \_ VC} - {Signal}_{Ave \_ PC} ) \times 100.$$

Signalave_pc: The average signal for the positive controls across the plate.

Signalave_vc: The average signal for negative controls across the plate.

Calculate IC50 and Plot effect-dose curve of cmpds:

Calculate IC50 by fitting % Inhibition values and log of compound concentrations to nonlinear regression (dose response−variable slope) with Graphpad 8.0.

The results are summarized in Table 2

TABLE 2
The relative biological activity of
compounds in HEK-Bule cell Assay

| Example | IC50, nM |
|---|---|
| 1 | 208.6 |
| 5 | 332.7 |
| 12 | 316.8 |
| 13 | 239.2 |
| 36 | 153.8 |
| 108 | 102.3 |
| 135 | 18.9 |
| 136 | 48.95 |
| 152 | 7.489 |
| 160 | 42.06 |
| 161 | 43.26 |
| 162 | 35.66 |
| 164 | 72.59 |
| 165 | 127.3 |
| 166 | 54.5 |
| 167 | 30.43 |
| 169 | 18.19 |
| 189 | 225.7 |
| 190 | 367 |
| 191 | 170.9 |
| 192 | 267.4 |
| 193 | 39.7 |
| 194 | 564.3 |
| 195 | 130.7 |
| 197 | 286.9 |
| 198 | 215.8 |
| 200 | 199.6 |
| 201 | 443.2 |
| 203 | 140.8 |
| 204 | 46.62 |
| 206 | 369 |
| 207 | 92.89 |
| 208 | 289.6 |
| 210 | 250.7 |
| 211 | 259 |
| 212 | 213.4 |
| 213 | 112.5 |
| 214 | 334.7 |
| 215 | 40.7 |
| 217 | 199.1 |
| 218 | 221.6 |
| 219 | 275.5 |
| 220 | 181.1 |
| 221 | 116.2 |
| 222 | 255.1 |
| 223 | 130.1 |
| 224 | 202.1 |
| 225 | 44.88 |
| 226 | 48.9 |
| 228 | 27.5 |
| 230 | 242 |
| 231 | 115.8 |
| 232 | 77.1 |
| 235 | 71.9 |
| 236 | 14.9 |
| 237 | 26.08 |
| 239 | 31.06 |
| 240 | 48.33 |
| 241 | 15.87 |
| 243 | 121.9 |
| 244 | 13.48 |
| 245 | 22.02 |
| 246 | 18.19 |
| 247 | 30.43 |
| 248 | 24.38 |
| 249 | 99.35 |

### Example 3. Pharmacokinetics of the Compounds of the Application

Compounds of the present application were formulated in 40% PEG400/10% solutol/2% Tween 80/48% saline (v/v/v/v), and administered via oral gavage (PO) at the dosages of 10 mg per kilogram body weight in fasted SD rats. Plasma samples were collected at 0.25, 0.5, 1, 2, 4, 8, and 24 hours post dosing. Compound concentration was determined by LC-MS and pharmacokinetics parameters were calculated by WinNonlin 8.2 using Non-Compartmental Analysis model. PK parameters of representative compounds were listed in Table 3.

TABLE 3

| Compound | AUC, ng · h/m | F (%) |
|---|---|---|
| 161 | 9703 | 63.5 |
| 204 | 18980 | 34.8 |
| 225 | 7840 | 57.6 |
| 248 | 6093 | 70.7 |

What is claimed is:

1. A compound of Formula I: 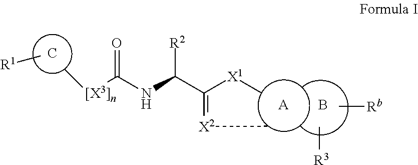
pharmaceutically acceptable salts thereof, deuterium substitutes thereof, and isomers thereof,
wherein Ring C is aryl, 5-6 membered heteroaryl, C₃₋₁₀ cycloalkyl, C₃₋₁₀ heterocyclyl, R^{x}—C₃₋₁₀ cycloalkyl, R^{x}—C₃₋₁₀ heterocyclyl, R^{x}-aryl and R^{x}-5-6 membered heteroaryl; wherein R^{x} is halo or —C₁₋₆ alkyl;
wherein Ring C is optionally substituted with one or more R¹;
wherein each R¹ is independently selected from the group consisting of OH, CN, C₁₋₆ alkyl, C₁₋₆ haloalkyl, C₂₋₆ alkenyl, C₂₋₆ alkynyl, —O—C₁₋₆ alkyl, —O—C₁₋₆ haloalkyl, —CO—C₁₋₆ alkyl, —CO—C₁₋₆ haloalkyl, —CO—NR^{3a}R^{3b}, C₃₋₁₀ cycloalkyl, C₃₋₁₀ heterocyclyl, C₆₋₁₀ aryl, and 5-10 membered heteroaryl;
wherein each R¹ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —S(O)—C₁₋₆ alkyl, —S(O)₂—C₁₋₆ alkyl and —S—C₁₋₆ alkyl;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X¹ is NH, O, or S;
wherein X² is N, O, or S; and wherein X² is optionally attached to an atom in Ring A and forms another ring next to Ring A;
wherein X³ is O or —CR^{c}R^{d}; and wherein each of R^{c} and R^{d} is independently selected from the group consisting of H, halo and —C₁₋₆ alkyl;
wherein Ring A is selected from the group consisting of 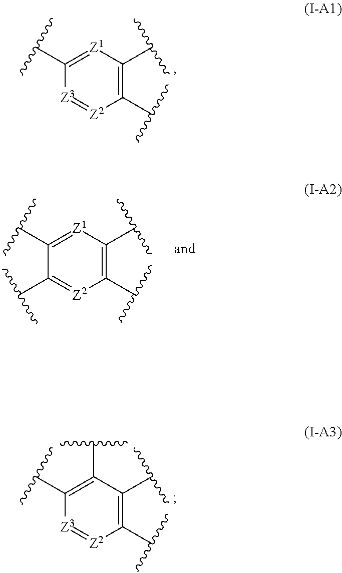
wherein each of Z¹, Z² and Z³ is independently selected from the group consisting of —N═, —N(═O)═ and —C(R^{a})═, wherein R^{a} is selected from the group consisting of H, F, Cl, Br, I, —C₁₋₆ alkyl, C₁₋₆ haloalkyl, C₂₋₆ alkenyl, C₂₋₆ alkynyl, OH, CN, —O—C₁₋₆ alkyl, —O—C₁₋₆ haloalkyl, C₃₋₁₀ cycloalkyl, C₃₋₁₀ heterocyclyl, aryl and 5-10 membered heteroaryl;
wherein Ring B, which is fused to Ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated or unsaturated bridged-ring;
wherein Ring B is optionally substituted with one or more R^{b}, each of R^{b} is independently selected from the group consisting of CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —NR^{3a}—CO—C₁₋₆ alkyl, —NR^{3a}—COOC₁₋₆ alkyl, —NR^{3a}—CO—C₁₋₆ haloalkyl, —NR^{3a}—CO—NR^{3a}R^{3b}, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more substituents each independently selected from the group consisting of halo, CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, C₂₋₆ alkenyl, C₂₋₆ alkynyl, C₃₋₁₀ cycloalkyl, heterocyclyl, C₆₋₁₀ aryl, and 5-10 membered heteroaryl;
wherein Ring B is substituted with R³, wherein R³ is selected from the group consisting of —CONR^{3a}R^{3b}, —S(O)—NR³R^{3b}, —S(O)₂—NR^{3a}R^{3b}, —C₀₋₆ alkyl-CONR^{3a}R^{3b}, —C₀₋₆ alkyl-NR^{3a}R^{3b}, —N(R^{3a})—COR^{3a}, —N(R^{3a})—CONR^{3a}R^{3b}, —C₀₋₆ alkyl-SONR^{3a}R^{3b}, —C₀₋₆ alkyl-SO₂NR^{3a}R^{3b}, —C(S)—NR^{3a}R^{3b}, CN, 5-10 membered heteroaryl, —COR^{3a}, —P(O)(OR^{3a})(OR^{3b}), —S(O)—C₁₋₆ alkyl, —S(O)₂—C₁₋₆ alkyl, C₁₋₆ alkyl, —C₁₋₆ alkyl-C₃₋₁₀ cycloalkyl, —O—C₁₋₆ alkyl, aryl, heterocyclyl, alkylheterocyclyl, alkylaryl and alkylcycloalkyl; wherein the 5-10 membered heteroaryl, the —S(O)—C₁₋₆ alkyl, the —S(O)₂—C₁₋₆ alkyl, the C₁₋₆ alkyl, the —C₁₋₆ alkyl-C₃₋₁₀ cycloalkyl, the —O—C₁₋₆ alkyl, the aryl, the heterocyclyl, the alkylheterocyclyl, the alkylaryl or the alkylcycloalkyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl;
wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; or R^{3a} and R^{3b} are optionally combined to form a C₃₋₁₀ cycloalkyl, a heterocyclyl, a C₆₋₁₀ aryl, or a 5-10 membered heteroaryl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, and C₁₋₆ haloalkyl.

2. The compound of claim 1, pharmaceutically acceptable salts thereof, deuterium substitutes thereof, and isomers thereof, wherein Ring B is fused to Ring A and selected from the group consisting of: 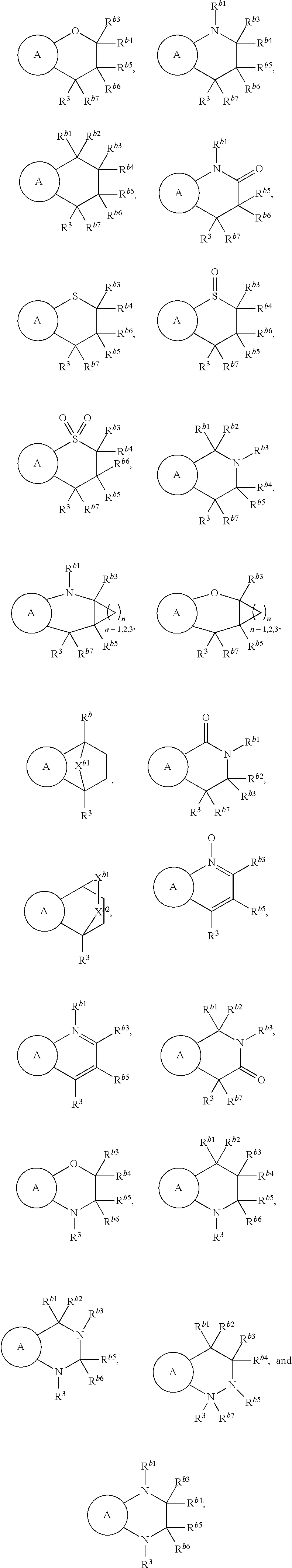
wherein each of R^{b1}, R^{b2}, R^{b3}, R^{b4}, R^{b5}, R^{b6} and R^{b7} is independently selected from the group consisting of —C₁₋₆ alkyl, —C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}—C₂₋₆ alkenyl, —C₂₋₆ alkynyl, —C₃₋₁₀ cycloalkyl, heterocyclyl, C₆₋₁₀ aryl, 5-10 membered heteroaryl, —C₁₋₆ alkyl-C₃₋₁₀ cycloalkyl, —C₁₋₆ alkyl-heterocyclyl, —C₁₋₆ alkyl-C₆₋₁₀ aryl, —C₁₋₆ alkyl-5-10 membered heteroaryl, H, F, —CH₃, —CHF₂, —CF₃, and OH; and wherein any two of R^{b1}, R^{b2}, R^{b3}, R^{b4}, R^{b5}, R^{b6}, R^{b7} and R³ are optionally combined to form a C₃₋₁₀ cycloalkyl or a heterocyclyl or a C₆₋₁₀ aryl or 5-10 membered heteroaryl; wherein each of X^{b1} and X^{b2} is independently C, N, O or S.

3. The compound of claim 2, pharmaceutically acceptable salts thereof, deuterium substitutes thereof, and isomers thereof, wherein each of R^{b1} and R^{b2} is independently selected from the group consisting of:

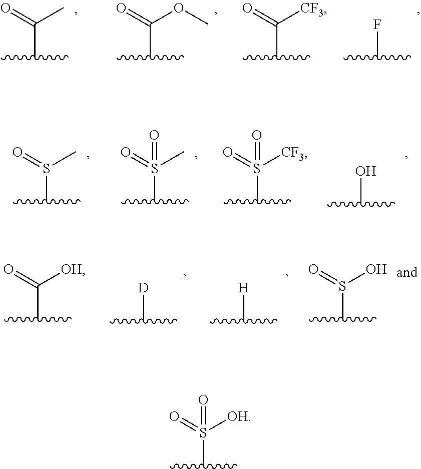

4. The compound of claim 1, pharmaceutically acceptable salts thereof, deuterium substitutes thereof, and isomers thereof, wherein the R³ group is selected from the group consisting of: —CONR^{3a}R^{3b}, —S(O)—NR^{3a}R^{3b}, —S(O)₂—NR^{3a}R^{3b}, —C₁₋₆ alkyl-CONR^{3a}R^{3b}, —C₁₋₆ alkyl-NR^{3a}R^{3b}, —N(R^{3a})—COR^{3a}, —N(R^{3a})—CONR^{3a}R^{3b}, —C₁₋₆ alkyl-SONR^{3a}R^{3b}, —C₁₋₆ alkyl-SO₂NR^{3a}R^{3b}, CN, —COR^{3a}, —O—C₁₋₆ alkyl; wherein the R³ is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

5. The compound of claim 4, pharmaceutically acceptable salts thereof, deuterium substitutes thereof, and isomers thereof, wherein the R³ group is selected from the group consisting of: —CONR^{3a}R^{3b}, —C₁₋₆ alkyl-CONR^{3a}R^{3b}, —C₁₋₆ alkyl-NR^{3a}R^{3b}, —N(R^{3a})—COR^{3b}, and —N(R^{3a})—CONR^{3a}R^{3b}, —COR^{3a}.

6. The compound of claim 1, pharmaceutically acceptable salts thereof, deuterium substitutes thereof, and isomers thereof, wherein the R^{3a} and R^{3b} are independently selected from the group consisting of H, C₁₋₆ alkyl, C₁₋₆ haloalkyl, —C₂₋₆ alkenyl, —C₂₋₆ alkynyl, C₀₋₆ alkyl-C₃₋₆ cycloalkyl, and 5-6 membered heterocyclyl; or R^{3a} and R^{3b} are optionally combined to form a C₃₋₁₀ cycloalkyl, a heterocyclyl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl and the heterocyclyl is optionally substituted with one or more substituents each independently selected from the group consisting of halo, C₁₋₆ alkyl, and C₁₋₆ haloalkyl.

7. The compound of claim 6, pharmaceutically acceptable salts thereof, deuterium substitutes thereof, and isomers thereof, wherein the R^{3a} and R^{3b} are independently selected from the group consisting of H, C₁₋₆ alkyl, and C₁₋₆ haloalkyl.

8. The compound of claim 6, pharmaceutically acceptable salts thereof, deuterium substitutes thereof, and isomers thereof, wherein the R^{3a} and R^{3b} are optionally combined to form a C₃₋₁₀ cycloalkyl, a heterocyclyl, a C₆₋₁₀ aryl, and a 5-10 membered heteroaryl; the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, —O—C₁₋₆ alkyl, and C₁₋₆ haloalkyl.

9. The compound of claim 1, pharmaceutically acceptable salts thereof, deuterium substitutes thereof, and isomers thereof, the R³ group is 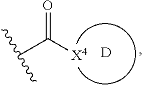
wherein the Ring D is C₃₋₁₀ cycloalkyl or 3-10 membered heterocyclyl; wherein X⁴ is N or CH; wherein the Ring D is optionally substituted with oxo, halo, C₁₋₆ alkyl, —O—C₁₋₆ alkyl, and C₁₋₆ haloalkyl; wherein heterocyclyl has one to four heteroatoms, each is independently N, O, or S.

10. The compound of claim 1, pharmaceutically acceptable salts thereof, deuterium substitutes thereof, and isomers thereof, wherein the R³ group is selected from the group consisting of

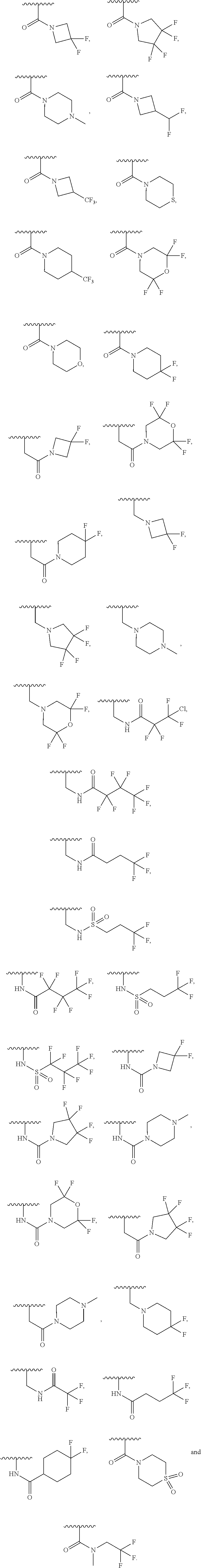

11. The compound of claim 1, pharmaceutically acceptable salts thereof, deuterium substitutes thereof, and isomers thereof, wherein Ring C is selected from the group consisting of aryl, 5-6 membered heteroaryl and C₃₋₁₀ cycloalkyl.

12. The compound of claim 11, pharmaceutically acceptable salts thereof, deuterium substitutes thereof, and isomers thereof, wherein Ring C is 5-6 membered heteroaryl.

13. The compound of claim 11, pharmaceutically acceptable salts thereof, deuterium substitutes thereof, and isomers thereof, wherein Ring C is selected from the group consisting of

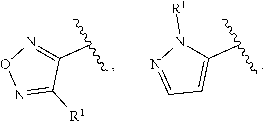

14. The compound of claim 13, pharmaceutically acceptable salts thereof, deuterium substitutes thereof, and isomers thereof, wherein Ring C is

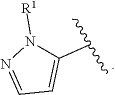

15. The compound of claim 1, pharmaceutically acceptable salts thereof, deuterium substitutes thereof, and isomers thereof, wherein Ring C is C₃₋₁₀ cycloalkyl; wherein the cycloalkyl is optionally substituted with one or more substituents each independently selected from the group consisting of halo, C₁₋₆ alkyl and C₁₋₆ haloalkyl.

16. The compound of claim 1, pharmaceutically acceptable salts thereof, deuterium substitutes thereof, and isomers thereof, wherein X³ is O.

17. The compound of claim 1, pharmaceutically acceptable salts thereof, deuterium substitutes thereof, and isomers thereof, wherein X³ is —CR^{c}R^{d}.

18. The compound of claim 1, pharmaceutically acceptable salts thereof, deuterium substitutes thereof, and isomers thereof, wherein R^{b} is independently selected from the group consisting of H, C₁₋₆ alkyl, C₁₋₆ haloalkyl, —O—C₁₋₆ alkyl, —O—C₁₋₆ haloalkyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, F and OH; wherein two R^{b} groups are optionally combined to form a C₃₋₁₀ cycloalkyl or a heterocyclyl; wherein each R^{b} is optionally substituted with one or more substituents each independently selected from the group consisting of halo, C₁₋₆ alkyl, C₁₋₆ haloalkyl, —O—C₁₋₆ alkyl, —O—C₁₋₆ haloalkyl, C₃₋₁₀ cycloalkyl, heterocyclyl.

19. The compound of claim 1, pharmaceutically acceptable salts thereof, deuterium substitutes thereof, and isomers thereof, wherein R^{b} is independently selected from the group consisting of H, C₁₋₆ alkyl, C₁₋₆ haloalkyl, —O—C₁₋₆ alkyl, —O—C₁₋₆ haloalkyl, C₃₋₁₀ cycloalkyl, heterocyclyl, —C₁₋₆ alkyl —C₃₋₁₀ cycloalkyl, —C₁₋₆ alkyl-heterocyclyl; wherein two R^{b} groups are optionally combined to form a C₃₋₁₀ cycloalkyl or a heterocyclyl; wherein each R^{b} is optionally substituted with one or more substituents each independently selected from the group consisting of halo, C₁₋₆ alkyl, C₁₋₆ haloalkyl, —O—C₁₋₆ alkyl, —O—C₁₋₆ haloalkyl, C₃₋₁₀ cycloalkyl, heterocyclyl.

20. The compound of claim 1, pharmaceutically acceptable salts thereof, deuterium substitutes thereof, and isomers thereof, wherein R¹ is independently selected from the group consisting of halogen, oxo, —C₁₋₆ alkyl, —C₁₋₆ alkoxy, —C₁₋₆ haloalkoxy, —C₁₋₆ haloalkyl and —C₃₋₁₀ cycloalkyl.

21. The compound of claim 20, pharmaceutically acceptable salts thereof, deuterium substitutes thereof, and isomers thereof, wherein R¹ is independently selected from the group consisting of halogen, —C₁₋₆ alkyl, —C₁₋₆ haloalkyl and —C₃₋₁₀ cycloalkyl.

22. The compound of claim 21, pharmaceutically acceptable salts thereof, deuterium substitutes thereof, and isomers thereof, wherein R¹ is independently selected from the group consisting of —C₁₋₆ alkyl, —C₁₋₆ haloalkyl and —C₃₋₁₀ cycloalkyl.

23. The compound of claim 1, pharmaceutically acceptable salts thereof, deuterium substitutes thereof, and isomers thereof, wherein R² is —C₀₋₁ alkyl-C₃₋₉ cycloalkyl; wherein the —C₀₋₁ alkyl-C₃₋₉ cycloalkyl is optionally substituted with one or more substituents each independently selected from the group consisting of C₃₋₆ cycloalkyl, halo, CH₃, CHF₂, CF₃, OH and oxo.

24. The compound of claim 23, pharmaceutically acceptable salts thereof, deuterium substitutes thereof, and isomers thereof, wherein R² is selected from the group consisting of: 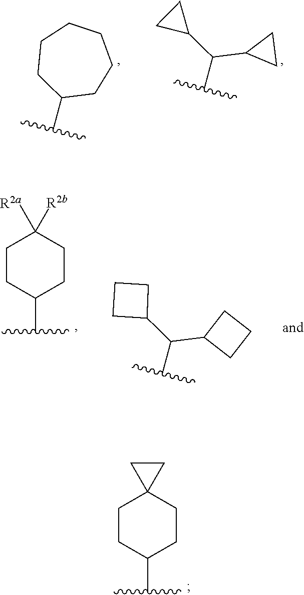
wherein R^{2a} and R^{2b} is selected from the group consisting of H, halo, C₁₋₃ alkyl;
And R² is optionally substituted with one or more halo and C₁₋₆ alkyl.

25. The compound of claim 24, pharmaceutically acceptable salts thereof, deuterium substitutes thereof, and isomers thereof, wherein R² is selected from the group consisting of

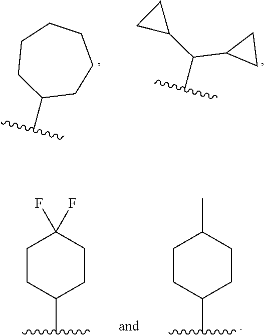

26. The compound of claim 25, pharmaceutically acceptable salts thereof, deuterium substitutes thereof, and isomers thereof, wherein R² is selected from the group consisting of

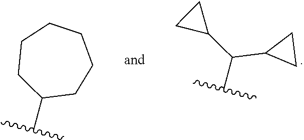 [SMILES: *C1CCCCCC1 ; *C(C1CC1)C1CC1]

27. The compound of claim 1, pharmaceutically acceptable salts thereof, deuterium substitutes thereof, and isomers thereof, wherein the compound has a structure selected from the group consisting of Formula I-A-1, Formula I-A-2, Formula I-A-3 and I-A-4:

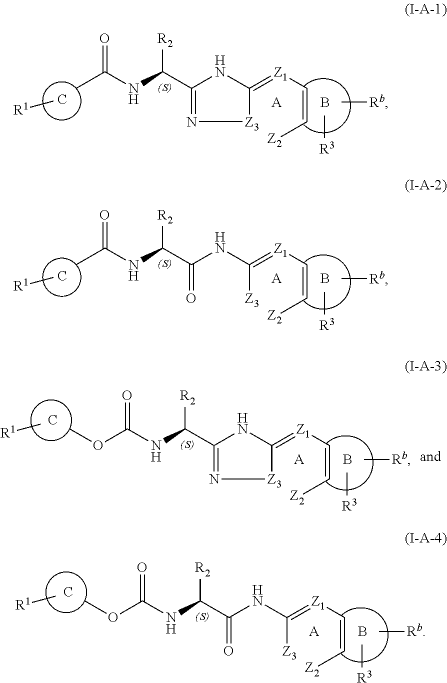

28. The compound of claim 1, pharmaceutically acceptable salts thereof, deuterium substitutes thereof, and isomers thereof, wherein the compound has a structure selected from the group consisting of Formula I-A-5, Formula I-A-6, Formula I-A-7 and Formula I-A-8: 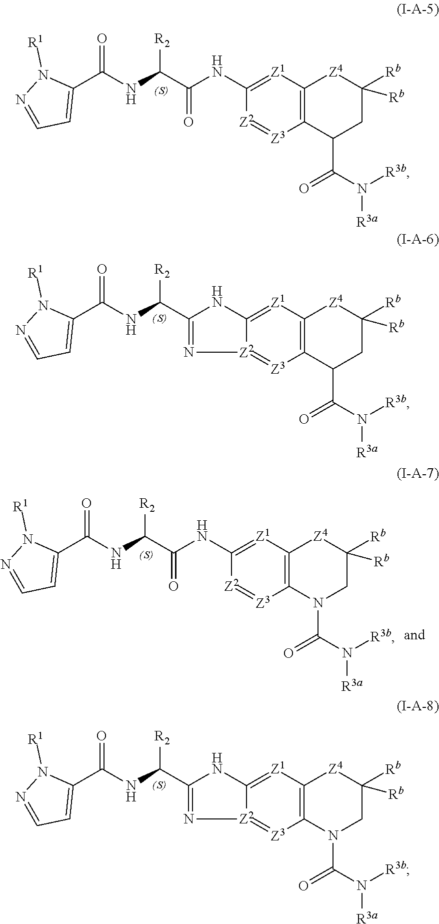
wherein Z⁴ is O, S, S═O, S(═O)₂, or NR^{b1};
wherein X¹ is O, S, or NH; and
wherein Z¹, Z² or Z³ is independently N or —C—R^{a}.

29. The compound of claim 1, pharmaceutically acceptable salts thereof, deuterium substitutes thereof, and isomers thereof, wherein the compound has a structure selected from the group consisting of Formula I-A-9, Formula I-A-10, Formula I-A-11 and Formula I-A-12: 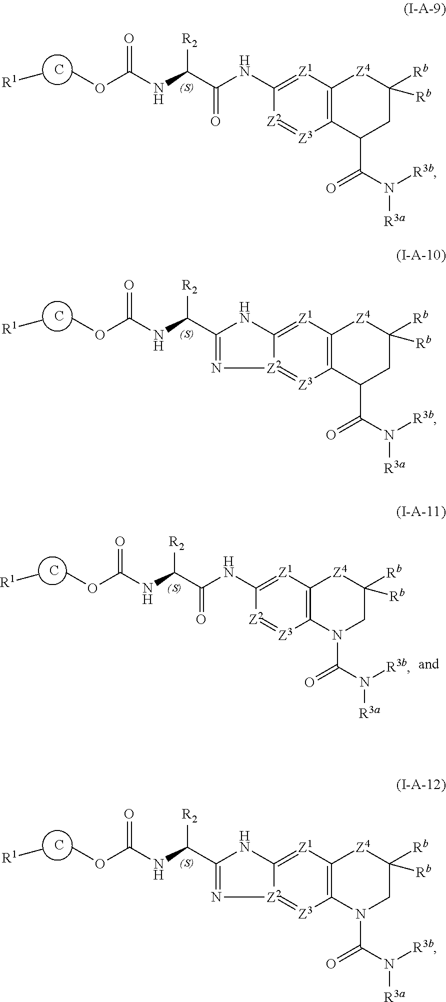
wherein Z⁴ is O, S, S═O, S(═O)₂, or NR^{b1};
wherein X¹ is O, S, or NH;
wherein Z¹, Z² or Z³ is independently N or —C—R^{a}; and
wherein Ring C is aryl, 5-6 membered heteroaryl, C₃₋₁₀ cycloalkyl, C₃₋₁₀ heterocyclyl, —C₁₋₆ alkyl-C₃₋₁₀ cycloalkyl, —C₁₋₆ alkyl-C₃₋₁₀ heterocyclyl, —C₁₋₆ alkyl-aryl, or —C₁₋₆ alkyl-5-6 membered heteroaryl; wherein the aryl, the 5-6 membered heteroaryl, the C₃₋₁₀ cycloalkyl, the C₃₋₁₀ heterocyclyl, the —C₁₋₆ alkyl-C₃₋₁₀ cycloalkyl, the —C₁₋₆ alkyl-C₃₋₁₀ heterocyclyl, the —C₁₋₆ alkyl-aryl, or the —C₁₋₆ alkyl-5-6 membered heteroaryl is optionally substituted with one or more substituents each independently selected from the group consisting of halo, C₁₋₆ alkyl, C₁₋₆ haloalkyl, C₁₋₆ haloalkoxy and C₁₋₆ alkoxy.

30. The compound of claim 1, pharmaceutically acceptable salts thereof, deuterium substitutes thereof, and isomers thereof, wherein the compound has a structure of Formula I-A-13: 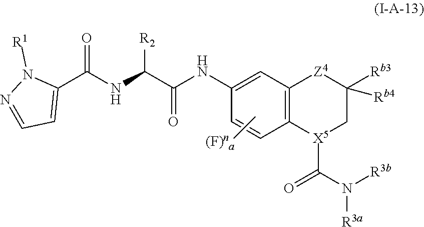
wherein Z⁴ is O, —C(R^{b1}R^{b2})—, —N(R^{b1})—;
X⁵ is N or CH;
wherein R^{b1}, R^{b2}, R^{b3} and R^{b4} is independently selected from the group consisting of —C₁₋₆ alkyl, —C₁₋₆ haloalkyl, O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —C₂₋₆ alkenyl, —C₂₋₆ alkynyl, —C₃₋₁₀ cycloalkyl, heterocyclyl, C₆₋₁₀ aryl, 5-10 membered heteroaryl, —C₁₋₆ alkyl-C₃₋₁₀ cycloalkyl, —C₁₋₆ alkyl-heterocyclyl, —C₁₋₆ alkyl-C₆₋₁₀ aryl, —C₁₋₆ alkyl-5-10 membered heteroaryl, H, F, —CH₃, —CHF₂, —CF₃, and OH; and R^{b3} and R^{b4} are optionally combined to form a C₃₋₁₀ cycloalkyl or a heterocyclyl; wherein the C₃₋₁₀ cycloalkyl or heterocyclyl is optionally substituted with one or more substituents each independently selected from the group consisting of oxo, halo, C₁₋₆ alkyl, C₁₋₆ haloalkyl, —O—C₁₋₆ alkyl and —O—C₁₋₆ haloalkyl;
wherein the R^{3a} and R^{3b} is independently selected from the group consisting of H, C₁₋₆ alkyl, C₁₋₆ haloalkyl, —C₂₋₆ alkenyl, —C₂₋₆ alkynyl, C₀₋₆ alkyl-C₃₋₆ cycloalkyl, and 5-6 membered heterocyclyl; or R^{3a} and R^{3b} are optionally combined to form a C₃₋₁₀ cycloalkyl, a heterocyclyl; wherein the C₁₋₆ alkyl, the C₁₋₆ haloalkyl, the —C₂₋₆ alkenyl, the —C₂₋₆ alkynyl, the CO-2 alkyl-C₃₋₆ cycloalkyl, the 5-6 membered heterocyclyl, the C₃₋₁₀ cycloalkyl and the heterocyclyl is optionally substituted with one or more substituents each independently selected from the group consisting of halo, C₁₋₆ alkyl, and C₁₋₆ haloalkyl; and
wherein nₐ is 0, 1 and 2.

31. The compound of claim 30, pharmaceutically acceptable salts thereof, deuterium substitutes thereof, and isomers thereof, wherein R^{b3} and R^{b4} are independently selected from the group consisting of H, —C₁₋₆ alkyl, —C₁₋₆ haloalkyl, 0-C₁₋₆ alkyl, —O—C₁₋₆ haloalkyl, —C₃₋₁₀ cycloalkyl, heterocyclyl, —C₁₋₆ alkyl-C₃₋₁₀ cycloalkyl, —C₁₋₆ alkyl-heterocyclyl; and R^{b3} and R^{b4} are optionally combined to form a C₃₋₁₀ cycloalkyl or a heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

32. The compound of claim 30, pharmaceutically acceptable salts thereof, deuterium substitutes thereof, and isomers thereof, wherein R^{b4} is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, and —C₁₋₆ alkyl-heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

33. The compound of claim 30, pharmaceutically acceptable salts thereof, deuterium substitutes thereof, and isomers thereof, wherein R^{b4} is selected from the group consisting of —C₁₋₆ alkyl, —C₃₋₁₀ cycloalkyl and heterocyclyl; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

34. The compound of claim 30, pharmaceutically acceptable salts thereof, deuterium substitutes thereof, and isomers thereof, wherein X⁵ is N.

35. The compound of claim 30, pharmaceutically acceptable salts thereof, deuterium substitutes thereof, and isomers thereof, wherein X⁵ is CH.

36. The compound of claim 1, pharmaceutically acceptable salts thereof, deuterium substitutes thereof, and isomers thereof, wherein the compound has a structure of Formula I-A-18:

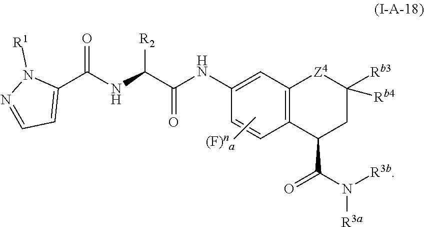

37. The compound of claim 29, pharmaceutically acceptable salts thereof, deuterium substitutes thereof, and isomers thereof, wherein Z⁴ is selected from the group consisting of O and CH².

38. The compound of claim 37, pharmaceutically acceptable salts thereof, deuterium substitutes thereof, and isomers thereof, wherein Z⁴ is O.

39. The compound of claim 37, pharmaceutically acceptable salts thereof, deuterium substitutes thereof, and isomers thereof, wherein Z⁴ is CH₂.

40. The compound of claim 1, pharmaceutically acceptable salts thereof, deuterium substitutes thereof, and isomers thereof, wherein the compound has a structure of Formula I-A-14:

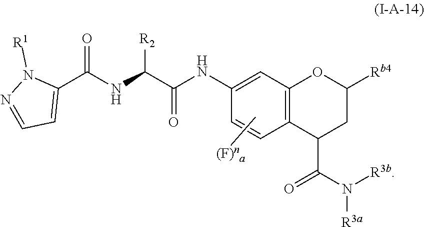

41. The compound of claim 40, pharmaceutically acceptable salts thereof, deuterium substitutes thereof, and isomers thereof, wherein nₐ is 0 and 1.

42. The compound of claim 40, pharmaceutically acceptable salts thereof, deuterium substitutes thereof, and isomers thereof, wherein R^{b4} is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, —C₁₋₆ alkyl-heterocyclyl, H, and F; wherein the C₃₋₁₀ cycloalkyl or heterocyclyl is optionally substituted with one or more substituents each independently selected from the group consisting of halo, oxo, C₁₋₆ alkyl, C₁₋₆ haloalkyl, —O—C₁₋₆ alkyl and —O—C₁₋₆ haloalkyl.

43. The compound of claim 1, pharmaceutically acceptable salts thereof, deuterium substitutes thereof, and isomers thereof, wherein the compound has a structure of Formula I-A-15: 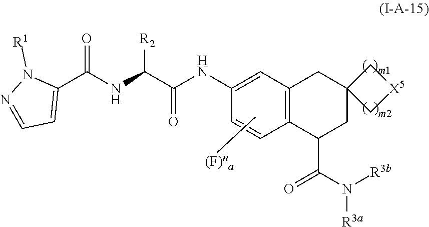
wherein X⁵ is selected from the group consisting of O, —N(R^{x5})—, —C(R^{X6}R^{X7})—, S, S(O) and S(O)₂;
m1 and m2 is 0, 1, 2 and 3;
nₐ is 0, 1 and 2;
wherein R^{x5} is selected from the group consisting of H, C₁₋₆ alkyl, C₁₋₆ haloalkyl, —C₃₋₁₀ cycloalkyl, —CO—C₁₋₆ alkyl and —CO—C₁₋₆ haloalkyl; and
R^{x6} and R^{x7} is independently selected from the group consisting of H, halo, C₁₋₆ alkyl, and C₁₋₆ haloalkyl.

44. The compound of claim 1, pharmaceutically acceptable salts thereof, deuterium substitutes thereof, and isomers thereof, wherein the compound has a structure of Formula I-A-16: 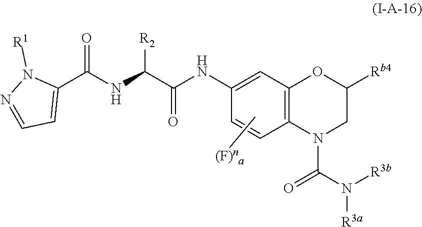
wherein nₐ is 0, 1 and 2.

45. The compound of claim 44, pharmaceutically acceptable salts thereof, deuterium substitutes thereof, and isomers thereof, wherein R^{b4} is selected from the group consisting of —C₁₋₆ alkyl, —C₁₋₆ haloalkyl, —C₃₋₁₀ cycloalkyl, heterocyclyl, —C₁₋₆ alkyl-C₃₋₁₀ cycloalkyl, —C₁₋₆ alkyl-heterocyclyl, H, and F; wherein the C₃₋₁₀ cycloalkyl or heterocyclyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl.

46. The compound of claim 1, pharmaceutically acceptable salts thereof, deuterium substitutes thereof, and isomers thereof, wherein the compound has a structure of Formula I-A-17: 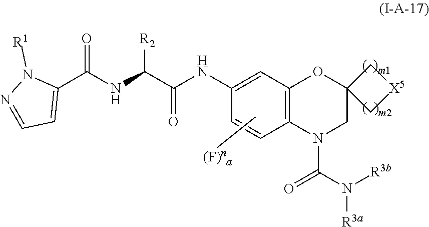
wherein X₅ is O, —N(R^{x5})—, —C(R^{X6}R^{X7})—, S, S(O) and S(O)₂;
m1 and m2 is 0, 1, 2 and 3;
nₐ is 0, 1 and 2;
wherein R^{x5} is H, C₁₋₆ alkyl, C₁₋₆ haloalkyl, —CO—C₁₋₆ alkyl, —C₃₋₁₀ cycloalkyl and —CO—C₁₋₆ haloalkyl; and
R^{x6} and R^{x7} is H, halo, C₁₋₆ alkyl, C₁₋₆ haloalkyl.

47. The compound of claim 1, pharmaceutically acceptable salts thereof, deuterium substitutes thereof, and isomers thereof, wherein the compound has a structure of Formula I-A-19:

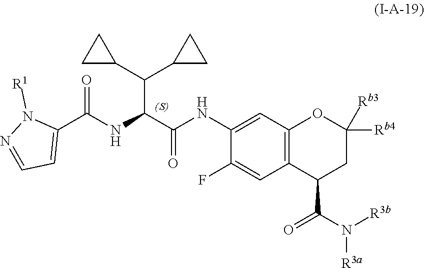

48. A compound of Formula II: 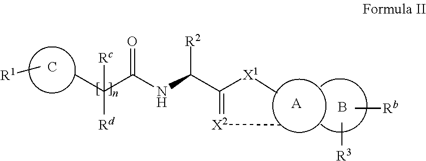
pharmaceutically acceptable salts thereof, deuterium substitutes thereof, and isomers thereof,
wherein Ring C is aryl or 5-6 membered heteroaryl;
wherein Ring C is optionally substituted with one or more R¹;
wherein R¹ is selected from the group consisting of C₁₋₆ alkyl, C₁₋₆ haloalkyl, C₂₋₆ alkenyl, C₂₋₆ alkynyl, C₃₋₁₀ cycloalkyl, C₃₋₁₀ heterocyclyl, C₆₋₁₀ aryl, and 5-10 membered heteroaryl;
wherein R¹ is optionally substituted with halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl;
wherein each of R^{c} and R^{d} is independently selected from the group consisting of H and halo;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X¹ is NH, O, or S;
wherein X² is N, O, or S; X² is optionally attached to an atom in ring A and forms another ring next to Ring A;
wherein Ring A is selected from the group consisting of 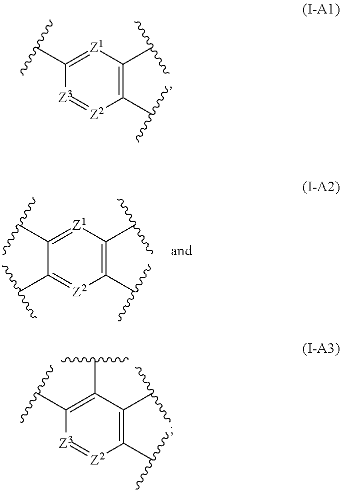
wherein each of Z¹, Z² and Z³ is independently selected from the group consisting of N, N═O, and —C—R^{a}, wherein R^{a} is selected from the group consisting of H, F, Cl, —CH₃, —CHF₂, —CF₃, OH, —OCH₃ and —OCF₃;
wherein Ring B, which is fused to ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated and unsaturated bridged-ring;
wherein Ring B is optionally substituted with one or more R^{b}, each of R^{b} is independently selected from the group consisting of C₁₋₆ alkyl, C₁₋₆ haloalkyl, —O—C₁₋₆ alkyl, —O—C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more halo;
wherein Ring B is substituted with R³, wherein R³ is selected from the group consisting of 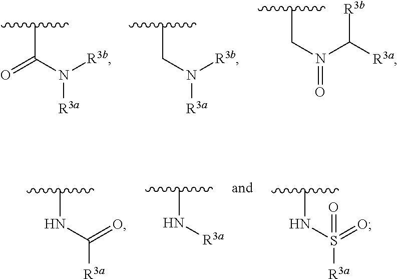
wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; wherein R^{3a} and R^{3b} are optionally combined to form a C₃₋₁₀ cycloalkyl, a heterocyclyl, a C₆₋₁₀ aryl, or a 5-10 membered heteroaryl.

49. A compound of Formula III: 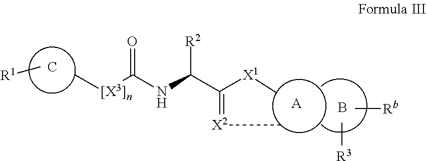
pharmaceutically acceptable salts thereof, deuterium substitutes thereof, and isomers thereof,
wherein Ring C is aryl, 5-6 membered heteroaryl, C₃₋₁₀ cycloalkyl, C₃₋₁₀ heterocyclyl, R^{x}—C₃₋₁₀ cycloalkyl, R^{x}—C₃₋₁₀ heterocyclyl, R^{x}-aryl and R^{x}-5-6 membered heteroaryl; wherein R^{x} is halo or —C₁₋₆ alkyl;
wherein Ring C is optionally substituted with one or more R¹;
wherein each R¹ is independently selected from the group consisting of OH, CN, C₁₋₆ alkyl, C₁₋₆ haloalkyl, C₂₋₆ alkenyl, C₂₋₆ alkynyl, —O—C₁₋₆ alkyl, —O—C₁₋₆ haloalkyl, —CO—C₁₋₆ alkyl, —CO—C₁₋₆ haloalkyl, —CO—NR^{3a}R^{3b}, C₃₋₁₀ cycloalkyl, C₃₋₁₀ heterocyclyl, C₆₋₁₀ aryl, and 5-10 membered heteroaryl;
wherein each R¹ is optionally substituted with one or more substituents each independently selected from the group consisting of halogen, oxo, —OH, —CN, —C₁₋₆ alkyl, —C₁₋₆ alkoxy, —C₁₋₆ haloalkoxy, —C₁₋₆ haloalkyl, —C₃₋₁₀ cycloalkyl, heterocyclyl, —C₆₋₁₀ aryl, 5-10 membered heteroaryl, —S(O)—C₁₋₆ alkyl, —S(O)₂—C₁₋₆ alkyl and —S—C₁₋₆ alkyl;
wherein n is 0 or 1;
wherein R² is selected from the group consisting of —C₀₋₁ alkyl-C₃₋₉ cycloalkyl, —C₁₋₆ alkyl and —C₁₋₂ alkyl-O—C₁₋₃ alkyl, wherein the —C₀₋₁ alkyl-C₃₋₉ cycloalkyl, the —C₁₋₆ alkyl or the —C₁₋₂ alkyl-O—C₁₋₃ alkyl is optionally substituted with one or more substituents each independently selected from the group consisting of C₃₋₆ cycloalkyl, halo, CH₃, CHF₂, CF₃, OH and oxo;
wherein X¹ is NH, O, or S;
wherein X² is N, O, or S; and wherein X² is optionally attached to an atom in Ring A and forms another ring next to Ring A;
wherein X³ is O or —CR^{c}R^{d}; and wherein each of R^{c} and R^{d} is independently selected from the group consisting of H, halo and —C₁₋₆ alkyl;
wherein Ring A is selected from the group consisting of 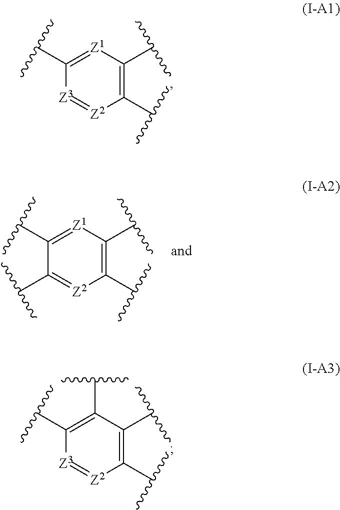
wherein each of Z¹, Z² and Z³ is independently selected from the group consisting of —N═, —N(═O)═ and —C(R^{a})═, wherein R^{a} is selected from the group consisting of H, F, Cl, Br, I, —C₁₋₆ alkyl, C₁₋₆ haloalkyl, C₂₋₆ alkenyl, C₂₋₆ alkynyl, OH, CN, —O—C₁₋₆ alkyl, —O—C₁₋₆ haloalkyl, C₃₋₁₀ cycloalkyl, C₃₋₁₀ heterocyclyl, aryl and 5-10 membered heteroaryl;
wherein Ring B, which is fused to Ring A, is selected from the group consisting of 4-7 membered heterocycloalkyl, 4-7 membered heterocycloaryl, 4-7 membered cycloalkyl, and 4-7 membered cycloalkenyl, wherein each of the 4-7 membered heterocycloalkyl, the 4-7 membered heterocycloaryl, the 4-7 membered cycloalkyl, and the 4-7 membered cycloalkenyl is optionally saturated or unsaturated bridged-ring;
wherein Ring B is optionally substituted with one or more R^{b}, each of R^{b} is independently selected from the group consisting of CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, —NR^{3a}—CO—C₁₋₆ alkyl, —NR^{3a}, —COOC₁₋₆ alkyl, —NR^{3a}—CO—C₁₋₆ haloalkyl, —NR^{3a}—CO—NR^{3a}R^{3b}, C₂₋₆ alkenyl, C₂₋₆ alkynyl, C₃₋₁₀ cycloalkyl, heterocyclyl, C₆₋₁₀ aryl, 5-10 membered heteroaryl, —C₁₋₆ alkyl —C₃₋₁₀ cycloalkyl, —C₁₋₆ alkyl-heterocyclyl, —C₁₋₆ alkyl —C₆₋₁₀ aryl, —C₁₋₆ alkyl-5-10 membered heteroaryl, F, —CH₃, —CHF₂, —CF₃, and OH; wherein two R^{b} groups are optionally combined to form a C₃₋₁₀ cycloalkyl or a heterocyclyl or a C₆₋₁₀ aryl or 5-10 membered heteroaryl; wherein each R^{b} is optionally substituted with one or more substituents each independently selected from the group consisting of halo, CN, C₁₋₆ alkyl, C₁₋₆ haloalkyl, —O—C₁₋₆ alkyl, —O—C₁₋₆ haloalkyl, —CO—C₁₋₆ alkyl, COOC₁₋₆ alkyl, —CO—C₁₋₆ haloalkyl, —CO—NR^{3a}R^{3b}, —CH₂—NR^{3a}R^{3b}, C₂₋₆ alkenyl, C₂₋₆ alkynyl, C₃₋₁₀ cycloalkyl, heterocyclyl, C₆₋₁₀ aryl, and 5-10 membered heteroaryl;
wherein Ring B is substituted with R³, wherein R³ is selected from the group consisting of
—CONR^{3a}R^{3b}, —S(O)—NR^{3a}R^{3b}, —S(O)₂—NR^{3a}R^{3b}, —C₀₋₆ alkyl-CONR^{3a}R^{3b}, —C₀₋₆ alkyl-NR^{3a}R^{3b}, —N(R^{3a})—COR^{3a}, —N(R^{3a})—CONR^{3a}R^{3b}, —C₀₋₆ alkyl-SONR^{3a}R^{3b}—C₀₋₆ alkyl-SO₂NR^{3a}R^{3b}, —C(S)—NR^{3a}R^{3b}, CN, 5-10 membered heteroaryl, —COR^{3a}, —P(O)(OR^{3a}OR^{3b}), —S(O)—C₁₋₆ alkyl, —S(O)₂—C₁₋₆ alkyl, C₁₋₆ alkyl, —C₁₋₆ alkyl- C₃₋₁₀ cycloalkyl, —O—C₁₋₆ alkyl, aryl, heterocyclyl, alkylheterocyclyl, alkylaryl and alkylcycloalkyl; wherein the 5-10 membered heteroaryl, the —S(O)—C₁₋₆ alkyl, the —S(O)₂—C₁₋₆ alkyl, the C₁₋₆ alkyl, the —C₁₋₆ alkyl-C₃₋₁₀ cycloalkyl, the —O—C₁₋₆ alkyl, the aryl, the heterocyclyl, the alkylheterocyclyl, the alkylaryl or the alkylcycloalkyl is optionally substituted with one or more substituents each independently selected from the group consisting of halo, C₁₋₆ alkyl, C₁₋₆ haloalkyl, —O—C₁₋₆ alkyl and —O—C₁₋₆ haloalkyl; wherein each of R^{3a} and R^{3b} is independently selected from the group consisting of C₁₋₆ alkyl, C₁₋₆ haloalkyl, —C₂₋₆ alkenyl, —C₂₋₆ alkynyl, C₀₋₂ alkyl-C₃₋₆ cycloalkyl, and 5-6 membered heterocyclyl; or R^{3a} and R^{3b} are optionally combined to form a C₃₋₁₀ cycloalkyl, a heterocyclyl, a C₆₋₁₀ aryl, or a 5-10 membered heteroaryl; wherein the C₁₋₆ alkyl, the C₁₋₆ haloalkyl, the —C₂₋₆ alkenyl, the —C₂₋₆ alkynyl, the C₀₋₂ alkyl-C₃₋₆ cycloalkyl, the 5-6 membered heterocyclyl, the C₃₋₁₀ cycloalkyl, the heterocyclyl, the C₆₋₁₀ aryl, or the 5-10 membered heteroaryl is optionally substituted with one or more substituents each independently selected from the group consisting of halo, C₁₋₆ alkyl, and C₁₋₆ haloalkyl.

50. A compound having a structure selected from the group consisting of: 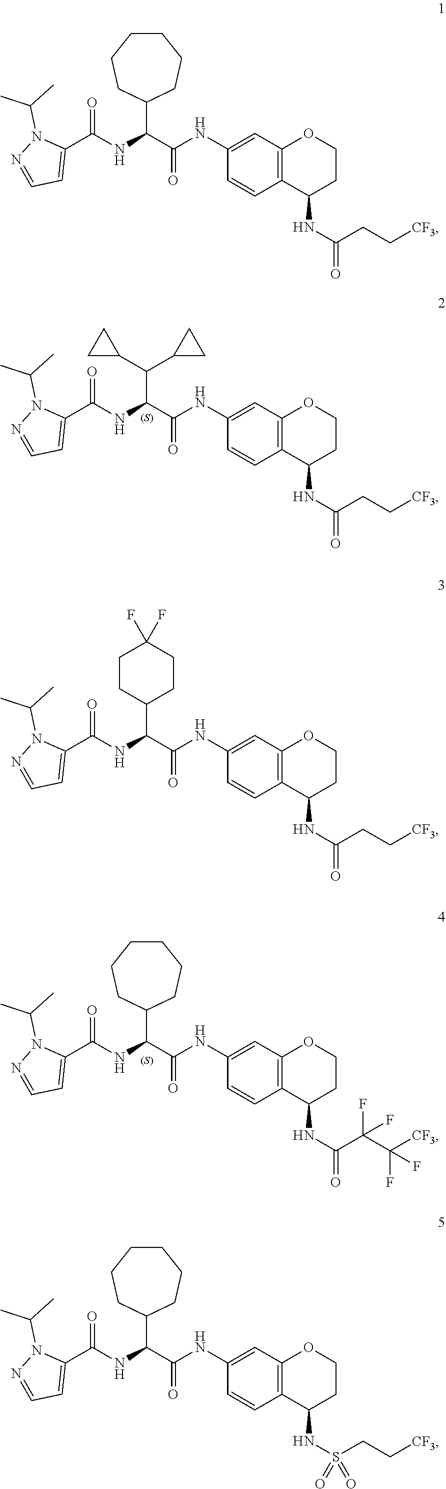 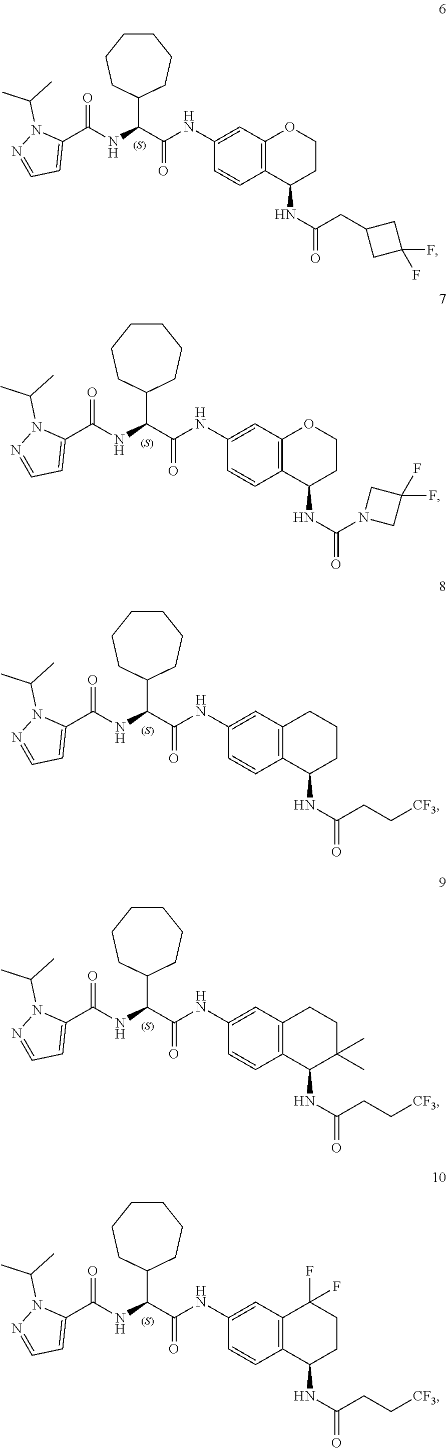 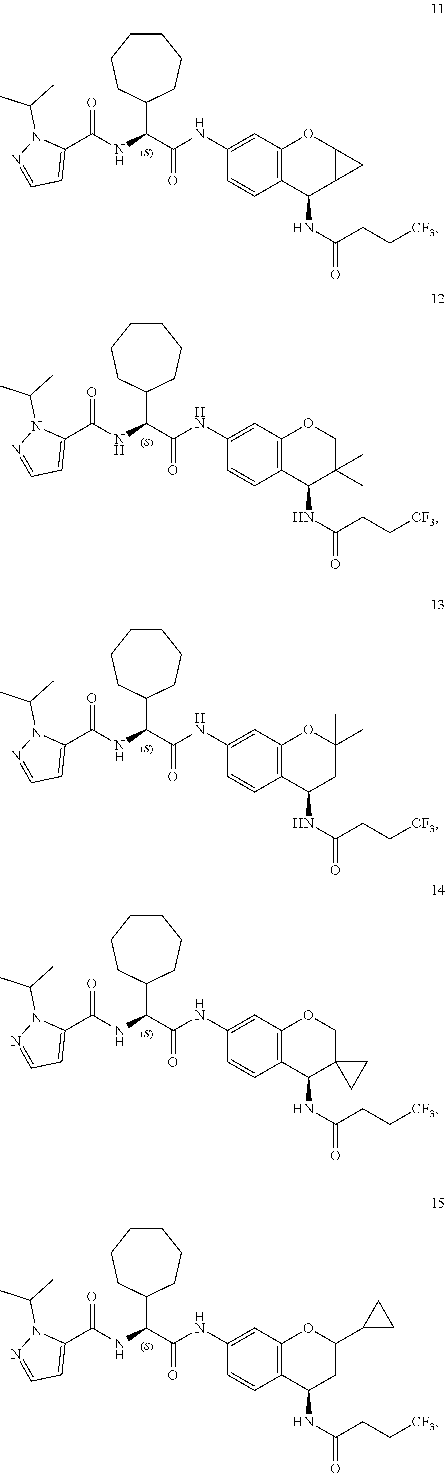 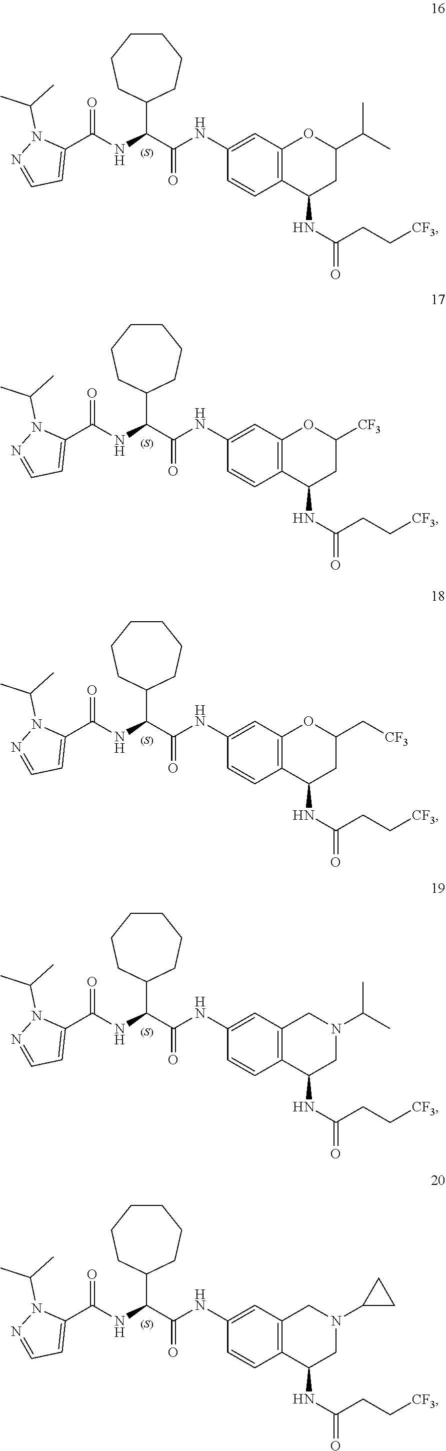 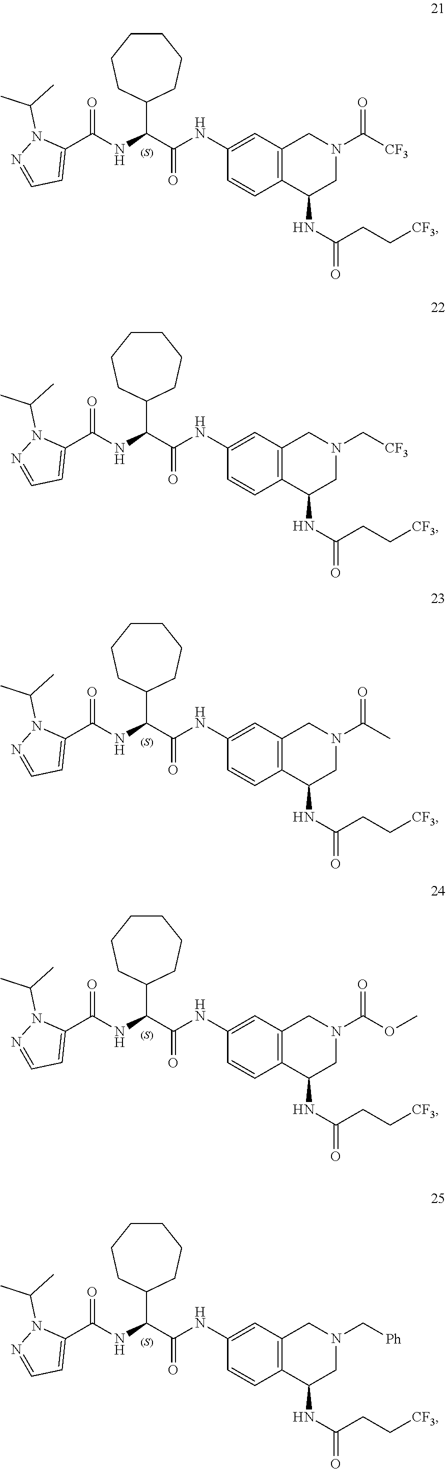 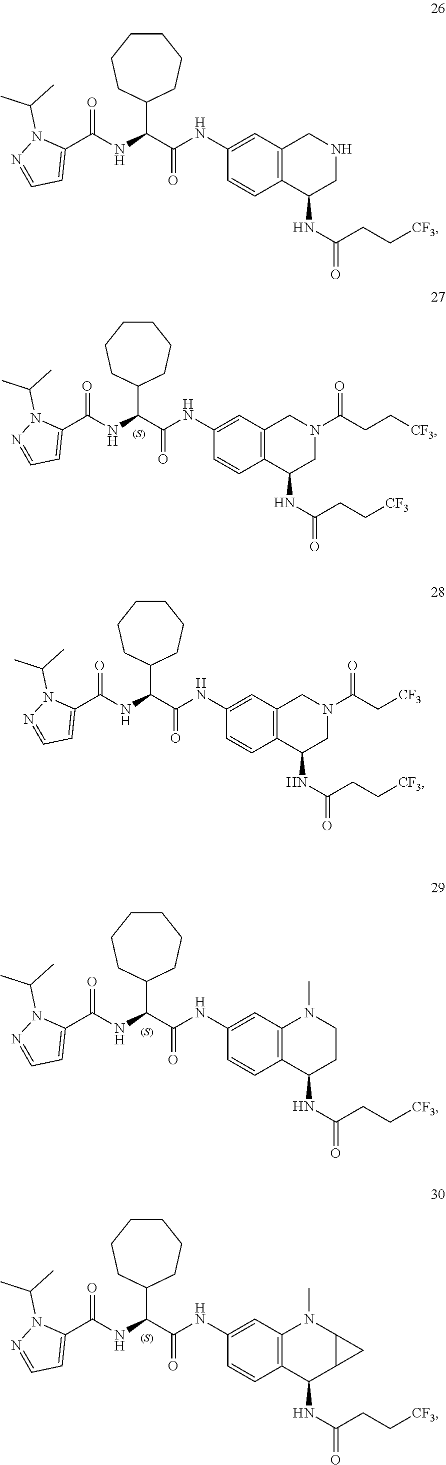 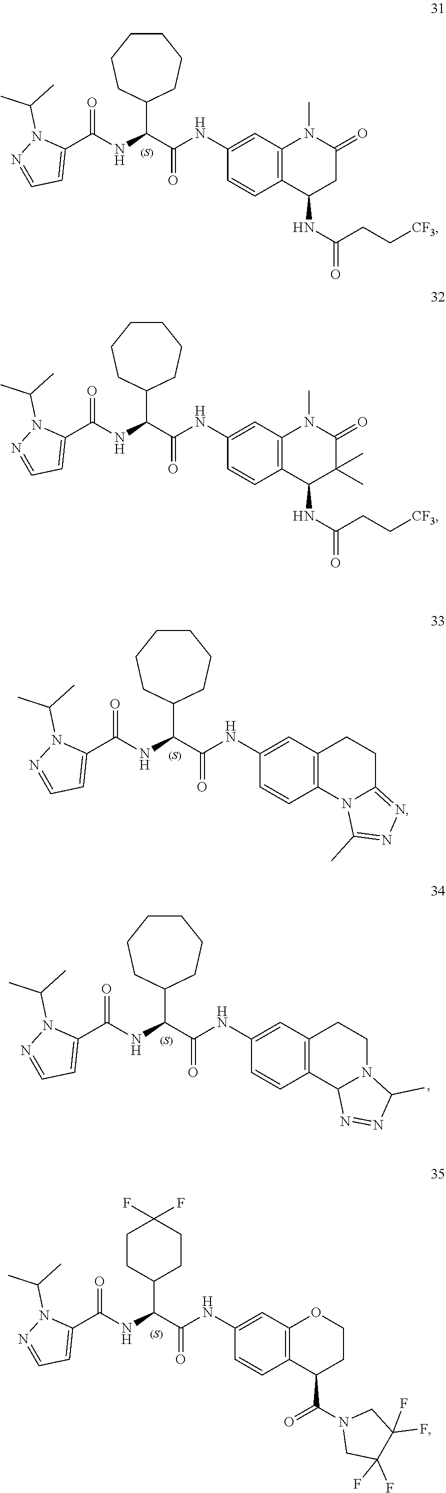 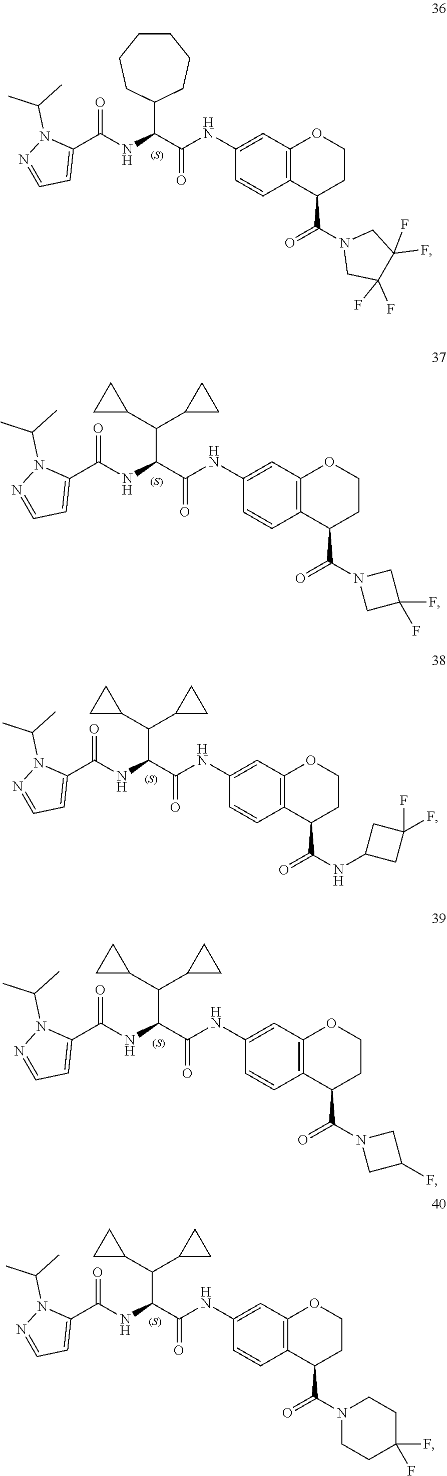 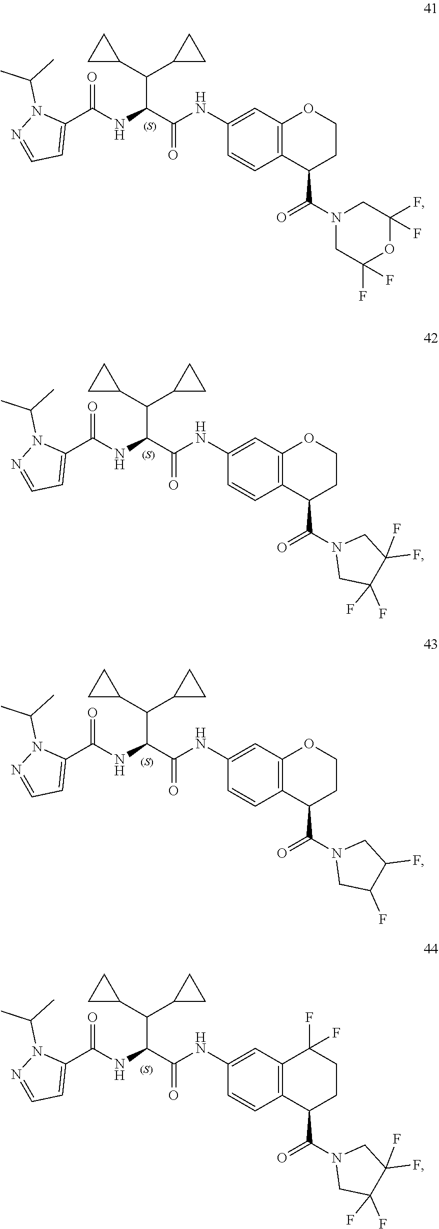 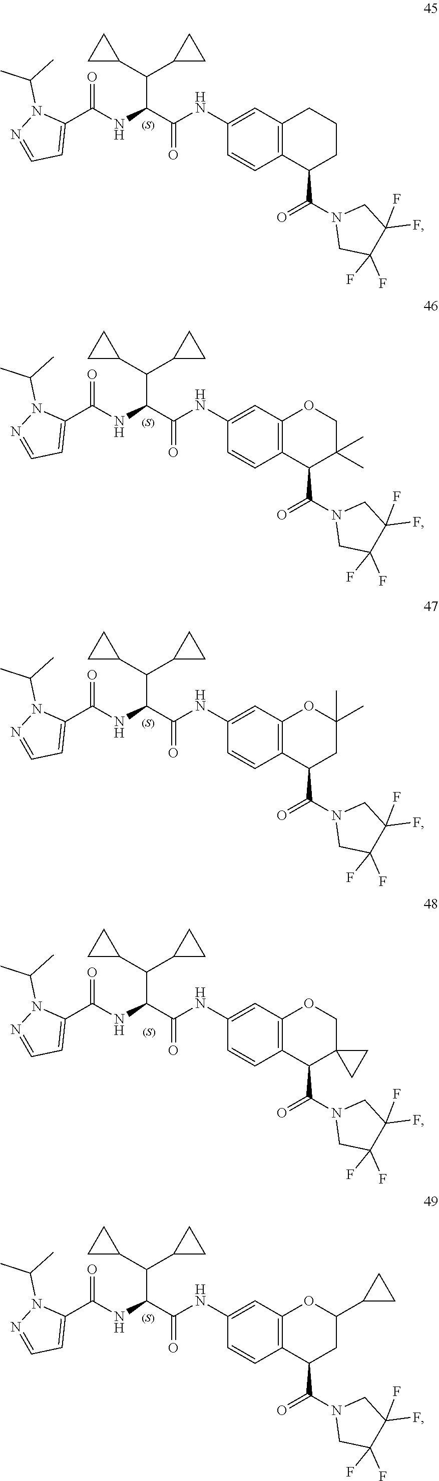 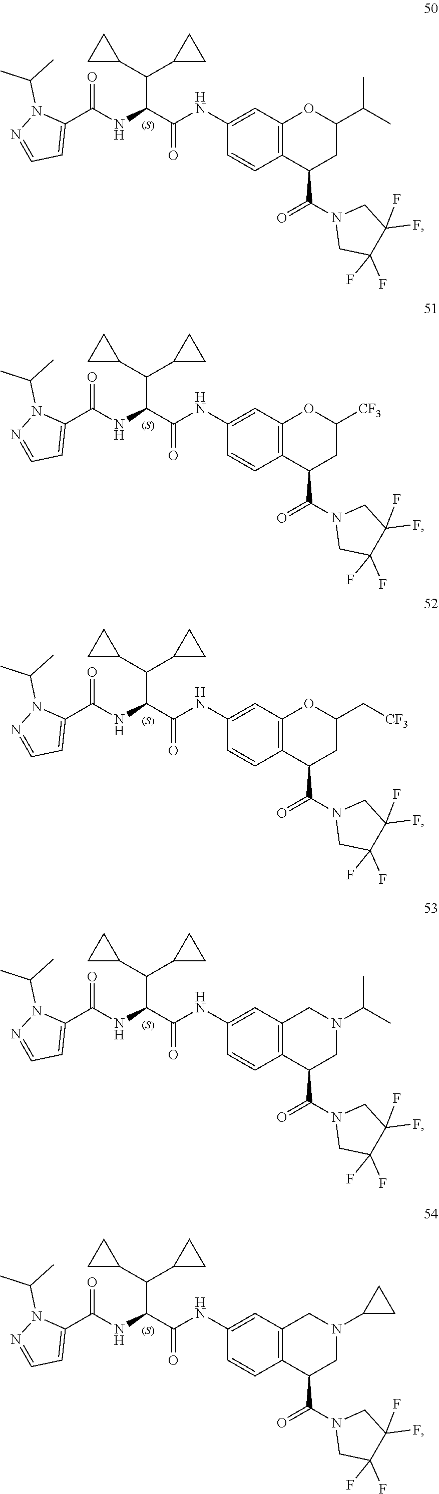 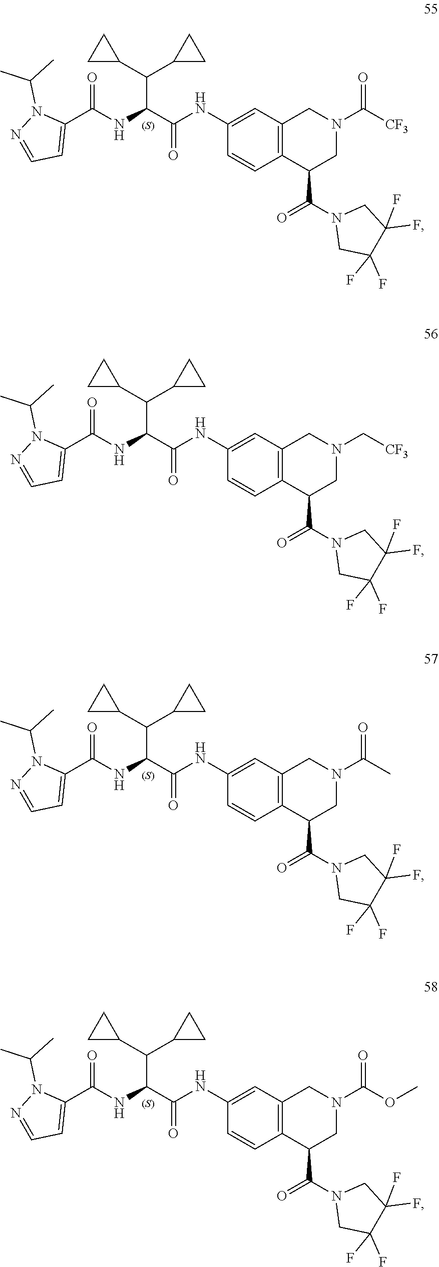 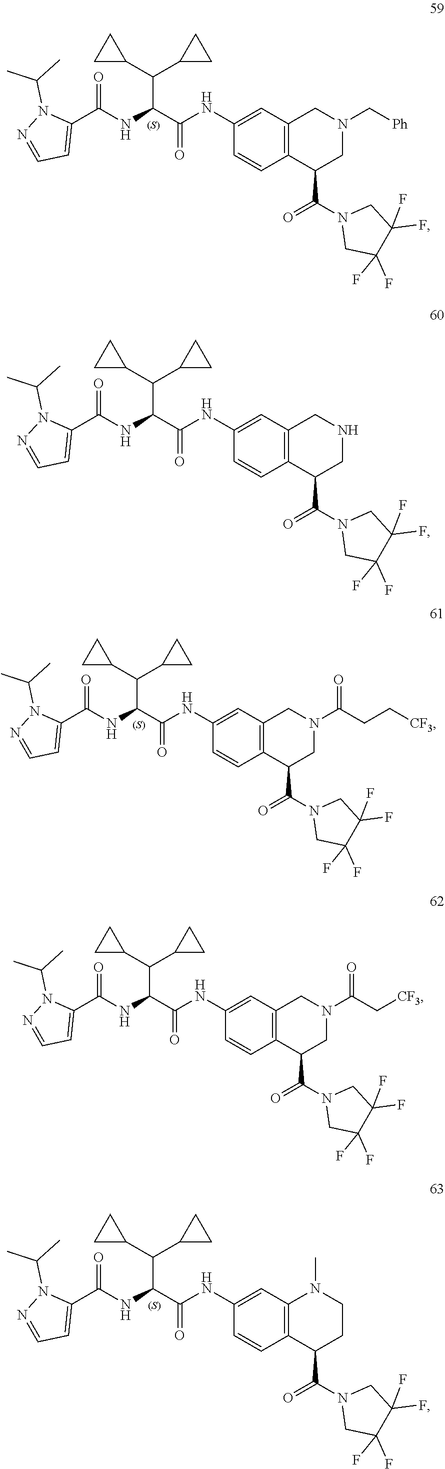 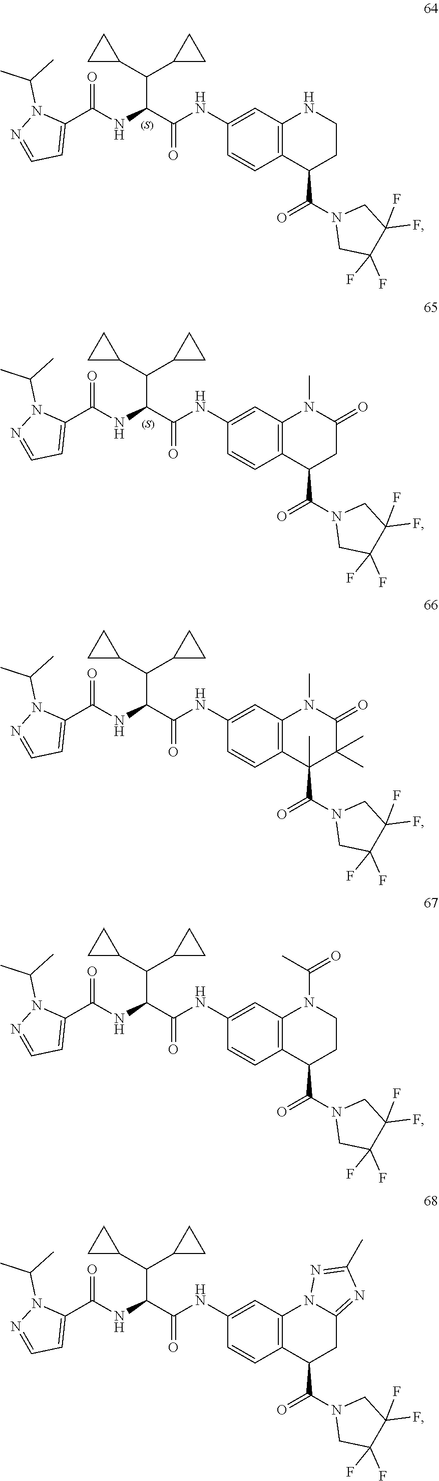 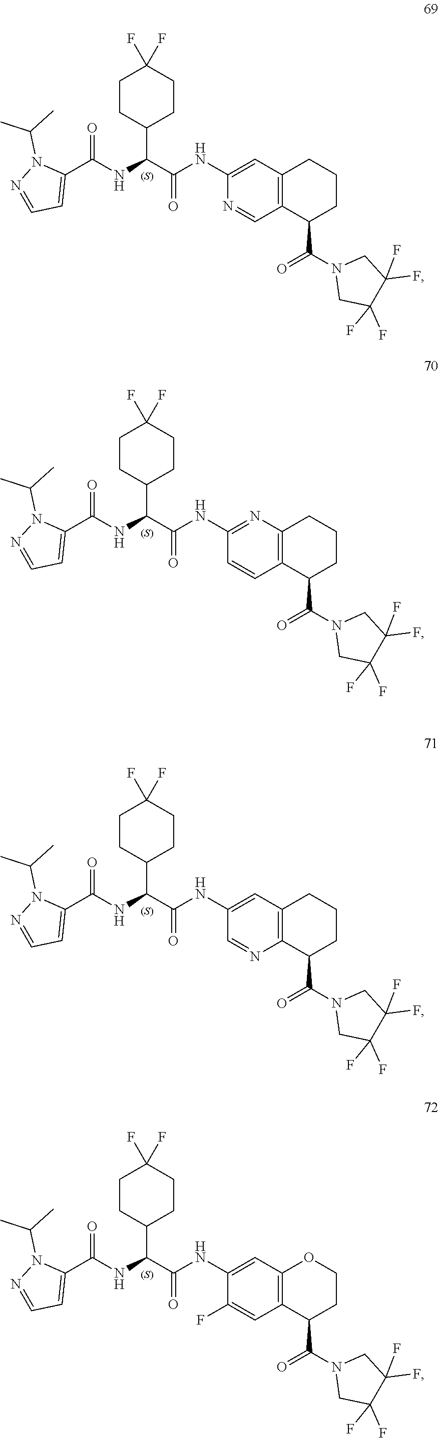 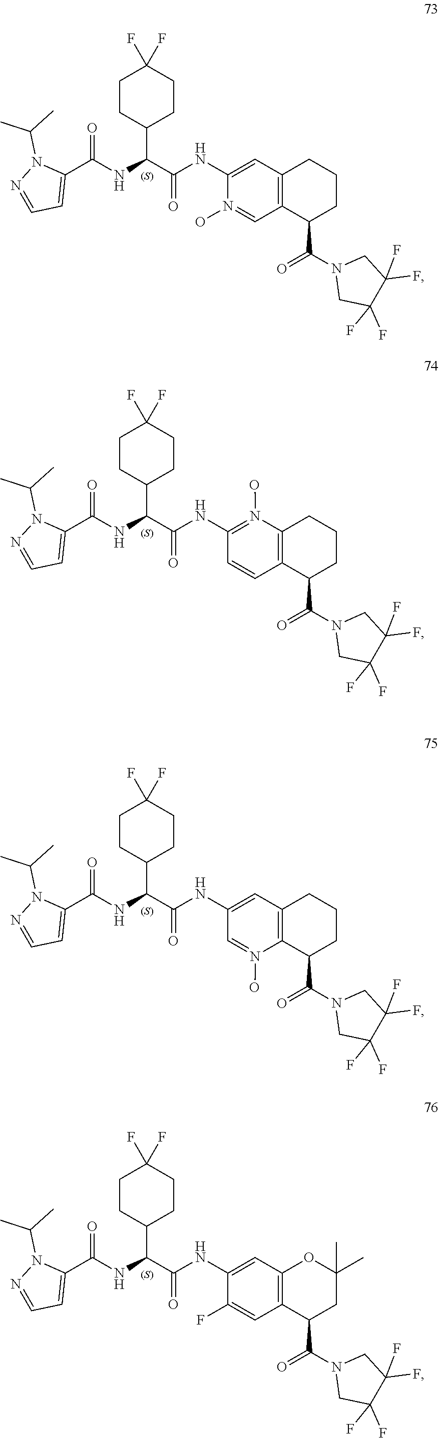 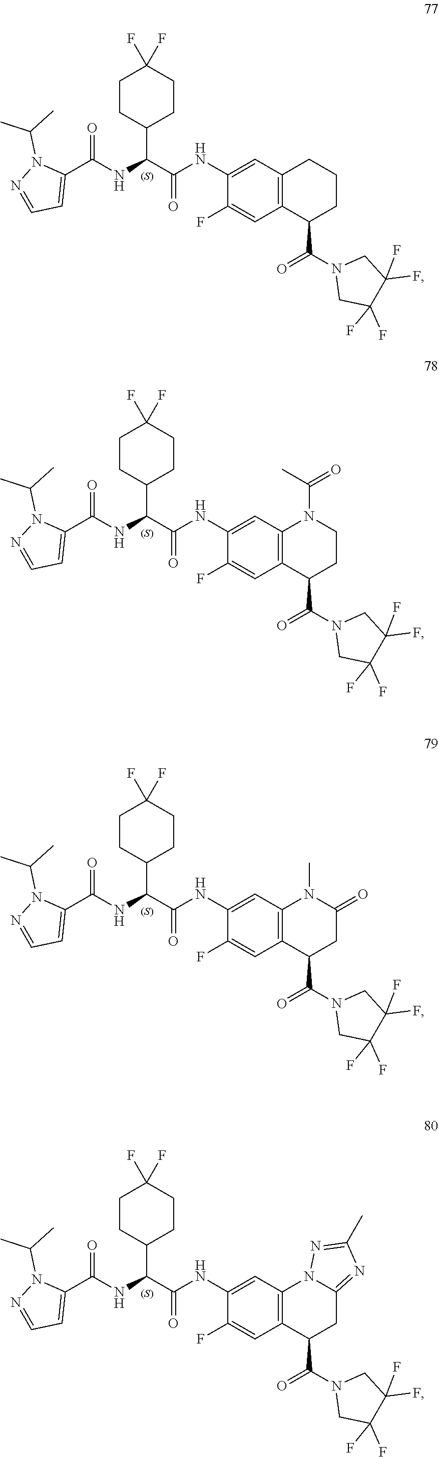 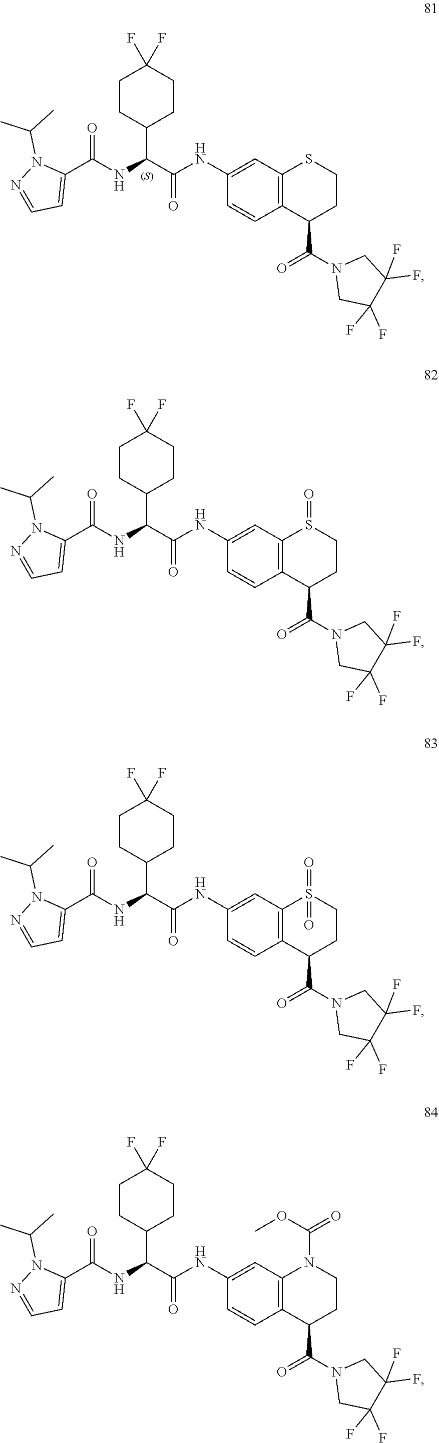 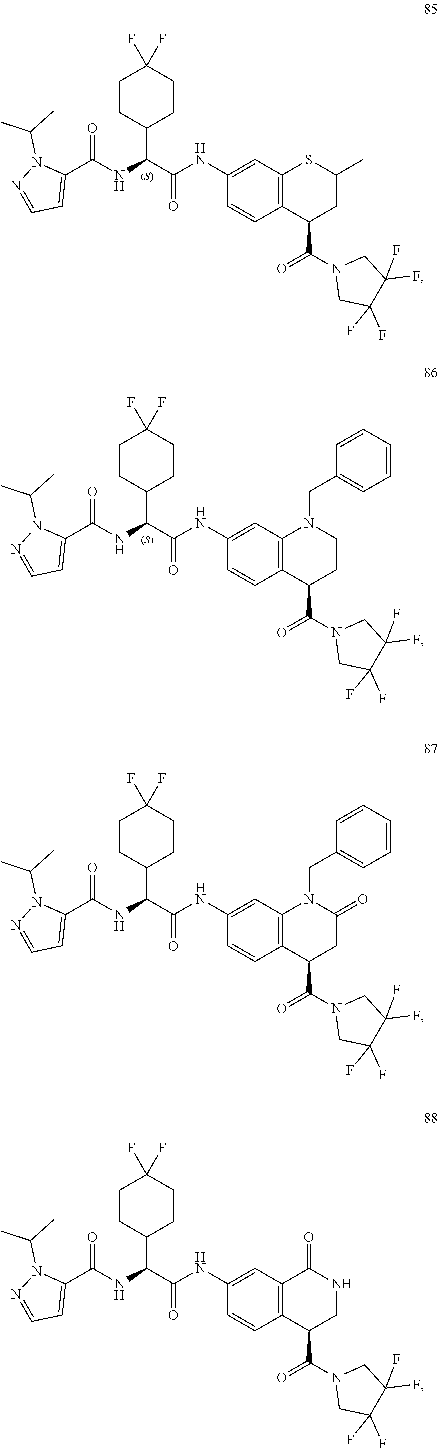 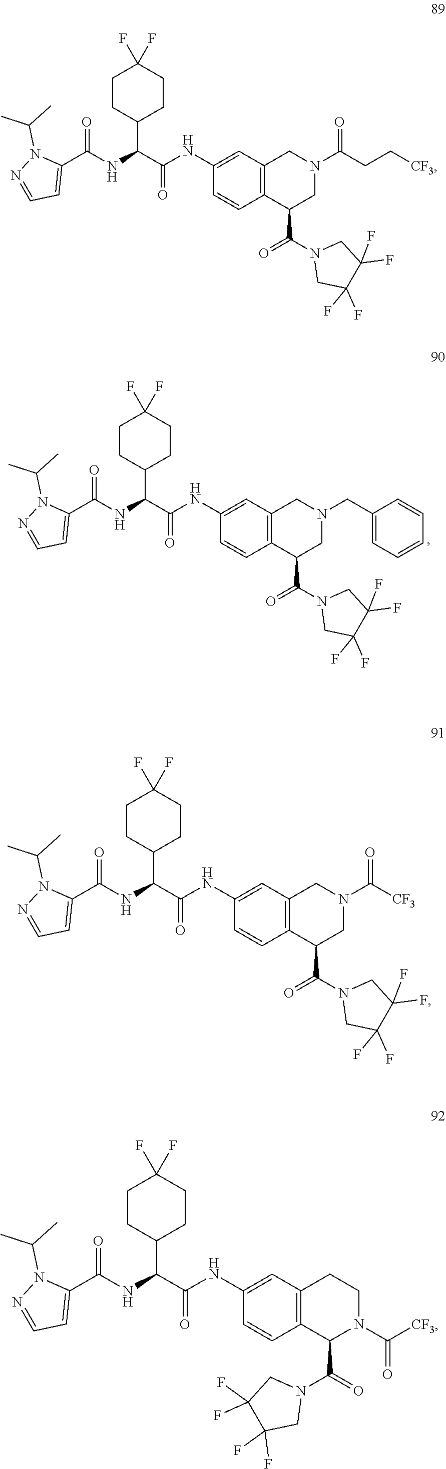 [SMILES: CC(C)n1nccc1C(=O)N[C@H](C(=O)Nc1ccc2c(c1)CN(C(=O)CCC(F)(F)F)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CN(Cc1ccccc1)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CN(C(=O)C(F)(F)F)C[C@H]2C(=O)N1CC(F)(F)C(F)(F)C1)C1CCC(F)(F)CC1 ; CC(C)n1nccc1C(=O)N[C@H](C(=O)Nc1ccc2c(c1)CCN(C(=O)C(F)(F)F)[C@H]2C(=O)N1CC(F)(F)C(F)(F)C1)C1CCC(F)(F)CC1] 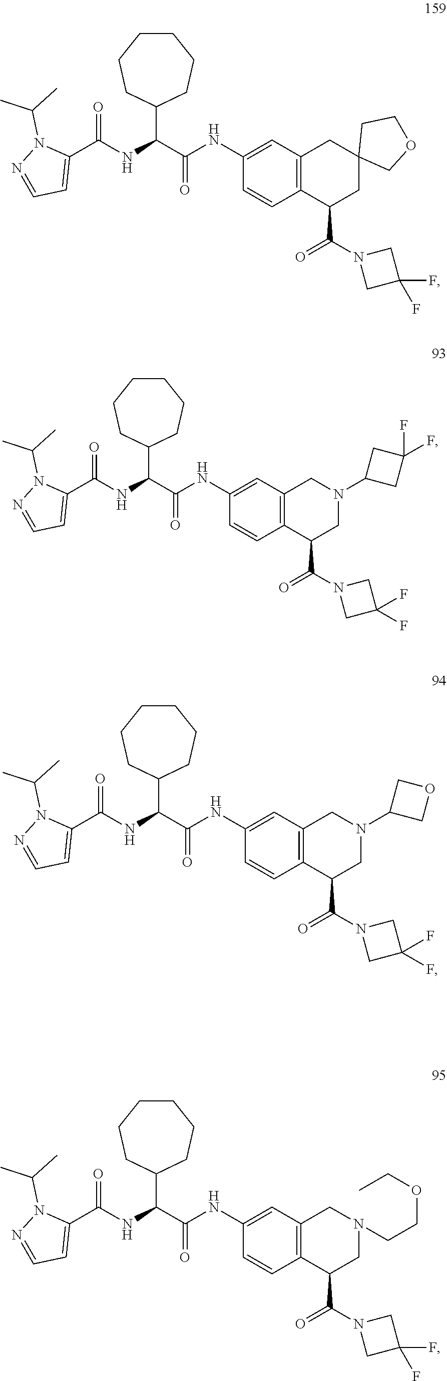 [SMILES: CC(C)n1nccc1C(=O)N[C@H](C(=O)Nc1ccc2c(c1)CC1(CCOC1)C[C@H]2C(=O)N1CC(F)(F)C1)C1CCCCCC1 ; CC(C)n1nccc1C(=O)N[C@H](C(=O)Nc1ccc2c(c1)CN(C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)CN(C1COC1)C[C@H]2C(=O)N1CC(F)(F)C1)C1CCCCCC1 ; CCOCCN1Cc2cc(NC(=O)[C@@H](NC(=O)c3ccnn3C(C)C)C3CCCCCC3)ccc2[C@H](C(=O)N2CC(F)(F)C2)C1] 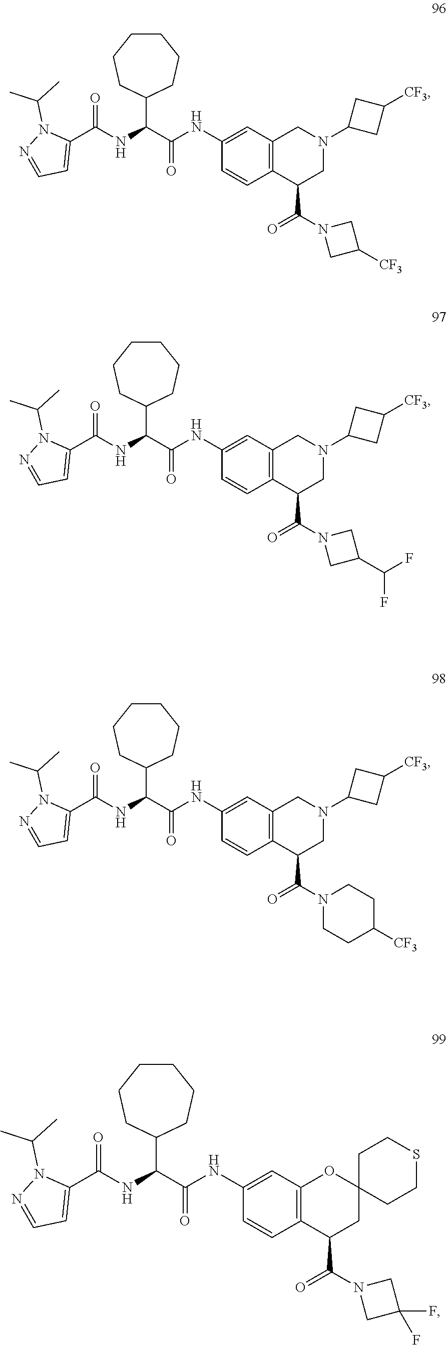 [SMILES: CC(C)n1nccc1C(=O)N[C@H](C(=O)Nc1ccc2c(c1)CN(C1CC(C(F)(F)F)C1)C[C@H]2C(=O)N1CC(C(F)(F)F)C1)C1CCCCCC1 ; CC(C)n1nccc1C(=O)N[C@H](C(=O)Nc1ccc2c(c1)CN(C1CC(C(F)(F)F)C1)C[C@H]2C(=O)N1CC(C(F)F)C1)C1CCCCCC1 ; CC(C)n1nccc1C(=O)N[C@H](C(=O)Nc1ccc2c(c1)CN(C1CC(C(F)(F)F)C1)C[C@H]2C(=O)N1CCC(C(F)(F)F)CC1)C1CCCCCC1 ; CC(C)n1nccc1C(=O)N[C@H](C(=O)Nc1ccc2c(c1)OC1(CCSCC1)C[C@H]2C(=O)N1CC(F)(F)C1)C1CCCCCC1] 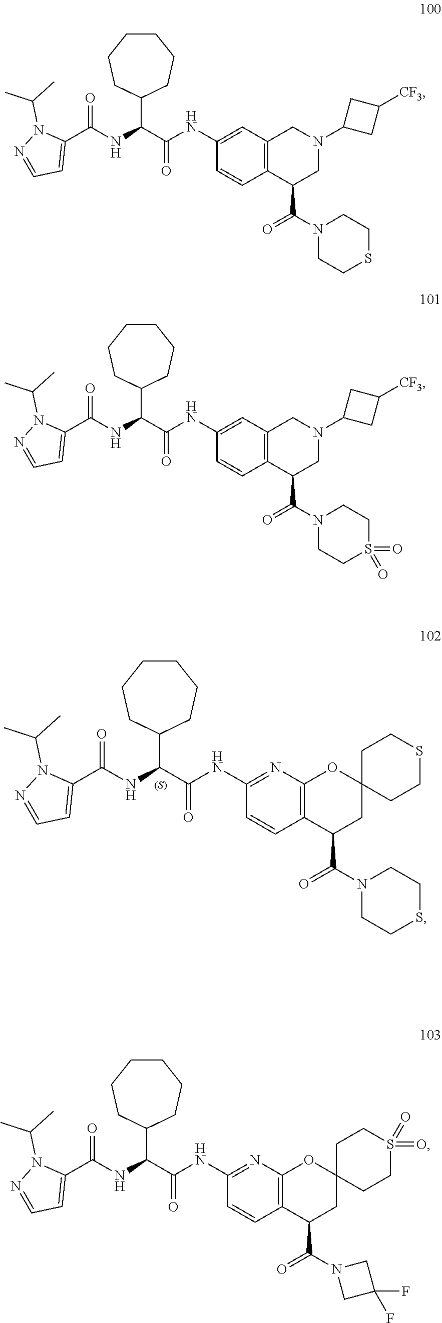 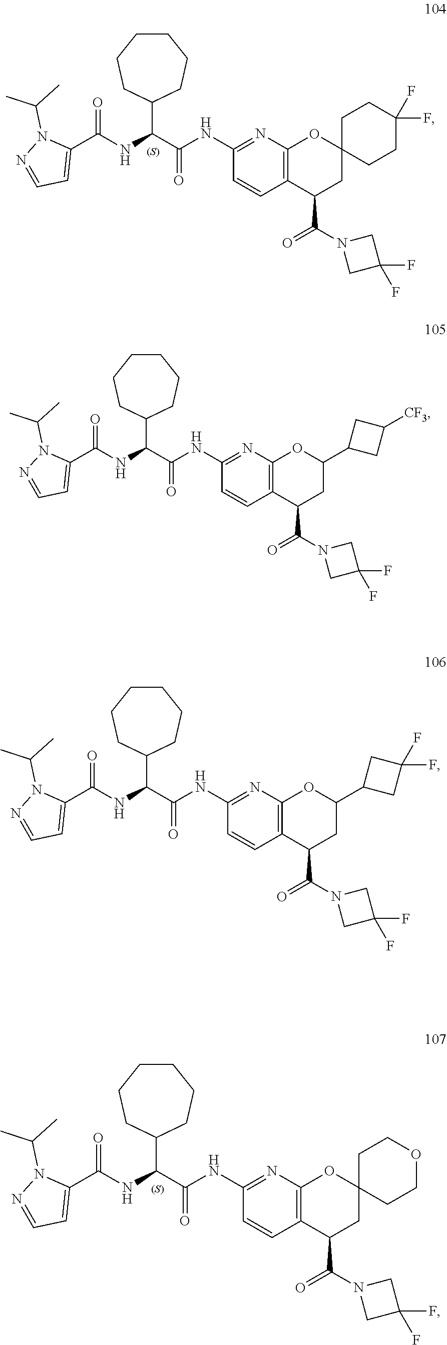 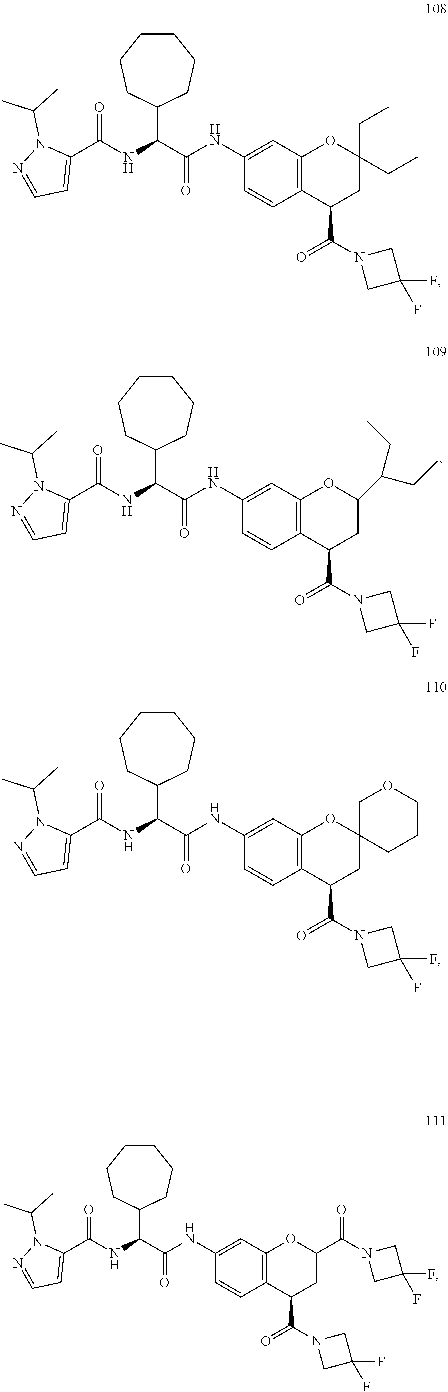 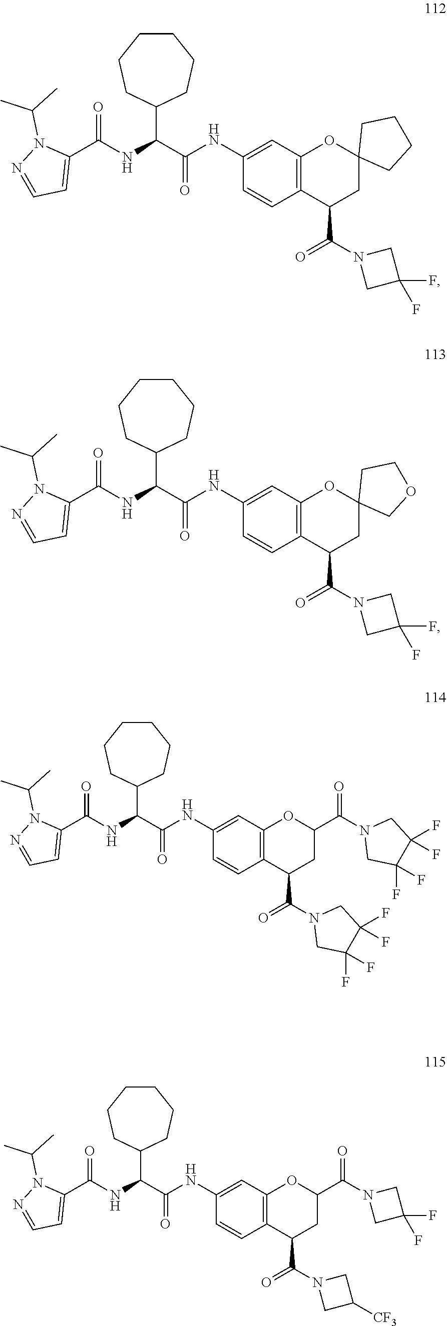 [SMILES: CC(C)n1nccc1C(=O)N[C@H](C(=O)Nc1ccc2c(c1)OC1(CCCC1)C[C@H]2C(=O)N1CC(F)(F)C1)C1CCCCCC1 ; CC(C)n1nccc1C(=O)N[C@H](C(=O)Nc1ccc2c(c1)OC1(CCOC1)C[C@H]2C(=O)N1CC(F)(F)C1)C1CCCCCC1 ; CC(C)n1nccc1C(=O)N[C@H](C(=O)Nc1ccc2c(c1)OC(C(=O)N1CC(F)(F)C(F)(F)C1)C[C@H]2C(=O)N1CC(F)(F)C(F)(F)C1)C1CCCCCC1 ; CC(C)n1nccc1C(=O)N[C@H](C(=O)Nc1ccc2c(c1)OC(C(=O)N1CC(F)(F)C1)C[C@H]2C(=O)N1CC(C(F)(F)F)C1)C1CCCCCC1] 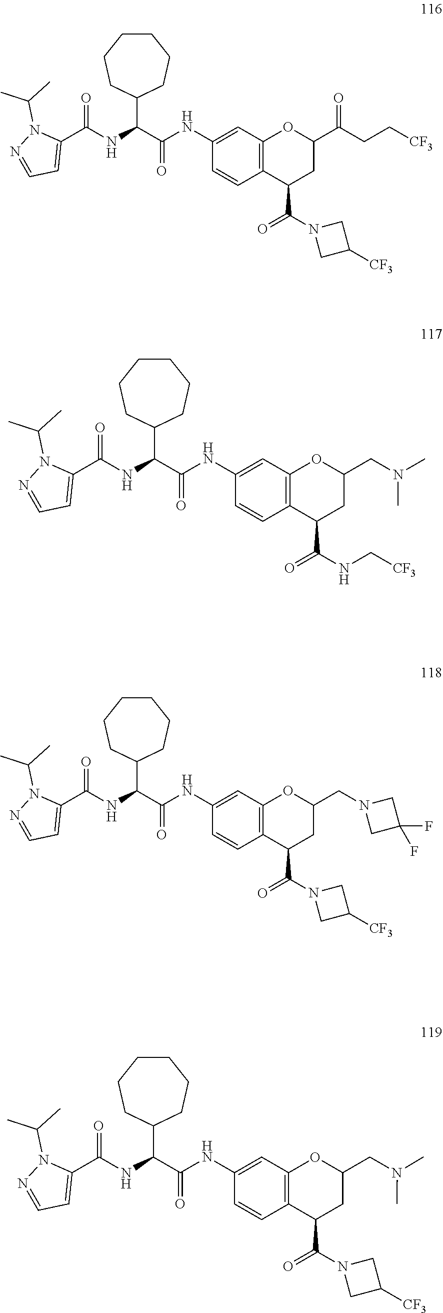 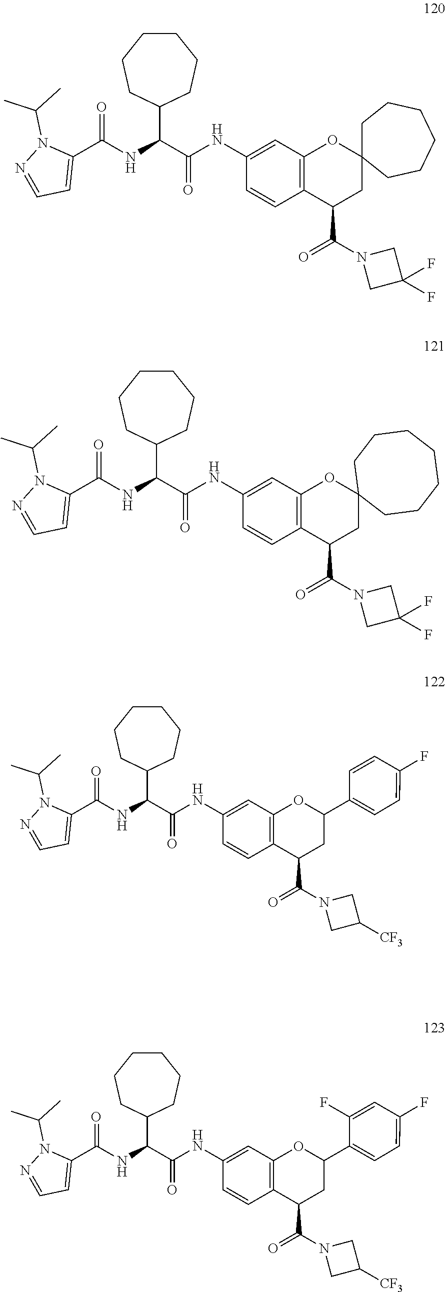 [SMILES: CC(C)n1nccc1C(=O)N[C@H](C(=O)Nc1ccc2c(c1)OC1(CCCCCC1)C[C@H]2C(=O)N1CC(F)(F)C1)C1CCCCCC1 ; CC(C)n1nccc1C(=O)N[C@H](C(=O)Nc1ccc2c(c1)OC1(CCCCCCC1)C[C@H]2C(=O)N1CC(F)(F)C1)C1CCCCCC1 ; CC(C)n1nccc1C(=O)N[C@H](C(=O)Nc1ccc2c(c1)OC(c1ccc(F)cc1)C[C@H]2C(=O)N1CC(C(F)(F)F)C1)C1CCCCCC1 ; CC(C)n1nccc1C(=O)N[C@H](C(=O)Nc1ccc2c(c1)OC(c1ccc(F)cc1F)C[C@H]2C(=O)N1CC(C(F)(F)F)C1)C1CCCCCC1] 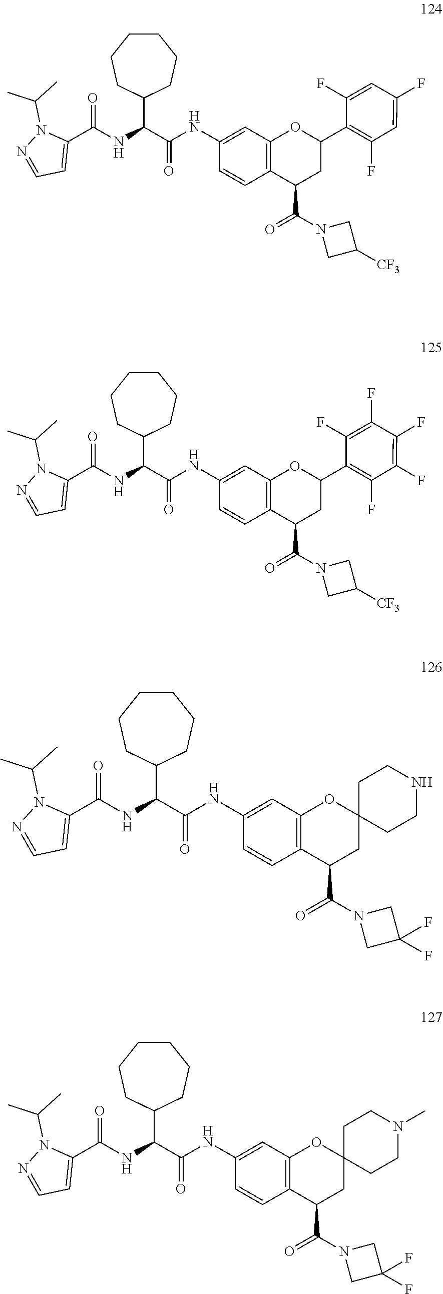 [SMILES: CC(C)n1nccc1C(=O)N[C@H](C(=O)Nc1ccc2c(c1)OC(c1c(F)cc(F)cc1F)C[C@H]2C(=O)N1CC(C(F)(F)F)C1)C1CCCCCC1 ; CC(C)n1nccc1C(=O)N[C@H](C(=O)Nc1ccc2c(c1)OC(c1c(F)c(F)c(F)c(F)c1F)C[C@H]2C(=O)N1CC(C(F)(F)F)C1)C1CCCCCC1 ; CC(C)n1nccc1C(=O)N[C@H](C(=O)Nc1ccc2c(c1)OC1(CCNCC1)C[C@H]2C(=O)N1CC(F)(F)C1)C1CCCCCC1 ; CC(C)n1nccc1C(=O)N[C@H](C(=O)Nc1ccc2c(c1)OC1(CCN(C)CC1)C[C@H]2C(=O)N1CC(F)(F)C1)C1CCCCCC1] 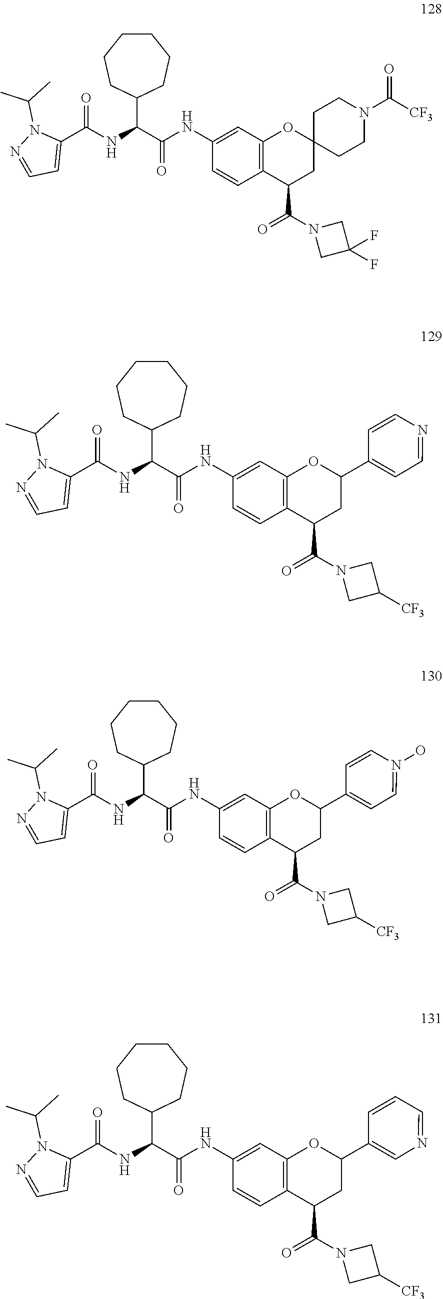 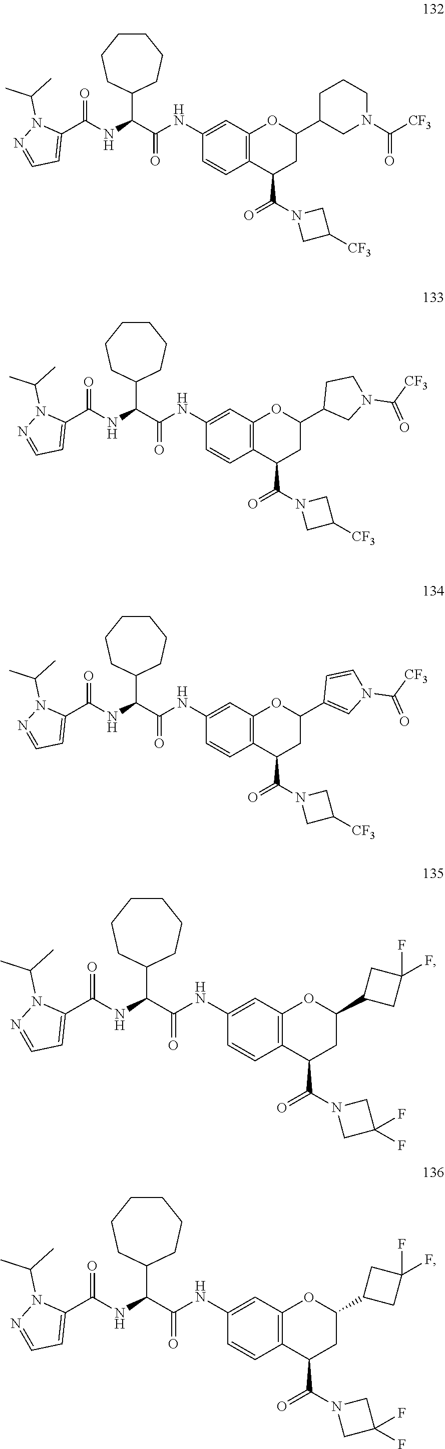 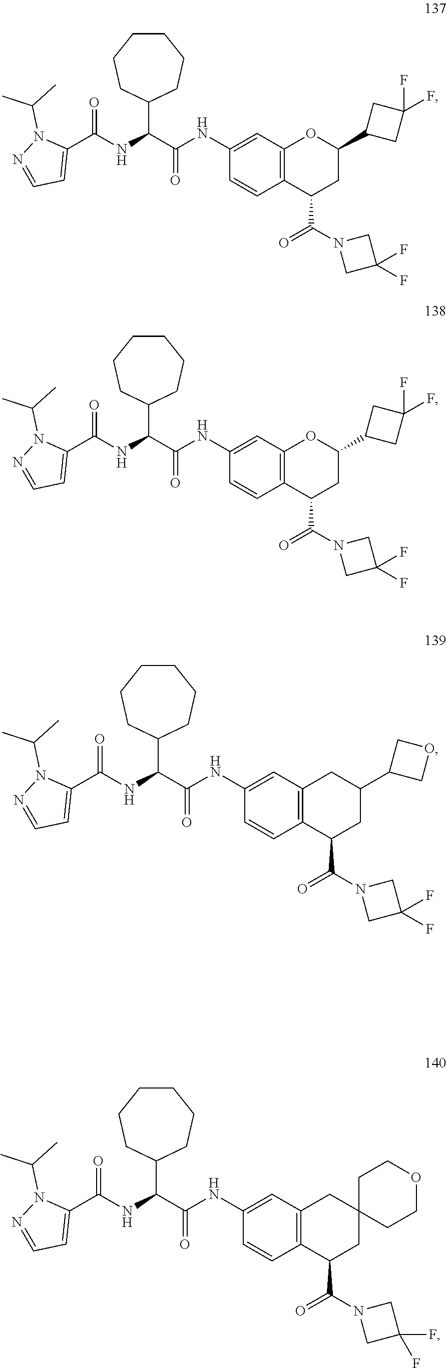 [SMILES: CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1 ; CC(C)n1nccc1C(=O)N[C@H](C(=O)Nc1ccc2c(c1)CC(C1COC1)C[C@H]2C(=O)N1CC(F)(F)C1)C1CCCCCC1 ; CC(C)n1nccc1C(=O)N[C@H](C(=O)Nc1ccc2c(c1)CC1(CCOCC1)C[C@H]2C(=O)N1CC(F)(F)C1)C1CCCCCC1] 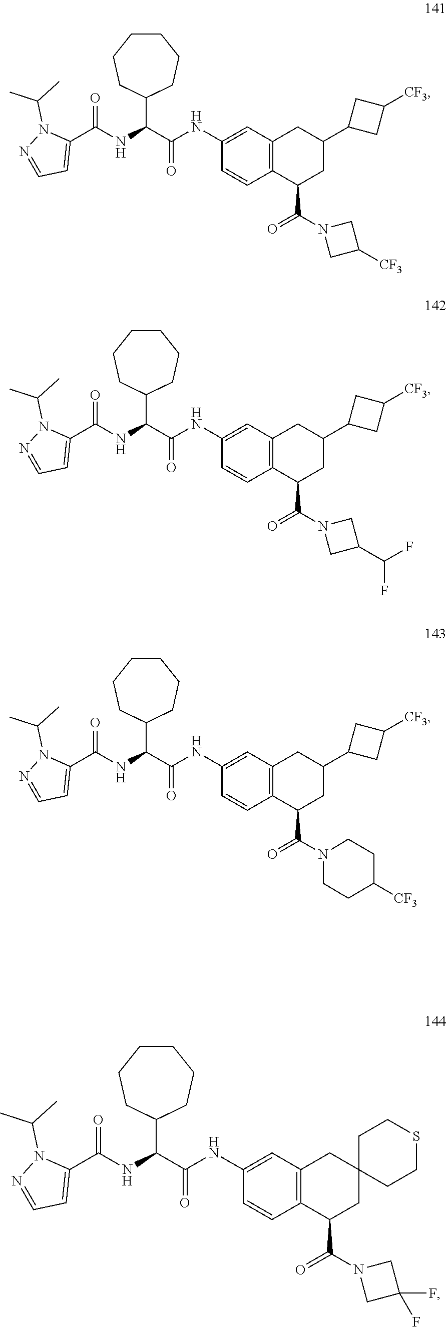 [SMILES: CC(C)n1nccc1C(=O)N[C@H](C(=O)Nc1ccc2c(c1)CC(C1CC(C(F)(F)F)C1)C[C@H]2C(=O)N1CC(C(F)(F)F)C1)C1CCCCCC1 ; CC(C)n1nccc1C(=O)N[C@H](C(=O)Nc1ccc2c(c1)CC(C1CC(C(F)(F)F)C1)C[C@H]2C(=O)N1CC(C(F)F)C1)C1CCCCCC1 ; CC(C)n1nccc1C(=O)N[C@H](C(=O)Nc1ccc2c(c1)CC(C1CC(C(F)(F)F)C1)C[C@H]2C(=O)N1CCC(C(F)(F)F)CC1)C1CCCCCC1 ; CC(C)n1nccc1C(=O)N[C@H](C(=O)Nc1ccc2c(c1)CC1(CCSCC1)C[C@H]2C(=O)N1CC(F)(F)C1)C1CCCCCC1] 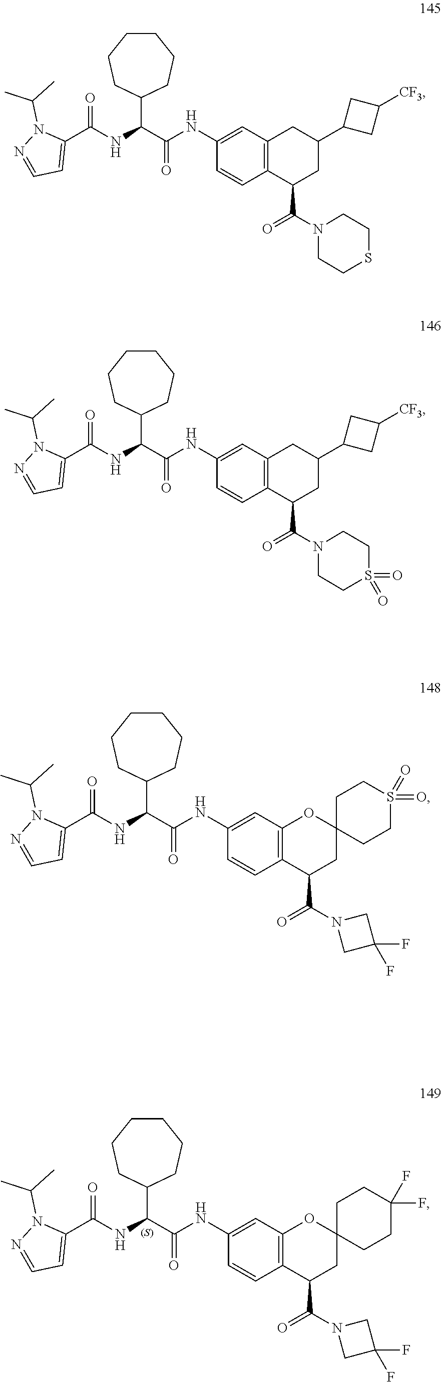 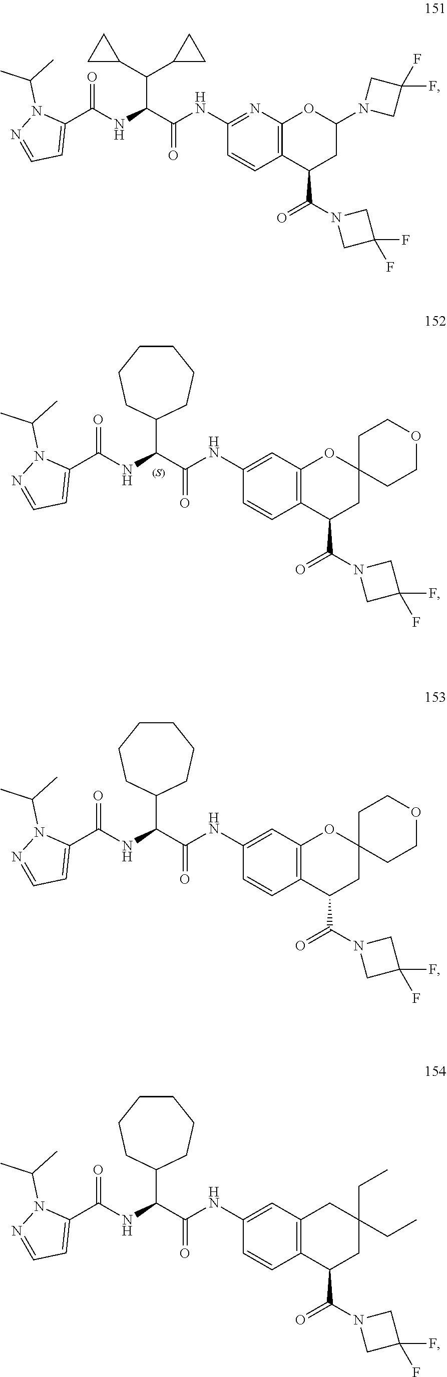 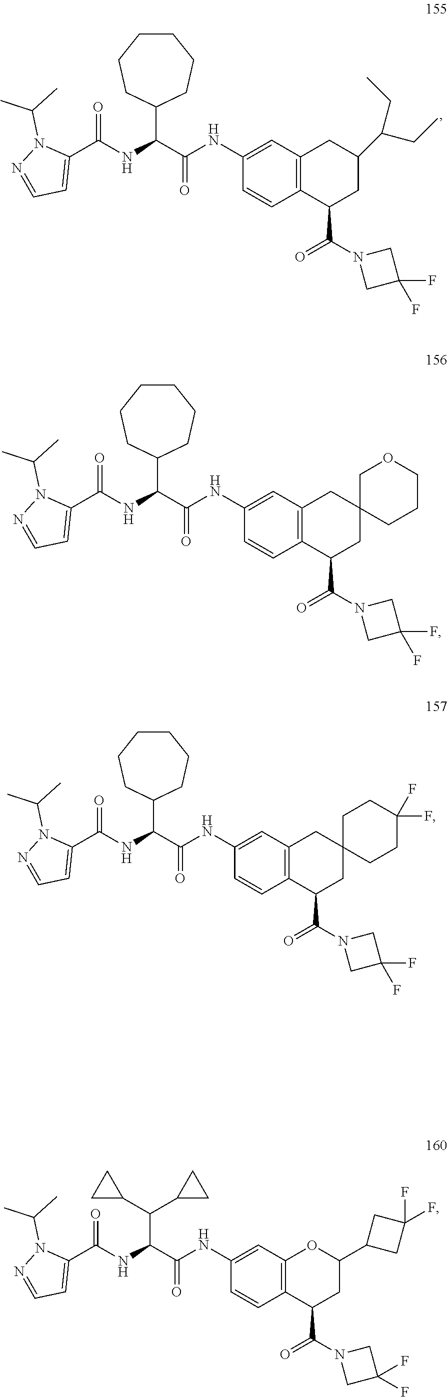 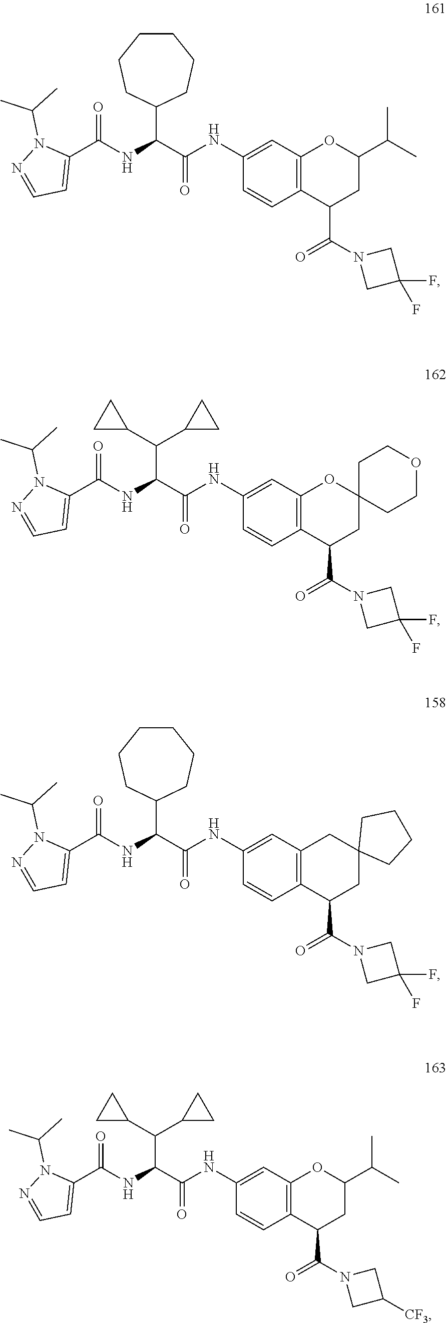 [SMILES: CC(C)C1CC(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCOCC1)C[C@H]2C(=O)N1CC(F)(F)C1)C(C1CC1)C1CC1 ; CC(C)n1nccc1C(=O)N[C@H](C(=O)Nc1ccc2c(c1)CC1(CCCC1)C[C@H]2C(=O)N1CC(F)(F)C1)C1CCCCCC1 ; CC(C)C1C[C@@H](C(=O)N2CC(C(F)(F)F)C2)c2ccc(NC(=O)[C@@H](NC(=O)c3ccnn3C(C)C)C(C3CC3)C3CC3)cc2O1] 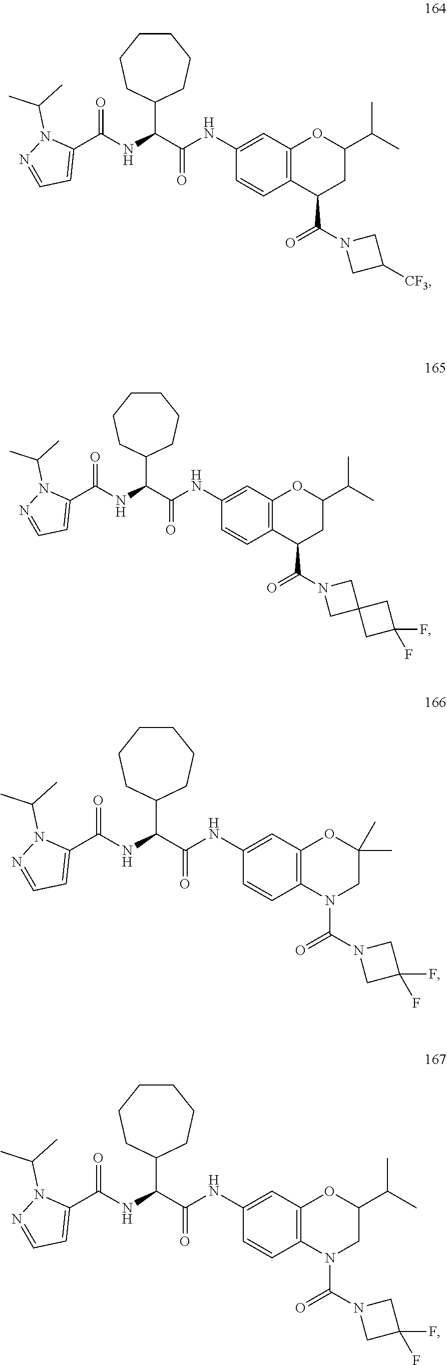 [SMILES: CC(C)C1C[C@@H](C(=O)N2CC(C(F)(F)F)C2)c2ccc(NC(=O)[C@@H](NC(=O)c3ccnn3C(C)C)C3CCCCCC3)cc2O1 ; CC(C)C1C[C@@H](C(=O)N2CC3(C2)CC(F)(F)C3)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC(C)(C)CN2C(=O)N1CC(F)(F)C1)C1CCCCCC1 ; CC(C)C1CN(C(=O)N2CC(F)(F)C2)c2ccc(NC(=O)[C@@H](NC(=O)c3ccnn3C(C)C)C3CCCCCC3)cc2O1] 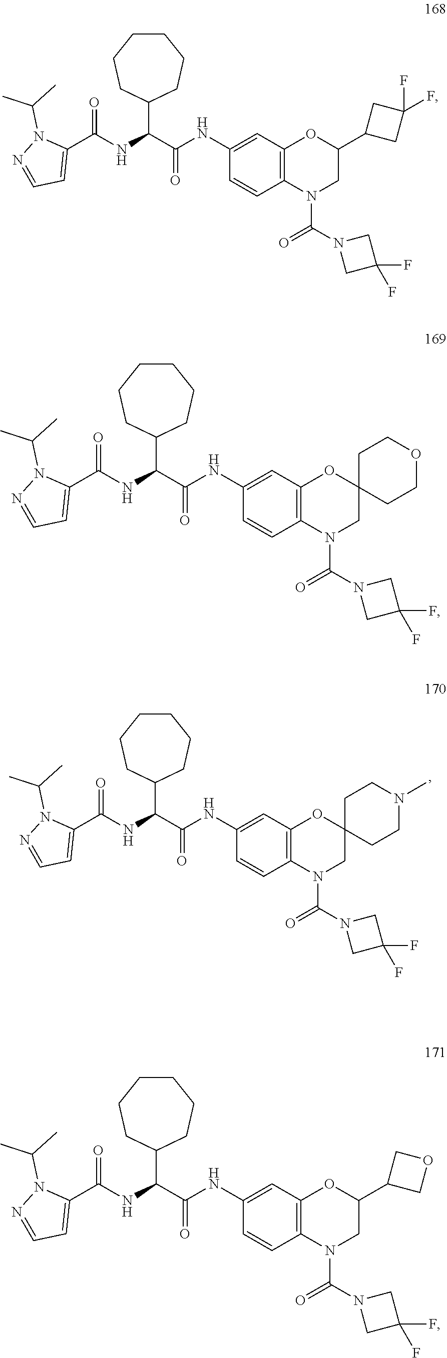 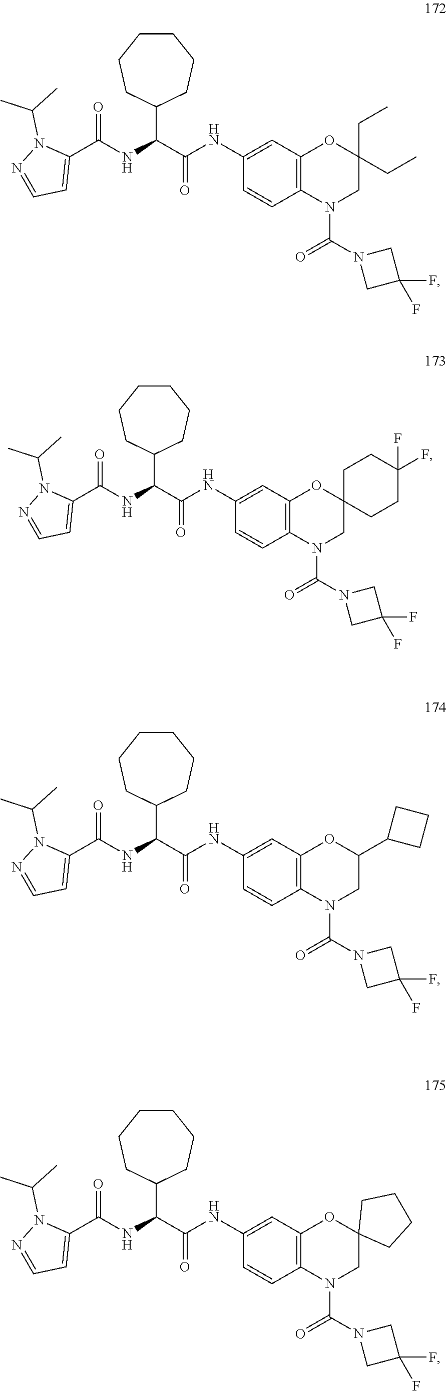 [SMILES: CCC1(CC)CN(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCC(F)(F)CC1)CN2C(=O)N1CC(F)(F)C1)C1CCCCCC1 ; CC(C)n1nccc1C(=O)N[C@H](C(=O)Nc1ccc2c(c1)OC(C1CCC1)CN2C(=O)N1CC(F)(F)C1)C1CCCCCC1 ; CC(C)n1nccc1C(=O)N[C@H](C(=O)Nc1ccc2c(c1)OC1(CCCC1)CN2C(=O)N1CC(F)(F)C1)C1CCCCCC1] 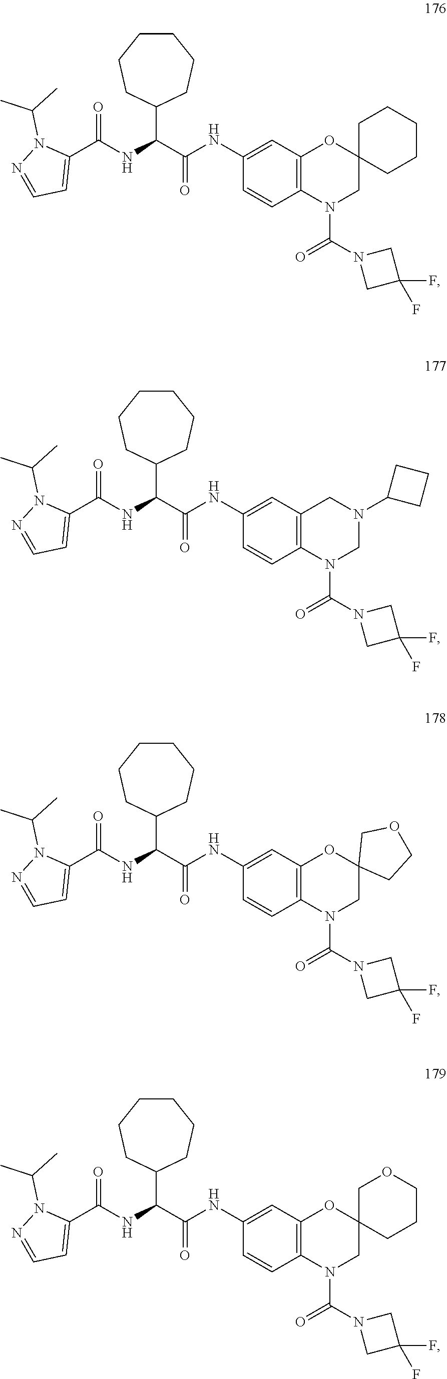 [SMILES: CC(C)n1nccc1C(=O)N[C@H](C(=O)Nc1ccc2c(c1)OC1(CCCCC1)CN2C(=O)N1CC(F)(F)C1)C1CCCCCC1 ; CC(C)n1nccc1C(=O)N[C@H](C(=O)Nc1ccc2c(c1)CN(C1CCC1)CN2C(=O)N1CC(F)(F)C1)C1CCCCCC1 ; CC(C)n1nccc1C(=O)N[C@H](C(=O)Nc1ccc2c(c1)OC1(CCOC1)CN2C(=O)N1CC(F)(F)C1)C1CCCCCC1 ; CC(C)n1nccc1C(=O)N[C@H](C(=O)Nc1ccc2c(c1)OC1(CCCOC1)CN2C(=O)N1CC(F)(F)C1)C1CCCCCC1] 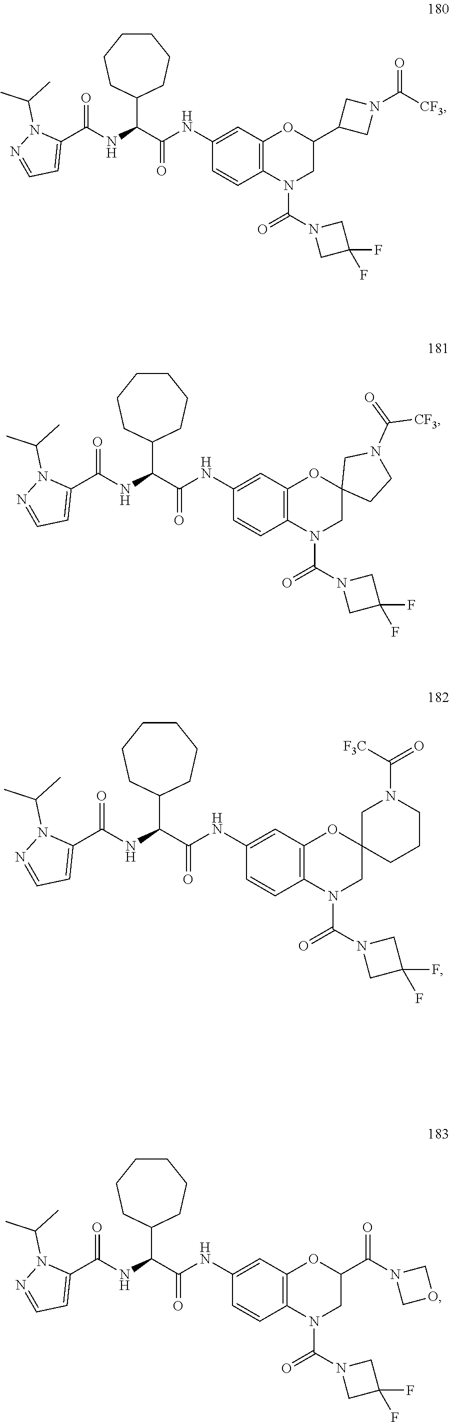 [SMILES: CC(C)n1nccc1C(=O)N[C@H](C(=O)Nc1ccc2c(c1)OC(C1CN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC(C(=O)N1COC1)CN2C(=O)N1CC(F)(F)C1)C1CCCCCC1] 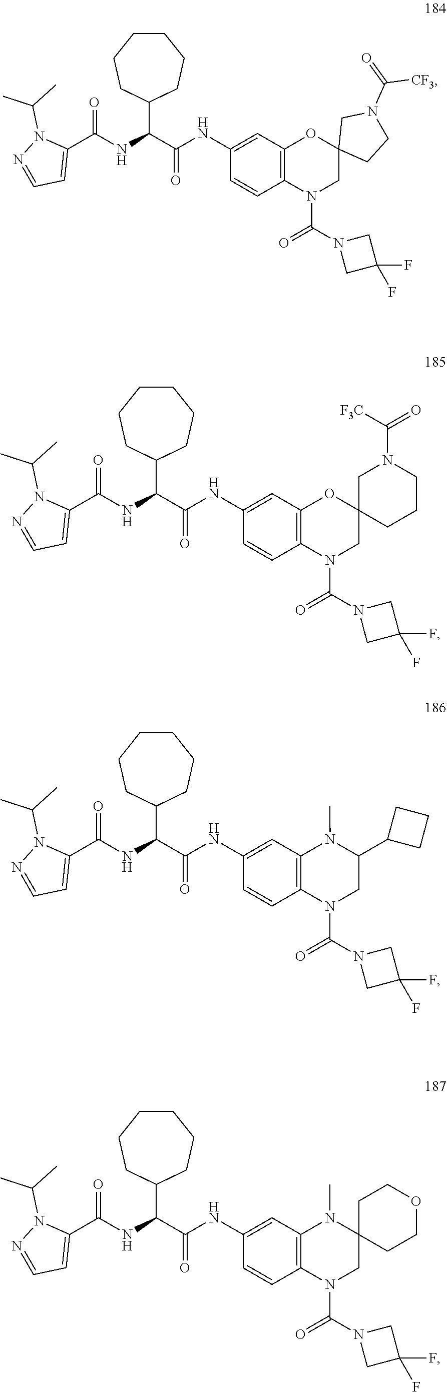 [SMILES: CC(C)n1nccc1C(=O)N[C@H](C(=O)Nc1ccc2c(c1)OC1(CCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)OC1(CCCN(C(=O)C(F)(F)F)C1)CN2C(=O)N1CC(F)(F)C1)C1CCCCCC1 ; CC(C)n1nccc1C(=O)N[C@H](C(=O)Nc1ccc2c(c1)N(C)C(C1CCC1)CN2C(=O)N1CC(F)(F)C1)C1CCCCCC1 ; CC(C)n1nccc1C(=O)N[C@H](C(=O)Nc1ccc2c(c1)N(C)C1(CCOCC1)CN2C(=O)N1CC(F)(F)C1)C1CCCCCC1] 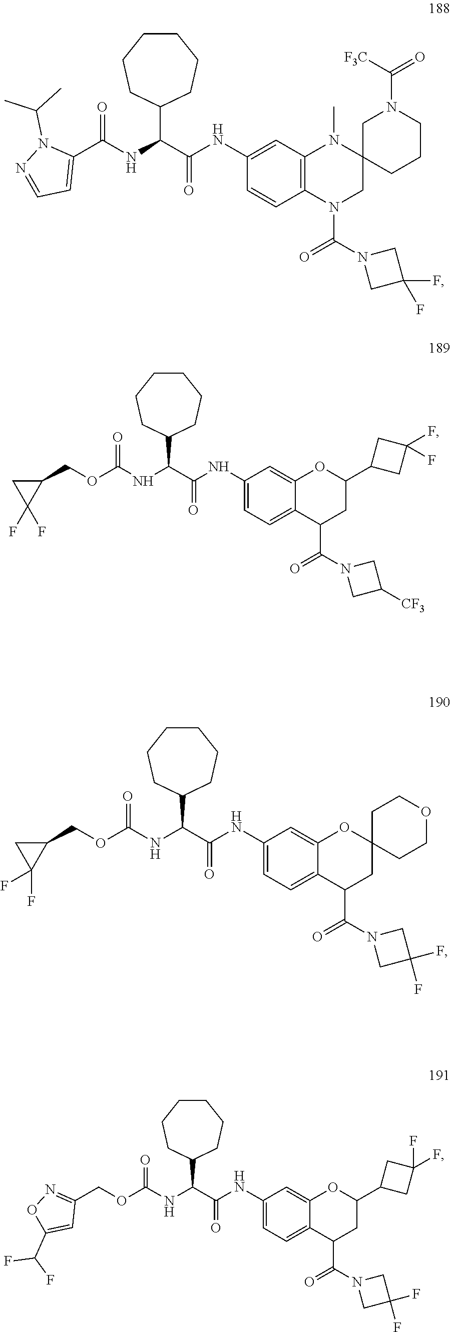 [SMILES: CC(C)n1nccc1C(=O)N[C@H](C(=O)Nc1ccc2c(c1)N(C)C1(CCCN(C(=O)C(F)(F)F)C1)CN2C(=O)N1CC(F)(F)C1)C1CCCCCC1 ; O=C(N[C@H](C(=O)Nc1ccc2c(c1)OC(C1CC(F)(F)C1)CC2C(=O)N1CC(C(F)(F)F)C1)C1CCCCCC1)OC[C@@H]1CC1(F)F ; O=C(N[C@H](C(=O)Nc1ccc2c(c1)OC1(CCOCC1)CC2C(=O)N1CC(F)(F)C1)C1CCCCCC1)OC[C@@H]1CC1(F)F ; O=C(N[C@H](C(=O)Nc1ccc2c(c1)OC(C1CC(F)(F)C1)CC2C(=O)N1CC(F)(F)C1)C1CCCCCC1)OCc1cc(C(F)F)on1] 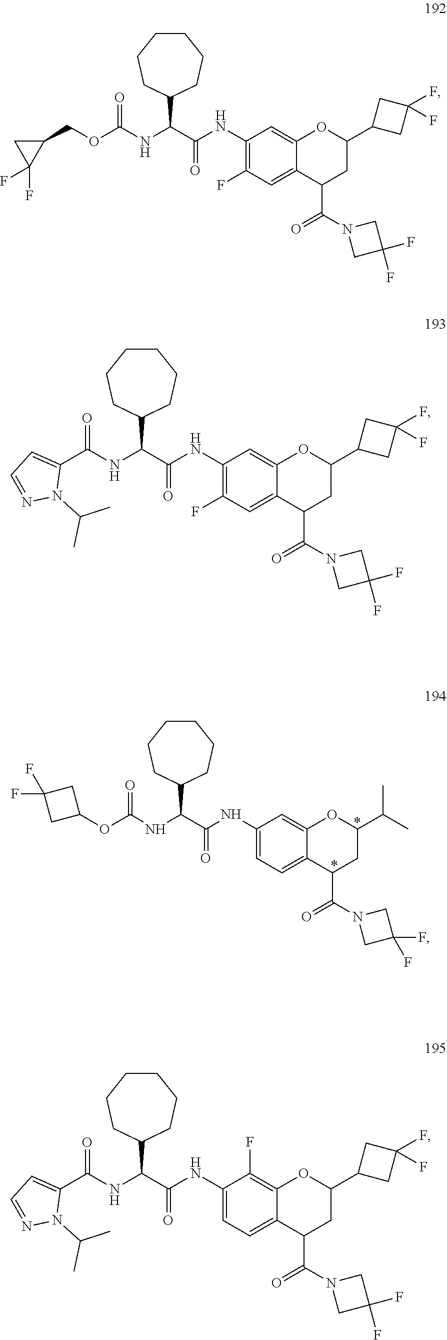 [SMILES: O=C(N[C@H](C(=O)Nc1cc2c(cc1F)C(C(=O)N1CC(F)(F)C1)CC(C1CC(F)(F)C1)O2)C1CCCCCC1)OC[C@@H]1CC1(F)F ; CC(C)n1nccc1C(=O)N[C@H](C(=O)Nc1cc2c(cc1F)C(C(=O)N1CC(F)(F)C1)CC(C1CC(F)(F)C1)O2)C1CCCCCC1 ; CC(C)C1CC(C(=O)N2CC(F)(F)C2)c2ccc(NC(=O)[C@@H](NC(=O)OC3CC(F)(F)C3)C3CCCCCC3)cc2O1 ; CC(C)n1nccc1C(=O)N[C@H](C(=O)Nc1ccc2c(c1F)OC(C1CC(F)(F)C1)CC2C(=O)N1CC(F)(F)C1)C1CCCCCC1] 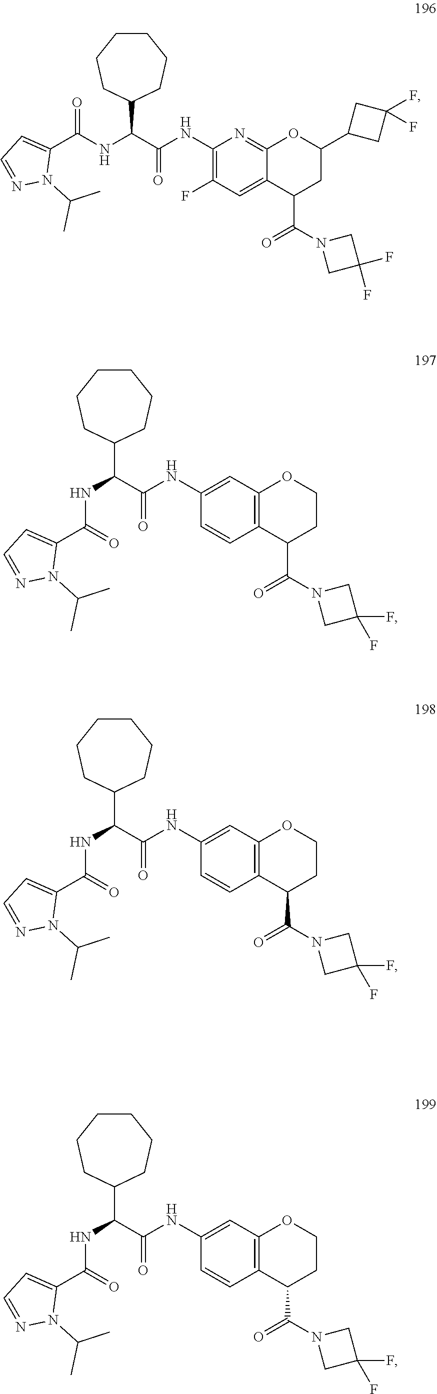 [SMILES: CC(C)n1nccc1C(=O)N[C@H](C(=O)Nc1nc2c(cc1F)C(C(=O)N1CC(F)(F)C1)CC(C1CC(F)(F)C1)O2)C1CCCCCC1 ; CC(C)n1nccc1C(=O)N[C@H](C(=O)Nc1ccc2c(c1)OCCC2C(=O)N1CC(F)(F)C1)C1CCCCCC1 ; CC(C)n1nccc1C(=O)N[C@H](C(=O)Nc1ccc2c(c1)OCC[C@H]2C(=O)N1CC(F)(F)C1)C1CCCCCC1 ; CC(C)n1nccc1C(=O)N[C@H](C(=O)Nc1ccc2c(c1)OCC[C@@H]2C(=O)N1CC(F)(F)C1)C1CCCCCC1] 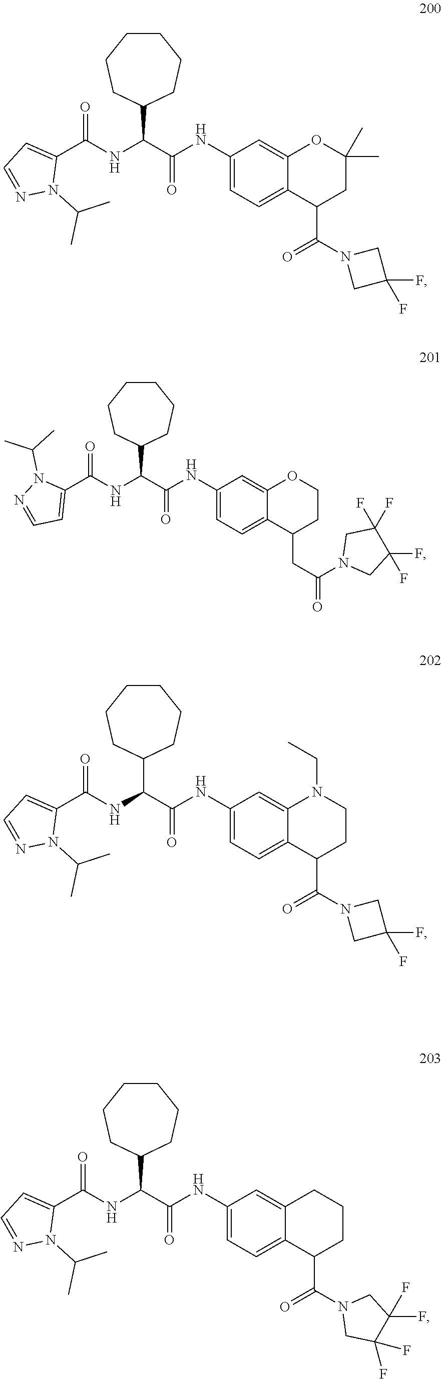 [SMILES: CC(C)n1nccc1C(=O)N[C@H](C(=O)Nc1ccc2c(c1)OC(C)(C)CC2C(=O)N1CC(F)(F)C1)C1CCCCCC1 ; CC(C)n1nccc1C(=O)N[C@H](C(=O)Nc1ccc2c(c1)OCCC2CC(=O)N1CC(F)(F)C(F)(F)C1)C1CCCCCC1 ; CCN1CCC(C(=O)N2CC(F)(F)C2)c2ccc(NC(=O)[C@@H](NC(=O)c3ccnn3C(C)C)C3CCCCCC3)cc21 ; CC(C)n1nccc1C(=O)N[C@H](C(=O)Nc1ccc2c(c1)CCCC2C(=O)N1CC(F)(F)C(F)(F)C1)C1CCCCCC1] 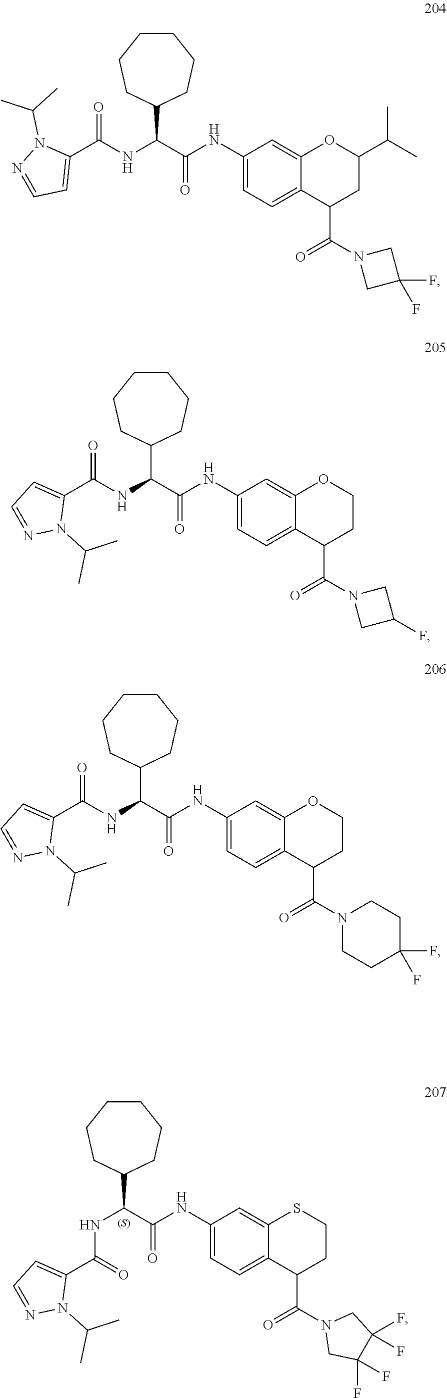 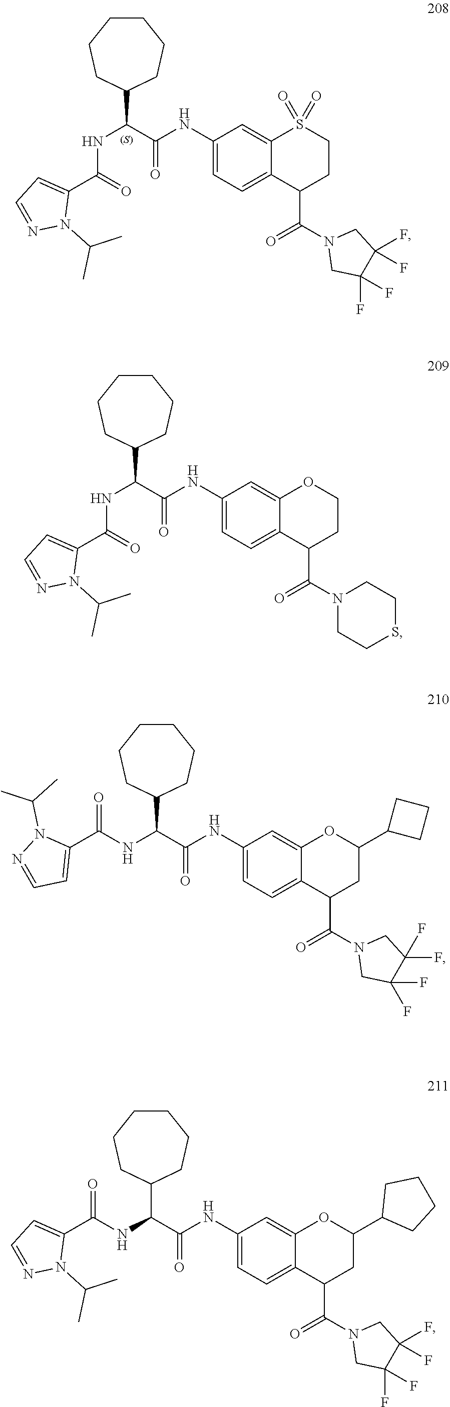 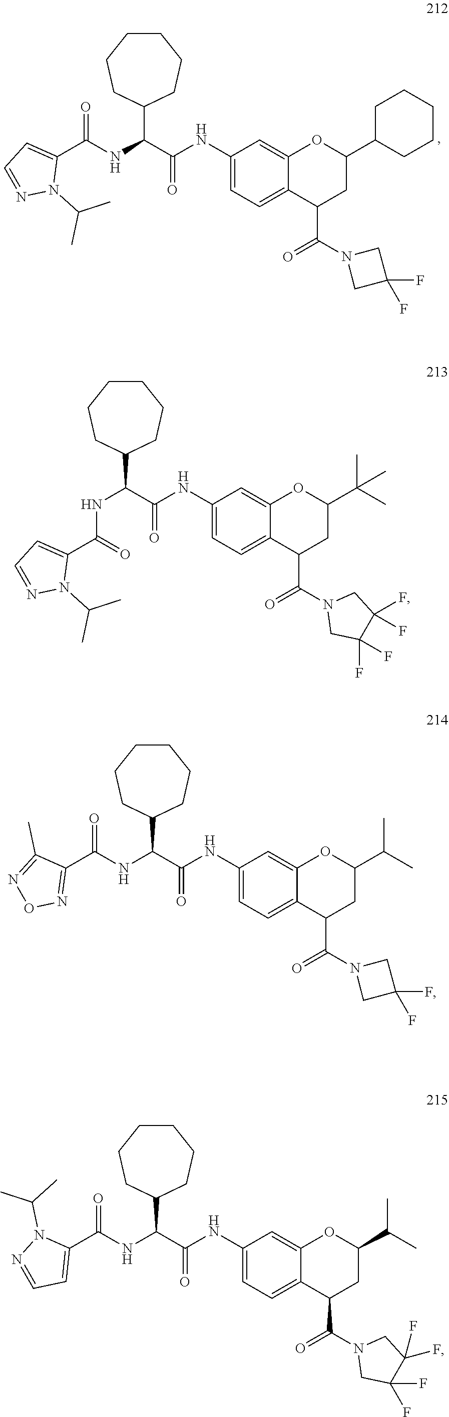 [SMILES: CC(C)n1nccc1C(=O)N[C@H](C(=O)Nc1ccc2c(c1)OC(C1CCCCC1)CC2C(=O)N1CC(F)(F)C1)C1CCCCCC1 ; CC(C)n1nccc1C(=O)N[C@H](C(=O)Nc1ccc2c(c1)OC(C(C)(C)C)CC2C(=O)N1CC(F)(F)C(F)(F)C1)C1CCCCCC1 ; Cc1nonc1C(=O)N[C@H](C(=O)Nc1ccc2c(c1)OC(C(C)C)CC2C(=O)N1CC(F)(F)C1)C1CCCCCC1 ; CC(C)[C@H]1C[C@@H](C(=O)N2CC(F)(F)C(F)(F)C2)c2ccc(NC(=O)[C@@H](NC(=O)c3ccnn3C(C)C)C3CCCCCC3)cc2O1] 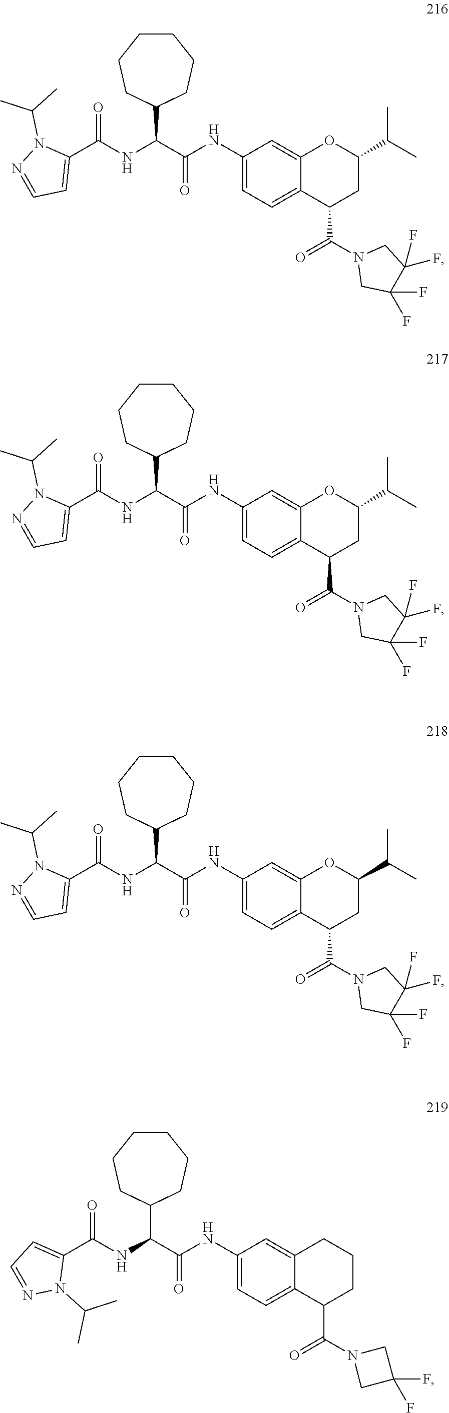 [SMILES: CC(C)[C@@H]1C[C@H](C(=O)N2CC(F)(F)C(F)(F)C2)c2ccc(NC(=O)[C@@H](NC(=O)c3ccnn3C(C)C)C3CCCCCC3)cc2O1 ; CC(C)[C@@H]1C[C@@H](C(=O)N2CC(F)(F)C(F)(F)C2)c2ccc(NC(=O)[C@@H](NC(=O)c3ccnn3C(C)C)C3CCCCCC3)cc2O1 ; CC(C)[C@H]1C[C@H](C(=O)N2CC(F)(F)C(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)CCCC2C(=O)N1CC(F)(F)C1)C1CCCCCC1] 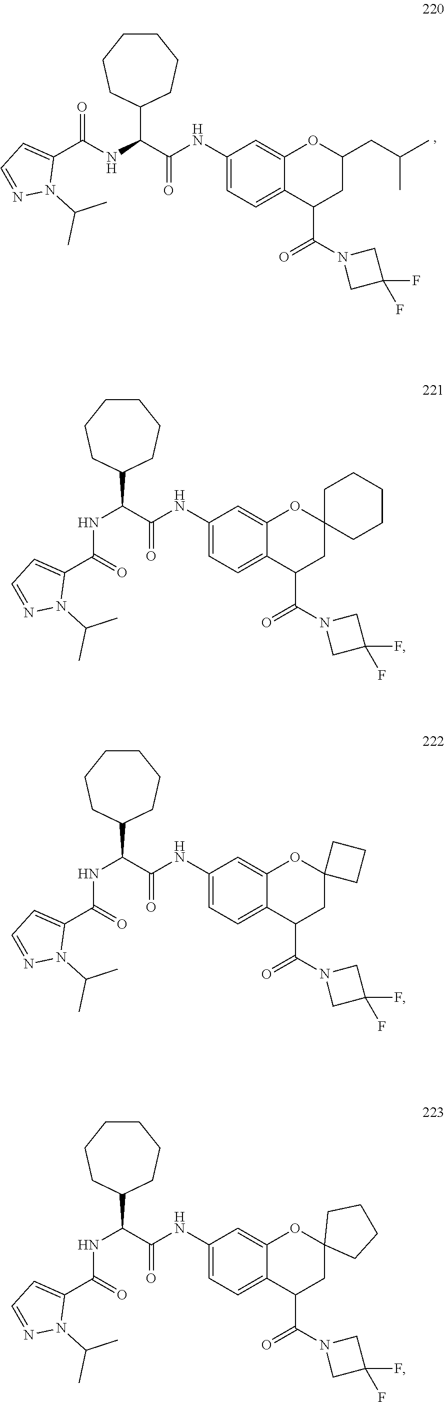 [SMILES: CC(C)CC1CC(C(=O)N2CC(F)(F)C2)c2ccc(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC1(CCCCC1)CC2C(=O)N1CC(F)(F)C1)C1CCCCCC1 ; CC(C)n1nccc1C(=O)N[C@H](C(=O)Nc1ccc2c(c1)OC1(CCC1)CC2C(=O)N1CC(F)(F)C1)C1CCCCCC1 ; CC(C)n1nccc1C(=O)N[C@H](C(=O)Nc1ccc2c(c1)OC1(CCCC1)CC2C(=O)N1CC(F)(F)C1)C1CCCCCC1] 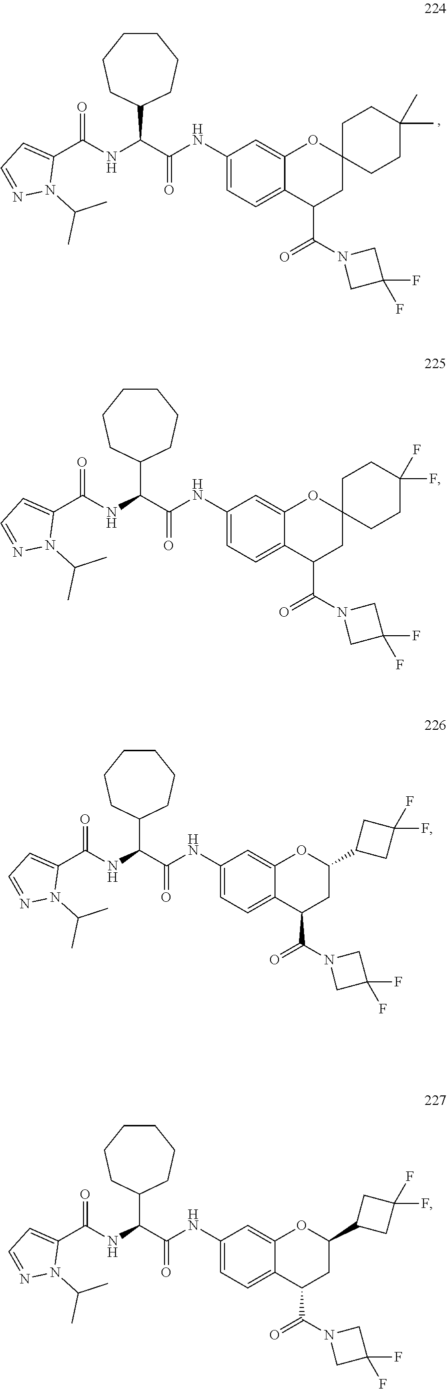 [SMILES: CC(C)n1nccc1C(=O)N[C@H](C(=O)Nc1ccc2c(c1)OC1(CCC(C)(C)CC1)CC2C(=O)N1CC(F)(F)C1)C1CCCCCC1 ; CC(C)n1nccc1C(=O)N[C@H](C(=O)Nc1ccc2c(c1)OC1(CCC(F)(F)CC1)CC2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)O[C@@H](C1CC(F)(F)C1)C[C@@H]2C(=O)N1CC(F)(F)C1)C1CCCCCC1] 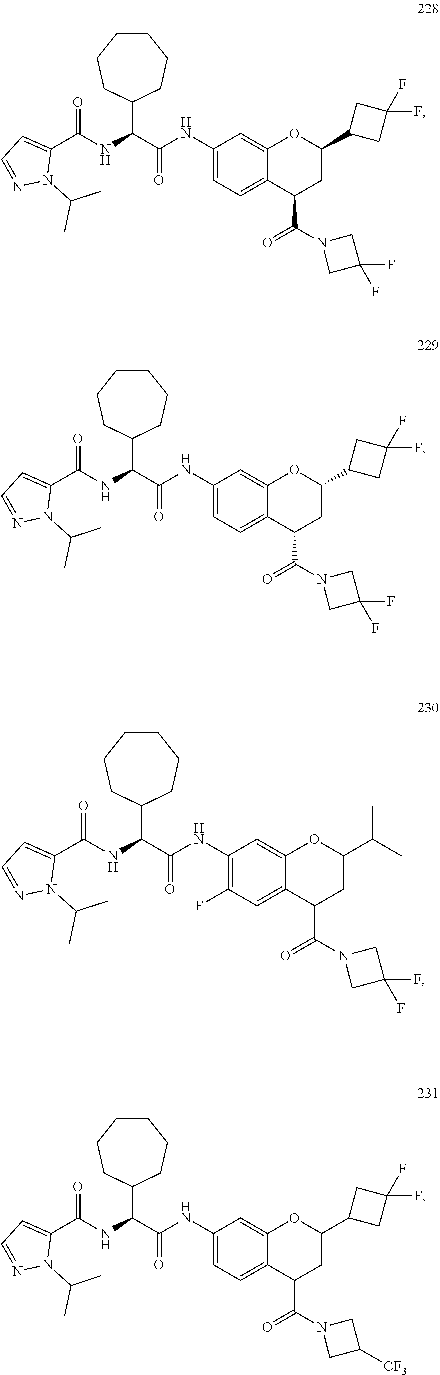 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCCCCC1 ; CC(C)n1nccc1C(=O)N[C@H](C(=O)Nc1ccc2c(c1)O[C@H](C1CC(F)(F)C1)C[C@@H]2C(=O)N1CC(F)(F)C1)C1CCCCCC1 ; CC(C)C1CC(C(=O)N2CC(F)(F)C2)c2cc(F)c(NC(=O)[C@@H](NC(=O)c3ccnn3C(C)C)C3CCCCCC3)cc2O1 ; CC(C)n1nccc1C(=O)N[C@H](C(=O)Nc1ccc2c(c1)OC(C1CC(F)(F)C1)CC2C(=O)N1CC(C(F)(F)F)C1)C1CCCCCC1] 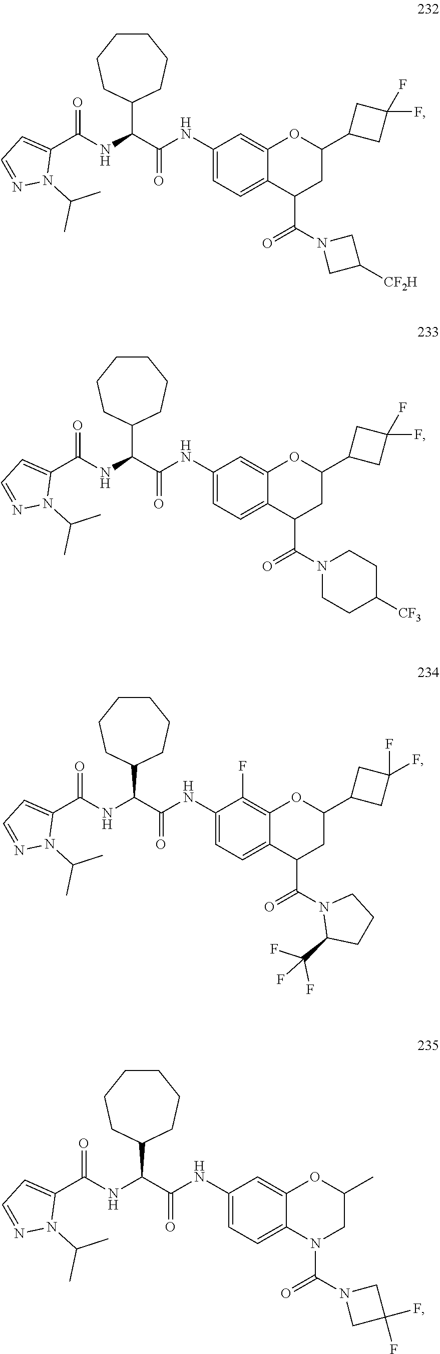 [SMILES: CC(C)n1nccc1C(=O)N[C@H](C(=O)Nc1ccc2c(c1)OC(C1CC(F)(F)C1)CC2C(=O)N1CC(C(F)F)C1)C1CCCCCC1 ; CC(C)n1nccc1C(=O)N[C@H](C(=O)Nc1ccc2c(c1)OC(C1CC(F)(F)C1)CC2C(=O)N1CCC(C(F)(F)F)CC1)C1CCCCCC1 ; CC(C)n1nccc1C(=O)N[C@H](C(=O)Nc1ccc2c(c1F)OC(C1CC(F)(F)C1)CC2C(=O)N1CCC[C@H]1C(F)(F)F)C1CCCCCC1 ; CC1CN(C(=O)N2CC(F)(F)C2)c2ccc(NC(=O)[C@@H](NC(=O)c3ccnn3C(C)C)C3CCCCCC3)cc2O1] 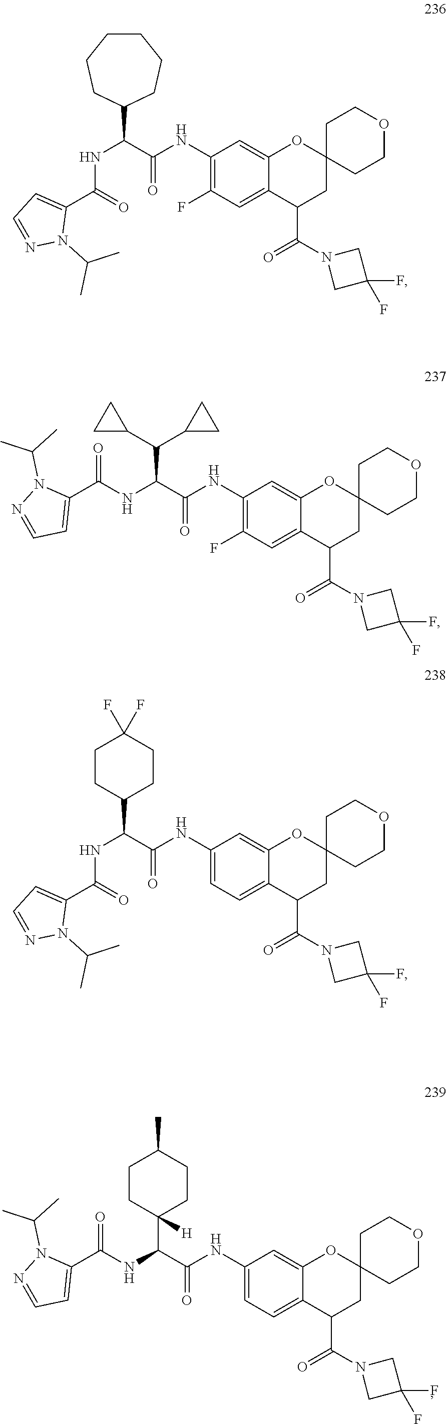 [SMILES: CC(C)n1nccc1C(=O)N[C@H](C(=O)Nc1cc2c(cc1F)C(C(=O)N1CC(F)(F)C1)CC1(CCOCC1)O2)C1CCCCCC1 ; CC(C)n1nccc1C(=O)N[C@H](C(=O)Nc1cc2c(cc1F)C(C(=O)N1CC(F)(F)C1)CC1(CCOCC1)O2)C(C1CC1)C1CC1 ; CC(C)n1nccc1C(=O)N[C@H](C(=O)Nc1ccc2c(c1)OC1(CCOCC1)CC2C(=O)N1CC(F)(F)C1)C1CCC(F)(F)CC1 ; CC(C)n1nccc1C(=O)N[C@H](C(=O)Nc1ccc2c(c1)OC1(CCOCC1)CC2C(=O)N1CC(F)(F)C1)[C@H]1CC[C@H](C)CC1] 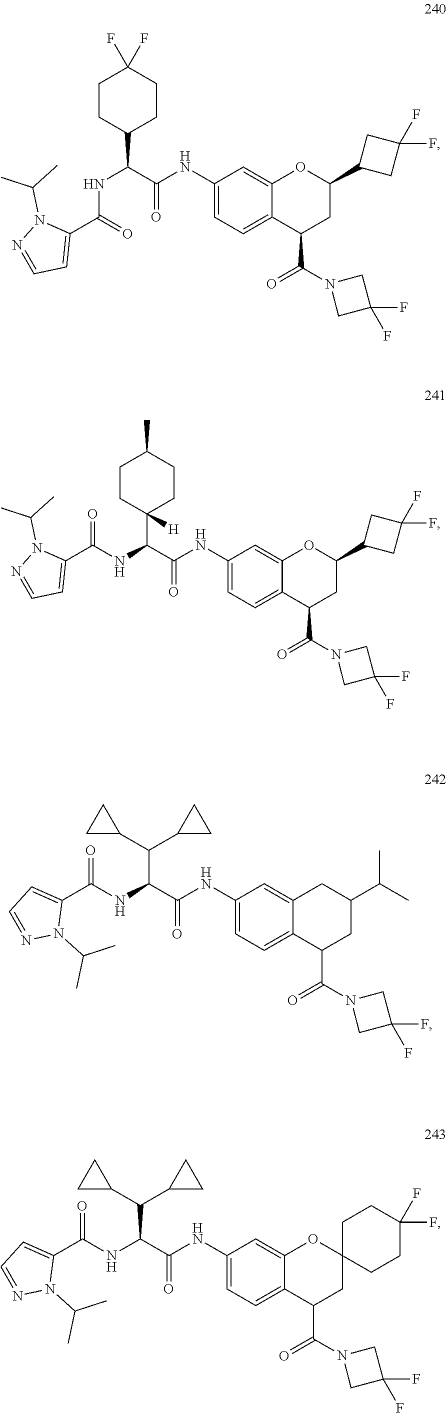 [SMILES: CC(C)n1nccc1C(=O)N[C@H](C(=O)Nc1ccc2c(c1)O[C@@H](C1CC(F)(F)C1)C[C@H]2C(=O)N1CC(F)(F)C1)C1CCC(F)(F)CC1 ; CC(C)n1nccc1C(=O)N[C@H](C(=O)Nc1ccc2c(c1)O[C@@H](C1CC(F)(F)C1)C[C@H]2C(=O)N1CC(F)(F)C1)[C@H]1CC[C@H](C)CC1 ; CC(C)C1Cc2cc(NC(=O)[C@@H](NC(=O)c3ccnn3C(C)C)C(C3CC3)C3CC3)ccc2C(C(=O)N2CC(F)(F)C2)C1 ; CC(C)n1nccc1C(=O)N[C@H](C(=O)Nc1ccc2c(c1)OC1(CCC(F)(F)CC1)CC2C(=O)N1CC(F)(F)C1)C(C1CC1)C1CC1] 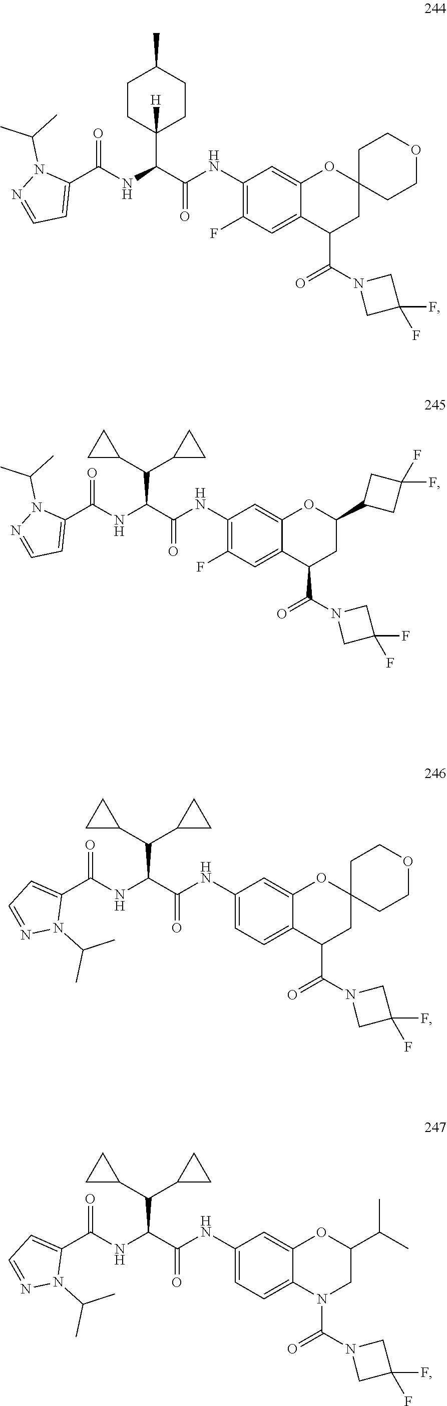 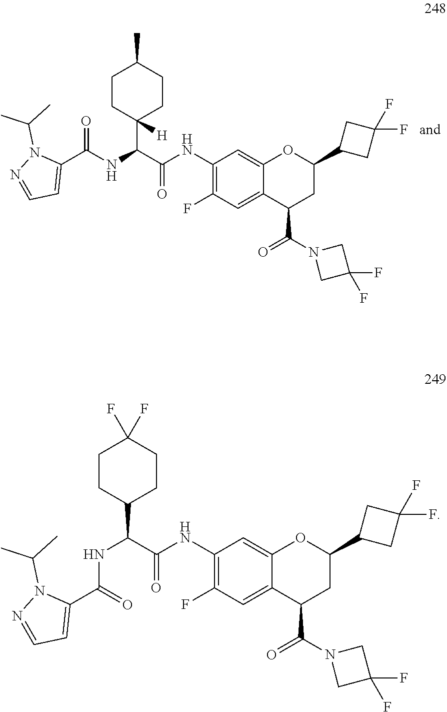 [SMILES: CC(C)n1nccc1C(=O)N[C@H](C(=O)Nc1cc2c(cc1F)[C@H](C(=O)N1CC(F)(F)C1)C[C@H](C1CC(F)(F)C1)O2)[C@H]1CC[C@H](C)CC1 ; CC(C)n1nccc1C(=O)N[C@H](C(=O)Nc1cc2c(cc1F)[C@H](C(=O)N1CC(F)(F)C1)C[C@H](C1CC(F)(F)C1)O2)C1CCC(F)(F)CC1]
pharmaceutically acceptable salts thereof, deuterium substitutes thereof, and isomers thereof.

51. A pharmaceutical composition, comprising the compound of claim 1, a pharmaceutically acceptable salt thereof, a deuterium substitute thereof, or an isomer thereof, and a pharmaceutically acceptable excipient.

52. A method of modulating IL-17A in a subject, comprising:
administering to the subject an effective amount of the compound of claim 1, a pharmaceutically acceptable salt thereof, a deuterium substitute thereof, or an isomer thereof.

53. A method of treating an inflammatory disease or condition in a subject, comprising:
administering to the subject an effective amount of the compound of claim 1, a pharmaceutically acceptable salt thereof, a deuterium substitute thereof, or an isomer thereof.

54. The method of claim 53, wherein the inflammatory disease or condition is selected from the group consisting of plaque psoriasis, guttate psoriasis, inverse psoriasis, pustular psoriasis, erythrodermic psoriasis, aspsoriatic arthritis, ankylosing spondylitis, hidradenitis suppurativa, rheumatoid arthritis, Palmoplantar Psoriasis, Spondyloarthritis, and Non-infectious Uveitis.
